(19) United States
(12) Patent Application Publication
(10) Pub. No.: US 2023/0270818 A1
(43) Pub. Date: Aug. 31, 2023

(54) TCF7L2 MEDIATED REMYELINATION IN THE BRAIN

(71) Applicant: University of Rochester, Rochester, NY (US)

(72) Inventors: Steven A. Goldman, Webster, NY (US); Abdellatif Benriass, Pittsford, NY (US); John Mariani, Rochester, NY (US)

(73) Assignee: University of Rochester, Rochester, NY (US)

(21) Appl. No.: 18/051,653

(22) Filed: Nov. 1, 2022

Related U.S. Application Data
(60) Provisional application No. 63/378,092, filed on Oct. 3, 2022.
(60) Provisional application No. 63/274,763, filed on Nov. 2, 2021.

(51) Int. Cl. A61K 38/17; C12N 5/079; A61K 48/00; A61P 25/28

(52) U.S. Cl. CPC A61K 38/1709; C12N 5/0622; A61K 48/0058; A61P 25/28; C12N 2506/08

(57) ABSTRACT

The present application relates to a method of treating a subject having a condition mediated by a deficiency in myelin. This method involves selecting a subject having a condition mediated by a deficiency in myelin and expressing a transcription factor 7-like 2 (TCF7L2) protein in the selected subject under conditions effective to treat the condition. Also disclosed is a method of increasing oligodendrocyte production from glial progenitor cells. This method involves providing a population of glial progenitor cells and expressing a TCF7L2 protein in the provided population of glial progenitor cells under conditions effective to increase oligodendrocyte production compared to oligodendrocyte production absent said administering. Also disclosed is a genetic construct suitable, inter alia, for carrying out these methods.

### CROSS REFERENCE TO RELATED APPLICATIONS

This application claims priority to U.S. Provisional Application No. 63/274,763 filed on Nov. 2, 2021 and U.S. Provisional Application No. 63/378,092 filed on Oct. 3, 2022. The contents of the applications are incorporated herein by reference in their entireties.

### GOVERNMENT INTERESTS

This invention was made with government support under NS110776, awarded by National Institutes of Health. The government has certain rights in the invention.

### REFERENCE TO A SEQUENCE LISTING

This application incorporates by reference the Sequence Listing submitted in Computer Readable Form as file 161118.03903, created on Nov. 1, 2022 and containing 158,503 bytes.

### FIELD

The present application relates to TCF7L2 mediated remyelination in the brain.

### BACKGROUND

The central nervous system (CNS) is organized into “gray matter,” which generally contains the cell bodies and dendrite networks of neurons, and “white matter,” which consists of axon bundles encased by myelin produced by oligodendrocytes. The myelin sheath has a high lipid fat content, which accounts for the whitish appearance. Myelin plays a critical role in neuronal communication. Impairment of oligodendrocytes disrupts white matter integrity and results in white matter degeneration (demyelination) and loss of neuronal communication within the brain and spinal cord. Myelin-related disorders, inherited or acquired, impact millions of people, levying a heavy burden on affected individuals and their families. The pathological processes underlying many of these disorders remain poorly understood and few disease-modifying therapies exist.

For example, Huntington's disease (HD) is a fatal autosomal dominant progressive neurodegenerative disease caused by an expansion of a CAG triple repeat in the Huntingtin (Htt) gene. The resulting polyglutamine expansion in the N-terminal region of the Htt protein triggers the formation of mutant Htt aggregates, and is associated with neurodegeneration occurring initially in the neostriatum, but ultimately involving much of the brain (de la Monte et al., “Morphometric Demonstration Of Atrophic Changes In The Cerebral Cortex, White Matter, And Neostriatum In Huntington's Disease,” Journal of Neuropath. and Exper Neurol 47: 516-525 (1988)). Traditionally, HD research has focused on the selective vulnerability of striatal and cortical neurons to the disease process. Recently though, a number of studies have also noted early white matter loss in HD, and have causally associated this process to disease progression in HD. In particular, the TRACK HD studies have identified discrete but progressive white matter atrophy in premanifest HD patients, long before the onset of any clinical symptoms (Paulsen et al., “Striatal And White Matter Predictors Of Estimated Diagnosis For Huntington Disease,” Brain Res Bull 82: 201-207 (2010); Phillips et al., “Deep White Matter In Huntington's Disease,” PloS one 9: e109676 (2014); Faria et al., “Linking White Matter And Deep Gray Matter Alterations In Premanifest Huntington Disease,” Neuroimage Clin 11: 450-460 (2016); Phillips et al., “Major Superficial White Matter Abnormalities in Huntington's Disease,” Front Neurosci 10: 197 (2016); Bourbon-Teles et al., “Myelin Breakdown in Human Huntington's Disease: Multi-Modal Evidence from Diffusion MM and Quantitative Magnetization Transfer,” Neuroscience 403: 79-92 (2019)). The role of this MRI-defined involution of the forebrain white matter in disease progression, and the extent to which these changes are primary or secondary to neuronal dysfunction, has remained unclear.

Previously, it has been shown that HD patient-derived glial progenitor cells (hGPCs), produced from human embryonic stem cells (hESCs), manifested a profound and systematic transcriptional downregulation of myelinogenic genes in vitro (Osipovitch et al., “Human ESC-Derived Chimeric Mouse Models of Huntington's Disease Reveal Cell-Intrinsic Defects in Glial Progenitor Cell Differentiation,” Cell Stem Cell 24: 107-122 e107 (2019)). Myelin synthesis could be rescued by induced expression of the critical myelinogenic drivers SOX10 and MYRF, indicating that the myelin biosynthetic machinery is intact in these cells; rather, it is the upstream regulators of these latter genes that appear deficient in HD GPCs, yielding impaired oligodendrocytic differentiation. The HD hGPCs also exhibited their impairment in oligodendrocytic maturation and myelinogenesis when transplanted into hypomyelinated mouse hosts, suggesting the cell-autonomous nature of their differentiation defect.

Further, in certain neurodegenerative disorders characterized by myelin loss, whether developmentally or as a failure of myelin maintenance or regeneration, the deficiency in myelinogenic competence stems from diminished TCF7L2-dependent transcription. TCF7L2 is a transcription factor that serves as a signal effector for the Wnt pathway, but it may also be driven through pathways independent of the canonical Wnt signaling, and Wnt-dependent transcription in turn can act through intermediates other than TCF7L2. Indeed, whether a myelination deficit occurs in adults and in vivo, and whether it stems from a downregulation of myelinogenic transcription factors, such as TCF7L2 is not clear.

There is need for therapeutics for treating disorders and conditions mediated by or characterized by deficiencies in myelin. The present disclosure is directed to overcoming these and other deficiencies in the art.

### SUMMARY

A first aspect of the present application relates to a method of treating a subject having a condition mediated by a deficiency in myelin.

In one embodiment, the method comprises introducing to the subject in need thereof a transcription factor 7-like 2 (TCF7L2) and expressing a transcription factor 7-like 2 (TCF7L2) protein in one or more cells of the selected subject. The method can be carried out by administering to the subject a genetic construct or expression vector encoding the TCF7L2 protein. Examples of the one or more cells include a glial progenitor cell, an oligodendrocyte progenitor cell, a glial cell, or an oligodendrocyte.

In another embodiment, the method comprises administering to the subject in need thereof a host cell comprising a genetic construct or expression vector encoding the TCF7L2 protein. Examples of the host cell include a glial progenitor cell, an oligodendrocyte progenitor cell, a glial cell, or an oligodendrocyte.

Another aspect of the present application relates to a method of increasing oligodendrocyte production from glial progenitor cells. This method comprises expressing a TCF7L2 protein in a population of glial progenitor cells, and maintaining the population of glial progenitor cells under conditions permitting development and differentiation thereof. The method can be carried out by administering to the population of glial progenitor cells a genetic construct or expression vector encoding the TCF7L2 protein.

The genetic construct described above may comprise (i) a nucleic acid molecule encoding the TCF7L2 protein and (ii) a promoter and/or enhancer. The nucleic acid molecule is operatively linked to and under the regulatory control of the promoter and/or enhancer. The promoter and/or enhancer can be one for a gene which is selectively or specifically expressed by glial progenitor cells. The gene selectively or specifically expressed by glial progenitor cells can be one selected from the group consisting of PDGFRA, ZNF488, GPR17, OLIG2, CSPG4, and SOX10.

The genetic construct can be administered in an expression vector, such as a viral vector, plasmid vector, or bacterial vector. The viral vector can be one selected from the group consisting of a lentiviral vector, an adenoviral vector, an adeno-associated viral vector, and a vaccinia vector.

In the methods described above, the genetic construct can be administered in association with a glial progenitor cell-targeted fusogen or a glial progenitor cell-selective surface-binding moiety. For example, the genetic construct can be in a particle comprising the progenitor cell-targeted fusogen or the glial progenitor cell-selective surface-binding moiety. The particle can be one selected from the group consisting of a virus, a virus-like particle, and a lipid particle. The glial progenitor cell-targeted fusogen or a glial progenitor cell-selective surface-binding moiety can be directed against CD140a, NG2/CSPG4, A2B5 gangliosides, 04 sulfatides, or CD133.

In the methods described above, the condition mediated by a deficiency in myelin of the subject can be one selected from the group consisting of pediatric leukodystrophies, lysosomal storage diseases, congenital dysmyelination, cerebral palsy, inflammatory demyelination, post-infectious and post-vaccinial leukoencephalitis, radiation- or chemotherapy-induced demyelination, and vascular demyelination. In one example, the subject has a condition with defect in myelination or remyelination. Examples of the condition include multiple sclerosis, neuromyelitis optica, transverse myelitis, optic neuritis, subcortical stroke, diabetic leukoencephalopathy, hypertensive leukoencephalopathy, age-related white matter disease, white matter dementia, Binswanger's disease, spinal cord injury, radiation- or chemotherapy induced demyelination, post-infectious and post-vaccinial leukoencephalitis, periventricular leukomalacia, and cerebral palsy. In one embodiment, the condition is a neurodegenerative disease, such as Huntington's disease. In another embodiment, the condition is a neuropsychiatric disease, such as schizophrenia. In some embodiment, the condition is characterized by downregulation of one or more genes selected from the group consisting of Myrf, Bcas1, Plp1, Mbp, and Mobp. For each of the above-described methods, the administering can be carried out using any suitable means, including intracerebral delivery, intrathecal delivery, intranasal delivery, or via direct infusion into brain ventricles. The subject can be mammalian, such as a human.

Another aspect of the present application relates to a genetic construct. This genetic construct comprises a nucleic acid molecule encoding a TCF7L2 protein and a promoter and/or enhancer for a gene selectively or specifically expressed by glial progenitor cells. The nucleic acid molecule is operatively linked to and under regulatory control of the promoter and/or enhancer. The gene selectively or specifically expressed by glial progenitor cells can be selected from the group consisting of PDGFRA, ZNF488, GPR17, OLIG2, CSPG4, and SOX10. The genetic construct can be in association with a glial progenitor cell-targeted fusogen or a glial progenitor cell-selective surface-binding moiety. The glial progenitor cell-targeted fusogen or a glial progenitor cell-selective surface-binding moiety can be directed against CD140a, NG2/CSPG4, A2B5 gangliosides, O4 sulfatides, or CD133.

Also provided is an expression vector comprising the genetic construct described above. The expression vector can be a viral vector, plasmid vector, or bacterial vector. Examples of the viral vector include a lentiviral vector, an adenoviral vector, an adeno-associated viral vector, and a vaccinia vector.

Also within scope of this disclosure is a host cell comprising the genetic construct or expression vector described above, or a progeny of the host cell. In some embodiments, the host cell is a stem cell or a progenitor cell. Example of the stem cell include without limit embryonic stem cells, ES-like stem cells, fetal stem cells, adult stem cells, pluripotent stem cells, induced pluripotent stem cells, multipotent stem cells, oligopotent stem cells, unipotent stem cells and others. In exemplary embodiments, the cell is a mammalian cell. The host cell can be used to express the TCF7L2 protein or used as a therapeutic cell/agent for treating the disorders or conditions described herein.

The present application relates to the discovery that in certain neurodegenerative disorders characterized by myelin loss, whether developmentally or as a failure of myelin maintenance or regeneration, the deficiency in myelinogenic competence stems from diminished TCF7L2-dependent transcription. TCF7L2 is a transcription factor that serves as a signal effector for the Wnt pathway, but it may also be driven through pathways independent of the canonical Wnt signaling, and Wnt-dependent transcription in turn can act through intermediates other than TCF7L2.

As a result of the above aspects of the application, it is possible to treat Huntington's disease or any neurodegenerative disease with myelin loss as well as for myelin disorders characterized by differentiation block of resident GPC, such as progressive multiple sclerosis and cerebral palsy as well as selective neuropsychiatric disorders. The transcription factor networks and predicted signaling pathways that are defective in GPCs derived from subjects with childhood-onset schizophrenia, which is also characterized by significant myelin loss and developmental hypomyelination, similarly exhibit a repression of TCF7L2-dependent transcription, so that GPC-targeted TCF7L2 may be expected to rescue hypomyelination in that disorder as well.

The data presented here indicates that HD, as manifested in two distinct transgenic mouse models of the disease, is associated with a progressive age-associated loss in forebrain myelin, relative to WT mice, as well as in impaired remyelination after adult demyelination. Together, these findings suggest a loss in homeostatic white matter maintenance. This was underlined by profound dysregulation of oligodendroglial lineage-associated gene expression predicted to be driven by upstream TCF7L2 signaling. Importantly, forced glial over-expression of TCF7L2 proved sufficient to restore the functional transcription of key myelinogenic and lipid biosynthetic genes, and to the in vivo restoration of myelin architecture and abundance. As such, this work provide a novel and effective strategy for the therapeutic rescue of glial dysfunction, and hence of both the synaptic and white matter pathology of HD.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A, 1B, 1C, 1D, 1E, 1F, 1G show myelination in presymptomatic and diseased R6/2 mice. FIG. 1A shows a representative transmission electron microscope micrograph of corpus callosum of 6 and 12 week WT and R6/2 mice. FIGS. 1B-1C show the linear regression analysis of the g-ratios. FIGS. 1D-1E show the frequency distribution of myelinated axons as a function of their diameter. FIGS. 1F-1G show the relative number of the myelinated axons in corpus callosum of treated mice as a function of genotype. ns: non-significant, Unpaired Welch's t test. Scale bar=0.5 μm.

FIGS. 2A, 2B, 2C, 2D, 2E, 2F, 2G, 2H, 2I, 2J, 2K, 2L, 2M, 2N show remyelination following cuprizone treatment in R6/2 mice. FIG. 2A shows the timeline and experimental design. FIG. 2B shows a representative transmission electron microscope micrograph of corpus callosum during or after the Cuprizone treatment. FIGS. 2C-2F show the linear regression analysis of the g-ratios. FIGS. 2G-2J show the frequency distribution of myelinated axons as a function of their diameter. FIGS. 2K-2N show the relative number of the myelinated axons in the corpus callosum of treated mice as a function of genotype. ns: non-significant, ** p<0.01, *** p<0.001, by Unpaired Welch's t test. Scale bar=0.5 μm.

FIGS. 3A, 3B, 3C, 3D, 3E show RNA-Seq of HD GPCs reveals stalled oligodendroglial differentiation. Striatal mouse GPCs were isolated from PDGFRa-EGFP bred to either Q175 or R6/2 HD mice or their respective littermate controls and transcriptionally analyzed via RNA-Seq. FIG. 3A shows a principle component analysis of bulk GPC RNA-Seq samples which illustrates separation of HD glial populations in a time dependent manner. FIG. 3B shows an upset plot of the intersection of all four comparisons shows the conservation and independence of genes across models and timepoints. FIG. 3C shows the average expression of variance stabilized counts within each HD model was plotted against controls at the same timepoint to illustrate the increasing severity of DE between early and late timepoints. FIG. 3D shows a heatmap representation of DE genes of interest with indication of which comparisons a gene is significantly DE. FIG. 3E shows the significance of gene ontology enrichment of curated terms within each comparison.

FIGS. 4A, 4B, 4C show network analysis of HD GPCs identification of TCF7L2 and myelination-enriched module. Modules were determined via WGCNA and were filtered on differentially-expressed genes, as HD vs. WT GPCs, before being analyzed in IPA for GO enrichment. FIG. 4A shows three modules were found to be significantly enriched for terms dealing with myelination and oligodendrocyte differentiation with Black being the most prominent. FIG. 4B shows the Black module was also determined to be very enriched for genes that were differentially-expressed. FIG. 4C shows a gene ontology network representation of the Black module yielded five neighborhoods with representation of TCF7L2 signaling and myelin related terms.

FIG. 5 shows expression analysis of select genes following TCF7L2 overexpression in vivo. Ten weeks old R6/2 mice injected with LV-TetOn-TCF7L2 were split in two groups: treated with doxycycline or untreated controls. Both the control and doxycycline-treated mice were assayed for selected myelinogenic and metabolic genes; expression values were normalized to that of 18S RNA. LV-TetOn-TCF7L2 injected animals treated with doxycycline displayed increased level of myelinogenic and lipid biosynthetic genes, whereas more upstream components of TCF7L2 signaling were minimally affected. * p<0.05; ** p<0.01 by Welch's t-test.

FIGS. 6A, 6B, 6C, 6D, 6E show TCF7L2 overexpression rescued remyelination deficit in R6/2 mice. FIG. 6A shows a timeline of treatment with cuprizone (CZN) and lentiviral overexpression of TCF7L2 (LV-TCF7L2). FIG. 6B shows transmission electron microscope micrographs of corpus callosum of cuprizone-treated WT, R6/2 and R6/2 treated with LV-TCF7L2. FIG. 6C shows linear regression analysis of the g-ratio. FIG. 6D shows the percentage of myelinated axons as a function of their diameter and treatment. FIG. 6E shows the percentage of remyelinated axons as a function of genotype. ns: non-significant, * p<0.05, *** p<0.001, by 2-way ANOVA (FIG. 6D) or 1-way ANOVA (FIG. 6E) with Tukey multi-test. Scale bar=0.5 μm.

FIG. 7A, 7B, 7C, 7D shows that large diameter axonal fibers are preferentially remyelinated in wildtype mice, relative to R6/2. Distribution of remyelinated axonal fiber is shown as a function of their diameter in mice treated with cuprizone from 6 week to 12 weeks. A cohort of mice was sampled while the mice were on cuprizone diet at 10 weeks (four weeks into diet, FIG. 7A), at the end of the 6 weeks treatment (FIG. 7B), after two weeks recovery (FIG. 7C) or four weeks recovery (FIG. 7D). * p>0.05, ** p>0.01 by 2-way ANOVA.

FIG. 8 shows delayed remyelination of R6/2 HD white matter after cuprizone demyelination. Linear regression analysis of a number of remyelinated callosal axons as a function of recovery time, comparing cuprizone-treated R6/2 and WT mice. *p<0.0001, (F(2,116)=16.15).

FIGS. 9A, 9B, 9C, 9D show oligodendrocytes progenitor cells are isolated from R6/2 and zQ175 mice by FACS. Striata from 4-8 R6/2-PDGFRa-EGFP or zQ175-PDGFRa-EGFP mice were dissociated and GPCs sorted by FACS based on their EGFP expression. Cytometry plots of EGFP⁺ at early and late timepoints for R6/2 (FIG. 9A) and zQ175 (FIG. 9B). Relative distribution and total cell collected following cell sorting of GPCs for R6/2 (FIG. 9C) and zQ175 (FIG. 9D) samples. ****p>0.0001; ns: not significant; 2-way ANOVA, Tukey test for multiple comparisons.

FIGS. 10A, 10B, 10C, 10E, 10F, 10G show EGFP expression is reduced in the striata of aged mice. EGFP and NG2 immunostaining of striata of 12-week old (FIG. 10A) and 1-year old (FIG. 10B) zQ175 mice, along with their respective color splits. FIG. 10C shows EGFP⁺ cell density was significantly reduced in the striata of 1 year old mice (p<0.0001 for the age effect, 2 way ANOVA). Stereological count of Olig2⁺ cell densities in R6/2 (FIG. 10D) or zQ175 (FIG. 10E) mice. Striatal volumes of R6/2 (FIG. 10F) or zQ175 (FIG. 10G) mice. * p>0.05, **** p>0.0001, ns: not significant by 2 way ANOVA with Tukey multi-test). Scale (FIGS. 10A-10B): 50 μm.

FIG. 11 shows expression of glial progenitor and other CNS markers by striatal PDGFRA-EGFP sorted GPCs. FACS isolated PDGFRA-EGFP striatal cells displayed high expression of GPC markers accompanying low expression of off-target CNS cell type markers in all mice analyzed. Counts were batch corrected and normalized via variance-stabilizing transformation.

FIG. 12 shows significantly dysregulated TCF7L2 signaling-related genes in R6/2 and zQ175 mice. Ingenuity pathway analysis (IPA) curated TCF7L2 signaling associated genes that were found to be significant in both R6/2 and zQ175 GPCs (FDR <0.01) are displayed here as gene Z-scores of corrected variance-stabilizing transformed counts. The timepoint and model in which they were differentially expressed is indicated in the heatmap on the right.

FIG. 13 shows TCF7L2 isoforms of human GPCs. To identify the optimal Tcf712 splice isoform for local over-expression, reference was made to previously unpublished isoform data obtained in a broader gene expression analysis of human HD and control ESC-derived GPCs, as assessed in hGPCs generated in vitro (Osipovitch et al., “Human ESC-Derived Chimeric Mouse Models of Huntington's Disease Reveal Cell-Intrinsic Defects in Glial Progenitor Cell Differentiation,” Cell Stem Cell 24: 107-122 e107 (2019), which is hereby incorporated by reference in its entirety). Ensemble transcript identifiers are provided for all expressed isoforms.

FIGS. 14A, 14B, 14C, and 14D show the transgene expression pattern of LV-TCF7L2-EGFP in the callosum of cuprizone-treated mice. FIG. 14A shows that a doxycycline-regulated lentiviral vector allowed the DOX-expression of TCF7L2. Six week-old mice were fed cuprizone for four weeks before receiving intra-callosal injection of LV-TCF7L2-EGFP. The mice were sacrificed one week after during which they were still on cuprizone diet. EGFP reporter was expressed in oligodendrocytes lineage cells (FIG. 14B), GPCs (FIG. 14C) and astrocytes (FIG. 14D) in the cuprizone induced lesion. Scale: 25μm.

FIGS. 15A, 15B, 15C, 15D, 15E, and 15F show that HD callosal white matter display a dysregulated proteome. Corpus callosum of 12 week old R6/2 and 1 year old zQ175 (and their respective WT littermate control) mice were dissected and processed for mass spectrometry. FIG. 15A shows the principal component analysis of HD and WT callosal white matter. FIG. 15B shows a Venn diagram of shared peptides. FIGS. 15C and 15D show volcano plots of differentially expressed peptides (p<0.05). FIG. 15E shows a heat map of select myelin proteins HD mice and respective WT controls. FIG. 15F shows an ingenuity pathway analysis of differentially expressed peptides. Significant terms of interest were curated for display (p<0.01).

FIGS. 16A, 16B, and 16C show that striatal GPCs exhibited HD-dependent dysregulation of protein expression congruent with that of gene expression. Twelve-week-old HD and WT littermate control mice were killed, their striata dissected, dissociated, and their GPCs isolated via A2B5-based FACS for mass spectrometry. FIG. 16A shows a principal component analysis of R6/2 and WT striatal GPCs. FIG. 16B shows a volcano plot of differentially-expressed peptides in R6/2 vs WT striatal GPCs (p<0.05). FIG. 16C shows a scatter plot of log 2-fold changes of genes and proteins that were significantly differentially expressed in both the bulk RNA-Seq and Mass Spec.

FIGS. 17A and 17B show that protein level and mobility of TCF7L2 in HD mouse corpus callosum did not differ from WT. FIG. 17A shows a western blot of 12 week old corpus callosum for TCF7L2 and B-Actin. FIG. 17B shows a bar graph of beta-actin normalized TCF7L2 in R6/2 and littermate WT control corpus callosum. ns: non-significant, by unpaired Welch's t test.

FIG. 18 shows a graphical overview of the myelination defects in Huntington's disease mouse models and their rescue by Tcf712 expression. Top: R6/2 Huntington's disease mice exhibit a developmental defect in callosal myelination and exhibit slower and less complete remyelination after demyelination. Middle: the myelination defect of Huntington's disease glial progenitor cells reflect deficient Tcf712 expression in model mice (R6/2 or zQ175). Bottom: Tcf712 expression rescues myelin gene expression and myelination of R6/2 mice in vivo.

### DETAILED DESCRIPTION

A first aspect of the present application relates to a method of treating a subject having a condition mediated by a deficiency in myelin. This method involves selecting a subject having a condition mediated by a deficiency in myelin and expressing a transcription factor 7-like 2 (TCF7L2) protein in the selected subject under conditions effective to treat the condition.

Another aspect of the present application relates to a method of increasing oligodendrocyte production from glial progenitor cells. This method involves providing a population of glial progenitor cells and expressing a TCF7L2 protein in the provided population of glial progenitor cells under conditions effective to increase oligodendrocyte production compared to oligodendrocyte production absent said administering.

Another aspect of the present application relates to a genetic construct. This genetic construct comprises a nucleic acid molecule encoding a TCF7L2 protein and a promoter and/or enhancer for a gene selectively or specifically expressed by glial progenitor cells. The nucleic acid molecule is under regulatory control of the promoter and/or enhancer.

The present application describes a genetic construct comprising a nucleic acid molecule encoding a TCF7L2 protein and a promoter and/or enhancer for a gene selectively expressed by glial progenitor cells, said nucleic acid molecule being under the regulatory control of the promoter and/or enhancer.

In an embodiment, the expressing a transcription factor 7-like 2 (TCF7L2) protein is carried out by administering a genetic construct. In some embodiments, the genetic construct comprises a nucleic acid molecule encoding the TCF7L2 protein and a promoter and/or enhancer for a gene which is selectively or specifically expressed by glial progenitor cells, said nucleic acid molecule being under the regulatory control of the promoter and/or enhancer.

As used here, the term “TCF7L2” refers to the transcription factor 7-like 2 protein.

In another embodiment, the gene selectively or specifically expressed by glial progenitor cells is selected from the group consisting of CNP1, GPR17, PDGFRA, ZNF488, OLIG2, CSPG4, and SOX10. In another embodiment, the gene selectivity or specifically expressed by glial cells is selected from the group consisting of CNP1, GPR17, PDGFRA, ZNF488, OLIG2, CSPG4, and SOX10. Listed below are exemplary promoters.

| Promoter region of CNP1 gene human promoter 2 (P2) | |
|---|---|
| (SEQ ID NO: 41) | |
| cccgcccgcccccagcccgcccgggtgcccgctccgccccccgggctatgtaaagcggccgggctcgggttgtgc | |
| caccgctggactcccgtgtccctccgcgcaggcgggcggccccggagcgctggtgccggcagaggcggcgacggt | |
| ggcgcccctcctcatcatggtgagaggccgggcggggccgggcacggggtagcaccagggcgggaaacgagtgtc | |
| ggggcccctcgggaggaagcgtcttgcaaacgaggacccggggctccgggttcgacttccagttttcttggtact | |
| cagggcggccctgaggtctgggagaagcggaggggttttggggtgccggagaggccgctgtaaagggaaacggtg | |
| gtgtttctcaggagggaactcgggaccgcagaagctgctgcgcctctgggagcctgggtgggcctgggccgggag | |
| cccgggagctgctcccgaccccacgcaggcccgcgctggggcagccgcttcggcgccccctccccgggcccagtc | |
| tcatcacgcggaaccgctgccccgcgcccgggtgcgggcgggaggtggagccagttcagaggaggggacccttta | |
| cggtaaaagctgccgtcccttgtggcttctagaagataggaaaacaagtggctccgagaggcgcttctgacacac | |
| agcacagcggcgggtagagctggatcctaattgcaggtgccctgtgctgccctgttctggacaggcacaaagacc | |
| acgactttgtgggggccgggagagggtagaggaggaggtggcaggcttggtgaaagagggcctttgtccagtgct | |
| ctcctgccctgccccccaccagcccttctgtgggaccattgtacccgcatgccagacaagagagtattatctgtt | |
| gcggccattgttgggggaagggaggcagtcttggggtaacccttctccactcccccccttctctgtgcactcagc | |
| cgtggcagctgtggcttggcccagacacagagacccccacctccaaagaggacgtccttagtaggtgccaagcat | |
| ataagagtgaggccagtcccagaaccgcaggctcctggcgcgccccgcatgcctccagcacgtttacctttccga | |
| agtggcaggaatggggaaagcgcacgcttaggagagcttcagacaagcttccctcttcctcctccacgaccagaa | |
| gcggaaaggtgctcccggaccgaaagggaaagaaggtccagcactgccccgcttgggaaggcacccacaaccagt | |
| ctagggactaggggtaaggccggcggggagcccgcgaatgacctgggctgacatctcttcccctccttacacaga | |
| acagaggcttctcccgaaaaagccacacattcctgccca | |
| GPR17 (human promoter) | |
| (SEQ ID NO: 42) | |
| Ggatacggaagagatccaatcgcagacccaagatccccacccaggttatggtgggcagaccccagatgccagggc | |
| cacccattcagcatccctccctggaccccaggacctgctactgctgggtgtctggactccatcctGcacagcact | |
| gtgctccatctgccctggggtgtctcatcatcagctgtgtgcagggcaaggggcccaaacaaaggcccagcagtc | |
| actggctaagctgccgactggctctctgtgcctccccaagaccctatgtgcccagcAgggggcaacagctcaggg | |
| tcagctgaccgaatgcctcggtgaatgaatgactctacaagagaggaagggagcctcggtgggcatcatctcccc | |
| tcgactactggccagagccctggctcttacaccccagcgacgggaAgcagttgtggcctgtggcttcagtcttca | |
| tcaccacaatccctgaagcccacccttgcccagacacctgtgccccagccccaaccccaggccacctcctcagca | |
| ggtctggggctgagctgccccacctggcgcctatggGgccagcccatgccccctgcggtgcctctgtcccagac | |
| tcagcatgtaggccccatgaccccactccacattctggtgactcctcctgagcgtcaggacaacactcaacccac | |
| gaggaattatttctgtctcaaagatgcaggAatcagctcaacgcctcaaaactccatcaccacggtcaatgccct | |
| tgaagccatcgacagtgatcaccccaataacagaaggtctgtgagcccagaaatgccctgctcagggtggttagc | |
| ttcaagccaccacctttccaacCagcctgggccagttcttccagacagccgcctgcgggcacaacaggaaagaga | |
| cctgcgccccggctcagacacctcacacccagctggctctcaggccagacaaactgggaagcccatctctcttga | |
| aggaagtccagaTgggaaacagcttctcaacagaccagatcacagcatcagatctaaaggtggccttcagaattc | |
| tttttcaggttgaattaggatcaaatctaagaattctaaattcaaaatgcagcagaaaaacaaaacacacacaca | |
| cacgGagcctaagttctggagtgacatgtgcttgggttcaaatcctggctctgttgcttcctactgtttgttgat | |
| gggtgagtttcttcatttgcctgagcctcagtttccttgtctgtaaaatggggcaataatcccagctgcacaggg | |
| tgaTgtgaagagacaaatttaagacactgccccttaaatgctagccacatacatacagttttcaatgtttaaaca | |
| acaaaatgtaaagtcttttgaaaccaggaagggtgatttggtttcccatgttgctggatgtatcattttcagaaa | |
| Gacagagagaaatgaactttgttcactcagtctcagaggcggccgccggcagcattcaaaggcaccccagcccgg | |
| agccaccccagggaggagccccaggccagcggtcagattcatgggcttccgtgcagaaggggagctgCaccggcg | |
| agcacccggcctctgagctgagccgcatcctcacggacaggacagcgccccattatgaggctcctgcagctgttc | |
| ctcgctccagataaaggccatgatttattctgtgtgcccaaatggggcctcattatacaggGcaggacacaagga | |
| ccctacagcaagtgtcctcaaagagtcgcctctcactccgtgagcaagactcctcggcctcccaccctccgttca | |
| ccccatcagtaggaaatcatccccttctgaaacgtcctgttgtgtccctcagctccagcccaagccccccaccca | |
| gcccccgcctgctctgagtctctgagacagtcacacactcagactatgtGgccaagctgggggcggggggcatgg | |
| gctagggacacactagaatattcacgctccggtggcagcagcagcagcagccagaggagcagcccgacacaacaa | |
| gggacccctcaggaatgaagcagcctttcagggccagaggggctgtggtctcccttcctctccttaaatagcCAG | |
| CGTTCCACCCACAGCGGCAAGGG | |
| PDGFRA (Genbank: X80389) (SEQ ID NO: 43): | |
| 1 | gttgacctct cttctttgca cagtctctcc gcaaaatttt cccccaacca tttgctttct |
| 61 | tgcgccaccc cccaccgggg cactatccct ggagtcagct cctcaggcct ttaaaccttc |
| 121 | cagaatgtca cacatggaaa cctttagcaa atgtttgtta atgatcataa caaaggcatc |
| 181 | attcaaatta ggcaggtaat tactaccaga aggacaactg ggtgctcgct tgctcatcca |
| 241 | ttgctttctg ctttaaactc ccgaggagtc gctgcctgga gacactcccg cccggactgc |
| 301 | ttctcgggcc ccattaaacg gctctgaaat gaactgtgaa gttaccattt gtgggcggag |
| 361 | agcgctgggg ccctgcggac ggaagaccct ggcttgaccg gactaacagt gtcgccgaag |
| 421 | caaagcagcc cctctgggta ggctgtctgt ctcgcctcct accctctggg gcaactagcc |
| 481 | taaaaacccg gttctcaact taacagcatg tggcccagca cccgcttttg ccttttattc |
| 541 | cgcactctga tttttggggg gatcgtttgt tcccgctcat tttctacaaa cattacggta |
| 601 | tcctgttagc attccgaaca aggggctgtt catatatttg ccttcaatga tttcctgaag |
| 661 | ggacatgtgg aagtaatagc gagcggtgca gtcacccaag ctgaagatgc acgagagcgg |
| 721 | gtcgcgtaga agaactgcgg caatgggacc cccagcgcag ccccaggcgc cctgttttcg |
| 781 | aggctgacat cccaagatcc tgtcctggtt aggcacagca ccgcgcccgt tggagtactt |
| 841 | ctctgtgact ctcccctgtc cactgcactg tgtccaccct cctggtgaag gaggagacgg |
| 901 | ctgttggaca tcccagtccc aacctggcca ctttggccat tttcagagtg cgtgtgttgg |
| 961 | ggtggggttg agggagaagg atgaaggatg acccccaacc ccaacaggag aattcaaaaa |
| 1021 | tcagagagcg atgaaggtga acgcgctccc ctcccccgct gtcgctggga agccccactc |
| 1081 | tgcccaggtc tggttcctgg agcatcagcg ccccctttcc ctcgccggca ggatcgtttc |
| 1141 | ctcccgacac tggcctctcg ccttgggcgc agggtttggg gctcagctgt caattccaag |
| 1201 | gaattcgtag ggttcgaccc acgcggcggg agggaacgga gggagaatca aaaggcatcc |
| 1261 | tcccccacga cgcggaggcg gtggtacgat tcctcaaacg cccaagcttg agcttttttt |
| 1321 | gtatgcgaga tagaagccag ggcaacctcg cccagataac cccgaaaaca aaggcacgac |
| 1381 | cagataagtg acttcaaggg agtaaaggtc gttctatcgg agaacgttcc aaacctacac |
| 1441 | agaccctctt ttctttgtac cgtatttcag accacccagt cttgtacaca cacacacacc |
| 1501 | acacaaaaca aaaacccatc attgtcatat tggactcaac agtttgccca atcctattaa |
| 1561 | ataactagca actatatagc ccttccccta aaagacccct agttcaaaac ggcgcaaccg |
| 1621 | cttggagatt tctcccgagg gccctatttc tcgttgggcc gagtttgtag aagggacatt |
| 1681 | tcttgattct agatttatat tctctctggg tattaaatgc aattttgtat gttccttttc |
| 1741 | ttcttttcaa agagaataca aaactatagc agtctaagaa aataacccca aaatggggag |
| 1801 | gcaaactcat tctggaaatg atgggctgtt tgtagtttca caaaacctct ttcccggcag |
| 1861 | agcaccaaca cctccccctt ccaccacccc ccatcccatc tggtctgctt ctccccgccc |
| 1921 | cccagttgtt gtcgaagtct gggggttggg actggacccc ctgattgcgt aagagcaaaa |
| 1981 | agcgaaggcg caatctggac actgggagat tcggagcgca gggagtttga gagaaacttt |
| 2041 | tattttgaag agaccaaggt tgaggggggg cttatttcct gacagctatt tacttagagc |
| 2101 | aaatgattag ttttagaag |
| CSPG (GenBank: DQ241507.1) (SEQ ID NO: 44): | |
| 1 | cggaggggaa agtcaaaggt tcacagaaga ccctccatca aggggaggat gtgcagtagt |
| 61 | tgtatacaaa ccgtgaagta ctgggatgtg cagaatgaga gaggaagaag aaaaggcgat |
| 121 | atgagttgaa cacctactgt atgccaggcc ctaaggtagg catttgagac atattaaaac |
| 181 | acttaatgct tataaattca aacttgtaag tgaaaaactg aaccacagga aaaaaaggag |
| 241 | tagttaaagg ccagtcaagt aggagaattg ggatcaagaa cacatcagac caggcatggt |
| 301 | agtaagagca cagagcatgg gctcaatgat tcggcttgcc ctcgcctgga tcaactcacc |
| 361 | caggcccttt aatccctctc agcctcagtt tctctatcga aatggggctc ggttctatca |
| 421 | gaagtcatga gacgctggac ttgtgctatg attgttgctg ctcaggggta aggattggaa |
| 481 | agagggtgca cagtggaacg tgggcacagc agggagccca gtggagggat atgcgtgaaa |
| 541 | gaagagagga acgggagtgt tcctctatgc gtctcctaga aaaaacccag gccaggtccc |
| 601 | aatgcagctg gggctggcca aacacaggca cggggaaaag ccacagtaac tctgcttttt |
| 661 | ccagctcctg gttgggacta ggcaggctct tgcctgggaa ggagccagga ggagaaggcc |
| 721 | ccagcctgaa gctgttaggt aggagctaaa tccagggcca gatttccagg aggtggtggg |
| 781 | gcaagtgaaa acaccaagat ttgggggtca ggaggcctgg gatcctggca ggaaatggcc |
| 841 | aggcactacc tttgaccttg gattctgagc tgggctcctc ctccacaaaa tgaagggaag |
| 901 | ggtgctcagc ccctaccttg cagggttgtt ttgaagcatg tgttaccaca gggctgagct |
| 961 | tctgcaccac aagatccaga cctcttgtct ttacaggcag agaatgtaca gtctgtggaa |
| 1021 | agagaagggc aaacccagag ccctgcctag cggtctgctt tagtggaatc ctcggggacc |
| 1081 | tgtgaccttg aaggcaccta aacccctaca tcaccgcaag ggcagaagag ggctggcagc |
| 1141 | tctcactttt atttcccgtt ctcaagagat cctgtccaca gagttctggg ggtctaaaga |
| 1201 | cttaagagac attgtcccaa acccttaaaa tggggcatgg gtctgccttg gcaggaaagg |
| 1261 | tccttcaatc ctaaccagga gcaagagccc agcacggccc ctttaagaaa gttggaggtg |
| 1321 | ggagcagaca gagggagccc ccgcccaaaa tctaagcgcg ggtctggcgc ctgccccgcc |
| 1381 | cccggagtta agtactggac agtccttgcg ccctagcccg tgcgaggagc cctgctccct |
| 1441 | cgactttgga cttgaattgc gacttgcgac ttgcgactcg gtgctgtcca gattcagcac |
| 1501 | gctctgttcc ttcgcc |

As used herein, “treating” or “treatment” refers to any indication of success in amelioration of an injury, pathology, or condition, including any objective or subjective parameter such as abatement; remission; diminishing of symptoms or making the injury, pathology, or condition more tolerable to the patient; slowing the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluation. “Treating” includes the administration of glial progenitor cells to prevent or delay, to alleviate, or to arrest or inhibit development of the symptoms or conditions associated with the disease, condition or disorder.

“Therapeutic effect” refers to the reduction, elimination, or prevention of the disease, symptoms of the disease, or side effects of a disease, condition or disorder in the subject. Treatment may be prophylactic (to prevent or delay the onset or worsening of the disease, condition or disorder, or to prevent the manifestation of clinical or subclinical symptoms thereof) or therapeutic suppression or alleviation of symptoms after the manifestation of the disease, condition or disorder.

As used herein, “subject” refers to any living organism which may be treated with the present application. As such, the term “subject” may include, but is not limited to, any non-human mammal, primate, or human. In one embodiment, the subject is mammalian. In another embodiment, the subject is a mammal, such as mice, rats, other rodents, rabbits, dogs, cats, swine, sheep, horses, primates, or humans. In a further embodiment, the subject is human.

### 1. Condition Mediated by Deficiency in Myelin and Myelin-Related Disorders

One aspect of this disclosure relates to compositions and methods for treating a condition mediated by a deficiency in myelin or a myelin-related disorder. Such conditions or disorders include any diseases or conditions related to demyelination, insufficient myelination and remyelination, or dysmyelination in a subject. Such a disorder can be inherited or acquired or both. Demyelination in the CNS may occur in response to genetic mutation (leukodystrophies), autoimmune disease (e.g., multiple sclerosis), or trauma (e.g., traumatic brain injury, spinal cord injury, or ischemic stroke). Perturbation of myelin function may play a critical role in neurologic and psychiatric disorders such as Autism Spectrum Disorder (ASD), Alzheimer's disease, Huntington's disease, Multiple System Atrophy, Parkinson's disease, Fragile X syndrome, schizophrenia, and various leukodystrophies.

Leukodystrophies are a group of rare, primarily inherited neurological disorders that result from the abnormal production, processing, or development of myelin and are the result of genetic defects (mutations). Some forms are present at birth, while others may not produce symptoms until a child becomes a toddler. A few primarily affect adults. Leukodystrophies include Canavan disease, Pelizaeus-Merzbacher disease, Hypomyelination with Atrophy of the Basal Ganglia and Cerebellum, Krabbe disease (Globoid cell leukodystrophy), X-linked adrenoleukodystrophy, Metachromatic leukodystrophy, Pelizaeus-Merzbacher-like disease (or hypomyelinating leukodystrophy-2), Niemann-Pick disease type C (NPC), Autosomal dominant leukodystrophy with autonomic diseases (ADLD), 4H Leukodystrophy (Pol III-related leukodystrophy), Zellweger Spectrum Disorders (ZSD), Childhood ataxia with central nervous system hypomyelination or CACH (also called vanishing white matter disease or VWMD), Cerebrotendinous xanthomatosis (CTX), Alexander disease (AXD), SOX10-associated peripheral demyelinating neuropathy, central dysmyelinating leukodystrophy, Waardenburg syndrome, Hirschsprung disease (PCWH), Adult polyglucosan body disease (APBD), Hereditary diffuse leukoencephalopathy with spheroids (HDLS), Aicardi-Goutieres syndrome (AGS), and Adult Refsum disease.

Suitable subjects for treatment in accordance with the methods described herein include any human subject having a condition mediated by a deficiency in myelin.

In another embodiment, the condition mediated by a deficiency in myelin is selected from the group consisting of pediatric leukodystrophies, the lysosomal storage diseases, congenital dysmyelination, cerebral palsy, inflammatory demyelination, post-infectious and post-vaccinial leukoencephalitis, radiation- or chemotherapy induced demyelination, and vascular demyelination.

In a further embodiment, the condition mediated by a deficiency in myelin requires myelination.

In another embodiment, the condition mediated by a deficiency in myelin requires remyelination. In some embodiments, the condition requiring remyelination is selected from the group consisting of multiple sclerosis, neuromyelitis optica, transverse myelitis, optic neuritis, subcortical stroke, diabetic leukoencephalopathy, hypertensive leukoencephalopathy, age-related white matter disease, white matter dementia, Binswanger's disease, spinal cord injury, radiation- or chemotherapy induced demyelination, post-infectious and post-vaccinial leukoencephalitis, periventricular leukomalacia, and cerebral palsy.

In a further embodiment, the condition mediated by a deficiency in myelin is neurodegenerative disease. In some embodiments, the neurodegenerative disease is Huntington's disease.

Huntington's disease is an autosomal dominant neurodegenerative disease characterized by a relentlessly progressive movement disorder with devastating psychiatric and cognitive deterioration. Huntington's disease is associated with a consistent and severe atrophy of the neostriatum which is related to a marked loss of the GABAergic medium-sized spiny projection neurons, the major output neurons of the striatum. Huntington's disease is characterized by abnormally long CAG repeat expansions in the first exon of the Huntingtin gene. The encoded polyglutamine expansions of mutant huntingtin protein disrupt its normal functions and protein-protein interactions, ultimately yielding widespread neuropathology, most rapidly evident in the neostriatum.

Other neurodegenerative diseases treatable in accordance with the present application include frontotemporal dementia, Alzheimer's disease, Parkinson's disease, multisystem atrophy, and amyotrophic lateral sclerosis.

In an embodiment, the condition mediated by a deficiency in myelin is a neuropsychiatric disease. In some embodiments, the neuropsychiatric disease is schizophrenia.

Schizophrenia is a serious mental illness that affects how a person thinks, feels, and behaves. The symptoms of schizophrenia generally fall into the following three categories: 1) psychotic symptoms including altered perceptions, 2) negative symptoms including loss of motivation, disinterest and lack of enjoyment, and 3) cognitive symptoms including problems in attention, concentration, and memory.

Other neuropsychiatric diseases treatable in accordance with the present application include autism spectrum disorder and bipolar disorder

In another embodiment, the gene construct is administered in an expression vector. Suitable expression vectors include a viral vector, plasmid vector, or bacterial vector. In another embodiment, the expression vector is a viral vector selected from the group consisting of a lentiviral vector, an adenoviral vector, an adeno-associated viral vector, and a vaccinia vector.

In another embodiment, the genetic construct is administered in association with a glial progenitor cell-targeted fusogen or a glial progenitor cell-selective surface-binding moiety. The glial progenitor cell-targeted fusogen or a glial progenitor cell-selective surface-binding moiety can be directed against CD140a, NG2/CSPG4, A2B5 gangliosides, 04 sulfatides, or CD133.

As used herein, the term “C140a” also refers to platelet derived growth factor receptor alpha or PDGFRa or PDGFRα.

As used herein, the term “NG2/CSPG4” refers to neuron glial antigen 2 or chondroitin sulphate proteoglycan 4.

As used herein, the term “CD133” also refers to prominin-1.

The glial progenitor cells of the administered preparation can optionally be genetically modified to express proteins of interest other than TCF712. For example, the glial progenitor cells may be modified to express a therapeutic biological molecule, an exogenous targeting moiety, an exogenous marker (for example, for imaging purposes), or the like. The glial progenitor cells of the preparations can be optionally modified to overexpress an endogenous biological molecule, targeting moiety, and/or marker.

As used herein, “Glial progenitor cells” refers to cells having the potential to differentiate into cells of the glial lineage such as oligodendrocytes and astrocytes. Glia progenitor cells may be astrocyte-biased. Glial progenitor cells may be oligodendrocyte biased. As used herein, the term “glial cells” refers to a population of non-neuronal cells that provide support and nutrition, maintain homeostasis, either form myelin or promote myelination, and participate in signal transmission in the nervous system. “Glial cells” as used herein encompasses fully differentiated cells of the glial lineage, such as oligodendrocytes or astrocytes, and well as glial progenitor cells, each of which can be referred to as macroglial cells. In some embodiments, glial progenitor cells are also known as oligodendrocyte progenitor cells or NG2 cells.

The glial progenitor cells of the administered preparation may be astrocyte-biased glial progenitor cells, oligodendrocyte-biased glial progenitor cells, unbiased glial progenitor cells, or a combination thereof. The glial progenitor cells of the administered preparation express one or more markers of the glial cell lineage. For example, in one embodiment, the glial progenitor cells of the administered preparation may express A2B5+. In another embodiment, glial progenitor cells of the administered preparation are positive for a PDGFαR marker. The PDGFαR marker is optionally a PDGFαR ectodomain, such as CD140a. PDGFαR and CD140a are markers of an oligodendrocyte-biased glial progenitor cells. In another embodiment, glial progenitor cells of the administered preparation are CD44+. CD44 is a marker of an astrocyte-biased glial progenitor cell. In another embodiment, glial progenitor cells of the administered preparation are positive for a CD9 marker. The CD9 marker is optionally a CD9 ectodomain. In one embodiment, the glial progenitor cells of the preparation are A2B5+, CD140a+, and/or CD44+. The aforementioned glial progenitor cell surface markers can be used to identify, separate, and/or enrich the preparation for glial progenitor cells prior to administration.

The administered glial progenitor cell preparation is optionally negative for a PSA-NCAM marker and/or other neuronal lineage markers, and/or negative for one or more inflammatory cell markers, e.g., negative for a CD11 marker, negative for a CD32 marker, and/or negative for a CD36 marker (which are markers for microglia). Optionally, the preparation of glial progenitor cells are negative for any combination or subset of these additional markers. Thus, for example, the preparation of glial progenitor cells is negative for any one, two, three, or four of these additional markers.

The human glial progenitor cells administered in accordance with the present application may be derived from any suitable source of glial cells, such as, for example and without limitation, human induced pluripotent stem cells (iPSCs), embryonic stem cells, fetal tissue, and/or astrocytes as described in more detail below.

iPSCs are pluripotent cells that are derived from non-pluripotent cells, such as somatic cells. For example, and without limitation, iPSCs can be derived from tissue, peripheral blood, umbilical cord blood, and bone marrow (see e.g., Cai et al., “Generation of Human Induced Pluripotent Stem Cells from Umbilical Cord Matrix and Amniotic Membrane Mesenchymal Cells,” J. Biol. Chem. 285(15):112227-11234 (2110); Giorgetti et al., “Generation of Induced Pluripotent Stem Cells from Human Cord Blood Cells with only Two Factors: Oct4 and Sox2,” Nat. Protocol. 5(4):811-820 (2010); Streckfuss-Bomeke et al., “Comparative Study of Human-Induced Pluripotent Stem Cells Derived from Bone Marrow Cells, Hair Keratinocytes, and Skin Fibroblasts,” Eur. Heart doi:10.1093/eurheartj/ehs203 (Jul. 12, 2012); Hu et al., “Efficient Generation of Transgene-Free Induced Pluripotent Stem Cells from Normal and Neoplastic Bone Marrow and Cord Blood Mononuclear Cells,” Blood doi:10.1182/blood-2010-07-298331 (Feb. 4, 2011); Sommer et al., “Generation of Human Induced Pluripotent Stem Cells from Peripheral Blood using the STEMCCA Lentiviral Vector,” J. Vis. Exp. 68:e4327 doi:10.3791/4327 (2012), which are hereby incorporated by reference in their entirety). The somatic cells are reprogrammed to an embryonic stem cell-like state using genetic manipulation. Exemplary somatic cells suitable for the formation of iPSCs include fibroblasts (see e.g., Streckfuss-Bomeke et al., “Comparative Study of Human-Induced Pluripotent Stem Cells Derived from Bone Marrow Cells, Hair Keratinocytes, and Skin Fibroblasts,” Eur. Heart doi:10.1093/eurheartj/ehs203 (2012), which is hereby incorporated by reference in its entirety), such as dermal fibroblasts obtained by a skin sample or biopsy, synoviocytes from synovial tissue, keratinocytes, mature B cells, mature T cells, pancreatic β cells, melanocytes, hepatocytes, foreskin cells, cheek cells, or lung fibroblasts.

Methods of producing induced pluripotent stem cells are known in the art and typically involve expressing a combination of reprogramming factors in a somatic cell. Suitable reprogramming factors that promote and induce iPSC generation include one or more of Oct4, Klf4, Sox2, c-Myc, Nanog, C/EBPα, Esrrb, Lin28, and Nr5a2. In certain embodiments, at least two reprogramming factors are expressed in a somatic cell to successfully reprogram the somatic cell. In other embodiments, at least three reprogramming factors are expressed in a somatic cell to successfully reprogram the somatic cell.

iPSCs may be derived by methods known in the art, including the use integrating viral vectors (e.g., lentiviral vectors, inducible lentiviral vectors, and retroviral vectors), excisable vectors (e.g., transposon and foxed lentiviral vectors), and non-integrating vectors (e.g., adenoviral and plasmid vectors) to deliver the genes that promote cell reprogramming (see e.g., Takahashi and Yamanaka, “Induction of Pluripotent Stem Cells from Mouse Embryonic and Adult Fibroblast Cultures by Defined Factors,” Cell 126:663-676 (2006); Okita. et al., “Generation of Germline-Competent Induced Pluripotent Stem Cells,” Nature 448:313-317 (2007); Nakagawa et al., “Generation of Induced Pluripotent Stem Cells without Myc from Mouse and Human Fibroblasts,” Nat. Biotechnol. 26:101-106 (2008); Takahashi et al., Cell 131:1-12 (2007); Meissner et al., “Direct Reprogramming of Genetically Unmodified Fibroblasts into Pluripotent Stem Cells,” Nat. Biotech. 25:1177-1181 (2007); Yu et al., “Induced Pluripotent Stem Cell Lines Derived from Human Somatic Cells,” Science 318:1917-1920 (2007); Park et al., “Reprogramming of Human Somatic Cells to Pluripotency with Defined Factors,” Nature 451:141-146 (2008); and U.S. Patent Application Publication No. 2008/0233610, which are hereby incorporated by reference in their entirety). Other methods for generating IPS cells include those disclosed in WO2007/069666, WO2009/006930, WO2009/006997, WO2009/007852, WO2008/118820, U.S. Patent Application Publication No. 2011/0200568 to Ikeda et al., U.S. Patent Application Publication No 2010/0156778 to Egusa et al., U.S. Patent Application Publication No 2012/0276070 to Musick, and U.S. Patent Application Publication No 2012/0276636 to Nakagawa, Shi et al., Cell Stem Cell 3(5):568-574 (2008), Kim et al., Nature 454:646-650 (2008), Kim et al., Cell 136(3):411-419 (2009), Huangfu et al., Nat. Biotechnol. 26:1269-1275 (2008), Zhao et al., Cell Stem Cell 3:475-479 (2008), Feng et al., Nat. Cell Biol. 11:197-203 (2009), and Hanna et al., Cell 133(2):250-264 (2008) which are hereby incorporated by reference in their entirety.

The methods of iPSC generation described above can be modified to include small molecules that enhance reprogramming efficiency or even substitute for a reprogramming factor. These small molecules include, without limitation, epigenetic modulators such as, the DNA methyltransferase inhibitor 5′-azacytidine, the histone deacetylase inhibitor VPA, and the G9a histone methyltransferase inhibitor BIX-01294 together with BayK8644, an L-type calcium channel agonist. Other small molecule reprogramming factors include those that target signal transduction pathways, such as TGF-β inhibitors and kinase inhibitors (e.g., kenpaullone) (see review by Sommer and Mostoslaysky, “Experimental Approaches for the Generation of Induced Pluripotent Stem Cells,” Stem Cell Res. Ther. 1:26 doi:10.1186/scrt26 (Aug. 10, 2010), which is hereby incorporated by reference in its entirety).

Methods of obtaining highly enriched preparations of glial progenitor cells from the iPSCs that are suitable for the methods described herein are disclosed in WO2014/124087 to Goldman and Wang, and Wang et al., “Human iPSC-Derived Oligodendrocyte Progenitors Can Myelinate and Rescue a Mouse Model of Congenital Hypomyelination,” Cell Stem Cell 12(2):252-264 (2013), which are hereby incorporated by reference in their entirety.

In another embodiment, the human glial progenitor cells are derived from embryonic stem cells. Human embryonic stem cells provide a virtually unlimited source of clonal/genetically modified cells potentially useful for tissue replacement therapies. Methods of obtaining highly enriched preparations of glial progenitor cells from embryonic cells that are suitable for use in the methods of the present disclosure are described in Wang et al., “Human iPSC-derived Oligodendrocyte Progenitor Cells Can Myelinate and Rescue a Mouse Model of Congenital Hypomyelination,” Cell Stem Cell 12:252-264 (2013), which is hereby incorporated by reference in its entirety.

In another embodiment, the human glial progenitor cells are derived from human fetal tissue. Glial progenitor cells can be extracted from fetal brain tissue containing a mixed population of cells directly by using the promoter specific separation technique as described in U.S. Patent Application Publication Nos. 2004/0029269 and 2003/0223972 to Goldman, which are hereby incorporated by reference in their entirety. This method involves selecting a promoter which functions specifically in glial progenitor cells, and introducing a nucleic acid encoding a marker protein under the control of said promoter into the mixed population cells. The mixed population of cells is allowed to express the marker protein and the cells expressing the marker protein are separated from the population of cells, with the separated cells being the glial progenitor cells. Human glial progenitor cells can be isolated from ventricular or subventricular zones of the brain or from the subcortical white matter.

Glial specific promoters that can be used for isolating glial progenitor cells from a mixed population of cells include the CNP promoter (Scherer et al., Neuron 12:1363-75 (1994), which is hereby incorporated by reference in its entirety), an NCAM promoter (Holst et al., J. Biol. Chem. 269:22245-52 (1994), which is hereby incorporated by reference in its entirety), a myelin basic protein promoter (Wrabetz et al., J. Neurosci. Res. 36:455-71 (1993), which is hereby incorporated by reference in its entirety), a JC virus minimal core promoter (Krebs et al., J. Virol. 69:2434-42 (1995), which is hereby incorporated by reference in its entirety), a myelin-associated glycoprotein promoter (Laszkiewicz et al., “Structural Characterization of Myelin-associated Glycoprotein Gene Core Promoter,” J. Neurosci. Res. 50(6): 928-36 (1997), which is hereby incorporated by reference in its entirety), or a proteolipid protein promoter (Cook et al., “Regulation of Rodent Myelin Proteolipid Protein Gene Expression,” Neurosci. Lett. 137(1): 56-60 (1992); Wight et al., “Regulation of Murine Myelin Proteolipid Protein Gene Expression,” J. Neurosci. Res. 50(6): 917-27 (1997); and Cambi et al., Neurochem. Res. 19:1055-60 (1994), which are hereby incorporated by reference in their entirety). See also U.S. Pat. No. 6,245,564 to Goldman et al., which is hereby incorporated by reference in its entirety.

The glial progenitor cell population derived from fetal tissue can be enriched for by first removing neurons or neural progenitor cells from the mixed cell population. Where neuronal progenitor cells are to be separated from the mixed population of cells, they can be removed based on their surface expression of NCAM, PSA-NCAM, or any other surface moiety specific to neurons or neural progenitor cells. Neurons or neural progenitor cells may also be separated from a mixed population of cells using the promoter based separation technique. Neuron or neural progenitor specific promoters that can be used for separating neural cells from a mixed population of cells include the Tal tubulin promoter (Gloster et al., J. Neurosci. 14:7319-30 (1994) which is hereby incorporated by reference in its entirety), a Hu promoter (Park et al., “Analysis of Upstream Elements in the HuC Promoter Leads to the Establishment of Transgenic Zebrafish with Fluorescent Neurons,” Dev. Biol. 227(2): 279-93 (2000), which is hereby incorporated by reference in its entirety), an ELAV promoter (Yao et al., “Neural Specificity of ELAV Expression: Defining a Drosophila Promoter for Directing Expression to the Nervous System,” J. Neurochem. 63(1): 41-51 (1994), which is hereby incorporated by reference in its entirety), a MAP-1B promoter (Liu et al., Gene 171:307-08 (1996), which is hereby incorporated by reference in its entirety), or a GAP-43 promoter. Techniques for introducing the nucleic acid molecules of the construct into the plurality of cells and then sorting the cells are described in U.S. Pat. No. 6,245,564 to Goldman et al., and U.S. Patent Application Publication No. 2004/0029269 to Goldman et al., which are hereby incorporated by reference in their entirety.

As an alternative to using promoter-based cell sorting to recover glial progenitor cells from a mixed population of cells, an immunoseparation procedure can be utilized. In a positive immunoseparation technique, the desired cells (i.e., glial progenitor cells) are isolated based on proteinaceous surface markers naturally present on the progenitor cells. For example, the surface marker A2B5 is an initially expressed early marker of glial progenitor cells (Nunes et al., “Identification and Isolation of Multipotential Neural Progenitor Cells from the Adult Human White Matter,” Soc. Neurosci. Abstr. (2001), which is hereby incorporated by reference in its entirety). Using an antibody specific to A2B5, glial progenitor cells can be separated from a mixed population of cell types. Similarly, the surface marker CD44 identifies astrocyte-biased glial progenitor cells (Liu et al., “CD44 Expression Identifies Astrocyte-Restricted Precursor Cells,” Dev. Biol. 276:31-46 (2004), which is hereby incorporated by reference in its entirety). Using CD44-conjugated microbead technology, astroctye-biased glial progenitor cells can be separated from a mixed population of cell types. Oligodendrocyte-biased glial progenitor cells can be separated from a mixed population of cell types based on expression of PDGFαR, the PDGFαR ectodomain CD140a, or CD9. Cells expressing markers of non-glial cell types (e.g., neurons, inflammatory cells, etc.) can be removed from the preparation of glial cells to further enrich the preparation for the desired glial cell type using immunoseparation techniques. For example, the glial progenitor cell population is preferably negative for a PSA-NCAM marker and/or other markers for cells of neuronal lineage, negative for one or more inflammatory cell markers, e.g., negative for a CD11 marker, negative for a CD32 marker, and/or negative for a CD36 marker, which are markers for microglia. Exemplary microbead technologies include MACS® Microbeads, MACS® Columns, and MACS® Separators. Additional examples of immunoseparation are described in Wang et al., “Prospective Identification, Direct Isolation, and Expression Profiling of a Telomerase Expressing Subpopulation of Human Neural Stem Cells, Using Sox2 Enhancer-Directed FACS,” J. Neurosci. 30:14635-14648 (2010); Keyoung et al., “High-Yield Selection and Extraction of Two Promoter-Defined Phenotypes of Neural Stem Cells from the Fetal Human Brain,” Nat. Biotechnol. 19:843-850 (2001); and Windrem et al., “Neonatal Chimerization with Human Glial Progenitor Cells can both Remyelinate and Rescue the Otherwise Lethally Hypomyelinated Shiverer Mouse,” Cell Stem Cell 2:553-565 (2008), which are hereby incorporated by reference in their entirety.

In accordance with the methods described herein, the selected preparation of administered human glial progenitor cells comprise at least about 80% glial progenitor cells, including, for example, about 80%, 85%, 90%, 95%, 96%, 97%, 98%, 99%, 100% glial progenitor cells. The selected preparation of glial progenitor cells can be relatively devoid (e.g., containing less than 20, 15, 10, 9, 8, 7, 6, 5, 4, 3, 2, or 1%) of other cells types such as neurons or cells of neuronal lineage, fibrous astrocytes and cells of fibrous astrocyte lineage, and pluripotential stem cells (like ES cells). Optionally, example cell populations are substantially pure populations of glial progenitor cells.

### 2. Transcription Factor 7-Like 2 Protein

Transcription factor 7-like 2, also known as TCF7L2 or TCF4, is a protein acting as a transcription factor that, in humans, is encoded by the TCF7L2 gene. The human TCF7L2 gene is located on chromosome 10q25.2-q25.3, contains 17 exons. As a member of the TCF family, TCF7L2 can form a bipartite transcription factor and influence several biological pathways, including the Wnt signaling pathway.

The full-length human TCF7L2 protein contains in the N terminal, catenin-binding domain, Groucho-binding sequence, HMG box-DNA-binding domain (HGM-DBD), cysteine clamp (C clamp), and C terminal. See, e.g., Li et al., Front Cardiovasc Med. 2021 Sep. 9; 8:701279. doi: 10.3389/fcvm.2021.701279. eCollection 2021. With the help of HGM-DBD, TCF7L2 can recognize specific DNA subsequences (5′-xCTTTGATx-3′) in the doublehelix dimple and trigger transcription factor activity. The C clamp has been considered to assist the binding of HGM-DBD with certain DNA sequences, although the C clamp contains an alternative DNA-binding domain (5′-xTGCCGCx-3′) without transcription regulatory activity. TCF7L2 exerts dual transcription regulatory effects on target genes influenced by the transcriptional co-activator β-catenin or transcriptional co-repressor transducin-like enhancer of split (TLE)/Groucho. With Wnt signaling stimulation, increased amounts of b-catenin are imported into the nucleus, where they subsequently assemble into the β-catenin/TCF7L2 complex. In addition, β-catenin functions as a scaffold to assist the binding of the β-catenin/TCF7L2 complex to the promoter of target genes and thus enhance promoter activity. In the absence of Wnt/β-catenin signaling, the co-repressor TLEs preferentially occupy TCF7L2 by the glutamine-rich (Q) domain and recruit histone methyltransferases or histone deacetylases to silence downstream genes. Taken together, TCF7L2 contains two DNA-binding domains (HGM-DBD and C clamp), but only HGM-DBD can activate transcription. Furthermore, TCF7L2 is subject to dual regulation by the transcriptional co-activator β-catenin or transcriptional co-repressor TLE/Groucho. Li et al., Front Cardiovasc Med. 2021 Sep. 9; 8:701279. doi: 10.3389/fcvm.2021.701279. eCollection 2021.

Human or mouse TCF7L2 has multiple splice variants or isoforms that exhibit different expression patterns or play different roles during development (Helgason et al., Nature Genetics 39: 218-225 (2007). Shown below are some of human TCF7L2 variants/isoforms. All of these human splice variants/isoforms can be used in the expression cassette, genetic construct, vector, composition, or method disclosed herein. Listed below are some exemplary Tcf712 Human isoforms, related nucleic acid sequences, and related amino acid sequences.

Tcf7l2 Human isoforms:

| Gene ID | Transcript | Length (nt) | Protein | Length (aa) | Isoform |
|---|---|---|---|---|---|
| 6934 | NM_001146274.2 | 4025 | NP_001139746.1 | 602 | 1 |
| 6934 | NM_030756.5 | 4007 | NP_110383.2 | 596 | 2 |
| 6934 | NM_001146283.2 | 4024 | NP_001139755.1 | 489 | 3 |
| 6934 | NM_001146284.2 | 3922 | NP_001139756.1 | 455 | 4 |
| 6934 | NM_001146285.2 | 3956 | NP_001139757.1 | 579 | 5 |
| 6934 | NM_001146286.2 | 3883 | NP_001139758.1 | 442 | 6 |
| 6934 | NM_001198525.2 | 4095 | NP_001185454.1 | 476 | 7 |
| 6934 | NM_001198526.2 | 3956 | NP_001185455.1 | 579 | 8 |
| 6934 | NM_001198527.2 | 4020 | NP_001185456.1 | 455 | 9 |
| 6934 | NM_001198528.2 | 4032 | NP_001185457.1 | 459 | 10 |
| 6934 | NM_001198529.2 | 3934 | NP_001185458.1 | 459 | 11 |
| 6934 | NM_001198530.2 | 3781 | NP_001185459.1 | 408 | 12 |
| 6934 | NM_001198531.2 | 3952 | NP_001185460.1 | 465 | 13 |
| 6934 | NM_001349870.2 | 3756 | NP_001336799.1 | 339 | 14 |
| 6934 | NM_001349871.1 | 3288 | NP_001336800.1 | 299 | 15 |
| 6934 | NM_001363501.2 | 4003 | NP_001350430.1 | 482 | 16 |
| 6934 | NM_001367943.1 | 4076 | NP_001354872.1 | 619 | 17 |

>NM_001146274.2 Homo sapiens transcription
factor 7 like 2 (TCF7L2), transcript variant
1, (SEQ ID NO: 45)
GTCAATAATCTCCGCTCCCAGACTACTCCGTTCCTCCGGATTTCGATCCCCCTTTTTCTATCTGTCAATC
AGCGCCGCCTTTGAACTGAAAAGCTCTCAGTCTAACTTCAACTCACTCAAATCCGAGCGGCACGAGCACC
TCCTGTATCTTCGGCTTCCCCCCCCCTTTGCTCTTTATATCTGACTTCTTGTTGTTGTTGGTGTTTTTTT
TTTTTTTACCCCCCTTTTTTATTTATTATTTTTTTGCACATTGATCGGATCCTTGGGAACGAGAGAAAAA
AGAAACCCAAACTCACGCGTGCAGAAGATCTCCCCCCCCTTCCCCTCCCCTCCTCCCTCTTTTCCCCTCC
CCAGGAGAAAAAGACCCCCAAGCAGAAAAAAGTTCACCTTGGACTCGTCTTTTTCTTGCAATATTTTTTG
GGGGGGCAAAACTTTTTGGGGGTGATTTTTTTTGGCTTTTCTTCCTCCTTCATTTTTCTTCCAAAATTGC
TGCTGGTGGGTGAAAAAAAAATGCCGCAGCTGAACGGCGGTGGAGGGGATGACCTAGGCGCCAACGACGA
ACTGATTTCCTTCAAAGACGAGGGCGAACAGGAGGAGAAGAGCTCCGAAAACTCCTCGGCAGAGAGGGAT
TTAGCTGATGTCAAATCGTCTCTAGTCAATGAATCAGAAACGAATCAAAACAGCTCCTCCGATTCCGAGG
CGGAAAGACGGCCTCCGCCTCGCTCCGAAAGTTTCCGAGACAAATCCCGGGAAAGTTTGGAAGAAGCGGC
CAAGAGGCAAGATGGAGGGCTCTTTAAGGGGCCACCGTATCCCGGCTACCCCTTCATCATGATCCCCGAC
CTGACGAGCCCCTACCTCCCCAACGGATCGCTCTCGCCCACCGCCCGAACCCTCCATTTTCAGTCCGGCA
GCACACATTACTCTGCGTACAAAACGATTGAACACCAGATTGCAGTTCAGTATCTCCAGATGAAATGGCC
ACTGCTTGATGTCCAGGCAGGGAGCCTCCAGAGTAGACAAGCCCTCAAGGATGCCCGGTCCCCATCACCG
GCACACATTGTCTCTAACAAAGTGCCAGTGGTGCAGCACCCTCACCATGTCCACCCCCTCACGCCTCTTA
TCACGTACAGCAATGAACACTTCACGCCGGGAAACCCACCTCCACACTTACCAGCCGACGTAGACCCCAA
AACAGGAATCCCACGGCCTCCGCACCCTCCAGATATATCCCCGTATTACCCACTATCGCCTGGCACCGTA
GGACAAATCCCCCATCCGCTAGGATGGTTAGTACCACAGCAAGGTCAACCAGTGTACCCAATCACGACAG
GAGGATTCAGACACCCCTACCCCACAGCTCTGACCGTCAATGCTTCCATGTCCAGGTTCCCTCCCCATAT
GGTCCCACCACATCATACGCTACACACGACGGGCATTCCGCATCCGGCCATAGTCACACCAACAGTCAAA
CAGGAATCGTCCCAGAGTGATGTCGGCTCACTCCATAGTTCAAAGCATCAGGACTCCAAAAAGGAAGAAG
AAAAGAAGAAGCCCCACATAAAGAAACCTCTTAATGCATTCATGTTGTATATGAAGGAAATGAGAGCAAA
GGTCGTAGCTGAGTGCACGTTGAAAGAAAGCGCGGCCATCAACCAGATCCTTGGGCGGAGGTGGCATGCA
CTGTCCAGAGAAGAGCAAGCGAAATACTACGAGCTGGCCCGGAAGGAGCGACAGCTTCATATGCAACTGT
ACCCCGGCTGGTCCGCGCGGGATAACTATGGAAAGAAGAAGAAGAGGAAAAGGGACAAGCAGCCGGGAGA
GACCAATGATGCAAATACTCCAAAGAAGTGTCGGGCACTGTTCGGGCTTGACCGACAGACTTTATGGTGC
AAACCGTGCAGGAGAAAAAAAAAGTGCGTTCGCTACATACAAGGTGAAGGCAGCTGCCTCAGCCCACCCT
CTTCAGATGGAAGCTTACTAGATTCGCCTCCCCCCTCCCCGAACCTGCTAGGCTCCCCTCCCCGAGACGC
CAAGTCACAGACTGAGCAGACCCAGCCTCTGTCGCTGTCCCTGAAGCCCGACCCCCTGGCCCACCTGTCC
ATGATGCCTCCGCCACCCGCCCTCCTGCTCGCTGAGGCCACCCACAAGGCCTCCGCCCTCTGTCCCAACG
GGGCCCTGGACCTGCCCCCAGCCGCTTTGCAGCCTGCCGCCCCCTCCTCATCAATTGCACAGCCGTCGAC
TTCTTCCTTACATTCCCACAGCTCCCTGGCCGGGACCCAGCCCCAGCCGCTGTCGCTCGTCACCAAGTCT
TTAGAATAGCTTTAGCGTCGTGAACCCCGCTGCTTTGTTTATGGTTTTGTTTCACTTTTCTTAATTTGCC
CCCCACCCCCACCTTGAAAGGTTTTGTTTTGTACTCTCTTAATTTTGTGCCATGTGGCTACATTAGTTGA
TGTTTATCGAGTTCATTGGTCAATATTTGACCCATTCTTATTTCAATTTCTCCTTTTAAATATGTAGATG
AGAGAAGAACCTCATGATTCTACCAAAATTTTTATCAACAGCTGTTTAAAGTCTTTGTAGCGTTTAAAAA
ATATATATATATACATAACTGTTATGTAGTTCGGATAGCTTAGTTTTAAAAGACTGATTAAAAAACAAAA
AGAAAAAAAAAGCAATTTTGAAGCAGCCCTCCAGAAGGAGTTGGTTCTGTATTATTTGTATTAAATACGA
GCTTGCGAACCAATCATTTTACATCTGGTTTTTAAACCGTAAGGGCACCATGAATGCAGTGCCGTTACTT
TTTTTTTTTTTTTCTGTGTGAAACAACTCTTATTGTGATGTTACTTGTTATTGTTTAAATGTACAGAAAC
AAAGGGTAAAAATGTGTTAATATACCTTGTTCCATGGTGTTGTTCTTTTGGGGGGAGGGGACGCTACTCA
ACACTTAATAGAATCACAACGCTGTTGGGCCAGTAGTATTTATTGCTTTAGAGATTGCTTGTCGTACCTG
TATGTCGTCCCTTTTTAAATATGTTTTCCTTTTTCTTGAAACTGTATAAAGTTTTTTTCCCCCTTAGCAT
AAGCATCTTATATATAACAACTCATTTGTACAAGGTTTTTAAGTTTATATATAAAATGTGTATATATATT
TTTGTTTCCCCTTTTTGACTTTTTTTTTTCTGTATGAAACCCAGATGTCACCAAATGGACATTAATAGTT
GCATTAAGGATCAGTAGCATTAACAAAAGTTGCTTTAAAAGCCATTATGTAAAACAAGACTTGAAAATGA
GTGAGGGAATTTTAGCGACACTGTCTGAGCAGCAGTGGGAACCATCTTCGTTTCCCCTTTGAACTCCCAG
TGGGATGCCCTACCCTGCGCCCTTAGGACCCGGACTGACCGTGTACAAAACTTTACGTGCCAAAATTCTC
AGTGAATTTAGCTTTCTCCCTCTTTTTGATGCTGTAATTTTTGTTCATCATGTTTTGCTGTGATGTTACA
TAGGTAGATTTGTATGTAGTTTTAATGTCACCTATAACAAAATGTGTTTGGTAGCAGATTGTCCAGAAAG
CATTTTAAATGAAGAGGTATAAACCCTTAAGGGCCAAAATTCTGTATATTAGATTACTCTTAAACGAAAA
ACCAGCTGCCGCTTTTATGTACACATATTACATACGAGTAGGCAGCAGACTTTAAAAATAAAAAAAACCT
AGGCATGTTGATGTTGCAAAATGCTGTATAAAGCTGAAACCTGTTCATTCAGTGCCATTGTAGTTGACAT
GAAGCGATTGTAAAACTGTCTCCGATTTTTCTCTGGTTTATTAAAATGCTAACTATAACATTTTTTGTGA
ATACTTTGAATGTTTCCTAACAGTTGTGATGTTACTGTTCCGTTTTATGCTCTTATTCCAAGTTCATTTT
TAATGGTTTGGAAGCCATTTTTGTAATGAATAAATGTTCATGCTGTACAGTATCTGTAGCATGCCGTTCT
GGATTAATAAAAGCAACTTAGTATGTGCAGATAAA
Protein sequence (SEQ ID NO: 46):
MPQLNGGGGDDLGANDELISFKDEGEQEEKSSENSSAERDLADVKSSLVNESETNONSSSDSEAERRPPPRSESF
RDKSRESLEEAAKRQDGGLFKGPPYPGYPFIMIPDLTSPYLPNGSLSPTARTLHFQSGSTHYSAYKTIEHQIAVQ
YLQMKWPLLDVQAGSLQSRQALKDARSPSPAHIVSNKVPVVQHPHHVHPLTPLITYSNEHFTPGNPPPHLPADVD
PKTGIPRPPHPPDISPYYPLSPGTVGQIPHPLGWLVPQQGQPVYPITTGGFRHPYPTALTVNASMSRFPPHMVPP
HHTLHTTGIPHPAIVTPTVKQESSQSDVGSLHSSKHQDSKKEEEKKKPHIKKPLNAFMLYMKEMRAKVVAECTLK
ESAAINQILGRRWHALSREEQAKYYELARKERQLHMQLYPGWSARDNYGKKKKRKRDKQPGETNDANTPKKCRAL
FGLDRQTLWCKPCRRKKKCVRYIQGEGSCLSPPSSDGSLLDSPPPSPNLLGSPPRDAKSQTEQTQPLSLSLKPDP
LAHLSMMPPPPALLLAEATHKASALCPNGALDLPPAALQPA
APSSSIAQPSTSSLHSHSSLAGTQPQPLSLVTKSLE
>NM_030756.5 Homo sapiens transcription
factor 7 like 2 (TCF7L2), transcript variant 2
(SEQ ID NO: 47)
GTCAATAATCTCCGCTCCCAGACTACTCCGTTCCTCCGGATTTCGATCCCCCTTTTTCTATCTGTCAATC
AGCGCCGCCTTTGAACTGAAAAGCTCTCAGTCTAACTTCAACTCACTCAAATCCGAGCGGCACGAGCACC
TCCTGTATCTTCGGCTTCCCCCCCCCTTTGCTCTTTATATCTGACTTCTTGTTGTTGTTGGTGTTTTTTT
TTTTTTTACCCCCCTTTTTTATTTATTATTTTTTTGCACATTGATCGGATCCTTGGGAACGAGAGAAAAA
AGAAACCCAAACTCACGCGTGCAGAAGATCTCCCCCCCCTTCCCCTCCCCTCCTCCCTCTTTTCCCCTCC
CCAGGAGAAAAAGACCCCCAAGCAGAAAAAAGTTCACCTTGGACTCGTCTTTTTCTTGCAATATTTTTTG
GGGGGGCAAAACTTTTTGGGGGTGATTTTTTTTGGCTTTTCTTCCTCCTTCATTTTTCTTCCAAAATTGC
TGCTGGTGGGTGAAAAAAAAATGCCGCAGCTGAACGGCGGTGGAGGGGATGACCTAGGCGCCAACGACGA
ACTGATTTCCTTCAAAGACGAGGGCGAACAGGAGGAGAAGAGCTCCGAAAACTCCTCGGCAGAGAGGGAT
TTAGCTGATGTCAAATCGTCTCTAGTCAATGAATCAGAAACGAATCAAAACAGCTCCTCCGATTCCGAGG
CGGAAAGACGGCCTCCGCCTCGCTCCGAAAGTTTCCGAGACAAATCCCGGGAAAGTTTGGAAGAAGCGGC
CAAGAGGCAAGATGGAGGGCTCTTTAAGGGGCCACCGTATCCCGGCTACCCCTTCATCATGATCCCCGAC
CTGACGAGCCCCTACCTCCCCAACGGATCGCTCTCGCCCACCGCCCGAACCTATCTCCAGATGAAATGGC
CACTGCTTGATGTCCAGGCAGGGAGCCTCCAGAGTAGACAAGCCCTCAAGGATGCCCGGTCCCCATCACC
GGCACACATTGTCTCTAACAAAGTGCCAGTGGTGCAGCACCCTCACCATGTCCACCCCCTCACGCCTCTT
ATCACGTACAGCAATGAACACTTCACGCCGGGAAACCCACCTCCACACTTACCAGCCGACGTAGACCCCA
AAACAGGAATCCCACGGCCTCCGCACCCTCCAGATATATCCCCGTATTACCCACTATCGCCTGGCACCGT
AGGACAAATCCCCCATCCGCTAGGATGGTTAGTACCACAGCAAGGTCAACCAGTGTACCCAATCACGACA
GGAGGATTCAGACACCCCTACCCCACAGCTCTGACCGTCAATGCTTCCATGTCCAGGTTCCCTCCCCATA
TGGTCCCACCACATCATACGCTACACACGACGGGCATTCCGCATCCGGCCATAGTCACACCAACAGTCAA
ACAGGAATCGTCCCAGAGTGATGTCGGCTCACTCCATAGTTCAAAGCATCAGGACTCCAAAAAGGAAGAA
GAAAAGAAGAAGCCCCACATAAAGAAACCTCTTAATGCATTCATGTTGTATATGAAGGAAATGAGAGCAA
AGGTCGTAGCTGAGTGCACGTTGAAAGAAAGCGCGGCCATCAACCAGATCCTTGGGCGGAGGTGGCATGC
ACTGTCCAGAGAAGAGCAAGCGAAATACTACGAGCTGGCCCGGAAGGAGCGACAGCTTCATATGCAACTG
TACCCCGGCTGGTCCGCGCGGGATAACTATGGAAAGAAGAAGAAGAGGAAAAGGGACAAGCAGCCGGGAG
AGACCAATGAACACAGCGAATGTTTCCTAAATCCTTGCCTTTCACTTCCTCCGATTACAGACCTGAGCGC
TCCTAAGAAATGCCGAGCGCGCTTTGGCCTTGATCAACAGAATAACTGGTGCGGCCCTTGCAGGAGAAAA
AAAAAGTGCGTTCGCTACATACAAGGTGAAGGCAGCTGCCTCAGCCCACCCTCTTCAGATGGAAGCTTAC
TAGATTCGCCTCCCCCCTCCCCGAACCTGCTAGGCTCCCCTCCCCGAGACGCCAAGTCACAGACTGAGCA
GACCCAGCCTCTGTCGCTGTCCCTGAAGCCCGACCCCCTGGCCCACCTGTCCATGATGCCTCCGCCACCC
GCCCTCCTGCTCGCTGAGGCCACCCACAAGGCCTCCGCCCTCTGTCCCAACGGGGCCCTGGACCTGCCCC
CAGCCGCTTTGCAGCCTGCCGCCCCCTCCTCATCAATTGCACAGCCGTCGACTTCTTCCTTACATTCCCA
CAGCTCCCTGGCCGGGACCCAGCCCCAGCCGCTGTCGCTCGTCACCAAGTCTTTAGAATAGCTTTAGCGT
CGTGAACCCCGCTGCTTTGTTTATGGTTTTGTTTCACTTTTCTTAATTTGCCCCCCACCCCCACCTTGAA
AGGTTTTGTTTTGTACTCTCTTAATTTTGTGCCATGTGGCTACATTAGTTGATGTTTATCGAGTTCATTG
GTCAATATTTGACCCATTCTTATTTCAATTTCTCCTTTTAAATATGTAGATGAGAGAAGAACCTCATGAT
TCTACCAAAATTTTTATCAACAGCTGTTTAAAGTCTTTGTAGCGTTTAAAAAATATATATATATACATAA
CTGTTATGTAGTTCGGATAGCTTAGTTTTAAAAGACTGATTAAAAAACAAAAAGAAAAAAAAAGCAATTT
TGAAGCAGCCCTCCAGAAGGAGTTGGTTCTGTATTATTTGTATTAAATACGAGCTTGCGAACCAATCATT
TTACATCTGGTTTTTAAACCGTAAGGGCACCATGAATGCAGTGCCGTTACTTTTTTTTTTTTTTTCTGTG
TGAAACAACTCTTATTGTGATGTTACTTGTTATTGTTTAAATGTACAGAAACAAAGGGTAAAAATGTGTT
AATATACCTTGTTCCATGGTGTTGTTCTTTTGGGGGGAGGGGACGCTACTCAACACTTAATAGAATCACA
ACGCTGTTGGGCCAGTAGTATTTATTGCTTTAGAGATTGCTTGTCGTACCTGTATGTCGTCCCTTTTTAA
ATATGTTTTCCTTTTTCTTGAAACTGTATAAAGTTTTTTTCCCCCTTAGCATAAGCATCTTATATATAAC
AACTCATTTGTACAAGGTTTTTAAGTTTATATATAAAATGTGTATATATATTTTTGTTTCCCCTTTTTGA
CTTTTTTTTTTCTGTATGAAACCCAGATGTCACCAAATGGACATTAATAGTTGCATTAAGGATCAGTAGC
ATTAACAAAAGTTGCTTTAAAAGCCATTATGTAAAACAAGACTTGAAAATGAGTGAGGGAATTTTAGCGA
CACTGTCTGAGCAGCAGTGGGAACCATCTTCGTTTCCCCTTTGAACTCCCAGTGGGATGCCCTACCCTGC
GCCCTTAGGACCCGGACTGACCGTGTACAAAACTTTACGTGCCAAAATTCTCAGTGAATTTAGCTTTCTC
CCTCTTTTTGATGCTGTAATTTTTGTTCATCATGTTTTGCTGTGATGTTACATAGGTAGATTTGTATGTA
GTTTTAATGTCACCTATAACAAAATGTGTTTGGTAGCAGATTGTCCAGAAAGCATTTTAAATGAAGAGGT
ATAAACCCTTAAGGGCCAAAATTCTGTATATTAGATTACTCTTAAACGAAAAACCAGCTGCCGCTTTTAT
GTACACATATTACATACGAGTAGGCAGCAGACTTTAAAAATAAAAAAAACCTAGGCATGTTGATGTTGCA
AAATGCTGTATAAAGCTGAAACCTGTTCATTCAGTGCCATTGTAGTTGACATGAAGCGATTGTAAAACTG
TCTCCGATTTTTCTCTGGTTTATTAAAATGCTAACTATAACATTTTTTGTGAATACTTTGAATGTTTCCT
AACAGTIGTGATGTTACTGTTCCGTTTTATGCTCTTATTCCAAGTTCATTTTTAATGGTTTGGAAGCCAT
TTTTGTAATGAATAAATGTTCATGCTGTACAGTATCTGTAGCATGCCGTTCTGGATTAATAAAAGCAACT
TAGTATGTGCAGATAAA,
Protein sequence (SEQ ID NO: 48):
MPQLNGGGGDDLGANDELISFKDEGEQEEKSSENSSAERDLADVKSSLVNESETNQNSSSDSEAERRPPPRSESF
RDKSRESLEEAAKRODGGLFKGPPYPGYPFIMIPDLTSPYLPNGSLSPTARTYLQMKWPLLDVQAGSLQSRQALK
DARSPSPAHIVSNKVPVVQHPHHVHPLTPLITYSNEHFTPGNPPPHLPADVDPKTGIPRPPHPPDISPYYPLSPG
TVGQIPHPLGWLVPQQGQPVYPITTGGFRHPYPTALTVNASMSRFPPHMVPPHHTLHTTGIPHPAIVTPTVKQES
SQSDVGSLHSSKHQDSKKEEEKKKPHIKKPLNAFMLYMKEMRAKVVAECTLKESAAINQILGRRWHALSREEQAK
YYELARKERQLHMQLYPGWSARDNYGKKKKRKRDKQPGETNEHSECFLNPCLSLPPITDLSAPKKCRARFGLDQQ
NNWCGPCRRKKKCVRYIQGEGSCLSPPSSDGSLLDSPPPSPNLLGSPPRDAKSQTEQTQPLSLSLKPDPLAHLSM
MPPPPALLLAEATHKASALCPNGALDLPPAALQPAAPSSSIAQPSTSSLHSHSSLAGTQPQPLSLVTKSLE″
>NM_001146283.2 Homo sapiens transcription
factor 7 like 2 (TCF7L2), transcript variant 3
(SEQ ID NO: 49)
GTCAATAATCTCCGCTCCCAGACTACTCCGTTCCTCCGGATTTCGATCCCCCTTTTTCTATCTGTCAATC
AGCGCCGCCTTTGAACTGAAAAGCTCTCAGTCTAACTTCAACTCACTCAAATCCGAGCGGCACGAGCACC
TCCTGTATCTTCGGCTTCCCCCCCCCTTTGCTCTTTATATCTGACTTCTTGTTGTTGTTGGTGTTTTTTT
TTTTTTTACCCCCCTTTTTTATTTATTATTTTTTTGCACATTGATCGGATCCTTGGGAACGAGAGAAAAA
AGAAACCCAAACTCACGCGTGCAGAAGATCTCCCCCCCCTTCCCCTCCCCTCCTCCCTCTTTTCCCCTCC
CCAGGAGAAAAAGACCCCCAAGCAGAAAAAAGTTCACCTTGGACTCGTCTTTTTCTTGCAATATTTTTTG
GGGGGGCAAAACTTTTTGGGGGTGATTTTTTTTGGCTTTTCTTCCTCCTTCATTTTTCTTCCAAAATTGC
TGCTGGTGGGTGAAAAAAAAATGCCGCAGCTGAACGGCGGTGGAGGGGATGACCTAGGCGCCAACGACGA
ACTGATTTCCTTCAAAGACGAGGGCGAACAGGAGGAGAAGAGCTCCGAAAACTCCTCGGCAGAGAGGGAT
TTAGCTGATGTCAAATCGTCTCTAGTCAATGAATCAGAAACGAATCAAAACAGCTCCTCCGATTCCGAGG
CGGAAAGACGGCCTCCGCCTCGCTCCGAAAGTTTCCGAGACAAATCCCGGGAAAGTTTGGAAGAAGCGGC
CAAGAGGCAAGATGGAGGGCTCTTTAAGGGGCCACCGTATCCCGGCTACCCCTTCATCATGATCCCCGAC
CTGACGAGCCCCTACCTCCCCAACGGATCGCTCTCGCCCACCGCCCGAACCTATCTCCAGATGAAATGGC
CACTGCTTGATGTCCAGGCAGGGAGCCTCCAGAGTAGACAAGCCCTCAAGGATGCCCGGTCCCCATCACC
GGCACACATTGTCAGCCCCCTCCCTTGCTGCACTCAGGGACATGACTGTCAGCACTTCTACCCCCCCTCA
GACTTCACTGTCAGCACTCAAGTCTTCAGGGACATGAAAAGGAGCCACTCCTTACAAAAAGTTGGGGAGC
CCTGGTGTATTGAGTCTAACAAAGTGCCAGTGGTGCAGCACCCTCACCATGTCCACCCCCTCACGCCTCT
TATCACGTACAGCAATGAACACTTCACGCCGGGAAACCCACCTCCACACTTACCAGCCGACGTAGACCCC
AAAACAGGAATCCCACGGCCTCCGCACCCTCCAGATATATCCCCGTATTACCCACTATCGCCTGGCACCG
TAGGACAAATCCCCCATCCGCTAGGATGGTTAGTACCACAGCAAGGTCAACCAGTGTACCCAATCACGAC
AGGAGGATTCAGACACCCCTACCCCACAGCTCTGACCGTCAATGCTTCCATGTCCAGGTTCCCTCCCCAT
ATGGTCCCACCACATCATACGCTACACACGACGGGCATTCCGCATCCGGCCATAGTCACACCAACAGTCA
AACAGGAATCGTCCCAGAGTGATGTCGGCTCACTCCATAGTTCAAAGCATCAGGACTCCAAAAAGGAAGA
AGAAAAGAAGAAGCCCCACATAAAGAAACCTCTTAATGCATTCATGTTGTATATGAAGGAAATGAGAGCA
AAGGTCGTAGCTGAGTGCACGTTGAAAGAAAGCGCGGCCATCAACCAGATCCTTGGGCGGAGGTGGCATG
CACTGTCCAGAGAAGAGCAAGCGAAATACTACGAGCTGGCCCGGAAGGAGCGACAGCTTCATATGCAACT
GTACCCCGGCTGGTCCGCGCGGGATAACTATGGAAAGAAGAAGAAGAGGAAAAGGGACAAGCAGCCGGGA
GAGACCAATGGAGAAAAAAAAAGTGCGTTCGCTACATACAAGGTGAAGGCAGCTGCCTCAGCCCACCCTC
TTCAGATGGAAGCTTACTAGATTCGCCTCCCCCCTCCCCGAACCTGCTAGGCTCCCCTCCCCGAGACGCC
AAGTCACAGACTGAGCAGACCCAGCCTCTGTCGCTGTCCCTGAAGCCCGACCCCCTGGCCCACCTGTCCA
TGATGCCTCCGCCACCCGCCCTCCTGCTCGCTGAGGCCACCCACAAGGCCTCCGCCCTCTGTCCCAACGG
GGCCCTGGACCTGCCCCCAGCCGCTTTGCAGCCTGCCGCCCCCTCCTCATCAATTGCACAGCCGTCGACT
TCTTCCTTACATTCCCACAGCTCCCTGGCCGGGACCCAGCCCCAGCCGCTGTCGCTCGTCACCAAGTCTT
TAGAATAGCTTTAGCGTCGTGAACCCCGCTGCTTTGTTTATGGTTTTGTTTCACTTTTCTTAATTTGCCC
CCCACCCCCACCTTGAAAGGTTTTGTTTTGTACTCTCTTAATTTTGTGCCATGTGGCTACATTAGTTGAT
GTTTATCGAGTTCATTGGTCAATATTTGACCCATTCTTATTTCAATTTCTCCTTTTAAATATGTAGATGA
GAGAAGAACCTCATGATTCTACCAAAATTTTTATCAACAGCTGTTTAAAGTCTTTGTAGCGTTTAAAAAA
TATATATATATACATAACTGTTATGTAGTTCGGATAGCTTAGTTTTAAAAGACTGATTAAAAAACAAAAA
GAAAAAAAAAGCAATTTTGAAGCAGCCCTCCAGAAGGAGTTGGTTCTGTATTATTTGTATTAAATACGAG
CTTGCGAACCAATCATTTTACATCTGGTTTTTAAACCGTAAGGGCACCATGAATGCAGTGCCGTTACTTT
TTTTTTTTTTTTCTGTGTGAAACAACTCTTATTGTGATGTTACTTGTTATTGTTTAAATGTACAGAAACA
AAGGGTAAAAATGTGTTAATATACCTTGTTCCATGGTGTTGTTCTTTTGGGGGGAGGGGACGCTACTCAA
CACTTAATAGAATCACAACGCTGTTGGGCCAGTAGTATTTATTGCTTTAGAGATTGCTTGTCGTACCTGT
ATGTCGTCCCTTTTTAAATATGTTTTCCTTTTTCTTGAAACTGTATAAAGTTTTTTTCCCCCTTAGCATA
AGCATCTTATATATAACAACTCATTTGTACAAGGTTTTTAAGTTTATATATAAAATGTGTATATATATTT
TTGTTTCCCCTTTTTGACTTTTTTTTTTCTGTATGAAACCCAGATGTCACCAAATGGACATTAATAGTTG
CATTAAGGATCAGTAGCATTAACAAAAGTTGCTTTAAAAGCCATTATGTAAAACAAGACTTGAAAATGAG
TGAGGGAATTTTAGCGACACTGTCTGAGCAGCAGTGGGAACCATCTTCGTTTCCCCTTTGAACTCCCAGT
GGGATGCCCTACCCTGCGCCCTTAGGACCCGGACTGACCGTGTACAAAACTTTACGTGCCAAAATTCTCA
GTGAATTTAGCTTTCTCCCTCTTTTTGATGCTGTAATTTTTGTTCATCATGTTTTGCTGTGATGTTACAT
AGGTAGATTTGTATGTAGTTTTAATGTCACCTATAACAAAATGTGTTTGGTAGCAGATTGTCCAGAAAGC
ATTTTAAATGAAGAGGTATAAACCCTTAAGGGCCAAAATTCTGTATATTAGATTACTCTTAAACGAAAAA
CCAGCTGCCGCTTTTATGTACACATATTACATACGAGTAGGCAGCAGACTTTAAAAATAAAAAAAACCTA
GGCATGTTGATGTTGCAAAATGCTGTATAAAGCTGAAACCTGTTCATTCAGTGCCATTGTAGTTGACATG
AAGCGATTGTAAAACTGTCTCCGATTTTTCTCTGGTTTATTAAAATGCTAACTATAACATTTTTTGTGAA
TACTTTGAATGTTTCCTAACAGTTGTGATGTTACTGTTCCGTTTTATGCTCTTATTCCAAGTTCATTTTT
AATGGTTTGGAAGCCATTTTTGTAATGAATAAATGTTCATGCTGTACAGTATCTGTAGCATGCCGTTCTG
GATTAATAAAAGCAACTTAGTATGTGCAGATAAA,
Protein sequence (SEQ ID NO: 50):
MPQLNGGGGDDLGANDELISFKDEGEQEEKSSENSSAERDLADVKSSLVNESETNONSSSDSEAERRPPPRSESF
RDKSRESLEEAAKRQDGGLFKGPPYPGYPFIMIPDLTSPYLPNGSLSPTARTYLQMKWPLLDVQAGSLQSRQALK
DARSPSPAHIVSPLPCCTQGHDCQHFYPPSDFTVSTQVFRDMKRSHSLQKVGEPWCIESNKVPVVQHPHHVHPLT
PLITYSNEHFTPGNPPPHLPADVDPKTGIPRPPHPPDISPYYPLSPGTVGQIPHPLGWLVPQQGQPVYPITTGGF
RHPYPTALTVNASMSRFPPHMVPPHHTLHTTGIPHPAIVTPTVKQESSQSDVGSLHSSKHQDSKKEEEKKKPHIK
KPLNAFMLYMKEMRAKVVAECTLKESAAINQILGRRWHALSREEQAKYYELARKERQLHMQLYPGWSARDNYGKK
KKRKRDKQPGETNGEKKSAFATYKVKAAASAHPLQMEAY
>NM_001146284.2 Homo sapiens transcription
factor 7 like 2 (TCF7L2), transcript variant 4
(SEQ ID NO: 51)
GTCAATAATCTCCGCTCCCAGACTACTCCGTTCCTCCGGATTTCGATCCCCCTTTTTCTATCTGTCAATC
AGCGCCGCCTTTGAACTGAAAAGCTCTCAGTCTAACTTCAACTCACTCAAATCCGAGCGGCACGAGCACC
TCCTGTATCTTCGGCTTCCCCCCCCCTTTGCTCTTTATATCTGACTTCTTGTTGTTGTTGGTGTTTTTTT
TTTTTTTACCCCCCTTTTTTATTTATTATTTTTTTGCACATTGATCGGATCCTTGGGAACGAGAGAAAAA
AGAAACCCAAACTCACGCGTGCAGAAGATCTCCCCCCCCTTCCCCTCCCCTCCTCCCTCTTTTCCCCTCC
CCAGGAGAAAAAGACCCCCAAGCAGAAAAAAGTTCACCTTGGACTCGTCTTTTTCTTGCAATATTTTTTG
GGGGGGCAAAACTTTTTGGGGGTGATTTTTTTTGGCTTTTCTTCCTCCTTCATTTTTCTTCCAAAATTGC
TGCTGGTGGGTGAAAAAAAAATGCCGCAGCTGAACGGCGGTGGAGGGGATGACCTAGGCGCCAACGACGA
ACTGATTTCCTTCAAAGACGAGGGCGAACAGGAGGAGAAGAGCTCCGAAAACTCCTCGGCAGAGAGGGAT
TTAGCTGATGTCAAATCGTCTCTAGTCAATGAATCAGAAACGAATCAAAACAGCTCCTCCGATTCCGAGG
CGGAAAGACGGCCTCCGCCTCGCTCCGAAAGTTTCCGAGACAAATCCCGGGAAAGTTTGGAAGAAGCGGC
CAAGAGGCAAGATGGAGGGCTCTTTAAGGGGCCACCGTATCCCGGCTACCCCTTCATCATGATCCCCGAC
CTGACGAGCCCCTACCTCCCCAACGGATCGCTCTCGCCCACCGCCCGAACCTATCTCCAGATGAAATGGC
CACTGCTTGATGTCCAGGCAGGGAGCCTCCAGAGTAGACAAGCCCTCAAGGATGCCCGGTCCCCATCACC
GGCACACATTGTCTCTAACAAAGTGCCAGTGGTGCAGCACCCTCACCATGTCCACCCCCTCACGCCTCTT
ATCACGTACAGCAATGAACACTTCACGCCGGGAAACCCACCTCCACACTTACCAGCCGACGTAGACCCCA
AAACAGGAATCCCACGGCCTCCGCACCCTCCAGATATATCCCCGTATTACCCACTATCGCCTGGCACCGT
AGGACAAATCCCCCATCCGCTAGGATGGCAAGGTCAACCAGTGTACCCAATCACGACAGGAGGATTCAGA
CACCCCTACCCCACAGCTCTGACCGTCAATGCTTCCATGTCCAGGTTCCCTCCCCATATGGTCCCACCAC
ATCATACGCTACACACGACGGGCATTCCGCATCCGGCCATAGTCACACCAACAGTCAAACAGGAATCGTC
CCAGAGTGATGTCGGCTCACTCCATAGTTCAAAGCATCAGGACTCCAAAAAGGAAGAAGAAAAGAAGAAG
CCCCACATAAAGAAACCTCTTAATGCATTCATGTTGTATATGAAGGAAATGAGAGCAAAGGTCGTAGCTG
AGTGCACGTTGAAAGAAAGCGCGGCCATCAACCAGATCCTTGGGCGGAGGTGGCATGCACTGTCCAGAGA
AGAGCAAGCGAAATACTACGAGCTGGCCCGGAAGGAGCGACAGCTTCATATGCAACTGTACCCCGGCTGG
TCCGCGCGGGATAACTATGGAAAGAAGAAGAAGAGGAAAAGGGACAAGCAGCCGGGAGAGACCAATGAAC
ACAGCGAATGTTTCCTAAATCCTTGCCTTTCACTTCCTCCGATTACAGGAGAAAAAAAAAGTGCGTTCGC
TACATACAAGGTGAAGGCAGCTGCCTCAGCCCACCCTCTTCAGATGGAAGCTTACTAGATTCGCCTCCCC
CCTCCCCGAACCTGCTAGGCTCCCCTCCCCGAGACGCCAAGTCACAGACTGAGCAGACCCAGCCTCTGTC
GCTGTCCCTGAAGCCCGACCCCCTGGCCCACCTGTCCATGATGCCTCCGCCACCCGCCCTCCTGCTCGCT
GAGGCCACCCACAAGGCCTCCGCCCTCTGTCCCAACGGGGCCCTGGACCTGCCCCCAGCCGCTTTGCAGC
CTGCCGCCCCCTCCTCATCAATTGCACAGCCGTCGACTTCTTCCTTACATTCCCACAGCTCCCTGGCCGG
GACCCAGCCCCAGCCGCTGTCGCTCGTCACCAAGTCTTTAGAATAGCTTTAGCGTCGTGAACCCCGCTGC
TTTGTTTATGGTTTTGTTTCACTTTTCTTAATTTGCCCCCCACCCCCACCTTGAAAGGTTTTGTTTTGTA
CTCTCTTAATTTTGTGCCATGTGGCTACATTAGTTGATGTTTATCGAGTTCATTGGTCAATATTTGACCC
ATTCTTATTTCAATTTCTCCTTTTAAATATGTAGATGAGAGAAGAACCTCATGATTCTACCAAAATTTTT
ATCAACAGCTGTTTAAAGTCTTTGTAGCGTTTAAAAAATATATATATATACATAACTGTTATGTAGTTCG
GATAGCTTAGTTTTAAAAGACTGATTAAAAAACAAAAAGAAAAAAAAAGCAATTTTGAAGCAGCCCTCCA
GAAGGAGTTGGTTCTGTATTATTTGTATTAAATACGAGCTTGCGAACCAATCATTTTACATCTGGTTTTT
AAACCGTAAGGGCACCATGAATGCAGTGCCGTTACTTTTTTTTTTTTTTTCTGTGTGAAACAACTCTTAT
TGTGATGTTACTTGTTATTGTTTAAATGTACAGAAACAAAGGGTAAAAATGTGTTAATATACCTTGTTCC
ATGGTGTTGTTCTTTTGGGGGGAGGGGACGCTACTCAACACTTAATAGAATCACAACGCTGTTGGGCCAG
TAGTATTTATTGCTTTAGAGATTGCTTGTCGTACCTGTATGTCGTCCCTTTTTAAATATGTTTTCCTTTT
TCTTGAAACTGTATAAAGTTTTTTTCCCCCTTAGCATAAGCATCTTATATATAACAACTCATTTGTACAA
GGTTTTTAAGTTTATATATAAAATGTGTATATATATTTTTGTTTCCCCTTTTTGACTTTTTTTTTTCTGT
ATGAAACCCAGATGTCACCAAATGGACATTAATAGTTGCATTAAGGATCAGTAGCATTAACAAAAGTTGC
TTTAAAAGCCATTATGTAAAACAAGACTTGAAAATGAGTGAGGGAATTTTAGCGACACTGTCTGAGCAGC
AGTGGGAACCATCTTCGTTTCCCCTTTGAACTCCCAGTGGGATGCCCTACCCTGCGCCCTTAGGACCCGG
ACTGACCGTGTACAAAACTTTACGTGCCAAAATTCTCAGTGAATTTAGCTTTCTCCCTCTTTTTGATGCT
GTAATTTTTGTTCATCATGTTTTGCTGTGATGTTACATAGGTAGATTTGTATGTAGTTTTAATGTCACCT
ATAACAAAATGTGTTTGGTAGCAGATTGTCCAGAAAGCATTTTAAATGAAGAGGTATAAACCCTTAAGGG
CCAAAATTCTGTATATTAGATTACTCTTAAACGAAAAACCAGCTGCCGCTTTTATGTACACATATTACAT
ACGAGTAGGCAGCAGACTTTAAAAATAAAAAAAACCTAGGCATGTTGATGTTGCAAAATGCTGTATAAAG
CTGAAACCTGTTCATTCAGTGCCATTGTAGTTGACATGAAGCGATTGTAAAACTGTCTCCGATTTTTCTC
TGGTTTATTAAAATGCTAACTATAACATTTTTTGTGAATACTTTGAATGTTTCCTAACAGTTGTGATGTT
ACTGTTCCGTTTTATGCTCTTATTCCAAGTTCATTTTTAATGGTTTGGAAGCCATTTTTGTAATGAATAA
ATGTTCATGCTGTACAGTATCTGTAGCATGCCGTTCTGGATTAATAAAAGCAACTTAGTATGTGCAGATA
AA,
Protein sequence (SEQ ID NO: 52):
MPQLNGGGGDDLGANDELISFKDEGEQEEKSSENSSAERDLADVKSSLVNESETNONSSSDSEAERRPPPRSESF
RDKSRESLEEAAKRQDGGLFKGPPYPGYPFIMIPDLTSPYLPNGSLSPTARTYLQMKWPLLDVQAGSLQSRQALK
DARSPSPAHIVSNKVPVVQHPHHVHPLTPLITYSNEHFTPGNPPPHLPADVDPKTGIPRPPHPPDISPYYPLSPG
TVGQIPHPLGWQGQPVYPITTGGFRHPYPTALTVNASMSRFPPHMVPPHHTLHTTGIPHPAIVTPTVKQESSQSD
VGSLHSSKHQDSKKEEEKKKPHIKKPLNAFMLYMKEMRAKVVAECTLKESAAINQILGRRWHALSREEQAKYYEL
ARKERQLHMQLYPGWSARDNYGKKKKRKRDKQPGETNEHSECFLNPCLSLPPITGEKKSAFATYKVKAAASAHPL
QMEAY
>NM_001146285.2 Homo sapiens transcription
factor 7 like 2 (TCF7L2), transcript variant 5
(SEQ ID NO: 53)
GTCAATAATCTCCGCTCCCAGACTACTCCGTTCCTCCGGATTTCGATCCCCCTTTTTCTATCTGTCAATC
AGCGCCGCCTTTGAACTGAAAAGCTCTCAGTCTAACTTCAACTCACTCAAATCCGAGCGGCACGAGCACC
TCCTGTATCTTCGGCTTCCCCCCCCCTTTGCTCTTTATATCTGACTTCTTGTTGTTGTTGGTGTTTTTTT
TTTTTTTACCCCCCTTTTTTATTTATTATTTTTTTGCACATTGATCGGATCCTTGGGAACGAGAGAAAAA
AGAAACCCAAACTCACGCGTGCAGAAGATCTCCCCCCCCTTCCCCTCCCCTCCTCCCTCTTTTCCCCTCC
CCAGGAGAAAAAGACCCCCAAGCAGAAAAAAGTTCACCTTGGACTCGTCTTTTTCTTGCAATATTTTTTG
GGGGGGCAAAACTTTTTGGGGGTGATTTTTTTTGGCTTTTCTTCCTCCTTCATTTTTCTTCCAAAATTGC
TGCTGGTGGGTGAAAAAAAAATGCCGCAGCTGAACGGCGGTGGAGGGGATGACCTAGGCGCCAACGACGA
ACTGATTTCCTTCAAAGACGAGGGCGAACAGGAGGAGAAGAGCTCCGAAAACTCCTCGGCAGAGAGGGAT
TTAGCTGATGTCAAATCGTCTCTAGTCAATGAATCAGAAACGAATCAAAACAGCTCCTCCGATTCCGAGG
CGGAAAGACGGCCTCCGCCTCGCTCCGAAAGTTTCCGAGACAAATCCCGGGAAAGTTTGGAAGAAGCGGC
CAAGAGGCAAGATGGAGGGCTCTTTAAGGGGCCACCGTATCCCGGCTACCCCTTCATCATGATCCCCGAC
CTGACGAGCCCCTACCTCCCCAACGGATCGCTCTCGCCCACCGCCCGAACCTATCTCCAGATGAAATGGC
CACTGCTTGATGTCCAGGCAGGGAGCCTCCAGAGTAGACAAGCCCTCAAGGATGCCCGGTCCCCATCACC
GGCACACATTGTCTCTAACAAAGTGCCAGTGGTGCAGCACCCTCACCATGTCCACCCCCTCACGCCTCTT
ATCACGTACAGCAATGAACACTTCACGCCGGGAAACCCACCTCCACACTTACCAGCCGACGTAGACCCCA
AAACAGGAATCCCACGGCCTCCGCACCCTCCAGATATATCCCCGTATTACCCACTATCGCCTGGCACCGT
AGGACAAATCCCCCATCCGCTAGGATGGTTAGTACCACAGCAAGGTCAACCAGTGTACCCAATCACGACA
GGAGGATTCAGACACCCCTACCCCACAGCTCTGACCGTCAATGCTTCCATGTCCAGGTTCCCTCCCCATA
TGGTCCCACCACATCATACGCTACACACGACGGGCATTCCGCATCCGGCCATAGTCACACCAACAGTCAA
ACAGGAATCGTCCCAGAGTGATGTCGGCTCACTCCATAGTTCAAAGCATCAGGACTCCAAAAAGGAAGAA
GAAAAGAAGAAGCCCCACATAAAGAAACCTCTTAATGCATTCATGTTGTATATGAAGGAAATGAGAGCAA
AGGTCGTAGCTGAGTGCACGTTGAAAGAAAGCGCGGCCATCAACCAGATCCTTGGGCGGAGGTGGCATGC
ACTGTCCAGAGAAGAGCAAGCGAAATACTACGAGCTGGCCCGGAAGGAGCGACAGCTTCATATGCAACTG
TACCCCGGCTGGTCCGCGCGGGATAACTATGGAAAGAAGAAGAAGAGGAAAAGGGACAAGCAGCCGGGAG
AGACCAATGACCTGAGCGCTCCTAAGAAATGCCGAGCGCGCTTTGGCCTTGATCAACAGAATAACTGGTG
CGGCCCTTGCAGGAGAAAAAAAAAGTGCGTTCGCTACATACAAGGTGAAGGCAGCTGCCTCAGCCCACCC
TCTTCAGATGGAAGCTTACTAGATTCGCCTCCCCCCTCCCCGAACCTGCTAGGCTCCCCTCCCCGAGACG
CCAAGTCACAGACTGAGCAGACCCAGCCTCTGTCGCTGTCCCTGAAGCCCGACCCCCTGGCCCACCTGTC
CATGATGCCTCCGCCACCCGCCCTCCTGCTCGCTGAGGCCACCCACAAGGCCTCCGCCCTCTGTCCCAAC
GGGGCCCTGGACCTGCCCCCAGCCGCTTTGCAGCCTGCCGCCCCCTCCTCATCAATTGCACAGCCGTCGA
CTTCTTCCTTACATTCCCACAGCTCCCTGGCCGGGACCCAGCCCCAGCCGCTGTCGCTCGTCACCAAGTC
TTTAGAATAGCTTTAGCGTCGTGAACCCCGCTGCTTTGTTTATGGTTTTGTTTCACTTTTCTTAATTTGC
CCCCCACCCCCACCTTGAAAGGTTTTGTTTTGTACTCTCTTAATTTTGTGCCATGTGGCTACATTAGTTG
ATGTTTATCGAGTTCATTGGTCAATATTTGACCCATTCTTATTTCAATTTCTCCTTTTAAATATGTAGAT
GAGAGAAGAACCTCATGATTCTACCAAAATTTTTATCAACAGCTGTTTAAAGTCTTTGTAGCGTTTAAAA
AATATATATATATACATAACTGTTATGTAGTTCGGATAGCTTAGTTTTAAAAGACTGATTAAAAAACAAA
AAGAAAAAAAAAGCAATTTTGAAGCAGCCCTCCAGAAGGAGTTGGTTCTGTATTATTTGTATTAAATACG
AGCTTGCGAACCAATCATTTTACATCTGGTTTTTAAACCGTAAGGGCACCATGAATGCAGTGCCGTTACT
TTTTTTTTTTTTTTCTGTGTGAAACAACTCTTATTGTGATGTTACTTGTTATTGTTTAAATGTACAGAAA
CAAAGGGTAAAAATGTGTTAATATACCTTGTTCCATGGTGTTGTTCTTTTGGGGGGAGGGGACGCTACTC
AACACTTAATAGAATCACAACGCTGTTGGGCCAGTAGTATTTATTGCTTTAGAGATTGCTTGTCGTACCT
GTATGTCGTCCCTTTTTAAATATGTTTTCCTTTTTCTTGAAACTGTATAAAGTTTTTTTCCCCCTTAGCA
TAAGCATCTTATATATAACAACTCATTTGTACAAGGTTTTTAAGTTTATATATAAAATGTGTATATATAT
TTTTGTTTCCCCTTTTTGACTTTTTTTTTTCTGTATGAAACCCAGATGTCACCAAATGGACATTAATAGT
TGCATTAAGGATCAGTAGCATTAACAAAAGTTGCTTTAAAAGCCATTATGTAAAACAAGACTTGAAAATG
AGTGAGGGAATTTTAGCGACACTGTCTGAGCAGCAGTGGGAACCATCTTCGTTTCCCCTTTGAACTCCCA
GTGGGATGCCCTACCCTGCGCCCTTAGGACCCGGACTGACCGTGTACAAAACTTTACGTGCCAAAATTCT
CAGTGAATTTAGCTTTCTCCCTCTTTTTGATGCTGTAATTTTTGTTCATCATGTTTTGCTGTGATGTTAC
ATAGGTAGATTTGTATGTAGTTTTAATGTCACCTATAACAAAATGTGTTTGGTAGCAGATTGTCCAGAAA
GCATTTTAAATGAAGAGGTATAAACCCTTAAGGGCCAAAATTCTGTATATTAGATTACTCTTAAACGAAA
AACCAGCTGCCGCTTTTATGTACACATATTACATACGAGTAGGCAGCAGACTTTAAAAATAAAAAAAACC
TAGGCATGTTGATGTTGCAAAATGCTGTATAAAGCTGAAACCTGTTCATTCAGTGCCATTGTAGTTGACA
TGAAGCGATTGTAAAACTGTCTCCGATTTTTCTCTGGTTTATTAAAATGCTAACTATAACATTTTTTGTG
AATACTTTGAATGTTTCCTAACAGTTGTGATGTTACTGTTCCGTTTTATGCTCTTATTCCAAGTTCATTT
TTAATGGTTTGGAAGCCATTTTTGTAATGAATAAATGTTCATGCTGTACAGTATCTGTAGCATGCCGTTC
TGGATTAATAAAAGCAACTTAGTATGTGCAGATAAA,
Protein sequence (SEQ ID NO: 54):
MPQLNGGGGDDLGANDELISFKDEGEQEEKSSENSSAERDLADVKSSLVNESETNONSSSDSEAERRPPPRSESF
RDKSRESLEEAAKRQDGGLFKGPPYPGYPFIMIPDLTSPYLPNGSLSPTARTYLQMKWPLLDVQAGSLQSRQALK
DARSPSPAHIVSNKVPVVQHPHHVHPLTPLITYSNEHFTPGNPPPHLPADVDPKTGIPRPPHPPDISPYYPLSPG
TVGQIPHPLGWLVPQQGQPVYPITTGGFRHPYPTALTVNASMSRFPPHMVPPHHTLHTTGIPHPAIVTPTVKQES
SQSDVGSLHSSKHQDSKKEEEKKKPHIKKPLNAFMLYMKEMRAKVVAECTLKESAAINQILGRRWHALSREEQAK
YYELARKERQLHMQLYPGWSARDNYGKKKKRKRDKQPGETNDLSAPKKCRARFGLDQQNNWCGPCRRKKKCVRYI
QGEGSCLSPPSSDGSLLDSPPPSPNLLGSPPRDAKSQTEQTQPLSLSLKPDPLAHLSMMPPPPALLLAEATHKAS
ALCPNGALDLPPAALQPAAPSSSIAQPSTSSLHSHSSLAGT
QPQPLSLVTKSLE
>NM_001146286.2 Homo sapiens transcription
factor 7 like 2 (TCF7L2), transcript variant 6
(SEQ ID NO: 55)
GTCAATAATCTCCGCTCCCAGACTACTCCGTTCCTCCGGATTTCGATCCCCCTTTTTCTATCTGTCAATC
AGCGCCGCCTTTGAACTGAAAAGCTCTCAGTCTAACTTCAACTCACTCAAATCCGAGCGGCACGAGCACC
TCCTGTATCTTCGGCTTCCCCCCCCCTTTGCTCTTTATATCTGACTTCTTGTTGTTGTTGGTGTTTTTTT
TTTTTTTACCCCCCTTTTTTATTTATTATTTTTTTGCACATTGATCGGATCCTTGGGAACGAGAGAAAAA
AGAAACCCAAACTCACGCGTGCAGAAGATCTCCCCCCCCTTCCCCTCCCCTCCTCCCTCTTTTCCCCTCC
CCAGGAGAAAAAGACCCCCAAGCAGAAAAAAGTTCACCTTGGACTCGTCTTTTTCTTGCAATATTTTTTG
GGGGGGCAAAACTTTTTGGGGGTGATTTTTTTTGGCTTTTCTTCCTCCTTCATTTTTCTTCCAAAATTGC
TGCTGGTGGGTGAAAAAAAAATGCCGCAGCTGAACGGCGGTGGAGGGGATGACCTAGGCGCCAACGACGA
ACTGATTTCCTTCAAAGACGAGGGCGAACAGGAGGAGAAGAGCTCCGAAAACTCCTCGGCAGAGAGGGAT
TTAGCTGATGTCAAATCGTCTCTAGTCAATGAATCAGAAACGAATCAAAACAGCTCCTCCGATTCCGAGG
CGGAAAGACGGCCTCCGCCTCGCTCCGAAAGTTTCCGAGACAAATCCCGGGAAAGTTTGGAAGAAGCGGC
CAAGAGGCAAGATGGAGGGCTCTTTAAGGGGCCACCGTATCCCGGCTACCCCTTCATCATGATCCCCGAC
CTGACGAGCCCCTACCTCCCCAACGGATCGCTCTCGCCCACCGCCCGAACCTATCTCCAGATGAAATGGC
CACTGCTTGATGTCCAGGCAGGGAGCCTCCAGAGTAGACAAGCCCTCAAGGATGCCCGGTCCCCATCACC
GGCACACATTGTCTCTAACAAAGTGCCAGTGGTGCAGCACCCTCACCATGTCCACCCCCTCACGCCTCTT
ATCACGTACAGCAATGAACACTTCACGCCGGGAAACCCACCTCCACACTTACCAGCCGACGTAGACCCCA
AAACAGGAATCCCACGGCCTCCGCACCCTCCAGATATATCCCCGTATTACCCACTATCGCCTGGCACCGT
AGGACAAATCCCCCATCCGCTAGGATGGTTAGTACCACAGCAAGGTCAACCAGTGTACCCAATCACGACA
GGAGGATTCAGACACCCCTACCCCACAGCTCTGACCGTCAATGCTTCCATGTCCAGGTTCCCTCCCCATA
TGGTCCCACCACATCATACGCTACACACGACGGGCATTCCGCATCCGGCCATAGTCACACCAACAGTCAA
ACAGGAATCGTCCCAGAGTGATGTCGGCTCACTCCATAGTTCAAAGCATCAGGACTCCAAAAAGGAAGAA
GAAAAGAAGAAGCCCCACATAAAGAAACCTCTTAATGCATTCATGTTGTATATGAAGGAAATGAGAGCAA
AGGTCGTAGCTGAGTGCACGTTGAAAGAAAGCGCGGCCATCAACCAGATCCTTGGGCGGAGGTGGCATGC
ACTGTCCAGAGAAGAGCAAGCGAAATACTACGAGCTGGCCCGGAAGGAGCGACAGCTTCATATGCAACTG
TACCCCGGCTGGTCCGCGCGGGATAACTATGGAAAGAAGAAGAAGAGGAAAAGGGACAAGCAGCCGGGAG
AGACCAATGGAGAAAAAAAAAGTGCGTTCGCTACATACAAGGTGAAGGCAGCTGCCTCAGCCCACCCTCT
TCAGATGGAAGCTTACTAGATTCGCCTCCCCCCTCCCCGAACCTGCTAGGCTCCCCTCCCCGAGACGCCA
AGTCACAGACTGAGCAGACCCAGCCTCTGTCGCTGTCCCTGAAGCCCGACCCCCTGGCCCACCTGTCCAT
GATGCCTCCGCCACCCGCCCTCCTGCTCGCTGAGGCCACCCACAAGGCCTCCGCCCTCTGTCCCAACGGG
GCCCTGGACCTGCCCCCAGCCGCTTTGCAGCCTGCCGCCCCCTCCTCATCAATTGCACAGCCGTCGACTT
CTTCCTTACATTCCCACAGCTCCCTGGCCGGGACCCAGCCCCAGCCGCTGTCGCTCGTCACCAAGTCTTT
AGAATAGCTTTAGCGTCGTGAACCCCGCTGCTTTGTTTATGGTTTTGTTTCACTTTTCTTAATTTGCCCC
CCACCCCCACCTTGAAAGGTTTTGTTTTGTACTCTCTTAATTTTGTGCCATGTGGCTACATTAGTTGATG
TTTATCGAGTTCATTGGTCAATATTTGACCCATTCTTATTTCAATTTCTCCTTTTAAATATGTAGATGAG
AGAAGAACCTCATGATTCTACCAAAATTTTTATCAACAGCTGTTTAAAGTCTTTGTAGCGTTTAAAAAAT
ATATATATATACATAACTGTTATGTAGTTCGGATAGCTTAGTTTTAAAAGACTGATTAAAAAACAAAAAG
AAAAAAAAAGCAATTTTGAAGCAGCCCTCCAGAAGGAGTTGGTTCTGTATTATTTGTATTAAATACGAGC
TTGCGAACCAATCATTTTACATCTGGTTTTTAAACCGTAAGGGCACCATGAATGCAGTGCCGTTACTTTT
TTTTTTTTTTTCTGTGTGAAACAACTCTTATTGTGATGTTACTTGTTATTGTTTAAATGTACAGAAACAA
AGGGTAAAAATGTGTTAATATACCTTGTTCCATGGTGTTGTTCTTTTGGGGGGAGGGGACGCTACTCAAC
ACTTAATAGAATCACAACGCTGTTGGGCCAGTAGTATTTATTGCTTTAGAGATTGCTTGTCGTACCTGTA
TGTCGTCCCTTTTTAAATATGTTTTCCTTTTTCTTGAAACTGTATAAAGTTTTTTTCCCCCTTAGCATAA
GCATCTTATATATAACAACTCATTTGTACAAGGTTTTTAAGTTTATATATAAAATGTGTATATATATTTT
TGTTTCCCCTTTTTGACTTTTTTTTTTCTGTATGAAACCCAGATGTCACCAAATGGACATTAATAGTTGC
ATTAAGGATCAGTAGCATTAACAAAAGTTGCTTTAAAAGCCATTATGTAAAACAAGACTTGAAAATGAGT
GAGGGAATTTTAGCGACACTGTCTGAGCAGCAGTGGGAACCATCTTCGTTTCCCCTTTGAACTCCCAGTG
GGATGCCCTACCCTGCGCCCTTAGGACCCGGACTGACCGTGTACAAAACTTTACGTGCCAAAATTCTCAG
TGAATTTAGCTTTCTCCCTCTTTTTGATGCTGTAATTTTTGTTCATCATGTTTTGCTGTGATGTTACATA
GGTAGATTTGTATGTAGTTTTAATGTCACCTATAACAAAATGTGTTTGGTAGCAGATTGTCCAGAAAGCA
TTTTAAATGAAGAGGTATAAACCCTTAAGGGCCAAAATTCTGTATATTAGATTACTCTTAAACGAAAAAC
CAGCTGCCGCTTTTATGTACACATATTACATACGAGTAGGCAGCAGACTTTAAAAATAAAAAAAACCTAG
GCATGTTGATGTTGCAAAATGCTGTATAAAGCTGAAACCTGTTCATTCAGTGCCATTGTAGTTGACATGA
AGCGATTGTAAAACTGTCTCCGATTTTTCTCTGGTTTATTAAAATGCTAACTATAACATTTTTTGTGAAT
ACTTTGAATGTTTCCTAACAGTTGTGATGTTACTGTTCCGTTTTATGCTCTTATTCCAAGTTCATTTTTA
ATGGTTTGGAAGCCATTTTTGTAATGAATAAATGTTCATGCTGTACAGTATCTGTAGCATGCCGTTCTGG
ATTAATAAAAGCAACTTAGTATGTGCAGATAAA,
Protein sequence (SEQ ID NO: 56):
MPQLNGGGGDDLGANDELISFKDEGEQEEKSSENSSAERDLADVKSSLVNESETNQNSSSDSEAERRPPPRSESF
RDKSRESLEEAAKRQDGGLFKGPPYPGYPFIMIPDLTSPYLPNGSLSPTARTYLQMKWPLLDVQAGSLQSRQALK
DARSPSPAHIVSNKVPVVQHPHHVHPLTPLITYSNEHFTPGNPPPHLPADVDPKTGIPRPPHPPDISPYYPLSPG
TVGQIPHPLGWLVPQQGQPVYPITTGGFRHPYPTALTVNASMSRFPPHMVPPHHTLHTTGIPHPAIVTPTVKQES
SQSDVGSLHSSKHQDSKKEEEKKKPHIKKPLNAFMLYMKEMRAKVVAECTLKESAAINQILGRRWHALSREEQAK
YYELARKERQLHMQLYPGWSARDNYGKKKKRKRDKQPGETNGEKKSAFATYKVKAAASAHPLQMEAY
>NM_001198525.2 Homo sapiens transcription
factor 7 like 2 (TCF7L2), transcript variant 7
(SEQ ID NO: 57)
GTCAATAATCTCCGCTCCCAGACTACTCCGTTCCTCCGGATTTCGATCCCCCTTTTTCTATCTGTCAATC
AGCGCCGCCTTTGAACTGAAAAGCTCTCAGTCTAACTTCAACTCACTCAAATCCGAGCGGCACGAGCACC
TCCTGTATCTTCGGCTTCCCCCCCCCTTTGCTCTTTATATCTGACTTCTTGTTGTTGTTGGTGTTTTTTT
TTTTTTTACCCCCCTTTTTTATTTATTATTTTTTTGCACATTGATCGGATCCTTGGGAACGAGAGAAAAA
AGAAACCCAAACTCACGCGTGCAGAAGATCTCCCCCCCCTTCCCCTCCCCTCCTCCCTCTTTTCCCCTCC
CCAGGAGAAAAAGACCCCCAAGCAGAAAAAAGTTCACCTTGGACTCGTCTTTTTCTTGCAATATTTTTTG
GGGGGGCAAAACTTTTTGGGGGTGATTTTTTTTGGCTTTTCTTCCTCCTTCATTTTTCTTCCAAAATTGC
TGCTGGTGGGTGAAAAAAAAATGCCGCAGCTGAACGGCGGTGGAGGGGATGACCTAGGCGCCAACGACGA
ACTGATTTCCTTCAAAGACGAGGGCGAACAGGAGGAGAAGAGCTCCGAAAACTCCTCGGCAGAGAGGGAT
TTAGCTGATGTCAAATCGTCTCTAGTCAATGAATCAGAAACGAATCAAAACAGCTCCTCCGATTCCGAGG
CGGAAAGACGGCCTCCGCCTCGCTCCGAAAGTTTCCGAGACAAATCCCGGGAAAGTTTGGAAGAAGCGGC
CAAGAGGCAAGATGGAGGGCTCTTTAAGGGGCCACCGTATCCCGGCTACCCCTTCATCATGATCCCCGAC
CTGACGAGCCCCTACCTCCCCAACGGATCGCTCTCGCCCACCGCCCGAACCTATCTCCAGATGAAATGGC
CACTGCTTGATGTCCAGGCAGGGAGCCTCCAGAGTAGACAAGCCCTCAAGGATGCCCGGTCCCCATCACC
GGCACACATTGTCTCTAACAAAGTGCCAGTGGTGCAGCACCCTCACCATGTCCACCCCCTCACGCCTCTT
ATCACGTACAGCAATGAACACTTCACGCCGGGAAACCCACCTCCACACTTACCAGCCGACGTAGACCCCA
AAACAGGAATCCCACGGCCTCCGCACCCTCCAGATATATCCCCGTATTACCCACTATCGCCTGGCACCGT
AGGACAAATCCCCCATCCGCTAGGATGGTTAGTACCACAGCAAGGTCAACCAGTGTACCCAATCACGACA
GGAGGATTCAGACACCCCTACCCCACAGCTCTGACCGTCAATGCTTCCATGTCCAGCTTTCTGTCTTCTA
GGTTCCCTCCCCATATGGTCCCACCACATCATACGCTACACACGACGGGCATTCCGCATCCGGCCATAGT
CACACCAACAGTCAAACAGGAATCGTCCCAGAGTGATGTCGGCTCACTCCATAGTTCAAAGCATCAGGAC
TCCAAAAAGGAAGAAGAAAAGAAGAAGCCCCACATAAAGAAACCTCTTAATGCATTCATGTTGTATATGA
AGGAAATGAGAGCAAAGGTCGTAGCTGAGTGCACGTTGAAAGAAAGCGCGGCCATCAACCAGATCCTTGG
GCGGAGGTGGCATGCACTGTCCAGAGAAGAGCAAGCGAAATACTACGAGCTGGCCCGGAAGGAGCGACAG
CTTCATATGCAACTGTACCCCGGCTGGTCCGCGCGGGATAACTATGGAAAGAAGAAGAAGAGGAAAAGGG
ACAAGCAGCCGGGAGAGACCAATGAACACAGCGAATGTTTCCTAAATCCTTGCCTTTCACTTCCTCCGAT
TACAGACCTGAGCGCTCCTAAGAAATGCCGAGCGCGCTTTGGCCTTGATCAACAGAATAACTGGTGCGGC
CCTTGCAGATGCAAATACTCCAAAGAAGTGTCGGGCACTGTTCGGGCTTGACCGACAGACTTTATGGTGC
AAACCGTGCAGGAGAAAAAAAAAGTGCGTTCGCTACATACAAGGTGAAGGCAGCTGCCTCAGCCCACCCT
CTTCAGATGGAAGCTTACTAGATTCGCCTCCCCCCTCCCCGAACCTGCTAGGCTCCCCTCCCCGAGACGC
CAAGTCACAGACTGAGCAGACCCAGCCTCTGTCGCTGTCCCTGAAGCCCGACCCCCTGGCCCACCTGTCC
ATGATGCCTCCGCCACCCGCCCTCCTGCTCGCTGAGGCCACCCACAAGGCCTCCGCCCTCTGTCCCAACG
GGGCCCTGGACCTGCCCCCAGCCGCTTTGCAGCCTGCCGCCCCCTCCTCATCAATTGCACAGCCGTCGAC
TTCTTCCTTACATTCCCACAGCTCCCTGGCCGGGACCCAGCCCCAGCCGCTGTCGCTCGTCACCAAGTCT
TTAGAATAGCTTTAGCGTCGTGAACCCCGCTGCTTTGTTTATGGTTTTGTTTCACTTTTCTTAATTTGCC
CCCCACCCCCACCTTGAAAGGTTTTGTTTTGTACTCTCTTAATTTTGTGCCATGTGGCTACATTAGTTGA
TGTTTATCGAGTTCATTGGTCAATATTTGACCCATTCTTATTTCAATTTCTCCTTTTAAATATGTAGATG
AGAGAAGAACCTCATGATTCTACCAAAATTTTTATCAACAGCTGTTTAAAGTCTTTGTAGCGTTTAAAAA
ATATATATATATACATAACTGTTATGTAGTTCGGATAGCTTAGTTTTAAAAGACTGATTAAAAAACAAAA
AGAAAAAAAAAGCAATTTTGAAGCAGCCCTCCAGAAGGAGTTGGTTCTGTATTATTTGTATTAAATACGA
GCTTGCGAACCAATCATTTTACATCTGGTTTTTAAACCGTAAGGGCACCATGAATGCAGTGCCGTTACTT
TTTTTTTTTTTTTCTGTGTGAAACAACTCTTATTGTGATGTTACTTGTTATTGTTTAAATGTACAGAAAC
AAAGGGTAAAAATGTGTTAATATACCTTGTTCCATGGTGTTGTTCTTTTGGGGGGAGGGGACGCTACTCA
ACACTTAATAGAATCACAACGCTGTTGGGCCAGTAGTATTTATTGCTTTAGAGATTGCTTGTCGTACCTG
TATGTCGTCCCTTTTTAAATATGTTTTCCTTTTTCTTGAAACTGTATAAAGTTTTTTTCCCCCTTAGCAT
AAGCATCTTATATATAACAACTCATTTGTACAAGGTTTTTAAGTTTATATATAAAATGTGTATATATATT
TTTGTTTCCCCTTTTTGACTTTTTTTTTTCTGTATGAAACCCAGATGTCACCAAATGGACATTAATAGTT
GCATTAAGGATCAGTAGCATTAACAAAAGTTGCTTTAAAAGCCATTATGTAAAACAAGACTTGAAAATGA
GTGAGGGAATTTTAGCGACACTGTCTGAGCAGCAGTGGGAACCATCTTCGTTTCCCCTTTGAACTCCCAG
TGGGATGCCCTACCCTGCGCCCTTAGGACCCGGACTGACCGTGTACAAAACTTTACGTGCCAAAATTCTC
AGTGAATTTAGCTTTCTCCCTCTTTTTGATGCTGTAATTTTTGTTCATCATGTTTTGCTGTGATGTTACA
TAGGTAGATTTGTATGTAGTTTTAATGTCACCTATAACAAAATGTGTTTGGTAGCAGATTGTCCAGAAAG
CATTTTAAATGAAGAGGTATAAACCCTTAAGGGCCAAAATTCTGTATATTAGATTACTCTTAAACGAAAA
ACCAGCTGCCGCTTTTATGTACACATATTACATACGAGTAGGCAGCAGACTTTAAAAATAAAAAAAACCT
AGGCATGTTGATGTTGCAAAATGCTGTATAAAGCTGAAACCTGTTCATTCAGTGCCATTGTAGTTGACAT
GAAGCGATTGTAAAACTGTCTCCGATTTTTCTCTGGTTTATTAAAATGCTAACTATAACATTTTTTGTGA
ATACTTTGAATGTTTCCTAACAGTTGTGATGTTACTGTTCCGTTTTATGCTCTTATTCCAAGTTCATTTT
TAATGGTTTGGAAGCCATTTTTGTAATGAATAAATGTTCATGCTGTACAGTATCTGTAGCATGCCGTTCT
GGATTAATAAAAGCAACTTAGTATGTGCAGATAAA,
Protein sequence (SEQ ID NO: 58):
MPQLNGGGGDDLGANDELISFKDEGEQEEKSSENSSAERDLADVKSSLVNESETNONSSSDSEAERRPPPRSESF
RDKSRESLEEAAKRQDGGLFKGPPYPGYPFIMIPDLTSPYLPNGSLSPTARTYLQMKWPLLDVQAGSLQSRQALK
DARSPSPAHIVSNKVPVVQHPHHVHPLTPLITYSNEHFTPGNPPPHLPADVDPKTGIPRPPHPPDISPYYPLSPG
TVGQIPHPLGWLVPQQGQPVYPITTGGFRHPYPTALTVNASMSSFLSSRFPPHMVPPHHTLHTTGIPHPAIVTPT
VKQESSQSDVGSLHSSKHQDSKKEEEKKKPHIKKPLNAFMLYMKEMRAKVVAECTLKESAAINQILGRRWHALSR
EEQAKYYELARKERQLHMQLYPGWSARDNYGKKKKRKRDKQPGETNEHSECFLNPCLSLPPITDLSAPKKCRARF
GLDQQNNWCGPCRCKYSKEVSGTVRA
>NM_001198526.2 Homo sapiens transcription
factor 7 like 2 (TCF7L2), transcript variant 8
(SEQ ID NO: 59)
GTCAATAATCTCCGCTCCCAGACTACTCCGTTCCTCCGGATTTCGATCCCCCTTTTTCTATCTGTCAATC
AGCGCCGCCTTTGAACTGAAAAGCTCTCAGTCTAACTTCAACTCACTCAAATCCGAGCGGCACGAGCACC
TCCTGTATCTTCGGCTTCCCCCCCCCTTTGCTCTTTATATCTGACTTCTTGTTGTTGTTGGTGTTTTTTT
TTTTTTTACCCCCCTTTTTTATTTATTATTTTTTTGCACATTGATCGGATCCTTGGGAACGAGAGAAAAA
AGAAACCCAAACTCACGCGTGCAGAAGATCTCCCCCCCCTTCCCCTCCCCTCCTCCCTCTTTTCCCCTCC
CCAGGAGAAAAAGACCCCCAAGCAGAAAAAAGTTCACCTTGGACTCGTCTTTTTCTTGCAATATTTTTTG
GGGGGGCAAAACTTTTTGGGGGTGATTTTTTTTGGCTTTTCTTCCTCCTTCATTTTTCTTCCAAAATTGC
TGCTGGTGGGTGAAAAAAAAATGCCGCAGCTGAACGGCGGTGGAGGGGATGACCTAGGCGCCAACGACGA
ACTGATTTCCTTCAAAGACGAGGGCGAACAGGAGGAGAAGAGCTCCGAAAACTCCTCGGCAGAGAGGGAT
TTAGCTGATGTCAAATCGTCTCTAGTCAATGAATCAGAAACGAATCAAAACAGCTCCTCCGATTCCGAGG
CGGAAAGACGGCCTCCGCCTCGCTCCGAAAGTTTCCGAGACAAATCCCGGGAAAGTTTGGAAGAAGCGGC
CAAGAGGCAAGATGGAGGGCTCTTTAAGGGGCCACCGTATCCCGGCTACCCCTTCATCATGATCCCCGAC
CTGACGAGCCCCTACCTCCCCAACGGATCGCTCTCGCCCACCGCCCGAACCTATCTCCAGATGAAATGGC
CACTGCTTGATGTCCAGGCAGGGAGCCTCCAGAGTAGACAAGCCCTCAAGGATGCCCGGTCCCCATCACC
GGCACACATTGTCTCTAACAAAGTGCCAGTGGTGCAGCACCCTCACCATGTCCACCCCCTCACGCCTCTT
ATCACGTACAGCAATGAACACTTCACGCCGGGAAACCCACCTCCACACTTACCAGCCGACGTAGACCCCA
AAACAGGAATCCCACGGCCTCCGCACCCTCCAGATATATCCCCGTATTACCCACTATCGCCTGGCACCGT
AGGACAAATCCCCCATCCGCTAGGATGGTTAGTACCACAGCAAGGTCAACCAGTGTACCCAATCACGACA
GGAGGATTCAGACACCCCTACCCCACAGCTCTGACCGTCAATGCTTCCATGTCCAGGTTCCCTCCCCATA
TGGTCCCACCACATCATACGCTACACACGACGGGCATTCCGCATCCGGCCATAGTCACACCAACAGTCAA
ACAGGAATCGTCCCAGAGTGATGTCGGCTCACTCCATAGTTCAAAGCATCAGGACTCCAAAAAGGAAGAA
GAAAAGAAGAAGCCCCACATAAAGAAACCTCTTAATGCATTCATGTTGTATATGAAGGAAATGAGAGCAA
AGGTCGTAGCTGAGTGCACGTTGAAAGAAAGCGCGGCCATCAACCAGATCCTTGGGCGGAGGTGGCATGC
ACTGTCCAGAGAAGAGCAAGCGAAATACTACGAGCTGGCCCGGAAGGAGCGACAGCTTCATATGCAACTG
TACCCCGGCTGGTCCGCGCGGGATAACTATGGAAAGAAGAAGAAGAGGAAAAGGGACAAGCAGCCGGGAG
AGACCAATGATGCAAATACTCCAAAGAAGTGTCGGGCACTGTTCGGGCTTGACCGACAGACTTTATGGTG
CAAACCGTGCAGGAGAAAAAAAAAGTGCGTTCGCTACATACAAGGTGAAGGCAGCTGCCTCAGCCCACCC
TCTTCAGATGGAAGCTTACTAGATTCGCCTCCCCCCTCCCCGAACCTGCTAGGCTCCCCTCCCCGAGACG
CCAAGTCACAGACTGAGCAGACCCAGCCTCTGTCGCTGTCCCTGAAGCCCGACCCCCTGGCCCACCTGTC
CATGATGCCTCCGCCACCCGCCCTCCTGCTCGCTGAGGCCACCCACAAGGCCTCCGCCCTCTGTCCCAAC
GGGGCCCTGGACCTGCCCCCAGCCGCTTTGCAGCCTGCCGCCCCCTCCTCATCAATTGCACAGCCGTCGA
CTTCTTCCTTACATTCCCACAGCTCCCTGGCCGGGACCCAGCCCCAGCCGCTGTCGCTCGTCACCAAGTC
TTTAGAATAGCTTTAGCGTCGTGAACCCCGCTGCTTTGTTTATGGTTTTGTTTCACTTTTCTTAATTTGC
CCCCCACCCCCACCTTGAAAGGTTTTGTTTTGTACTCTCTTAATTTTGTGCCATGTGGCTACATTAGTTG
ATGTTTATCGAGTTCATTGGTCAATATTTGACCCATTCTTATTTCAATTTCTCCTTTTAAATATGTAGAT
GAGAGAAGAACCTCATGATTCTACCAAAATTTTTATCAACAGCTGTTTAAAGTCTTTGTAGCGTTTAAAA
AATATATATATATACATAACTGTTATGTAGTTCGGATAGCTTAGTTTTAAAAGACTGATTAAAAAACAAA
AAGAAAAAAAAAGCAATTTTGAAGCAGCCCTCCAGAAGGAGTTGGTTCTGTATTATTTGTATTAAATACG
AGCTTGCGAACCAATCATTTTACATCTGGTTTTTAAACCGTAAGGGCACCATGAATGCAGTGCCGTTACT
TTTTTTTTTTTTTTCTGTGTGAAACAACTCTTATTGTGATGTTACTTGTTATTGTTTAAATGTACAGAAA
CAAAGGGTAAAAATGTGTTAATATACCTTGTTCCATGGTGTTGTTCTTTTGGGGGGAGGGGACGCTACTC
AACACTTAATAGAATCACAACGCTGTTGGGCCAGTAGTATTTATTGCTTTAGAGATTGCTTGTCGTACCT
GTATGTCGTCCCTTTTTAAATATGTTTTCCTTTTTCTTGAAACTGTATAAAGTTTTTTTCCCCCTTAGCA
TAAGCATCTTATATATAACAACTCATTTGTACAAGGTTTTTAAGTTTATATATAAAATGTGTATATATAT
TTTTGTTTCCCCTTTTTGACTTTTTTTTTTCTGTATGAAACCCAGATGTCACCAAATGGACATTAATAGT
TGCATTAAGGATCAGTAGCATTAACAAAAGTTGCTTTAAAAGCCATTATGTAAAACAAGACTTGAAAATG
AGTGAGGGAATTTTAGCGACACTGTCTGAGCAGCAGTGGGAACCATCTTCGTTTCCCCTTTGAACTCCCA
GTGGGATGCCCTACCCTGCGCCCTTAGGACCCGGACTGACCGTGTACAAAACTTTACGTGCCAAAATTCT
CAGTGAATTTAGCTTTCTCCCTCTTTTTGATGCTGTAATTTTTGTTCATCATGTTTTGCTGTGATGTTAC
ATAGGTAGATTTGTATGTAGTTTTAATGTCACCTATAACAAAATGTGTTTGGTAGCAGATTGTCCAGAAA
GCATTTTAAATGAAGAGGTATAAACCCTTAAGGGCCAAAATTCTGTATATTAGATTACTCTTAAACGAAA
AACCAGCTGCCGCTTTTATGTACACATATTACATACGAGTAGGCAGCAGACTTTAAAAATAAAAAAAACC
TAGGCATGTTGATGTTGCAAAATGCTGTATAAAGCTGAAACCTGTTCATTCAGTGCCATTGTAGTTGACA
TGAAGCGATTGTAAAACTGTCTCCGATTTTTCTCTGGTTTATTAAAATGCTAACTATAACATTTTTTGTG
AATACTTTGAATGTTTCCTAACAGTTGTGATGTTACTGTTCCGTTTTATGCTCTTATTCCAAGTTCATTT
TTAATGGTTTGGAAGCCATTTTTGTAATGAATAAATGTTCATGCTGTACAGTATCTGTAGCATGCCGTTC
TGGATTAATAAAAGCAACTTAGTATGTGCAGATAAA,
Protein sequence (SEQ ID NO: 60):
MPQLNGGGGDDLGANDELISFKDEGEQEEKSSENSSAERDLADVKSSLVNESETNONSSSDSEAERRPPPRSESF
RDKSRESLEEAAKRQDGGLFKGPPYPGYPFIMIPDLTSPYLPNGSLSPTARTYLQMKWPLLDVQAGSLQSRQALK
DARSPSPAHIVSNKVPVVQHPHHVHPLTPLITYSNEHFTPGNPPPHLPADVDPKTGIPRPPHPPDISPYYPLSPG
TVGQIPHPLGWLVPQQGQPVYPITTGGFRHPYPTALTVNASMSRFPPHMVPPHHTLHTTGIPHPAIVTPTVKQES
SQSDVGSLHSSKHQDSKKEEEKKKPHIKKPLNAFMLYMKEMRAKVVAECTLKESAAINQILGRRWHALSREEQAK
YYELARKERQLHMQLYPGWSARDNYGKKKKRKRDKQPGETNDANTPKKCRALFGLDRQTLWCKPCRRKKKCVRYI
QGEGSCLSPPSSDGSLLDSPPPSPNLLGSPPRDAKSQTEQTQPLSLSLKPDPLAHLSMMPPPPALLLAEATHKAS
ALCPNGALDLPPAALQPAAPSSSIAQPSTSSLHSHSSLAGTQPQPLSLVTKSLE
>NM_001198527.2 Homo sapiens transcription
factor 7 like 2 (TCF7L2), transcript variant 9
(SEQ ID NO: 61)
GTCAATAATCTCCGCTCCCAGACTACTCCGTTCCTCCGGATTTCGATCCCCCTTTTTCTATCTGTCAATC
AGCGCCGCCTTTGAACTGAAAAGCTCTCAGTCTAACTTCAACTCACTCAAATCCGAGCGGCACGAGCACC
TCCTGTATCTTCGGCTTCCCCCCCCCTTTGCTCTTTATATCTGACTTCTTGTTGTTGTTGGTGTTTTTTT
TTTTTTTACCCCCCTTTTTTATTTATTATTTTTTTGCACATTGATCGGATCCTTGGGAACGAGAGAAAAA
AGAAACCCAAACTCACGCGTGCAGAAGATCTCCCCCCCCTTCCCCTCCCCTCCTCCCTCTTTTCCCCTCC
CCAGGAGAAAAAGACCCCCAAGCAGAAAAAAGTTCACCTTGGACTCGTCTTTTTCTTGCAATATTTTTTG
GGGGGGCAAAACTTTTTGGGGGTGATTTTTTTTGGCTTTTCTTCCTCCTTCATTTTTCTTCCAAAATTGC
TGCTGGTGGGTGAAAAAAAAATGCCGCAGCTGAACGGCGGTGGAGGGGATGACCTAGGCGCCAACGACGA
ACTGATTTCCTTCAAAGACGAGGGCGAACAGGAGGAGAAGAGCTCCGAAAACTCCTCGGCAGAGAGGGAT
TTAGCTGATGTCAAATCGTCTCTAGTCAATGAATCAGAAACGAATCAAAACAGCTCCTCCGATTCCGAGG
CGGAAAGACGGCCTCCGCCTCGCTCCGAAAGTTTCCGAGACAAATCCCGGGAAAGTTTGGAAGAAGCGGC
CAAGAGGCAAGATGGAGGGCTCTTTAAGGGGCCACCGTATCCCGGCTACCCCTTCATCATGATCCCCGAC
CTGACGAGCCCCTACCTCCCCAACGGATCGCTCTCGCCCACCGCCCGAACCTATCTCCAGATGAAATGGC
CACTGCTTGATGTCCAGGCAGGGAGCCTCCAGAGTAGACAAGCCCTCAAGGATGCCCGGTCCCCATCACC
GGCACACATTGTCTCTAACAAAGTGCCAGTGGTGCAGCACCCTCACCATGTCCACCCCCTCACGCCTCTT
ATCACGTACAGCAATGAACACTTCACGCCGGGAAACCCACCTCCACACTTACCAGCCGACGTAGACCCCA
AAACAGGAATCCCACGGCCTCCGCACCCTCCAGATATATCCCCGTATTACCCACTATCGCCTGGCACCGT
AGGACAAATCCCCCATCCGCTAGGATGGCAAGGTCAACCAGTGTACCCAATCACGACAGGAGGATTCAGA
CACCCCTACCCCACAGCTCTGACCGTCAATGCTTCCATGTCCAGGTTCCCTCCCCATATGGTCCCACCAC
ATCATACGCTACACACGACGGGCATTCCGCATCCGGCCATAGTCACACCAACAGTCAAACAGGAATCGTC
CCAGAGTGATGTCGGCTCACTCCATAGTTCAAAGCATCAGGACTCCAAAAAGGAAGAAGAAAAGAAGAAG
CCCCACATAAAGAAACCTCTTAATGCATTCATGTTGTATATGAAGGAAATGAGAGCAAAGGTCGTAGCTG
AGTGCACGTTGAAAGAAAGCGCGGCCATCAACCAGATCCTTGGGCGGAGGTGGCATGCACTGTCCAGAGA
AGAGCAAGCGAAATACTACGAGCTGGCCCGGAAGGAGCGACAGCTTCATATGCAACTGTACCCCGGCTGG
TCCGCGCGGGATAACTATGGAAAGAAGAAGAAGAGGAAAAGGGACAAGCAGCCGGGAGAGACCAATGAAC
ACAGCGAATGTTTCCTAAATCCTTGCCTTTCACTTCCTCCGATTACAGACCTGAGCGCTCCTAAGAAATG
CCGAGCGCGCTTTGGCCTTGATCAACAGAATAACTGGTGCGGCCCTTGCAGTCTTTGAATTTGGAATATT
ACAATGGAGAAAAAAAAAGTGCGTTCGCTACATACAAGGTGAAGGCAGCTGCCTCAGCCCACCCTCTTCA
GATGGAAGCTTACTAGATTCGCCTCCCCCCTCCCCGAACCTGCTAGGCTCCCCTCCCCGAGACGCCAAGT
CACAGACTGAGCAGACCCAGCCTCTGTCGCTGTCCCTGAAGCCCGACCCCCTGGCCCACCTGTCCATGAT
GCCTCCGCCACCCGCCCTCCTGCTCGCTGAGGCCACCCACAAGGCCTCCGCCCTCTGTCCCAACGGGGCC
CTGGACCTGCCCCCAGCCGCTTTGCAGCCTGCCGCCCCCTCCTCATCAATTGCACAGCCGTCGACTTCTT
CCTTACATTCCCACAGCTCCCTGGCCGGGACCCAGCCCCAGCCGCTGTCGCTCGTCACCAAGTCTTTAGA
ATAGCTTTAGCGTCGTGAACCCCGCTGCTTTGTTTATGGTTTTGTTTCACTTTTCTTAATTTGCCCCCCA
CCCCCACCTTGAAAGGTTTTGTTTTGTACTCTCTTAATTTTGTGCCATGTGGCTACATTAGTTGATGTTT
ATCGAGTTCATTGGTCAATATTTGACCCATTCTTATTTCAATTTCTCCTTTTAAATATGTAGATGAGAGA
AGAACCTCATGATTCTACCAAAATTTTTATCAACAGCTGTTTAAAGTCTTTGTAGCGTTTAAAAAATATA
TATATATACATAACTGTTATGTAGTTCGGATAGCTTAGTTTTAAAAGACTGATTAAAAAACAAAAAGAAA
AAAAAAGCAATTTTGAAGCAGCCCTCCAGAAGGAGTTGGTTCTGTATTATTTGTATTAAATACGAGCTTG
CGAACCAATCATTTTACATCTGGTTTTTAAACCGTAAGGGCACCATGAATGCAGTGCCGTTACTTTTTTT
TTTTTTTTCTGTGTGAAACAACTCTTATTGTGATGTTACTTGTTATTGTTTAAATGTACAGAAACAAAGG
GTAAAAATGTGTTAATATACCTTGTTCCATGGTGTTGTTCTTTTGGGGGGAGGGGACGCTACTCAACACT
TAATAGAATCACAACGCTGTTGGGCCAGTAGTATTTATTGCTTTAGAGATTGCTTGTCGTACCTGTATGT
CGTCCCTTTTTAAATATGTTTTCCTTTTTCTTGAAACTGTATAAAGTTTTTTTCCCCCTTAGCATAAGCA
TCTTATATATAACAACTCATTTGTACAAGGTTTTTAAGTTTATATATAAAATGTGTATATATATTTTTGT
TTCCCCTTTTTGACTTTTTTTTTTCTGTATGAAACCCAGATGTCACCAAATGGACATTAATAGTTGCATT
AAGGATCAGTAGCATTAACAAAAGTTGCTTTAAAAGCCATTATGTAAAACAAGACTTGAAAATGAGTGAG
GGAATTTTAGCGACACTGTCTGAGCAGCAGTGGGAACCATCTTCGTTTCCCCTTTGAACTCCCAGTGGGA
TGCCCTACCCTGCGCCCTTAGGACCCGGACTGACCGTGTACAAAACTTTACGTGCCAAAATTCTCAGTGA
ATTTAGCTTTCTCCCTCTTTTTGATGCTGTAATTTTTGTTCATCATGTTTTGCTGTGATGTTACATAGGT
AGATTTGTATGTAGTTTTAATGTCACCTATAACAAAATGTGTTTGGTAGCAGATTGTCCAGAAAGCATTT
TAAATGAAGAGGTATAAACCCTTAAGGGCCAAAATTCTGTATATTAGATTACTCTTAAACGAAAAACCAG
CTGCCGCTTTTATGTACACATATTACATACGAGTAGGCAGCAGACTTTAAAAATAAAAAAAACCTAGGCA
TGTTGATGTTGCAAAATGCTGTATAAAGCTGAAACCTGTTCATTCAGTGCCATTGTAGTTGACATGAAGC
GATTGTAAAACTGTCTCCGATTTTTCTCTGGTTTATTAAAATGCTAACTATAACATTTTTTGTGAATACT
TTGAATGTTTCCTAACAGTTGTGATGTTACTGTTCCGTTTTATGCTCTTATTCCAAGTTCATTTTTAATG
GTTTGGAAGCCATTTTTGTAATGAATAAATGTTCATGCTGTACAGTATCTGTAGCATGCCGTTCTGGATT
AATAAAAGCAACTTAGTATGTGCAGATAAA,
Protein sequence (SEQ ID NO: 62):
MPQLNGGGGDDLGANDELISFKDEGEQEEKSSENSSAERDLADVKSSLVNESETNQNSSSDSEAERRPPPRSESF
RDKSRESLEEAAKRQDGGLFKGPPYPGYPFIMIPDLTSPYLPNGSLSPTARTYLQMKWPLLDVQAGSLQSRQALK
DARSPSPAHIVSNKVPVVQHPHHVHPLTPLITYSNEHFTPGNPPPHLPADVDPKTGIPRPPHPPDISPYYPLSPG
TVGQIPHPLGWQGQPVYPITTGGFRHPYPTALTVNASMSRFPPHMVPPHHTLHTTGIPHPAIVTPTVKQESSQSD
VGSLHSSKHQDSKKEEEKKKPHIKKPLNAFMLYMKEMRAKVVAECTLKESAAINQILGRRWHALSREEQAKYYEL
ARKERQLHMQLYPGWSARDNYGKKKKRKRDKQPGETNEHSECFLNPCLSLPPITDLSAPKKCRARFGLDQQNNWC
GPCSL
>NM_001198528.2 Homo sapiens transcription
factor 7 like 2 (TCF7L2), transcript variant
10 (SEQ ID NO: 63)
GTCAATAATCTCCGCTCCCAGACTACTCCGTTCCTCCGGATTTCGATCCCCCTTTTTCTATCTGTCAATC
AGCGCCGCCTTTGAACTGAAAAGCTCTCAGTCTAACTTCAACTCACTCAAATCCGAGCGGCACGAGCACC
TCCTGTATCTTCGGCTTCCCCCCCCCTTTGCTCTTTATATCTGACTTCTTGTTGTTGTTGGTGTTTTTTT
TTTTTTTACCCCCCTTTTTTATTTATTATTTTTTTGCACATTGATCGGATCCTTGGGAACGAGAGAAAAA
AGAAACCCAAACTCACGCGTGCAGAAGATCTCCCCCCCCTTCCCCTCCCCTCCTCCCTCTTTTCCCCTCC
CCAGGAGAAAAAGACCCCCAAGCAGAAAAAAGTTCACCTTGGACTCGTCTTTTTCTTGCAATATTTTTTG
GGGGGGCAAAACTTTTTGGGGGTGATTTTTTTTGGCTTTTCTTCCTCCTTCATTTTTCTTCCAAAATTGC
TGCTGGTGGGTGAAAAAAAAATGCCGCAGCTGAACGGCGGTGGAGGGGATGACCTAGGCGCCAACGACGA
ACTGATTTCCTTCAAAGACGAGGGCGAACAGGAGGAGAAGAGCTCCGAAAACTCCTCGGCAGAGAGGGAT
TTAGCTGATGTCAAATCGTCTCTAGTCAATGAATCAGAAACGAATCAAAACAGCTCCTCCGATTCCGAGG
CGGAAAGACGGCCTCCGCCTCGCTCCGAAAGTTTCCGAGACAAATCCCGGGAAAGTTTGGAAGAAGCGGC
CAAGAGGCAAGATGGAGGGCTCTTTAAGGGGCCACCGTATCCCGGCTACCCCTTCATCATGATCCCCGAC
CTGACGAGCCCCTACCTCCCCAACGGATCGCTCTCGCCCACCGCCCGAACCTATCTCCAGATGAAATGGC
CACTGCTTGATGTCCAGGCAGGGAGCCTCCAGAGTAGACAAGCCCTCAAGGATGCCCGGTCCCCATCACC
GGCACACATTGTCTCTAACAAAGTGCCAGTGGTGCAGCACCCTCACCATGTCCACCCCCTCACGCCTCTT
ATCACGTACAGCAATGAACACTTCACGCCGGGAAACCCACCTCCACACTTACCAGCCGACGTAGACCCCA
AAACAGGAATCCCACGGCCTCCGCACCCTCCAGATATATCCCCGTATTACCCACTATCGCCTGGCACCGT
AGGACAAATCCCCCATCCGCTAGGATGGTTAGTACCACAGCAAGGTCAACCAGTGTACCCAATCACGACA
GGAGGATTCAGACACCCCTACCCCACAGCTCTGACCGTCAATGCTTCCATGTCCAGGTTCCCTCCCCATA
TGGTCCCACCACATCATACGCTACACACGACGGGCATTCCGCATCCGGCCATAGTCACACCAACAGTCAA
ACAGGAATCGTCCCAGAGTGATGTCGGCTCACTCCATAGTTCAAAGCATCAGGACTCCAAAAAGGAAGAA
GAAAAGAAGAAGCCCCACATAAAGAAACCTCTTAATGCATTCATGTTGTATATGAAGGAAATGAGAGCAA
AGGTCGTAGCTGAGTGCACGTTGAAAGAAAGCGCGGCCATCAACCAGATCCTTGGGCGGAGGTGGCATGC
ACTGTCCAGAGAAGAGCAAGCGAAATACTACGAGCTGGCCCGGAAGGAGCGACAGCTTCATATGCAACTG
TACCCCGGCTGGTCCGCGCGGGATAACTATGGAAAGAAGAAGAAGAGGAAAAGGGACAAGCAGCCGGGAG
AGACCAATGAACACAGCGAATGTTTCCTAAATCCTTGCCTTTCACTTCCTCCGATTACAGACCTGAGCGC
TCCTAAGAAATGCCGAGCGCGCTTTGGCCTTGATCAACAGAATAACTGGTGCGGCCCTTGCAGTCTTTGA
ATTTGGAATATTACAATGGAGAAAAAAAAAGTGCGTTCGCTACATACAAGGTGAAGGCAGCTGCCTCAGC
CCACCCTCTTCAGATGGAAGCTTACTAGATTCGCCTCCCCCCTCCCCGAACCTGCTAGGCTCCCCTCCCC
GAGACGCCAAGTCACAGACTGAGCAGACCCAGCCTCTGTCGCTGTCCCTGAAGCCCGACCCCCTGGCCCA
CCTGTCCATGATGCCTCCGCCACCCGCCCTCCTGCTCGCTGAGGCCACCCACAAGGCCTCCGCCCTCTGT
CCCAACGGGGCCCTGGACCTGCCCCCAGCCGCTTTGCAGCCTGCCGCCCCCTCCTCATCAATTGCACAGC
CGTCGACTTCTTCCTTACATTCCCACAGCTCCCTGGCCGGGACCCAGCCCCAGCCGCTGTCGCTCGTCAC
CAAGTCTTTAGAATAGCTTTAGCGTCGTGAACCCCGCTGCTTTGTTTATGGTTTTGTTTCACTTTTCTTA
ATTTGCCCCCCACCCCCACCTTGAAAGGTTTTGTTTTGTACTCTCTTAATTTTGTGCCATGTGGCTACAT
TAGTTGATGTTTATCGAGTTCATTGGTCAATATTTGACCCATTCTTATTTCAATTTCTCCTTTTAAATAT
GTAGATGAGAGAAGAACCTCATGATTCTACCAAAATTTTTATCAACAGCTGTTTAAAGTCTTTGTAGCGT
TTAAAAAATATATATATATACATAACTGTTATGTAGTTCGGATAGCTTAGTTTTAAAAGACTGATTAAAA
AACAAAAAGAAAAAAAAAGCAATTTTGAAGCAGCCCTCCAGAAGGAGTTGGTTCTGTATTATTTGTATTA
AATACGAGCTTGCGAACCAATCATTTTACATCTGGTTTTTAAACCGTAAGGGCACCATGAATGCAGTGCC
GTTACTTTTTTTTTTTTTTTCTGTGTGAAACAACTCTTATTGTGATGTTACTTGTTATTGTTTAAATGTA
CAGAAACAAAGGGTAAAAATGTGTTAATATACCTTGTTCCATGGTGTTGTTCTTTTGGGGGGAGGGGACG
CTACTCAACACTTAATAGAATCACAACGCTGTTGGGCCAGTAGTATTTATTGCTTTAGAGATTGCTTGTC
GTACCTGTATGTCGTCCCTTTTTAAATATGTTTTCCTTTTTCTTGAAACTGTATAAAGTTTTTTTCCCCC
TTAGCATAAGCATCTTATATATAACAACTCATTTGTACAAGGTTTTTAAGTTTATATATAAAATGTGTAT
ATATATTTTTGTTTCCCCTTTTTGACTTTTTTTTTTCTGTATGAAACCCAGATGTCACCAAATGGACATT
AATAGTTGCATTAAGGATCAGTAGCATTAACAAAAGTTGCTTTAAAAGCCATTATGTAAAACAAGACTTG
AAAATGAGTGAGGGAATTTTAGCGACACTGTCTGAGCAGCAGTGGGAACCATCTTCGTTTCCCCTTTGAA
CTCCCAGTGGGATGCCCTACCCTGCGCCCTTAGGACCCGGACTGACCGTGTACAAAACTTTACGTGCCAA
AATTCTCAGTGAATTTAGCTTTCTCCCTCTTTTTGATGCTGTAATTTTTGTTCATCATGTTTTGCTGTGA
TGTTACATAGGTAGATTTGTATGTAGTTTTAATGTCACCTATAACAAAATGTGTTTGGTAGCAGATTGTC
CAGAAAGCATTTTAAATGAAGAGGTATAAACCCTTAAGGGCCAAAATTCTGTATATTAGATTACTCTTAA
ACGAAAAACCAGCTGCCGCTTTTATGTACACATATTACATACGAGTAGGCAGCAGACTTTAAAAATAAAA
AAAACCTAGGCATGTTGATGTTGCAAAATGCTGTATAAAGCTGAAACCTGTTCATTCAGTGCCATTGTAG
TTGACATGAAGCGATTGTAAAACTGTCTCCGATTTTTCTCTGGTTTATTAAAATGCTAACTATAACATTT
TTTGTGAATACTTTGAATGTTTCCTAACAGTTGTGATGTTACTGTTCCGTTTTATGCTCTTATTCCAAGT
TCATTTTTAATGGTTTGGAAGCCATTTTTGTAATGAATAAATGTTCATGCTGTACAGTATCTGTAGCATG
CCGTTCTGGATTAATAAAAGCAACTTAGTATGTGCAGATAAA,
Protein sequence (SEQ ID NO: 64):
MPQLNGGGGDDLGANDELISFKDEGEQEEKSSENSSAERDLADVKSSLVNESETNONSSSDSEAERRPPPRSESF
RDKSRESLEEAAKRQDGGLFKGPPYPGYPFIMIPDLTSPYLPNGSLSPTARTYLQMKWPLLDVQAGSLQSRQALK
DARSPSPAHIVSNKVPVVQHPHHVHPLTPLITYSNEHFTPGNPPPHLPADVDPKTGIPRPPHPPDISPYYPLSPG
TVGQIPHPLGWLVPQQGQPVYPITTGGFRHPYPTALTVNASMSRFPPHMVPPHHTLHTTGIPHPAIVTPTVKQES
SQSDVGSLHSSKHQDSKKEEEKKKPHIKKPLNAFMLYMKEMRAKVVAECTLKESAAINQILGRRWHALSREEQAK
YYELARKERQLHMQLYPGWSARDNYGKKKKRKRDKQPGETNEHSECFLNPCLSLPPITDLSAPKKCRARFGLDQQ
NNWCGPCSL
>NM_001198529.2 Homo sapiens transcription
factor 7 like 2 (TCF7L2), transcript variant
11 (SEQ ID NO: 65)
GTCAATAATCTCCGCTCCCAGACTACTCCGTTCCTCCGGATTTCGATCCCCCTTTTTCTATCTGTCAATC
AGCGCCGCCTTTGAACTGAAAAGCTCTCAGTCTAACTTCAACTCACTCAAATCCGAGCGGCACGAGCACC
TCCTGTATCTTCGGCTTCCCCCCCCCTTTGCTCTTTATATCTGACTTCTTGTTGTTGTTGGTGTTTTTTT
TTTTTTTACCCCCCTTTTTTATTTATTATTTTTTTGCACATTGATCGGATCCTTGGGAACGAGAGAAAAA
AGAAACCCAAACTCACGCGTGCAGAAGATCTCCCCCCCCTTCCCCTCCCCTCCTCCCTCTTTTCCCCTCC
CCAGGAGAAAAAGACCCCCAAGCAGAAAAAAGTTCACCTTGGACTCGTCTTTTTCTTGCAATATTTTTTG
GGGGGGCAAAACTTTTTGGGGGTGATTTTTTTTGGCTTTTCTTCCTCCTTCATTTTTCTTCCAAAATTGC
TGCTGGTGGGTGAAAAAAAAATGCCGCAGCTGAACGGCGGTGGAGGGGATGACCTAGGCGCCAACGACGA
ACTGATTTCCTTCAAAGACGAGGGCGAACAGGAGGAGAAGAGCTCCGAAAACTCCTCGGCAGAGAGGGAT
TTAGCTGATGTCAAATCGTCTCTAGTCAATGAATCAGAAACGAATCAAAACAGCTCCTCCGATTCCGAGG
CGGAAAGACGGCCTCCGCCTCGCTCCGAAAGTTTCCGAGACAAATCCCGGGAAAGTTTGGAAGAAGCGGC
CAAGAGGCAAGATGGAGGGCTCTTTAAGGGGCCACCGTATCCCGGCTACCCCTTCATCATGATCCCCGAC
CTGACGAGCCCCTACCTCCCCAACGGATCGCTCTCGCCCACCGCCCGAACCTATCTCCAGATGAAATGGC
CACTGCTTGATGTCCAGGCAGGGAGCCTCCAGAGTAGACAAGCCCTCAAGGATGCCCGGTCCCCATCACC
GGCACACATTGTCTCTAACAAAGTGCCAGTGGTGCAGCACCCTCACCATGTCCACCCCCTCACGCCTCTT
ATCACGTACAGCAATGAACACTTCACGCCGGGAAACCCACCTCCACACTTACCAGCCGACGTAGACCCCA
AAACAGGAATCCCACGGCCTCCGCACCCTCCAGATATATCCCCGTATTACCCACTATCGCCTGGCACCGT
AGGACAAATCCCCCATCCGCTAGGATGGTTAGTACCACAGCAAGGTCAACCAGTGTACCCAATCACGACA
GGAGGATTCAGACACCCCTACCCCACAGCTCTGACCGTCAATGCTTCCATGTCCAGGTTCCCTCCCCATA
TGGTCCCACCACATCATACGCTACACACGACGGGCATTCCGCATCCGGCCATAGTCACACCAACAGTCAA
ACAGGAATCGTCCCAGAGTGATGTCGGCTCACTCCATAGTTCAAAGCATCAGGACTCCAAAAAGGAAGAA
GAAAAGAAGAAGCCCCACATAAAGAAACCTCTTAATGCATTCATGTTGTATATGAAGGAAATGAGAGCAA
AGGTCGTAGCTGAGTGCACGTTGAAAGAAAGCGCGGCCATCAACCAGATCCTTGGGCGGAGGTGGCATGC
ACTGTCCAGAGAAGAGCAAGCGAAATACTACGAGCTGGCCCGGAAGGAGCGACAGCTTCATATGCAACTG
TACCCCGGCTGGTCCGCGCGGGATAACTATGGAAAGAAGAAGAAGAGGAAAAGGGACAAGCAGCCGGGAG
AGACCAATGAACACAGCGAATGTTTCCTAAATCCTTGCCTTTCACTTCCTCCGATTACAGGAGAAAAAAA
AAGTGCGTTCGCTACATACAAGGTGAAGGCAGCTGCCTCAGCCCACCCTCTTCAGATGGAAGCTTACTAG
ATTCGCCTCCCCCCTCCCCGAACCTGCTAGGCTCCCCTCCCCGAGACGCCAAGTCACAGACTGAGCAGAC
CCAGCCTCTGTCGCTGTCCCTGAAGCCCGACCCCCTGGCCCACCTGTCCATGATGCCTCCGCCACCCGCC
CTCCTGCTCGCTGAGGCCACCCACAAGGCCTCCGCCCTCTGTCCCAACGGGGCCCTGGACCTGCCCCCAG
CCGCTTTGCAGCCTGCCGCCCCCTCCTCATCAATTGCACAGCCGTCGACTTCTTCCTTACATTCCCACAG
CTCCCTGGCCGGGACCCAGCCCCAGCCGCTGTCGCTCGTCACCAAGTCTTTAGAATAGCTTTAGCGTCGT
GAACCCCGCTGCTTTGTTTATGGTTTTGTTTCACTTTTCTTAATTTGCCCCCCACCCCCACCTTGAAAGG
TTTTGTTTTGTACTCTCTTAATTTTGTGCCATGTGGCTACATTAGTTGATGTTTATCGAGTTCATTGGTC
AATATTTGACCCATTCTTATTTCAATTTCTCCTTTTAAATATGTAGATGAGAGAAGAACCTCATGATTCT
ACCAAAATTTTTATCAACAGCTGTTTAAAGTCTTTGTAGCGTTTAAAAAATATATATATATACATAACTG
TTATGTAGTTCGGATAGCTTAGTTTTAAAAGACTGATTAAAAAACAAAAAGAAAAAAAAAGCAATTTTGA
AGCAGCCCTCCAGAAGGAGTTGGTTCTGTATTATTTGTATTAAATACGAGCTTGCGAACCAATCATTTTA
CATCTGGTTTTTAAACCGTAAGGGCACCATGAATGCAGTGCCGTTACTTTTTTTTTTTTTTTCTGTGTGA
AACAACTCTTATTGTGATGTTACTTGTTATTGTTTAAATGTACAGAAACAAAGGGTAAAAATGTGTTAAT
ATACCTTGTTCCATGGTGTTGTTCTTTTGGGGGGAGGGGACGCTACTCAACACTTAATAGAATCACAACG
CTGTTGGGCCAGTAGTATTTATTGCTTTAGAGATTGCTTGTCGTACCTGTATGTCGTCCCTTTTTAAATA
TGTTTTCCTTTTTCTTGAAACTGTATAAAGTTTTTTTCCCCCTTAGCATAAGCATCTTATATATAACAAC
TCATTTGTACAAGGTTTTTAAGTTTATATATAAAATGTGTATATATATTTTTGTTTCCCCTTTTTGACTT
TTTTTTTTCTGTATGAAACCCAGATGTCACCAAATGGACATTAATAGTTGCATTAAGGATCAGTAGCATT
AACAAAAGTTGCTTTAAAAGCCATTATGTAAAACAAGACTTGAAAATGAGTGAGGGAATTTTAGCGACAC
TGTCTGAGCAGCAGTGGGAACCATCTTCGTTTCCCCTTTGAACTCCCAGTGGGATGCCCTACCCTGCGCC
CTTAGGACCCGGACTGACCGTGTACAAAACTTTACGTGCCAAAATTCTCAGTGAATTTAGCTTTCTCCCT
CTTTTTGATGCTGTAATTTTTGTTCATCATGTTTTGCTGTGATGTTACATAGGTAGATTTGTATGTAGTT
TTAATGTCACCTATAACAAAATGTGTTTGGTAGCAGATTGTCCAGAAAGCATTTTAAATGAAGAGGTATA
AACCCTTAAGGGCCAAAATTCTGTATATTAGATTACTCTTAAACGAAAAACCAGCTGCCGCTTTTATGTA
CACATATTACATACGAGTAGGCAGCAGACTTTAAAAATAAAAAAAACCTAGGCATGTTGATGTTGCAAAA
TGCTGTATAAAGCTGAAACCTGTTCATTCAGTGCCATTGTAGTTGACATGAAGCGATTGTAAAACTGTCT
CCGATTTTTCTCTGGTTTATTAAAATGCTAACTATAACATTTTTTGTGAATACTTTGAATGTTTCCTAAC
AGTIGTGATGTTACTGTTCCGTTTTATGCTCTTATTCCAAGTTCATTTTTAATGGTTTGGAAGCCATTTT
TGTAATGAATAAATGTTCATGCTGTACAGTATCTGTAGCATGCCGTTCTGGATTAATAAAAGCAACTTAG
TATGTGCAGATAAA,
Protein sequence (SEQ ID NO: 66):
MPQLNGGGGDDLGANDELISFKDEGEQEEKSSENSSAERDLADVKSSLVNESETNONSSSDSEAERRPPPRSESF
RDKSRESLEEAAKRQDGGLFKGPPYPGYPFIMIPDLTSPYLPNGSLSPTARTYLQMKWPLLDVQAGSLQSRQALK
DARSPSPAHIVSNKVPVVQHPHHVHPLTPLITYSNEHFTPGNPPPHLPADVDPKTGIPRPPHPPDISPYYPLSPG
TVGQIPHPLGWLVPQQGQPVYPITTGGFRHPYPTALTVNASMSRFPPHMVPPHHTLHTTGIPHPAIVTPTVKQES
SQSDVGSLHSSKHQDSKKEEEKKKPHIKKPLNAFMLYMKEMRAKVVAECTLKESAAINQILGRRWHALSREEQAK
YYELARKERQLHMQLYPGWSARDNYGKKKKRKRDKQPGETNEHSECFLNPCLSLPPITGEKKSAFATYKVKAAAS
AHPLQMEAY
>NM_001198530.2 Homo sapiens transcription
factor 7 like 2 (TCF7L2), transcript variant
12 (SEQ ID NO: 67)
GTCAATAATCTCCGCTCCCAGACTACTCCGTTCCTCCGGATTTCGATCCCCCTTTTTCTATCTGTCAATC
AGCGCCGCCTTTGAACTGAAAAGCTCTCAGTCTAACTTCAACTCACTCAAATCCGAGCGGCACGAGCACC
TCCTGTATCTTCGGCTTCCCCCCCCCTTTGCTCTTTATATCTGACTTCTTGTTGTTGTTGGTGTTTTTTT
TTTTTTTACCCCCCTTTTTTATTTATTATTTTTTTGCACATTGATCGGATCCTTGGGAACGAGAGAAAAA
AGAAACCCAAACTCACGCGTGCAGAAGATCTCCCCCCCCTTCCCCTCCCCTCCTCCCTCTTTTCCCCTCC
CCAGGAGAAAAAGACCCCCAAGCAGAAAAAAGTTCACCTTGGACTCGTCTTTTTCTTGCAATATTTTTTG
GGGGGGCAAAACTTTTTGGGGGTGATTTTTTTTGGCTTTTCTTCCTCCTTCATTTTTCTTCCAAAATTGC
TGCTGGTGGGTGAAAAAAAAATGCCGCAGCTGAACGGCGGTGGAGGGGATGACCTAGGCGCCAACGACGA
ACTGATTTCCTTCAAAGACGAGGGCGAACAGGAGGAGAAGAGCTCCGAAAACTCCTCGGCAGAGAGGGAT
TTAGCTGATGTCAAATCGTCTCTAGTCAATGAATCAGAAACGAATCAAAACAGCTCCTCCGATTCCGAGG
CGGAAAGACGGCCTCCGCCTCGCTCCGAAAGTTTCCGAGACAAATCCCGGGAAAGTTTGGAAGAAGCGGC
CAAGAGGCAAGATGGAGGGCTCTTTAAGGGGCCACCGTATCCCGGCTACCCCTTCATCATGATCCCCGAC
CTGACGAGCCCCTACCTCCCCAACGGATCGCTCTCGCCCACCGCCCGAACCTCTAACAAAGTGCCAGTGG
TGCAGCACCCTCACCATGTCCACCCCCTCACGCCTCTTATCACGTACAGCAATGAACACTTCACGCCGGG
AAACCCACCTCCACACTTACCAGCCGACGTAGACCCCAAAACAGGAATCCCACGGCCTCCGCACCCTCCA
GATATATCCCCGTATTACCCACTATCGCCTGGCACCGTAGGACAAATCCCCCATCCGCTAGGATGGTTAG
TACCACAGCAAGGTCAACCAGTGTACCCAATCACGACAGGAGGATTCAGACACCCCTACCCCACAGCTCT
GACCGTCAATGCTTCCATGTCCAGGTTCCCTCCCCATATGGTCCCACCACATCATACGCTACACACGACG
GGCATTCCGCATCCGGCCATAGTCACACCAACAGTCAAACAGGAATCGTCCCAGAGTGATGTCGGCTCAC
TCCATAGTTCAAAGCATCAGGACTCCAAAAAGGAAGAAGAAAAGAAGAAGCCCCACATAAAGAAACCTCT
TAATGCATTCATGTTGTATATGAAGGAAATGAGAGCAAAGGTCGTAGCTGAGTGCACGTTGAAAGAAAGC
GCGGCCATCAACCAGATCCTTGGGCGGAGGTGGCATGCACTGTCCAGAGAAGAGCAAGCGAAATACTACG
AGCTGGCCCGGAAGGAGCGACAGCTTCATATGCAACTGTACCCCGGCTGGTCCGCGCGGGATAACTATGG
AAAGAAGAAGAAGAGGAAAAGGGACAAGCAGCCGGGAGAGACCAATGGAGAAAAAAAAAGTGCGTTCGCT
ACATACAAGGTGAAGGCAGCTGCCTCAGCCCACCCTCTTCAGATGGAAGCTTACTAGATTCGCCTCCCCC
CTCCCCGAACCTGCTAGGCTCCCCTCCCCGAGACGCCAAGTCACAGACTGAGCAGACCCAGCCTCTGTCG
CTGTCCCTGAAGCCCGACCCCCTGGCCCACCTGTCCATGATGCCTCCGCCACCCGCCCTCCTGCTCGCTG
AGGCCACCCACAAGGCCTCCGCCCTCTGTCCCAACGGGGCCCTGGACCTGCCCCCAGCCGCTTTGCAGCC
TGCCGCCCCCTCCTCATCAATTGCACAGCCGTCGACTTCTTCCTTACATTCCCACAGCTCCCTGGCCGGG
ACCCAGCCCCAGCCGCTGTCGCTCGTCACCAAGTCTTTAGAATAGCTTTAGCGTCGTGAACCCCGCTGCT
TTGTTTATGGTTTTGTTTCACTTTTCTTAATTTGCCCCCCACCCCCACCTTGAAAGGTTTTGTTTTGTAC
TCTCTTAATTTTGTGCCATGTGGCTACATTAGTTGATGTTTATCGAGTTCATTGGTCAATATTTGACCCA
TTCTTATTTCAATTTCTCCTTTTAAATATGTAGATGAGAGAAGAACCTCATGATTCTACCAAAATTTTTA
TCAACAGCTGTTTAAAGTCTTTGTAGCGTTTAAAAAATATATATATATACATAACTGTTATGTAGTTCGG
ATAGCTTAGTTTTAAAAGACTGATTAAAAAACAAAAAGAAAAAAAAAGCAATTTTGAAGCAGCCCTCCAG
AAGGAGTTGGTTCTGTATTATTTGTATTAAATACGAGCTTGCGAACCAATCATTTTACATCTGGTTTTTA
AACCGTAAGGGCACCATGAATGCAGTGCCGTTACTTTTTTTTTTTTTTTCTGTGTGAAACAACTCTTATT
GTGATGTTACTTGTTATTGTTTAAATGTACAGAAACAAAGGGTAAAAATGTGTTAATATACCTTGTTCCA
TGGTGTTGTTCTTTTGGGGGGAGGGGACGCTACTCAACACTTAATAGAATCACAACGCTGTTGGGCCAGT
AGTATTTATTGCTTTAGAGATTGCTTGTCGTACCTGTATGTCGTCCCTTTTTAAATATGTTTTCCTTTTT
CTTGAAACTGTATAAAGTTTTTTTCCCCCTTAGCATAAGCATCTTATATATAACAACTCATTTGTACAAG
GTTTTTAAGTTTATATATAAAATGTGTATATATATTTTTGTTTCCCCTTTTTGACTTTTTTTTTTCTGTA
TGAAACCCAGATGTCACCAAATGGACATTAATAGTTGCATTAAGGATCAGTAGCATTAACAAAAGTTGCT
TTAAAAGCCATTATGTAAAACAAGACTTGAAAATGAGTGAGGGAATTTTAGCGACACTGTCTGAGCAGCA
GTGGGAACCATCTTCGTTTCCCCTTTGAACTCCCAGTGGGATGCCCTACCCTGCGCCCTTAGGACCCGGA
CTGACCGTGTACAAAACTTTACGTGCCAAAATTCTCAGTGAATTTAGCTTTCTCCCTCTTTTTGATGCTG
TAATTTTTGTTCATCATGTTTTGCTGTGATGTTACATAGGTAGATTTGTATGTAGTTTTAATGTCACCTA
TAACAAAATGTGTTTGGTAGCAGATTGTCCAGAAAGCATTTTAAATGAAGAGGTATAAACCCTTAAGGGC
CAAAATTCTGTATATTAGATTACTCTTAAACGAAAAACCAGCTGCCGCTTTTATGTACACATATTACATA
CGAGTAGGCAGCAGACTTTAAAAATAAAAAAAACCTAGGCATGTTGATGTTGCAAAATGCTGTATAAAGC
TGAAACCTGTTCATTCAGTGCCATTGTAGTTGACATGAAGCGATTGTAAAACTGTCTCCGATTTTTCTCT
GGTTTATTAAAATGCTAACTATAACATTTTTTGTGAATACTTTGAATGTTTCCTAACAGTTGTGATGTTA
CTGTTCCGTTTTATGCTCTTATTCCAAGTTCATTTTTAATGGTTTGGAAGCCATTTTTGTAATGAATAAA
TGTTCATGCTGTACAGTATCTGTAGCATGCCGTTCTGGATTAATAAAAGCAACTTAGTATGTGCAGATAA
A,
Protein sequence (SEQ ID NO: 68):
MPQLNGGGGDDLGANDELISFKDEGEQEEKSSENSSAERDLADVKSSLVNESETNONSSSDSEAERRPPPRSESF
RDKSRESLEEAAKRQDGGLFKGPPYPGYPFIMIPDLTSPYLPNGSLSPTARTSNKVPVVQHPHHVHPLTPLITYS
NEHFTPGNPPPHLPADVDPKTGIPRPPHPPDISPYYPLSPGTVGQIPHPLGWLVPQQGQPVYPITTGGFRHPYPT
ALTVNASMSRFPPHMVPPHHTLHTTGIPHPAIVTPTVKQESSQSDVGSLHSSKHQDSKKEEEKKKPHIKKPLNAF
MLYMKEMRAKVVAECTLKESAAINQILGRRWHALSREEQAKYYELARKERQLHMQLYPGWSARDNYGKKKKRKRD
KQPGETNGEKKSAFATYKVKAAASAHPLQMEA
>NM_001198531.2 Homo sapiens transcription
factor 7 like 2 (TCF7L2), transcript variant
13 (SEQ ID NO: 69)
GTCAATAATCTCCGCTCCCAGACTACTCCGTTCCTCCGGATTTCGATCCCCCTTTTTCTATCTGTCAATC
AGCGCCGCCTTTGAACTGAAAAGCTCTCAGTCTAACTTCAACTCACTCAAATCCGAGCGGCACGAGCACC
TCCTGTATCTTCGGCTTCCCCCCCCCTTTGCTCTTTATATCTGACTTCTTGTTGTTGTTGGTGTTTTTTT
TTTTTTTACCCCCCTTTTTTATTTATTATTTTTTTGCACATTGATCGGATCCTTGGGAACGAGAGAAAAA
AGAAACCCAAACTCACGCGTGCAGAAGATCTCCCCCCCCTTCCCCTCCCCTCCTCCCTCTTTTCCCCTCC
CCAGGAGAAAAAGACCCCCAAGCAGAAAAAAGTTCACCTTGGACTCGTCTTTTTCTTGCAATATTTTTTG
TGCTGGTGGGTGAAAAAAAAATGCCGCAGCTGAACGGCGGTGGAGGGGATGACCTAGGCGCCAACGACGA
ACTGATTTCCTTCAAAGACGAGGGCGAACAGGAGGAGAAGAGCTCCGAAAACTCCTCGGCAGAGAGGGAT
TTAGCTGATGTCAAATCGTCTCTAGTCAATGAATCAGAAACGAATCAAAACAGCTCCTCCGATTCCGAGG
CGGAAAGACGGCCTCCGCCTCGCTCCGAAAGTTTCCGAGACAAATCCCGGGAAAGTTTGGAAGAAGCGGC
CAAGAGGCAAGATGGAGGGCTCTTTAAGGGGCCACCGTATCCCGGCTACCCCTTCATCATGATCCCCGAC
CTGACGAGCCCCTACCTCCCCAACGGATCGCTCTCGCCCACCGCCCGAACCCTCCATTTTCAGTCCGGCA
GCACACATTACTCTGCGTACAAAACGATTGAACACCAGATTGCAGTTCAGTATCTCCAGATGAAATGGCC
ACTGCTTGATGTCCAGGCAGGGAGCCTCCAGAGTAGACAAGCCCTCAAGGATGCCCGGTCCCCATCACCG
GCACACATTGTCTCTAACAAAGTGCCAGTGGTGCAGCACCCTCACCATGTCCACCCCCTCACGCCTCTTA
TCACGTACAGCAATGAACACTTCACGCCGGGAAACCCACCTCCACACTTACCAGCCGACGTAGACCCCAA
AACAGGAATCCCACGGCCTCCGCACCCTCCAGATATATCCCCGTATTACCCACTATCGCCTGGCACCGTA
GGACAAATCCCCCATCCGCTAGGATGGTTAGTACCACAGCAAGGTCAACCAGTGTACCCAATCACGACAG
GAGGATTCAGACACCCCTACCCCACAGCTCTGACCGTCAATGCTTCCATGTCCAGGTTCCCTCCCCATAT
GGTCCCACCACATCATACGCTACACACGACGGGCATTCCGCATCCGGCCATAGTCACACCAACAGTCAAA
CAGGAATCGTCCCAGAGTGATGTCGGCTCACTCCATAGTTCAAAGCATCAGGACTCCAAAAAGGAAGAAG
AAAAGAAGAAGCCCCACATAAAGAAACCTCTTAATGCATTCATGTTGTATATGAAGGAAATGAGAGCAAA
GGTCGTAGCTGAGTGCACGTTGAAAGAAAGCGCGGCCATCAACCAGATCCTTGGGCGGAGGTGGCATGCA
CTGTCCAGAGAAGAGCAAGCGAAATACTACGAGCTGGCCCGGAAGGAGCGACAGCTTCATATGCAACTGT
ACCCCGGCTGGTCCGCGCGGGATAACTATGGAAAGAAGAAGAAGAGGAAAAGGGACAAGCAGCCGGGAGA
GACCAATGGAGAAAAAAAAAGTGCGTTCGCTACATACAAGGTGAAGGCAGCTGCCTCAGCCCACCCTCTT
CAGATGGAAGCTTACTAGATTCGCCTCCCCCCTCCCCGAACCTGCTAGGCTCCCCTCCCCGAGACGCCAA
GTCACAGACTGAGCAGACCCAGCCTCTGTCGCTGTCCCTGAAGCCCGACCCCCTGGCCCACCTGTCCATG
ATGCCTCCGCCACCCGCCCTCCTGCTCGCTGAGGCCACCCACAAGGCCTCCGCCCTCTGTCCCAACGGGG
CCCTGGACCTGCCCCCAGCCGCTTTGCAGCCTGCCGCCCCCTCCTCATCAATTGCACAGCCGTCGACTTC
TTCCTTACATTCCCACAGCTCCCTGGCCGGGACCCAGCCCCAGCCGCTGTCGCTCGTCACCAAGTCTTTA
GAATAGCTTTAGCGTCGTGAACCCCGCTGCTTTGTTTATGGTTTTGTTTCACTTTTCTTAATTTGCCCCC
CACCCCCACCTTGAAAGGTTTTGTTTTGTACTCTCTTAATTTTGTGCCATGTGGCTACATTAGTTGATGT
TTATCGAGTTCATTGGTCAATATTTGACCCATTCTTATTTCAATTTCTCCTTTTAAATATGTAGATGAGA
GAAGAACCTCATGATTCTACCAAAATTTTTATCAACAGCTGTTTAAAGTCTTTGTAGCGTTTAAAAAATA
TATATATATACATAACTGTTATGTAGTTCGGATAGCTTAGTTTTAAAAGACTGATTAAAAAACAAAAAGA
AAAAAAAAGCAATTTTGAAGCAGCCCTCCAGAAGGAGTTGGTTCTGTATTATTTGTATTAAATACGAGCT
TGCGAACCAATCATTTTACATCTGGTTTTTAAACCGTAAGGGCACCATGAATGCAGTGCCGTTACTTTTT
TTTTTTTTTTCTGTGTGAAACAACTCTTATTGTGATGTTACTTGTTATTGTTTAAATGTACAGAAACAAA
GGGTAAAAATGTGTTAATATACCTTGTTCCATGGTGTTGTTCTTTTGGGGGGAGGGGACGCTACTCAACA
CTTAATAGAATCACAACGCTGTTGGGCCAGTAGTATTTATTGCTTTAGAGATTGCTTGTCGTACCTGTAT
GTCGTCCCTTTTTAAATATGTTTTCCTTTTTCTTGAAACTGTATAAAGTTTTTTTCCCCCTTAGCATAAG
CATCTTATATATAACAACTCATTTGTACAAGGTTTTTAAGTTTATATATAAAATGTGTATATATATTTTT
GTTTCCCCTTTTTGACTTTTTTTTTTCTGTATGAAACCCAGATGTCACCAAATGGACATTAATAGTTGCA
TTAAGGATCAGTAGCATTAACAAAAGTTGCTTTAAAAGCCATTATGTAAAACAAGACTTGAAAATGAGTG
AGGGAATTTTAGCGACACTGTCTGAGCAGCAGTGGGAACCATCTTCGTTTCCCCTTTGAACTCCCAGTGG
GATGCCCTACCCTGCGCCCTTAGGACCCGGACTGACCGTGTACAAAACTTTACGTGCCAAAATTCTCAGT
GAATTTAGCTTTCTCCCTCTTTTTGATGCTGTAATTTTTGTTCATCATGTTTTGCTGTGATGTTACATAG
GTAGATTTGTATGTAGTTTTAATGTCACCTATAACAAAATGTGTTTGGTAGCAGATTGTCCAGAAAGCAT
TTTAAATGAAGAGGTATAAACCCTTAAGGGCCAAAATTCTGTATATTAGATTACTCTTAAACGAAAAACC
AGCTGCCGCTTTTATGTACACATATTACATACGAGTAGGCAGCAGACTTTAAAAATAAAAAAAACCTAGG
CATGTTGATGTTGCAAAATGCTGTATAAAGCTGAAACCTGTTCATTCAGTGCCATTGTAGTTGACATGAA
GCGATTGTAAAACTGTCTCCGATTTTTCTCTGGTTTATTAAAATGCTAACTATAACATTTTTTGTGAATA
CTTTGAATGTTTCCTAACAGTTGTGATGTTACTGTTCCGTTTTATGCTCTTATTCCAAGTTCATTTTTAA
TGGTTTGGAAGCCATTTTTGTAATGAATAAATGTTCATGCTGTACAGTATCTGTAGCATGCCGTTCTGGA
TTAATAAAAGCAACTTAGTATGTGCAGATAAA,
Protein sequence (SEQ ID NO: 70):
MPQLNGGGGDDLGANDELISFKDEGEQEEKSSENSSAERDLADVKSSLVNESETNONSSSDSEAERRPPPRSESF
RDKSRESLEEAAKRQDGGLFKGPPYPGYPFIMIPDLTSPYLPNGSLSPTARTLHFQSGSTHYSAYKTIEHQIAVQ
YLQMKWPLLDVQAGSLQSRQALKDARSPSPAHIVSNKVPVVQHPHHVHPLTPLITYSNEHFTPGNPPPHLPADVD
PKTGIPRPPHPPDISPYYPLSPGTVGQIPHPLGWLVPQQGQPVYPITTGGFRHPYPTALTVNASMSRFPPHMVPP
HHTLHTTGIPHPAIVTPTVKQESSQSDVGSLHSSKHQDSKKEEEKKKPHIKKPLNAFMLYMKEMRAKVVAECTLK
ESAAINQILGRRWHALSREEQAKYYELARKERQLHMQLYPGWSARDNYGKKKKRKRDKQPGETNGEKKSAFATYK
VKAAASAHPLQMEAY
>NM_001349870.2 Homo sapiens transcription
factor 7 like 2 (TCF7L2), transcript variant
14 (SEQ ID NO: 71)
AGATAAAGTGCCTGCCTCCTGCACTCGGTTGATCATTTTCCCTCATGGCTGACCGTAATTTCTTTGCTAC
TCTTAGCGGCAGCTCTGTCCCTGCTGCCCAAGGTAACGGGTGAGGGAGGGGACTTTGCTGAGGTGCCCTG
AGCTGGCTGGGGAACATGGACCTTCCCCCTTCCTTCACGGATTCTGTAGTGGCAAAGATTAGAAAGTAGA
AGGAGTTTTTAAAATTTTTTTTAAAAAATTAAATCTGCAACCAGCTGAACTCTAGATTATTAGTGGACTT
TTTTGTTTTTTATTTTTAAGATTTTTTTAGGGATGTGTGTACAGGGGGAAGGATTAACAAGATAATTCTA
GGCAATATGAGCGACAACTTTGAGCATTTTGAGCCTACTCGGCCAGGAATCTGGAGCCATTTCGATCTTT
TTAGATGCTTTTTTTCAGTTCTTTAAGTGAAAGGTTTAAAGGGAGGGGAAGCTGGGAGGAAAAAGAAAAG
TGAGAAAAGCAGAAAGGGAGGAGGAAGAAAAACTGGCCCAGTCCCATCTCGAAGGTACAGAGAAAGTTTT
TTTAGAGGTGGAGAGAGGGAAAACCAAGGCTGTTATGTACCCTAGGGACACAATTTTAGTGGGAATGGAA
GATTTGAGTGGTAAGGGAGGCGCAGTGGCCTCCGGGGTCTCTCTGTTCCAGCAGTTGGGCGGTTGGCACC
TCAATTTGAATTGCTGGTCTAACAAAGTGCCAGTGGTGCAGCACCCTCACCATGTCCACCCCCTCACGCC
TCTTATCACGTACAGCAATGAACACTTCACGCCGGGAAACCCACCTCCACACTTACCAGCCGACGTAGAC
CCCAAAACAGGAATCCCACGGCCTCCGCACCCTCCAGATATATCCCCGTATTACCCACTATCGCCTGGCA
CCGTAGGACAAATCCCCCATCCGCTAGGATGGTTAGTACCACAGCAAGGTCAACCAGTGTACCCAATCAC
GACAGGAGGATTCAGACACCCCTACCCCACAGCTCTGACCGTCAATGCTTCCATGTCCAGGTTCCCTCCC
CATATGGTCCCACCACATCATACGCTACACACGACGGGCATTCCGCATCCGGCCATAGTCACACCAACAG
TCAAACAGGAATCGTCCCAGAGTGATGTCGGCTCACTCCATAGTTCAAAGCATCAGGACTCCAAAAAGGA
AGAAGAAAAGAAGAAGCCCCACATAAAGAAACCTCTTAATGCATTCATGTTGTATATGAAGGAAATGAGA
GCAAAGGTCGTAGCTGAGTGCACGTTGAAAGAAAGCGCGGCCATCAACCAGATCCTTGGGCGGAGGTGGC
ATGCACTGTCCAGAGAAGAGCAAGCGAAATACTACGAGCTGGCCCGGAAGGAGCGACAGCTTCATATGCA
ACTGTACCCCGGCTGGTCCGCGCGGGATAACTATGGAAAGAAGAAGAAGAGGAAAAGGGACAAGCAGCCG
GGAGAGACCAATGAACACAGCGAATGTTTCCTAAATCCTTGCCTTTCACTTCCTCCGATTACAGACCTGA
GCGCTCCTAAGAAATGCCGAGCGCGCTTTGGCCTTGATCAACAGAATAACTGGTGCGGCCCTTGCAGTCT
TTGAATTTGGAATATTACAATGGAGAAAAAAAAAGTGCGTTCGCTACATACAAGGTGAAGGCAGCTGCCT
CAGCCCACCCTCTTCAGATGGAAGCTTACTAGATTCGCCTCCCCCCTCCCCGAACCTGCTAGGCTCCCCT
CCCCGAGACGCCAAGTCACAGACTGAGCAGACCCAGCCTCTGTCGCTGTCCCTGAAGCCCGACCCCCTGG
CCCACCTGTCCATGATGCCTCCGCCACCCGCCCTCCTGCTCGCTGAGGCCACCCACAAGGCCTCCGCCCT
CTGTCCCAACGGGGCCCTGGACCTGCCCCCAGCCGCTTTGCAGCCTGCCGCCCCCTCCTCATCAATTGCA
CAGCCGTCGACTTCTTCCTTACATTCCCACAGCTCCCTGGCCGGGACCCAGCCCCAGCCGCTGTCGCTCG
TCACCAAGTCTTTAGAATAGCTTTAGCGTCGTGAACCCCGCTGCTTTGTTTATGGTTTTGTTTCACTTTT
CTTAATTTGCCCCCCACCCCCACCTTGAAAGGTTTTGTTTTGTACTCTCTTAATTTTGTGCCATGTGGCT
ACATTAGTTGATGTTTATCGAGTTCATTGGTCAATATTTGACCCATTCTTATTTCAATTTCTCCTTTTAA
ATATGTAGATGAGAGAAGAACCTCATGATTCTACCAAAATTTTTATCAACAGCTGTTTAAAGTCTTTGTA
GCGTTTAAAAAATATATATATATACATAACTGTTATGTAGTTCGGATAGCTTAGTTTTAAAAGACTGATT
AAAAAACAAAAAGAAAAAAAAAGCAATTTTGAAGCAGCCCTCCAGAAGGAGTTGGTTCTGTATTATTTGT
ATTAAATACGAGCTTGCGAACCAATCATTTTACATCTGGTTTTTAAACCGTAAGGGCACCATGAATGCAG
TGCCGTTACTTTTTTTTTTTTTTTCTGTGTGAAACAACTCTTATTGTGATGTTACTTGTTATTGTTTAAA
TGTACAGAAACAAAGGGTAAAAATGTGTTAATATACCTTGTTCCATGGTGTTGTTCTTTTGGGGGGAGGG
GACGCTACTCAACACTTAATAGAATCACAACGCTGTTGGGCCAGTAGTATTTATTGCTTTAGAGATTGCT
TGTCGTACCTGTATGTCGTCCCTTTTTAAATATGTTTTCCTTTTTCTTGAAACTGTATAAAGTTTTTTTC
CCCCTTAGCATAAGCATCTTATATATAACAACTCATTTGTACAAGGTTTTTAAGTTTATATATAAAATGT
GTATATATATTTTTGTTTCCCCTTTTTGACTTTTTTTTTTCTGTATGAAACCCAGATGTCACCAAATGGA
CATTAATAGTTGCATTAAGGATCAGTAGCATTAACAAAAGTTGCTTTAAAAGCCATTATGTAAAACAAGA
CTTGAAAATGAGTGAGGGAATTTTAGCGACACTGTCTGAGCAGCAGTGGGAACCATCTTCGTTTCCCCTT
TGAACTCCCAGTGGGATGCCCTACCCTGCGCCCTTAGGACCCGGACTGACCGTGTACAAAACTTTACGTG
CCAAAATTCTCAGTGAATTTAGCTTTCTCCCTCTTTTTGATGCTGTAATTTTTGTTCATCATGTTTTGCT
GTGATGTTACATAGGTAGATTTGTATGTAGTTTTAATGTCACCTATAACAAAATGTGTTTGGTAGCAGAT
TGTCCAGAAAGCATTTTAAATGAAGAGGTATAAACCCTTAAGGGCCAAAATTCTGTATATTAGATTACTC
TTAAACGAAAAACCAGCTGCCGCTTTTATGTACACATATTACATACGAGTAGGCAGCAGACTTTAAAAAT
AAAAAAAACCTAGGCATGTTGATGTTGCAAAATGCTGTATAAAGCTGAAACCTGTTCATTCAGTGCCATT
GTAGTTGACATGAAGCGATTGTAAAACTGTCTCCGATTTTTCTCTGGTTTATTAAAATGCTAACTATAAC
ATTTTTTGTGAATACTTTGAATGTTTCCTAACAGTTGTGATGTTACTGTTCCGTTTTATGCTCTTATTCC
AAGTTCATTTTTAATGGTTTGGAAGCCATTTTTGTAATGAATAAATGTTCATGCTGTACAGTATCTGTAG
CATGCCGTTCTGGATTAATAAAAGCAACTTAGTATGTGCAGATAAA,
Protein sequence (SEQ ID NO: 72):
MYPRDTILVGMEDLSGKGGAVASGVSLFQQLGGWHLNLNCWSNKVPVVQHPHHVHPLTPLITYSNEHFTPGNPPP
HLPADVDPKTGIPRPPHPPDISPYYPLSPGTVGQIPHPLGWLVPQQGQPVYPITTGGFRHPYPTALTVNASMSRF
PPHMVPPHHTLHTTGIPHPAIVTPTVKQESSQSDVGSLHSSKHQDSKKEEEKKKPHIKKPLNAFMLYMKEMRAKV
VAECTLKESAAINQILGRRWHALSREEQAKYYELARKERQLHMQLYPGWSARDNYGKKKKRKRDKQPGETNEHSE
CFLNPCLSLPPITDLSAPKKCRARFGLDQQNNWCGPCS
>NM_001349871.1 Homo sapiens transcription
factor 7 like 2 (TCF7L2), transcript variant
15 (SEQ ID NO: 73)
ATTGTGCTCTTCCCTATCAAGCGAGATAATTCTGTGAATGGAACTGTGCGTGAGCATCTTGTCTGGCGGC
GCTGCTGGTGTGTGCTGCTCCCCTGTGCCGCGGGTGCGCGGCGGCGGCGCGGGCTGCAGGGCGGGTGCTG
CCCTCCAGTGGAGCCCGCGGCCGGCGCGGGCCCTGGGCAGCATCTGGGCGCAGGCAGCATGCCCGCGAGC
CGGCAGCGGGAAGGACAGCGCCGCAACCCTCTCTAGATGTCTAACAAAGTGCCAGTGGTGCAGCACCCTC
ACCATGTCCACCCCCTCACGCCTCTTATCACGTACAGCAATGAACACTTCACGCCGGGAAACCCACCTCC
ACACTTACCAGCCGACGTAGACCCCAAAACAGGAATCCCACGGCCTCCGCACCCTCCAGATATATCCCCG
TATTACCCACTATCGCCTGGCACCGTAGGACAAATCCCCCATCCGCTAGGATGGTTAGTACCACAGCAAG
GTCAACCAGTGTACCCAATCACGACAGGAGGATTCAGACACCCCTACCCCACAGCTCTGACCGTCAATGC
TTCCATGTCCAGGTTCCCTCCCCATATGGTCCCACCACATCATACGCTACACACGACGGGCATTCCGCAT
CCGGCCATAGTCACACCAACAGTCAAACAGGAATCGTCCCAGAGTGATGTCGGCTCACTCCATAGTTCAA
AGCATCAGGACTCCAAAAAGGAAGAAGAAAAGAAGAAGCCCCACATAAAGAAACCTCTTAATGCATTCAT
GTTGTATATGAAGGAAATGAGAGCAAAGGTCGTAGCTGAGTGCACGTTGAAAGAAAGCGCGGCCATCAAC
CAGATCCTTGGGCGGAGGTGGCATGCACTGTCCAGAGAAGAGCAAGCGAAATACTACGAGCTGGCCCGGA
AGGAGCGACAGCTTCATATGCAACTGTACCCCGGCTGGTCCGCGCGGGATAACTATGGAAAGAAGAAGAA
GAGGAAAAGGGACAAGCAGCCGGGAGAGACCAATGAACACAGCGAATGTTTCCTAAATCCTTGCCTTTCA
CTTCCTCCGATTACAGACCTGAGCGCTCCTAAGAAATGCCGAGCGCGCTTTGGCCTTGATCAACAGAATA
ACTGGTGCGGCCCTTGCAGTCTTTGAATTTGGAATATTACAATGGAGAAAAAAAAAGTGCGTTCGCTACA
TACAAGGTGAAGGCAGCTGCCTCAGCCCACCCTCTTCAGATGGAAGCTTACTAGATTCGCCTCCCCCCTC
CCCGAACCTGCTAGGCTCCCCTCCCCGAGACGCCAAGTCACAGACTGAGCAGACCCAGCCTCTGTCGCTG
TCCCTGAAGCCCGACCCCCTGGCCCACCTGTCCATGATGCCTCCGCCACCCGCCCTCCTGCTCGCTGAGG
CCACCCACAAGGCCTCCGCCCTCTGTCCCAACGGGGCCCTGGACCTGCCCCCAGCCGCTTTGCAGCCTGC
CGCCCCCTCCTCATCAATTGCACAGCCGTCGACTTCTTCCTTACATTCCCACAGCTCCCTGGCCGGGACC
CAGCCCCAGCCGCTGTCGCTCGTCACCAAGTCTTTAGAATAGCTTTAGCGTCGTGAACCCCGCTGCTTTG
TTTATGGTTTTGTTTCACTTTTCTTAATTTGCCCCCCACCCCCACCTTGAAAGGTTTTGTTTTGTACTCT
CTTAATTTTGTGCCATGTGGCTACATTAGTTGATGTTTATCGAGTTCATTGGTCAATATTTGACCCATTC
TTATTTCAATTTCTCCTTTTAAATATGTAGATGAGAGAAGAACCTCATGATTCTACCAAAATTTTTATCA
ACAGCTGTTTAAAGTCTTTGTAGCGTTTAAAAAATATATATATATACATAACTGTTATGTAGTTCGGATA
GCTTAGTTTTAAAAGACTGATTAAAAAACAAAAAGAAAAAAAAAGCAATTTTGAAGCAGCCCTCCAGAAG
GAGTTGGTTCTGTATTATTTGTATTAAATACGAGCTTGCGAACCAATCATTTTACATCTGGTTTTTAAAC
CGTAAGGGCACCATGAATGCAGTGCCGTTACTTTTTTTTTTTTTTTCTGTGTGAAACAACTCTTATTGTG
ATGTTACTTGTTATTGTTTAAATGTACAGAAACAAAGGGTAAAAATGTGTTAATATACCTTGTTCCATGG
TGTTGTTCTTTTGGGGGGAGGGGACGCTACTCAACACTTAATAGAATCACAACGCTGTTGGGCCAGTAGT
ATTTATTGCTTTAGAGATTGCTTGTCGTACCTGTATGTCGTCCCTTTTTAAATATGTTTTCCTTTTTCTT
GAAACTGTATAAAGTTTTTTTCCCCCTTAGCATAAGCATCTTATATATAACAACTCATTTGTACAAGGTT
TTTAAGTTTATATATAAAATGTGTATATATATTTTTGTTTCCCCTTTTTGACTTTTTTTTTTCTGTATGA
AACCCAGATGTCACCAAATGGACATTAATAGTTGCATTAAGGATCAGTAGCATTAACAAAAGTTGCTTTA
AAAGCCATTATGTAAAACAAGACTTGAAAATGAGTGAGGGAATTTTAGCGACACTGTCTGAGCAGCAGTG
GGAACCATCTTCGTTTCCCCTTTGAACTCCCAGTGGGATGCCCTACCCTGCGCCCTTAGGACCCGGACTG
ACCGTGTACAAAACTTTACGTGCCAAAATTCTCAGTGAATTTAGCTTTCTCCCTCTTTTTGATGCTGTAA
TTTTTGTTCATCATGTTTTGCTGTGATGTTACATAGGTAGATTTGTATGTAGTTTTAATGTCACCTATAA
CAAAATGTGTTTGGTAGCAGATTGTCCAGAAAGCATTTTAAATGAAGAGGTATAAACCCTTAAGGGCCAA
AATTCTGTATATTAGATTACTCTTAAACGAAAAACCAGCTGCCGCTTTTATGTACACATATTACATACGA
GTAGGCAGCAGACTTTAAAAATAAAAAAAACCTAGGCATGTTGATGTTGCAAAATGCTGTATAAAGCTGA
AACCTGTTCATTCAGTGCCATTGTAGTTGACATGAAGCGATTGTAAAACTGTCTCCGATTTTTCTCTGGT
TTATTAAAATGCTAACTATAACATTTTTTGTGAATACTTTGAATGTTTCCTAACAGTTGTGATGTTACTG
TTCCGTTTTATGCTCTTATTCCAAGTTCATTTTTAATGGTTTGGAAGCCATTTTTGTAATGAATAAATGT
TCATGCTGTACAGTATCTGTAGCATGCCGTTCTGGATTAATAAAAGCAACTTAGTATGTGCAGATAAA,
Protein sequence (SEQ ID NO: 74):
MSNKVPVVQHPHHVHPLTPLITYSNEHFTPGNPPPHLPADVDPKTGIPRPPHPPDISPYYPLSPGTVGQIPHPLG
WLVPQQGQPVYPITTGGFRHPYPTALTVNASMSRFPPHMVPPHHTLHTTGIPHPAIVTPTVKQESSQSDVGSLHS
SKHQDSKKEEEKKKPHIKKPLNAFMLYMKEMRAKVVAECTLKESAAINQILGRRWHALSREEQAKYYELARKERQ
LHMQLYPGWSARDNYGKKKKRKRDKQPGETNEHSECFLNPCLSLPPITDLSAPKKCRARFGLDQQNNWCGPCSL
>NM_001363501.2 Homo sapiens transcription
factor 7 like 2 (TCF7L2), transcript variant
16 (SEQ ID NO: 75)
GTCAATAATCTCCGCTCCCAGACTACTCCGTTCCTCCGGATTTCGATCCCCCTTTTTCTATCTGTCAATC
AGCGCCGCCTTTGAACTGAAAAGCTCTCAGTCTAACTTCAACTCACTCAAATCCGAGCGGCACGAGCACC
TCCTGTATCTTCGGCTTCCCCCCCCCTTTGCTCTTTATATCTGACTTCTTGTTGTTGTTGGTGTTTTTTT
TTTTTTTACCCCCCTTTTTTATTTATTATTTTTTTGCACATTGATCGGATCCTTGGGAACGAGAGAAAAA
AGAAACCCAAACTCACGCGTGCAGAAGATCTCCCCCCCCTTCCCCTCCCCTCCTCCCTCTTTTCCCCTCC
CCAGGAGAAAAAGACCCCCAAGCAGAAAAAAGTTCACCTTGGACTCGTCTTTTTCTTGCAATATTTTTTG
GGGGGGCAAAACTTTTTGGGGGTGATTTTTTTTGGCTTTTCTTCCTCCTTCATTTTTCTTCCAAAATTGC
TGCTGGTGGGTGAAAAAAAAATGCCGCAGCTGAACGGCGGTGGAGGGGATGACCTAGGCGCCAACGACGA
ACTGATTTCCTTCAAAGACGAGGGCGAACAGGAGGAGAAGAGCTCCGAAAACTCCTCGGCAGAGAGGGAT
TTAGCTGATGTCAAATCGTCTCTAGTCAATGAATCAGAAACGAATCAAAACAGCTCCTCCGATTCCGAGG
CGGAAAGACGGCCTCCGCCTCGCTCCGAAAGTTTCCGAGACAAATCCCGGGAAAGTTTGGAAGAAGCGGC
CAAGAGGCAAGATGGAGGGCTCTTTAAGGGGCCACCGTATCCCGGCTACCCCTTCATCATGATCCCCGAC
CTGACGAGCCCCTACCTCCCCAACGGATCGCTCTCGCCCACCGCCCGAACCCTCCATTTTCAGTCCGGCA
GCACACATTACTCTGCGTACAAAACGATTGAACACCAGATTGCAGTTCAGTATCTCCAGATGAAATGGCC
ACTGCTTGATGTCCAGGCAGGGAGCCTCCAGAGTAGACAAGCCCTCAAGGATGCCCGGTCCCCATCACCG
GCACACATTGTCTCTAACAAAGTGCCAGTGGTGCAGCACCCTCACCATGTCCACCCCCTCACGCCTCTTA
TCACGTACAGCAATGAACACTTCACGCCGGGAAACCCACCTCCACACTTACCAGCCGACGTAGACCCCAA
AACAGGAATCCCACGGCCTCCGCACCCTCCAGATATATCCCCGTATTACCCACTATCGCCTGGCACCGTA
GGACAAATCCCCCATCCGCTAGGATGGTTAGTACCACAGCAAGGTCAACCAGTGTACCCAATCACGACAG
GAGGATTCAGACACCCCTACCCCACAGCTCTGACCGTCAATGCTTCCATGTCCAGGTTCCCTCCCCATAT
GGTCCCACCACATCATACGCTACACACGACGGGCATTCCGCATCCGGCCATAGTCACACCAACAGTCAAA
CAGGAATCGTCCCAGAGTGATGTCGGCTCACTCCATAGTTCAAAGCATCAGGACTCCAAAAAGGAAGAAG
AAAAGAAGAAGCCCCACATAAAGAAACCTCTTAATGCATTCATGTTGTATATGAAGGAAATGAGAGCAAA
GGTCGTAGCTGAGTGCACGTTGAAAGAAAGCGCGGCCATCAACCAGATCCTTGGGCGGAGGTGGCATGCA
CTGTCCAGAGAAGAGCAAGCGAAATACTACGAGCTGGCCCGGAAGGAGCGACAGCTTCATATGCAACTGT
ACCCCGGCTGGTCCGCGCGGGATAACTATGGAAAGAAGAAGAAGAGGAAAAGGGACAAGCAGCCGGGAGA
GACCAATGAACACAGCGAATGTTTCCTAAATCCTTGCCTTTCACTTCCTCCGATTACAGGAGAAAAAAAA
AGTGCGTTCGCTACATACAAGGTGAAGGCAGCTGCCTCAGCCCACCCTCTTCAGATGGAAGCTTACTAGA
TTCGCCTCCCCCCTCCCCGAACCTGCTAGGCTCCCCTCCCCGAGACGCCAAGTCACAGACTGAGCAGACC
CAGCCTCTGTCGCTGTCCCTGAAGCCCGACCCCCTGGCCCACCTGTCCATGATGCCTCCGCCACCCGCCC
TCCTGCTCGCTGAGGCCACCCACAAGGCCTCCGCCCTCTGTCCCAACGGGGCCCTGGACCTGCCCCCAGC
CGCTTTGCAGCCTGCCGCCCCCTCCTCATCAATTGCACAGCCGTCGACTTCTTCCTTACATTCCCACAGC
TCCCTGGCCGGGACCCAGCCCCAGCCGCTGTCGCTCGTCACCAAGTCTTTAGAATAGCTTTAGCGTCGTG
AACCCCGCTGCTTTGTTTATGGTTTTGTTTCACTTTTCTTAATTTGCCCCCCACCCCCACCTTGAAAGGT
TTTGTTTTGTACTCTCTTAATTTTGTGCCATGTGGCTACATTAGTTGATGTTTATCGAGTTCATTGGTCA
ATATTTGACCCATTCTTATTTCAATTTCTCCTTTTAAATATGTAGATGAGAGAAGAACCTCATGATTCTA
CCAAAATTTTTATCAACAGCTGTTTAAAGTCTTTGTAGCGTTTAAAAAATATATATATATACATAACTGT
TATGTAGTTCGGATAGCTTAGTTTTAAAAGACTGATTAAAAAACAAAAAGAAAAAAAAAGCAATTTTGAA
GCAGCCCTCCAGAAGGAGTTGGTTCTGTATTATTTGTATTAAATACGAGCTTGCGAACCAATCATTTTAC
ATCTGGTTTTTAAACCGTAAGGGCACCATGAATGCAGTGCCGTTACTTTTTTTTTTTTTTTCTGTGTGAA
ACAACTCTTATTGTGATGTTACTTGTTATTGTTTAAATGTACAGAAACAAAGGGTAAAAATGTGTTAATA
TACCTTGTTCCATGGTGTTGTTCTTTTGGGGGGAGGGGACGCTACTCAACACTTAATAGAATCACAACGC
TGTTGGGCCAGTAGTATTTATTGCTTTAGAGATTGCTTGTCGTACCTGTATGTCGTCCCTTTTTAAATAT
GTTTTCCTTTTTCTTGAAACTGTATAAAGTTTTTTTCCCCCTTAGCATAAGCATCTTATATATAACAACT
CATTTGTACAAGGTTTTTAAGTTTATATATAAAATGTGTATATATATTTTTGTTTCCCCTTTTTGACTTT
TTTTTTTCTGTATGAAACCCAGATGTCACCAAATGGACATTAATAGTTGCATTAAGGATCAGTAGCATTA
ACAAAAGTTGCTTTAAAAGCCATTATGTAAAACAAGACTTGAAAATGAGTGAGGGAATTTTAGCGACACT
GTCTGAGCAGCAGTGGGAACCATCTTCGTTTCCCCTTTGAACTCCCAGTGGGATGCCCTACCCTGCGCCC
TTAGGACCCGGACTGACCGTGTACAAAACTTTACGTGCCAAAATTCTCAGTGAATTTAGCTTTCTCCCTC
TTTTTGATGCTGTAATTTTTGTTCATCATGTTTTGCTGTGATGTTACATAGGTAGATTTGTATGTAGTTT
TAATGTCACCTATAACAAAATGTGTTTGGTAGCAGATTGTCCAGAAAGCATTTTAAATGAAGAGGTATAA
ACCCTTAAGGGCCAAAATTCTGTATATTAGATTACTCTTAAACGAAAAACCAGCTGCCGCTTTTATGTAC
ACATATTACATACGAGTAGGCAGCAGACTTTAAAAATAAAAAAAACCTAGGCATGTTGATGTTGCAAAAT
GCTGTATAAAGCTGAAACCTGTTCATTCAGTGCCATTGTAGTTGACATGAAGCGATTGTAAAACTGTCTC
CGATTTTTCTCTGGTTTATTAAAATGCTAACTATAACATTTTTTGTGAATACTTTGAATGTTTCCTAACA
GTTGTGATGTTACTGTTCCGTTTTATGCTCTTATTCCAAGTTCATTTTTAATGGTTTGGAAGCCATTTTT
GTAATGAATAAATGTTCATGCTGTACAGTATCTGTAGCATGCCGTTCTGGATTAATAAAAGCAACTTAGT
ATGTGCAGATAAA,
Protein sequence (SEQ ID NO: 76):
MPQLNGGGGDDLGANDELISFKDEGEQEEKSSENSSAERDLADVKSSLVNESETNONSSSDSEAERRPPPRSESF
RDKSRESLEEAAKRQDGGLFKGPPYPGYPFIMIPDLTSPYLPNGSLSPTARTLHFQSGSTHYSAYKTIEHQIAVQ
YLQMKWPLLDVQAGSLQSRQALKDARSPSPAHIVSNKVPVVQHPHHVHPLTPLITYSNEHFTPGNPPPHLPADVD
PKTGIPRPPHPPDISPYYPLSPGTVGQIPHPLGWLVPQQGQPVYPITTGGFRHPYPTALTVNASMSRFPPHMVPP
HHTLHTTGIPHPAIVTPTVKQESSQSDVGSLHSSKHQDSKKEEEKKKPHIKKPLNAFMLYMKEMRAKVVAECTLK
ESAAINQILGRRWHALSREEQAKYYELARKERQLHMQLYPGWSARDNYGKKKKRKRDKQPGETNEHSECFLNPCL
SLPPITGEKKSAFATYKVKAAASAHPLQMEAY
>NM_001367943.1 Homo sapiens transcription
factor 7 like 2 (TCF7L2), transcript variant
17, mRNA ((SEQ ID NO: 77))
GTCAATAATCTCCGCTCCCAGACTACTCCGTTCCTCCGGATTTCGATCCCCCTTTTTCTATCTGTCAATC
AGCGCCGCCTTTGAACTGAAAAGCTCTCAGTCTAACTTCAACTCACTCAAATCCGAGCGGCACGAGCACC
TCCTGTATCTTCGGCTTCCCCCCCCCTTTGCTCTTTATATCTGACTTCTTGTTGTTGTTGGTGTTTTTTT
TTTTTTTACCCCCCTTTTTTATTTATTATTTTTTTGCACATTGATCGGATCCTTGGGAACGAGAGAAAAA
AGAAACCCAAACTCACGCGTGCAGAAGATCTCCCCCCCCTTCCCCTCCCCTCCTCCCTCTTTTCCCCTCC
CCAGGAGAAAAAGACCCCCAAGCAGAAAAAAGTTCACCTTGGACTCGTCTTTTTCTTGCAATATTTTTTG
GGGGGGCAAAACTTTTTGGGGGTGATTTTTTTTGGCTTTTCTTCCTCCTTCATTTTTCTTCCAAAATTGC
TGCTGGTGGGTGAAAAAAAAATGCCGCAGCTGAACGGCGGTGGAGGGGATGACCTAGGCGCCAACGACGA
ACTGATTTCCTTCAAAGACGAGGGCGAACAGGAGGAGAAGAGCTCCGAAAACTCCTCGGCAGAGAGGGAT
TTAGCTGATGTCAAATCGTCTCTAGTCAATGAATCAGAAACGAATCAAAACAGCTCCTCCGATTCCGAGG
CGGAAAGACGGCCTCCGCCTCGCTCCGAAAGTTTCCGAGACAAATCCCGGGAAAGTTTGGAAGAAGCGGC
CAAGAGGCAAGATGGAGGGCTCTTTAAGGGGCCACCGTATCCCGGCTACCCCTTCATCATGATCCCCGAC
CTGACGAGCCCCTACCTCCCCAACGGATCGCTCTCGCCCACCGCCCGAACCCTCCATTTTCAGTCCGGCA
GCACACATTACTCTGCGTACAAAACGATTGAACACCAGATTGCAGTTCAGTATCTCCAGATGAAATGGCC
ACTGCTTGATGTCCAGGCAGGGAGCCTCCAGAGTAGACAAGCCCTCAAGGATGCCCGGTCCCCATCACCG
GCACACATTGTCTCTAACAAAGTGCCAGTGGTGCAGCACCCTCACCATGTCCACCCCCTCACGCCTCTTA
TCACGTACAGCAATGAACACTTCACGCCGGGAAACCCACCTCCACACTTACCAGCCGACGTAGACCCCAA
AACAGGAATCCCACGGCCTCCGCACCCTCCAGATATATCCCCGTATTACCCACTATCGCCTGGCACCGTA
GGACAAATCCCCCATCCGCTAGGATGGTTAGTACCACAGCAAGGTCAACCAGTGTACCCAATCACGACAG
GAGGATTCAGACACCCCTACCCCACAGCTCTGACCGTCAATGCTTCCATGTCCAGGTTCCCTCCCCATAT
GGTCCCACCACATCATACGCTACACACGACGGGCATTCCGCATCCGGCCATAGTCACACCAACAGTCAAA
CAGGAATCGTCCCAGAGTGATGTCGGCTCACTCCATAGTTCAAAGCATCAGGACTCCAAAAAGGAAGAAG
AAAAGAAGAAGCCCCACATAAAGAAACCTCTTAATGCATTCATGTTGTATATGAAGGAAATGAGAGCAAA
GGTCGTAGCTGAGTGCACGTTGAAAGAAAGCGCGGCCATCAACCAGATCCTTGGGCGGAGGTGGCATGCA
CTGTCCAGAGAAGAGCAAGCGAAATACTACGAGCTGGCCCGGAAGGAGCGACAGCTTCATATGCAACTGT
ACCCCGGCTGGTCCGCGCGGGATAACTATGGAAAGAAGAAGAAGAGGAAAAGGGACAAGCAGCCGGGAGA
GACCAATGAACACAGCGAATGTTTCCTAAATCCTTGCCTTTCACTTCCTCCGATTACAGACCTGAGCGCT
CCTAAGAAATGCCGAGCGCGCTTTGGCCTTGATCAACAGAATAACTGGTGCGGCCCTTGCAGGAGAAAAA
AAAAGTGCGTTCGCTACATACAAGGTGAAGGCAGCTGCCTCAGCCCACCCTCTTCAGATGGAAGCTTACT
AGATTCGCCTCCCCCCTCCCCGAACCTGCTAGGCTCCCCTCCCCGAGACGCCAAGTCACAGACTGAGCAG
ACCCAGCCTCTGTCGCTGTCCCTGAAGCCCGACCCCCTGGCCCACCTGTCCATGATGCCTCCGCCACCCG
CCCTCCTGCTCGCTGAGGCCACCCACAAGGCCTCCGCCCTCTGTCCCAACGGGGCCCTGGACCTGCCCCC
AGCCGCTTTGCAGCCTGCCGCCCCCTCCTCATCAATTGCACAGCCGTCGACTTCTTCCTTACATTCCCAC
AGCTCCCTGGCCGGGACCCAGCCCCAGCCGCTGTCGCTCGTCACCAAGTCTTTAGAATAGCTTTAGCGTC
GTGAACCCCGCTGCTTTGTTTATGGTTTTGTTTCACTTTTCTTAATTTGCCCCCCACCCCCACCTTGAAA
GGTTTTGTTTTGTACTCTCTTAATTTTGTGCCATGTGGCTACATTAGTTGATGTTTATCGAGTTCATTGG
TCAATATTTGACCCATTCTTATTTCAATTTCTCCTTTTAAATATGTAGATGAGAGAAGAACCTCATGATT
CTACCAAAATTTTTATCAACAGCTGTTTAAAGTCTTTGTAGCGTTTAAAAAATATATATATATACATAAC
TGTTATGTAGTTCGGATAGCTTAGTTTTAAAAGACTGATTAAAAAACAAAAAGAAAAAAAAAGCAATTTT
GAAGCAGCCCTCCAGAAGGAGTTGGTTCTGTATTATTTGTATTAAATACGAGCTTGCGAACCAATCATTT
TACATCTGGTTTTTAAACCGTAAGGGCACCATGAATGCAGTGCCGTTACTTTTTTTTTTTTTTTCTGTGT
GAAACAACTCTTATTGTGATGTTACTTGTTATTGTTTAAATGTACAGAAACAAAGGGTAAAAATGTGTTA
ATATACCTTGTTCCATGGTGTTGTTCTTTTGGGGGGAGGGGACGCTACTCAACACTTAATAGAATCACAA
CGCTGTTGGGCCAGTAGTATTTATTGCTTTAGAGATTGCTTGTCGTACCTGTATGTCGTCCCTTTTTAAA
TATGTTTTCCTTTTTCTTGAAACTGTATAAAGTTTTTTTCCCCCTTAGCATAAGCATCTTATATATAACA
ACTCATTTGTACAAGGTTTTTAAGTTTATATATAAAATGTGTATATATATTTTTGTTTCCCCTTTTTGAC
TTTTTTTTTTCTGTATGAAACCCAGATGTCACCAAATGGACATTAATAGTTGCATTAAGGATCAGTAGCA
TTAACAAAAGTTGCTTTAAAAGCCATTATGTAAAACAAGACTTGAAAATGAGTGAGGGAATTTTAGCGAC
ACTGTCTGAGCAGCAGTGGGAACCATCTTCGTTTCCCCTTTGAACTCCCAGTGGGATGCCCTACCCTGCG
CCCTTAGGACCCGGACTGACCGTGTACAAAACTTTACGTGCCAAAATTCTCAGTGAATTTAGCTTTCTCC
CTCTTTTTGATGCTGTAATTTTTGTTCATCATGTTTTGCTGTGATGTTACATAGGTAGATTTGTATGTAG
TTTTAATGTCACCTATAACAAAATGTGTTTGGTAGCAGATTGTCCAGAAAGCATTTTAAATGAAGAGGTA
TAAACCCTTAAGGGCCAAAATTCTGTATATTAGATTACTCTTAAACGAAAAACCAGCTGCCGCTTTTATG
TACACATATTACATACGAGTAGGCAGCAGACTTTAAAAATAAAAAAAACCTAGGCATGTTGATGTTGCAA
AATGCTGTATAAAGCTGAAACCTGTTCATTCAGTGCCATTGTAGTTGACATGAAGCGATTGTAAAACTGT
CTCCGATTTTTCTCTGGTTTATTAAAATGCTAACTATAACATTTTTTGTGAATACTTTGAATGTTTCCTA
ACAGTTGTGATGTTACTGTTCCGTTTTATGCTCTTATTCCAAGTTCATTTTTAATGGTTTGGAAGCCATT
TTTGTAATGAATAAATGTTCATGCTGTACAGTATCTGTAGCATGCCGTTCTGGATTAATAAAAGCAACTT
AGTATGTGCAGATAAA
Protein sequence (SEQ ID NO: 78):
MPQLNGGGGDDLGANDELISFKDEGEQEEKSSENSSAERDLADVKSSLVNESETNONSSSDSEAERRPPPRSESF
RDKSRESLEEAAKRQDGGLFKGPPYPGYPFIMIPDLTSPYLPNGSLSPTARTLHFQSGSTHYSAYKTIEHQIAVQ
YLQMKWPLLDVQAGSLQSRQALKDARSPSPAHIVSNKVPVVQHPHHVHPLTPLITYSNEHFTPGNPPPHLPADVD
PKTGIPRPPHPPDISPYYPLSPGTVGQIPHPLGWLVPQQGQPVYPITTGGFRHPYPTALTVNASMSRFPPHMVPP
HHTLHTTGIPHPAIVTPTVKQESSQSDVGSLHSSKHQDSKKEEEKKKPHIKKPLNAFMLYMKEMRAKVVAECTLK
ESAAINQILGRRWHALSREEQAKYYELARKERQLHMQLYPGWSARDNYGKKKKRKRDKQPGETNEHSECFLNPCL
SLPPITDLSAPKKCRARFGLDQQNNWCGPCRRKKKCVRYIQGEGSCLSPPSSDGSLLDSPPPSPNLLGSPPRDAK
SQTEQTOPLSLSLKPDPLAHLSMMPPPPALLLAEATHKASA
LCPNGALDLPPAALQPAAPSSSIAQPSTSSLHSHSSLAGTQPQPLSLVTKSLE

In one example, the following Human TCF7L2-210 sequence is used. This is sequence is identical to that of variant 2 described above, except the underline/bold residue:

(SEQ ID NO: 79)
MPQLNGGGGDDLGANDELISFKDEGEREEKSSEN
SSAERDLADVKSSLVNESETNONSSSDSEAERRP
PPRSESFRDKSRESLEEAAKRQDGGLFKGPPYPG
YPFIMIPDLTSPYLPNGSLSPTARTYLQMKWPLL
DVQAGSLQSRQALKDARSPSPAHIVSNKVPVVQH
PHHVHPLTPLITYSNEHFTPGNPPPHLPADVDPK
TGIPRPPHPPDISPYYPLSPGTVGQIPHPLGWLV
PQQGQPVYPITTGGFRHPYPTALTVNASMSRFPP
HMVPPHHTLHTTGIPHPAIVTPTVKQESSQSDVG
SLHSSKHQDSKKEEEKKKPHIKKPLNAFMLYMKE
MRAKVVAECTLKESAAINQILGRRWHALSREEQA
KYYELARKERQLHMQLYPGWSARDNYGKKKKRKR
DKQPGETNEHSECFLNPCLSLPPITDLSAPKKCR
ARFGLDQQNNWCGPCRRKKKCVRYIQGEGSCLSP
PSSDGSLLDSPPPSPNLLGSPPRDAKSQTEQTQP
LSLSLKPDPLAHLSMMPPPPALLLAEATHKASAL
CPNGALDLPPAALQPAAPSSSIAQPSTSSLHSHS
SLAGTQPQPLSLVTKSLE.

Listed below are some exemplary Tcf712 mouse isoforms, related nucleic acid sequence information, and related amino acid sequence information. All of these variants/isoforms can be used in the expression cassette, genetic construct, vector, composition, or method disclosed herein.

Tcf7l2 mouse isoforms

| Gene ID | Transcript | Length (nt) | Protein | Length (aa) | Isoform |
|---|---|---|---|---|---|
| 21416 | NR_138565.1 | 4225 | | | |
| 21416 | XM_017318127.3 | 4175 | XP_017173616.1 | 460 | X16 |
| 21416 | XM_017318116.3 | 4255 | XP_017173605.1 | 487 | X9 |
| 21416 | XM_006526860.5 | 4230 | XP_006526923.1 | 626 | X1 |
| 21416 | XM_017318117.3 | 4157 | XP_017173606.1 | 487 | X10 |
| 21416 | XM_017318110.3 | 4179 | XP_017173599.1 | 609 | X3 |
| 21416 | XM_017318114.3 | 4288 | XP_017173603.1 | 494 | X7 |
| 21416 | XM_006526861.5 | 4218 | XP_006526924.1 | 622 | X2 |
| 21416 | XM_017318115.3 | 4276 | XP_017173604.1 | 490 | X8 |
| 21416 | XM_017318119.3 | 4228 | XP_017173608.1 | 478 | X11 |
| 21416 | XM_017318126.3 | 4177 | XP_017173615.1 | 461 | X15 |
| 21416 | XM_036161506.1 | 4234 | XP_036017399.1 | 476 | X12 |
| 21416 | XM_006526866.5 | 4161 | XP_006526929.1 | 603 | X4 |
| 21416 | XM_006526888.5 | 4088 | XP_006526951.1 | 464 | X14 |
| 21416 | XM_017318131.3 | 4135 | XP_017173620.1 | 447 | X18 |
| 21416 | XM_030250847.2 | 4110 | XP_030106707.1 | 586 | X6 |
| 21416 | XM_036161508.1 | 4168 | XP_036017401.1 | 454 | X17 |
| 21416 | XM_017318112.3 | 4149 | XP_017173601.1 | 599 | X5 |
| 21416 | XM_036161507.1 | 4207 | XP_036017400.1 | 467 | X13 |
| 21416 | NM_001331148.1 | 4221 | NP_001318077.1 | 482 | 22 |
| 21416 | NM_001331146.1 | 4196 | NP_001318075.1 | 621 | 20 |
| 21416 | NM_001331149.1 | 4123 | NP_001318078.1 | 482 | 23 |
| 21416 | NM_001331150.1 | 4170 | NP_001318079.1 | 465 | 24 |
| 21416 | NM_001331147.1 | 4145 | NP_001318076.1 | 604 | 21 |
| 21416 | NM_001142920.2 | 4026 | NP_001136392.1 | 435 | 4 |
| 21416 | NM_001331138.1 | 4184 | NP_001318067.1 | 617 | 12 |
| 21416 | NM_001331135.1 | 4111 | NP_001318064.1 | 478 | 9 |
| 21416 | NM_001331136.1 | 4167 | NP_001318065.1 | 464 | 10 |
| 21416 | NM_001142921.2 | 4018 | NP_001136393.1 | 447 | 5 |
| 21416 | NM_001331137.1 | 4200 | NP_001318066.1 | 471 | 11 |
| 21416 | NM_001142918.2 | 4152 | NP_001136390.1 | 459 | 1 |
| 21416 | NM_001142922.2 | 4127 | NP_001136394.1 | 598 | 6 |
| 21416 | NM_009333.4 | 4054 | NP_033359.2 | 459 | 3 |
| 21416 | NM_001142919.2 | 4101 | NP_001136391.1 | 442 | 2 |
| 21416 | NM_001331139.1 | 4076 | NP_001318068.1 | 581 | 13 |
| 21416 | NM_001142923.2 | 4003 | NP_001136395.1 | 442 | 7 |
| 21416 | NM_001331142.1 | 4140 | NP_001318071.1 | 455 | 16 |
| 21416 | NM_001331140.1 | 4115 | NP_001318069.1 | 594 | 14 |
| 21416 | NM_001331143.1 | 4042 | NP_001318072.1 | 455 | 17 |
| 21416 | NM_001331144.1 | 4137 | NP_001318073.1 | 450 | 18 |
| 21416 | NM_001331145.1 | 4089 | NP_001318074.1 | 438 | 19 |
| 21416 | NM_001331141.1 | 4064 | NP_001318070.1 | 577 | 15 |
| 21416 | XM_017318132.2 | 5283 | XP_017173621.1 | 438 | X22 |
| 21416 | XM_030250860.1 | 5210 | XP_030106720.1 | 299 | X28 |
| 21416 | XM_006526895.4 | 4392 | XP_006526958.1 | 443 | X20 |
| 21416 | XM_030250859.1 | 4402 | XP_030106719.1 | 299 | X27 |
| 21416 | XM_006526897.4 | 4377 | XP_006526960.1 | 438 | X23 |
| 21416 | XM_030250854.1 | 4365 | XP_030106714.1 | 434 | X25 |
| 21416 | XM_017318135.3 | 3822 | XP_017173624.1 | 295 | X29 |
| 21416 | XM_006526894.5 | 3679 | XP_006526957.1 | 443 | X19 |
| 21416 | XM_030250855.2 | 3628 | XP_030106715.1 | 426 | X26 |
| 21416 | XM_030250851.2 | 3667 | XP_030106711.1 | 439 | X21 |
| 21416 | XM_030250853.2 | 3652 | XP_030106713.1 | 434 | X24 |
| 21416 | NM_001142924.2 | 3454 | NP_001136396.1 | 299 | 8 |

Tcf712 isoform 6 [Mus musculus] (protein)
(SEQ ID NO: 80)
MPQLNGGGGDDLGANDELISFKDEGEQEEKSSENSSAERDLADVK
SSLVNESETNQNSSSDSEAERRPPPRSESFRDKSRESLEEAAKRQ
DGGLFKGPPYPGYPFIMIPDLTSPYLPNGSLSPTARTYLQMKWPL
LDVQAGSLQSRQALKDARSPSPAHIVSNKVPVVQHPHHVHPLTPL
ITYSNEHFTPGNPPPHLPADVDPKTGIPRPPHPPDISPYYPLSPG
TVGQIPHPLGWLVPQQGQPVYPITTGGFRHPYPTALTVNASMSRF
PPHMVPPHHTLHTTGIPHPAIVTPTVKQESSQSDVGSLHSSKHQD
SKKEEEKKKPHIKKPLNAFMLYMKEMRAKVVAECTLKESAAINQI
LGRRWHALSREEQAKYYELARKERQLHMQLYPGWSARDNYGKKKK
RKRDKQPGETNGEKKSAFATYKVKAAASAHPLQMEAY
Tcf712 isoform 6 [Mus musculus] (nucleic acid)
(SEQ ID NO: 81)
CTCGCCCTGTCAATAATCTCCGCTCCCAGACTACTCCGTTCCTCC
GGATTTCGATCCCCCTTTTTCTATCTGTCAATCAGCGCCGCCTTT
GAACTGAAAAGCTCAGTCTAACTTCAACTCACTCAAATCCGAGCG
GCACGAGATCCTTCGGTGTCTTCGGCTTCCCCCCCCTTTGCTCTT
ATATCTGACTTCTTCTTGTTGTTGTTGTTGTTTTTTTTACCCCCT
TTTTTATTTATTATTATTTTTTTTTTTTGCACATTGATCGGATCC
TTGGGAACGAGAGAAAAAAAGAAACCCAAACTCACGCGTGCAGAA
GATCTCCCCCCTTCCCCTCCCCTCCTCCCTCTTTTCCCCTCCCCA
GGAGAGAAAGACCCCAAAGCAGGAAAAAAAAAAGTTAACCTTGGA
CTCGTCTTTTTCTTGCAGTATTTTTTTGGGGGGGACTCGCAAAAC
TTTTTTTTTTTTTTTTTTTTTTTTTTTGCTTTTTTTCCTCCTTCA
TTTTTCTTCCAAAATTGCTGCTGCTGGTGGGTGAAAAATGCCGCA
GCTGAACGGCGGTGGAGGAGATGACCTAGGCGCTAACGACGAGCT
GATCTCCTTCAAAGACGAAGGCGAGCAGGAGGAGAAGAACTCGGA
AAACTCCTCGGCGGAAAGGGATTTAGCCGATGTCAAGTCCTCGCT
GGTCAATGAATCAGAGACGAATCAAAACAGCTCCTCCGATTCCGA
GGCGGAAAGACGGCCTCCGCCTCGCTCCGAAAGTTTCCGAGATAA
ATCCCGGGAAAGTTTGGAAGAAGCGGCCAAGAGGCAAGATGGAGG
GCTCTTTAAGGGGCCACCGTATCCCGGCTACCCCTTCATCATGAT
CCCCGACCTGACGAGCCCCTACCTCCCCAACGGATCGCTCTCGCC
CACCGCCCGAACCTATCTTCAGATGAAATGGCCACTGCTTGATGT
CCAAGCAGGAAGCCTCCAGAGCAGACAAACCCTCAAGGATGCTCG
TTCGCCGTCGCCAGCACACATCGTTTCGAACAAAGTACCGGTGGT
GCAACACCCCCACCATGTCCACCCACTCACGCCTCTCATCACGTA
CAGCAATGAACACTTCACCCCGGGAAATCCACCTCCGCACTTACC
AGCTGACGTAGACCCCAAAACAGGAATCCCAAGGCCTCCGCACCC
TCCAGATATCTCTCCATATTACCCGCTGTCGCCCGGCACCGTAGG
ACAAATCCCCCATCCGCTAGGATGGTTAGTACCACAGCAAGGTCA
GCCTGTGTACCCAATCACGACAGGAGGATTCAGACACCCCTACCC
CACAGCGCTGACAGTCAACGCATCTATGTCTAGGTTCCCTCCCCA
TATGGTCCCTCCCCATCACACTCTGCACACGACCGGCATCCCTCA
CCCGGCCATCGTCACACCGACAGTCAAGCAGGAATCCTCCCAGAG
TGACGTCGGCTCACTCCACAGCTCAAAGCATCAGGACTCCAAAAA
GGAAGAAGAGAAGAAGAAGCCCCACATAAAGAAGCCCCTTAATGC
ATTCATGTTGTATATGAAAGAGATGAGAGCGAAGGTGGTGGCCGA
ATGCACATTGAAAGAGAGTGCAGCCATCAACCAGATTCTCGGGCG
CAGGTGGCACGCCCTGTCCAGGGAAGAACAGGCAAAATACTACGA
GCTGGCCCGGAAGGAACGACAGCTTCACATGCAGCTGTACCCTGG
CTGGTCTGCACGGGATAACTATGGGAAGAAGAAGAAGAGAAAAAG
AGACAAGCAGCCGGGGGAAACCAACGAACACAGCGAATGTTTCCT
AAATCCTTGCCTTTCGCTTCCTCCGATCACAGACCTGAGCGCTCC
TAAGAAATGCCGAGCGCGCTTTGGCCTTGATCAACAGAATAACTG
GTGCGGCCCCTGCAGGAGAAAAAAAAAGTGCGTTCGCTACATACA
AGGTGAAGGCAGCTGCCTCAGCCCACCCTCTTCAGATGGAAGCTT
ACTAGACTCGCCTCCCCCCTCACCGCATCTGCTAGGCTCCCCTCC
CCAAGACGCCAAGTCACAGACTGAGCAGACCCAGCCGCTCTCGCT
GTCCCTGAAGCCTGATCCTCTGGCCCACCTGTCCATGATGCCTCC
GCCACCCGCGCTCCTGTTGGCCGAAGCTGCCCACGGCAAGGCCTC
TGCCCTCTGTCCCAATGGGGCTCTGGACCTGCCACCTGCCGCTCT
GCAGCCGTCCATGGTCCCTTCCTCATCGCTCGCACAACCATCAAC
TTCTTCCTTACATTCCCACAACTCGCTGGCTGGAACGCAACCCCA
GCCTCTGTCTCTGGTGACCAAGTCTTTAGAATAGCTCCGCCTCCT
CGTCCTCCATGCCCCTGCTTGATTGAAGTGTTTCCTTCTGGTTCT
TGGTTCGCCCTTCCCCGCTTGGAAAGCTTTTGTTTTGTACTCTTT
AATTTTGTGCCGCCGTGGCTACATGAGTTAATGTTTATGGAGTTC
ATTGGTCAATATTTGACCCATTCTTATTTCAATTTCTCCTTTTAA
ATATGTAGATGAGAAAAACCTCATGATTCTACCAAAATTTTTATC
AACAGCTGTTTAAAGTCTTTGTAGCGTTTAAAAAATATATTATAT
ATACATAACTGTTATGTAGTTCGGATAGCTTAGTTTTAAAAGACT
GATTTAAAAAACAAAAAGAAAAAAAAGAGAGAGAAGCAATTTTGA
AGCACCCTCCAGAAGGAGTTGGTTCTGTGTTATTTGTATTAAATA
CGAGCTTGCGAACCAATCACTTTACACCTTGGTATTTAAACCACG
AGGGCACAAGGAATGCAGTGCCGTTTCTTTCTTTCTTTTTTTCTT
TCTTTTTTTTTTCCTCCCAGTGTGAAACAACTTTTACCTTGATGT
TACTTGTTATTGTTTAAATGTACAGAAACAAAGGGTTAAAATGTG
TTAATATACCTTGTTCCATGGTGTTGTTCTTTTGGGGGGGAGGGG
ACACCACTCAACAATGAATGGAATCAACACTGTTGGACCAGTAGT
ATTTATTGCTTTAGAGGTTGCTTGCTGTACCTGTATGTCCGTCCA
ATTTTAAATATGTTTTCCCTTTTTTTTGAAACTGTGTAAAGTCCC
CCCCAACCTTAGCGTAAGCATCTTATATATAACAACTCATTTGTA
CAAGGTTTTTAAGTTTATATATAAAATGTGTATATATTTTTTTGT
TTCCTTTTTTTTTTTTTTTTGGCGTACGTTCGTTCGTTCGTTTGT
TTGTTTCTGTATAAAACCCAGATGCCACCAAATGGACATTGACAT
TCGCATTAAGGATCAGTAGCATTAACAAAACTTGCTTTAAAAGCC
ATTCTGTAAAACAAGACTTGAACATTAGCGAGAGGGTCTGAGCAG
CCGTCTGAGCAGCCGTGGGAACTGCCCCGTTTCCCCCTTGACCTC
CTAGTCAACTGCCCTGTGCTCTTAGAACATGGACTGACCGTGTGC
AAAACTTTTATGTGCCAAAATTCTCAGTGACTTTAGCTTTCTCCC
TCCTTTTTGATGCTGTACTTTCTGTTCGCCATGTTTTGCTGTGAT
GTTACATAGATAGATTTGTATGTAGTTTTAATGTCACCTATAACA
AAATGTGTTTGGTAGCAGACTGTCCAGAAAGCATTTTAAATGAAG
AGGTCTAAACCCTTAAGGGCCAAAAAAAATCCTGTATATTAGATT
ACTCTTAAATGAAAAAGAAAAAAAGAAAAAAAAAAACCCCAGCTG
CCGCTTTTATGTATGCATATTACATACAAGTAGGTAGTGACCTTT
AAAAACAAGAAAACCTAGGCATGCCGATGTTGTAAAATGCTGTAT
AAAGCTGAGACCTGTTCCTTCAGTGCCATCGTAGTTGACATGAAG
CGATTGTAAAACTGTCTCCGGTTTTCTCTGGTTTATTAAAATGCT
AACTATAACATTTTCTTTTCTTTTTTTTTTTTTGTGAATACTTTG
AATGTCTCCCTAACAGTTGTGGTGTTGCTGTTCTGTTCGATGCTT
ATTCCAAGTTCATTTTTTTGAATGGTTTTGAAGCCATTTTGTAAT
GAATTAAATGTCCATGCTGTACAGTACCTGTAGCATGCCTGCCGT
TCTGGATTAATAAAAGCAACTTAGTATGTGCA

TCF7L2 has highly conserved protein domains, conserved in several species including human, mouse, rat, chicken, fish and Drosophila. For example, the human TCF7L2 has a 90.5% homology with the murine transcript. Accordingly, TCF7L2s of non-human species can also be used in the expression cassette, genetic construct, vector, composition, or method disclosed herein.

The terms “TCF7L2” and “Transcription factor 7-like 2” also encompass functional fragments or derivatives that substantially retain transcription factor activity of the TCF7L2s described herein. Typically, a functional fragment or derivative retains at least 50% of 60%, 70%, 80%, 90%, 95%, 99% or 100% of its transcription factor activity. It is also intended that a TCF7L2 protein can include conservative amino acid substitutions that do not substantially alter its activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity. Conservative and non-conservative amino acid substitutions have been described herein.

As used herein, the term “conservative sequence modifications” refers to amino acid modifications that do not significantly affect or alter the activity of a TCF7L2. Conservative amino acid substitutions are ones in which the amino acid residue is replaced with an amino acid residue having a similar side chain. Families of amino acid residues having similar side chains have been known in the art. A conservative modification or functional equivalent of a peptide, polypeptide, or protein disclosed herein refers to a polypeptide derivative of the peptide, polypeptide, or protein, e.g., a protein having one or more substitutions, point mutations, insertions, deletions, truncations, a fusion protein, or a combination thereof. It retains substantially the activity to of the parent peptide, polypeptide, or protein (such as those disclosed herein). In general, a conservative modification or functional equivalent is at least 60% (e.g., any number between 60% and 100%, inclusive, e.g., 60%, 70%, 75%, 80%, 85%, 90%, 91%, 92%, 93%, 94%, 95%, 96%, 97%, 98%, and 99%) identical to a parent (e.g., one of the human or non-human TCF7L2 sequences disclosed herein).

Amino acid substitutions can be made, in some cases, by selecting substitutions that do not differ significantly in their effect on maintaining (a) the structure of the peptide backbone in the area of the substitution, (b) the charge or hydrophobicity of the molecule at the target sit; or (c) the bulk of the side chain. For example, naturally occurring residues can be divided into groups based on side-chain properties; (1) hydrophobic amino acids (norleucine, methionine, alanine, valine, leucine, and isoleucine); (2) neutral hydrophilic amino acids (cysteine, serine, threonine, asparagine, and glutamine,); (3) acidic amino acids (aspartic acid and glutamic acid); (4) basic amino acids (histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Examples of substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine. Exemplary substitutions are shown in the table below. Amino acid substitutions may be introduced into human TCF7L2 and the products screened for retention of the biological activity of human TCF7L2.

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

### 3. Genetic Constructs, Expression Cassettes and Expression Vectors

The disclosure also provides a genetic construct, such as an expression cassette, comprising or consisting of a nucleic acid encoding a transcription factor 7-like 2 protein. While such a nucleic acid may not already comprise a promoter, the expression cassette may additionally comprise a promoter or an enhancer. In that case, the nucleic acid is operatively linked to and under the regulatory control of the promoter and/or enhancer for a gene selectively or specifically expressed by glial progenitor cells. Thus, an expression cassette according to the present invention may comprise, in 5′ to 3′ direction, a promoter, a coding sequence, and optionally a terminator or other elements. The expression cassette allows an easy transfer of a nucleic acid sequence of interest into an organism, preferably a cell and preferably a disease cell.

The expression cassette of the present disclosure may be preferably comprised in a vector. Thus, the vector of the present disclosure allows to transform a cell with a nucleic acid sequence of interest. Correspondingly the disclosure provides a host cell comprising an expression cassette according to the present disclosure or a recombinant nucleic acid according to the present disclosure. The recombinant nucleic acid may also comprise a promoter or enhancer to allow for the expression of the nucleic acid sequence of interest.

Exogenous genetic material (e.g., a nucleic acid, an expression cassette, or an expression vector encoding one or more therapeutic agents) can be introduced into a target cell of interest in vivo by genetic transfer methods, such as transfection or transduction, to provide a genetically modified cell. Various expression vectors (i.e., vehicles for facilitating delivery of exogenous genetic material into a target cell) are known to one of ordinary skill in the art. As used herein, “exogenous genetic material” refers to a nucleic acid or an oligonucleotide, either natural or synthetic, that is not naturally found in the cells; or if it is naturally found in the cells, it is not transcribed or expressed at biologically significant levels by the cells. Thus, “exogenous genetic material” includes, for example, a non-naturally occurring nucleic acid that can be transcribed into an RNA.

As used herein, “transfection of cells” refers to the acquisition by a cell of new genetic material by incorporation of added nucleic acid (DNA, RNA, or a hybrid thereof) without use of a viral delivery vehicle. Thus, transfection refers to the introducing of nucleic acid into a cell using physical or chemical methods. Several transfection techniques are known to those of ordinary skill in the art including: calcium phosphate nucleic acid co-precipitation, strontium phosphate nucleic acid co-precipitation, DEAE-dextran, electroporation, cationic liposome-mediated transfection, and tungsten particle-facilitated microparticle bombardment. In contrast, “transduction of cells” refers to the process of transferring nucleic acid into a cell using a DNA or RNA virus. An RNA virus (e.g., a retrovirus) for transferring a nucleic acid into a cell can be used as a transducing chimeric virus. Exogenous genetic material contained within the virus can be incorporated into the genome of the transduced cell. A cell that has been transduced with a chimeric DNA virus (e.g., an adenovirus carrying a DNA encoding a therapeutic agent), may not have the exogenous genetic material incorporated into its genome but may be capable of expressing the exogenous genetic material that is retained extrachromosomally within the cell.

Typically, the exogenous genetic material may include a heterologous gene (coding for a therapeutic RNA or protein) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. The exogenous genetic material may be introduced into the cell genome immediately downstream from the promoter so that the promoter and coding sequence are operatively linked so as to permit transcription of the coding sequence. A viral expression vector may include an exogenous promoter element to control transcription of the inserted exogenous gene. Examples of such exogenous promoters include constitutive promoters, inducible promoters, and tissue or cell-type specific promoters.

Naturally-occurring constitutive promoters control the expression of essential cell functions. As a result, a gene under the control of a constitutive promoter is expressed under all conditions of cell growth. Exemplary constitutive promoters include the promoters for the following genes that encode certain constitutive or “housekeeping” functions: hypoxanthine phosphoribosyl transferase, dihydrofolate reductase, adenosine deaminase, phosphoglycerol kinase, pyruvate kinase, phosphoglycerol mutase, the actin promoter, ubiquitin, elongation factor-1 and other constitutive promoters known to those of skill in the art. In addition, many viral promoters function constitutively in eucaryotic cells. These include the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only in, or largely controlled by, the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. If the gene encoding the therapeutic agent is under the control of an inducible promoter, delivery of the therapeutic agent in situ can be triggered by exposing the genetically modified cell in situ to conditions for permitting transcription of the therapeutic agent, e.g., by injection of specific inducers of the inducible promoters which control transcription of the agent. For example, in situ expression by genetically modified cells of a therapeutic agent encoded by a gene under the control of the metallothionein promoter, is enhanced by contacting the genetically modified cells with a solution containing the appropriate (i.e., inducing) metal ions in situ.

Accordingly, the amount of therapeutic agent that is delivered in situ is regulated by controlling such factors as: (1) the nature of the promoter used to direct transcription of the inserted gene, (i.e., whether the promoter is constitutive or inducible, strong or weak); (2) the number of copies of the exogenous gene that are inserted into the cell; (3) the number of transduced/transfected cells that are administered (e.g., implanted) to the patient; (4) the size of the implant (e.g., graft or encapsulated expression system); (5) the number of implants; (6) the length of time the transduced/transfected cells or implants are left in place; and (7) the production rate of the therapeutic agent by the genetically modified cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector may include a selection gene, for example, a neomycin resistance gene or a fluorescent protein gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene, and/or signal sequence is deemed to be within the scope of one of ordinary skill in the art without undue experimentation.

A coding sequence of the present disclosure can be inserted into any type of target or host cell. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

As disclosed herein, the transcription factor 7-like 2 protein described above can be used for treating a disorder in a subject. In some embodiments, a polynucleotide encoding the protein can be inserted into, or encoded by, vectors such as plasmids or viral vectors. Preferably, the polynucleotide is inserted into, or encoded by, viral vectors. A variety of viral-derived vectors can be used for transfection and integration into a mammalian cell genome. Viruses, which are useful as vectors include, but are not limited to, retroviruses, adenoviruses, adeno-associated viruses (AAV), herpes viruses, and lentiviruses. In some embodiments, the protein may be encoded by a retroviral vector, such as a lentiviral vector (See, e.g., U.S. Pat. Nos. 5,399,346; 5,124,263; 4,650,764 and 4,980,289; the content of each of which is incorporated herein by reference in its entirety). In some specific embodiments, the viral vectors are AAV vectors.

Lentiviral Vectors

Lentiviruses, such as HIV, are “slow viruses.” Vectors derived from lentiviruses can be expressed long-term in the host cells after a few administrations to the patients, e.g., via ex vivo transduced stem cells or progenitor cells. For most diseases and disorders, including genetic diseases, cancer, and neurological disease, long-term expression is crucial to successful treatment. Regarding safety with lentiviral vectors, a number of strategies for eliminating the ability of lentiviral vectors to replicate have now been known in the art. See e.g., US 20210401868 and 20210403517, each of which is incorporated herein by reference in its entirety. For example, the deletion of promoter and enhancer elements from the U3 region of the long terminal repeat (LTR) are thought to have no LTR-directed transcription. The resulting vectors are called “self-inactivating” (SIN).

Lentiviral vectors are particularly suitable to achieving long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as CNS cells. They also have the added advantage of low immunogenicity. In general, a suitable vector contains an origin of replication functional in at least one organism, a promoter sequence, convenient restriction endonuclease sites, and one or more selectable markers, (e.g., WO01/96584 and WO01/29058; and U.S. Pat. No. 6,326,193). Several vector promoter sequences are available for expression of the transgenes. One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is EF1a. However, other constitutive promoter sequences can also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Inducible promoters include, but are not limited to a metallothionein promoter, a glucocorticoid promoter, a progesterone promoter, and a tetracycline promoter.

The present disclosure provides a recombinant lentivirus capable of infecting dividing and non-dividing cells, such oligodendrocytes or oligodendrocyte progenitor cells. The virus is useful for the in vivo and ex vivo transfer and expression of nucleic acid sequences. Lentiviral vectors of the present disclosure may be lentiviral transfer plasmids or infectious lentiviral particles. Construction of lentiviral vectors, helper constructs, envelope constructs, etc., for use in lentiviral transfer systems has been described in, e.g., US 20210401868 and 20210403517, each of which is incorporated herein by reference in its entirety.

Adenoviruses

Adenoviruses are eukaryotic DNA viruses that can be modified to efficiently deliver a nucleic acid to a variety of cell types in vivo, and have been used extensively in gene therapy protocols, including for targeting genes to neural cells and glial cells. Various replication defective adenovirus and minimum adenovirus vectors have been described for nucleic acid therapeutics (See, e.g., PCT Patent Publication Nos. WO199426914, WO 199502697, WO199428152, WO199412649, WO199502697 and WO199622378; the content of each of which is incorporated by reference in their entirety). Such adenoviral vectors may also be used to deliver therapeutic molecules of the present disclosure to cells.

Adeno-Associated Virus

The adeno-associated virus is a widely used gene therapy vector due to its clinical safety record, non-pathogenic nature, ability to infect non-dividing cells (like neurons), and ability to provide long-term gene expression after a single administration. Currently, many human and non-human primate AAV serotypes have been identified. AAV vectors have demonstrated safety in hundreds of clinical trials worldwide, and clinical efficacy has been shown in trials of hemophilia B, spinal muscular atrophy, alpha 1 antitrypsin, and Leber congenital amaurosis.

Because of their safety, nonpathogenic nature, and ability to infect neurons, AAVs such as AAV1, AAV2, AAV4, AAV5, AAV6, AAV8, and AAV9 are commonly used gene therapy vectors for CNS applications. However, after direct CNS infusion, these serotypes exhibit a dominant neuronal tropism and expression in oligodendrocytes is low, especially when gene expression is driven by a constitutive promoter, which restricts their potential for use in treating white matter diseases. AAV1/2, AAV2, and AAV8 have been shown transduce oligodendrocytes. Reliance on cell-specific promoters for expression specificity allows for the possibility of nonselective cellular uptake and leaky transgene expression through cryptic promoter activity in non-oligodendrocyte lineage cells.

The approach described herein to alleviate these issues includes using AAV serotypes with high tropism for oligodendrocytes or glial progenitor cells, such as oligodendrocyte progenitor cells. Recently, using DNA shuffling and directed evolution, a chimeric AAV capsid with strong selectivity for oligodendrocytes, AAV/Olig001, has been described (Powell et al., 2016, Gene Ther 23:807-814). Subsequently, AAV/Olig001 was shown to transduce neonatal oligodendrocytes in a mouse model of Canavan disease (Francis et al., 2021. Mol Ther Methods Clin Dev 20:520-534). Other approaches such as random mutagenesis and peptide library insertion can be used to generate capsid libraries that can be screened for tropism and selectivity for oligodendrocytes or glial progenitor cells.

As discussed above, the terms “adeno-associated virus” and/or “AAV” refer to parvoviruses with a linear single-stranded DNA genome and variants thereof. The term covers all subtypes and both naturally occurring and recombinant forms, except where required otherwise. Parvoviruses, including AAV, are useful as gene therapy vectors as they can penetrate a cell and introduce a nucleic acid (e.g., transgene) into the nucleus. In some embodiments, the introduced nucleic acid (e.g., rAAV vector genome) forms circular concatemers that persist as episomes in the nucleus of transduced cells. In some embodiments, a transgene is inserted in specific sites in the host cell genome. Site-specific integration, as opposed to random integration, is believed to likely result in a predictable long-term expression profile. The insertion site of AAV into the human genome is referred to as AAVS1. Once introduced into a cell, RNAs or polypeptides encoded by the nucleic acid can be expressed by the cell. Because AAV is not associated with any pathogenic disease in humans, a nucleic acid delivered by AAV can be used to express a therapeutic RNA or polypeptide for the treatment of a disease, disorder and/or condition in a human subject.

Multiple serotypes of AAV exist in nature with at least fifteen wild type serotypes having been identified from humans thus far (i.e., AAV1-AAV15). Naturally occurring and variant serotypes are distinguished by having a protein capsid that is serologically distinct from other AAV serotypes. Examples include AAV1, AAV2, AAV, AAV3 (including AAV3A and AAV3B), AAV4, AAV5, AAV6, AAV7, AAV8, AAV9, AAV10, AAV12, AAVrh10, AAVrh74 (see WO 2016/210170), avian AAV, bovine AAV, canine AAV, equine AAV, primate AAV, non-primate AAV, and ovine AAV, and recombinantly produced variants (e.g., capsid variants with insertions, deletions and substitutions, etc.), such as variants referred to as AAV2i8, NP4, NP22, NP66, DJ, DJ/8, DJ/9, LK3, RHM4-1, among many others. “Primate AAV” refers to AAV that infect primates, “non-primate AAV” refers to AAV that infect non-primate mammals, “bovine AAV” refers to AAV that infect bovine mammals, and so on.

Serotype distinctiveness is determined on the basis of the lack of cross-reactivity between antibodies to one AAV as compared to another AAV. Such cross-reactivity differences are usually due to differences in capsid protein sequences and antigenic determinants (e.g., due to VP1, VP2, and/or VP3 sequence differences of AAV serotypes). However, some naturally occurring AAV or man-made AAV mutants (e.g., recombinant AAV) may not exhibit serological difference with any of the currently known serotypes. These viruses may then be considered a subgroup of the corresponding type, or more simply a variant AAV. Thus, as used herein, the term “serotype” refers to both serologically distinct viruses, as well as viruses that are not serologically distinct but that may be within a subgroup or a variant of a given serotype.

A comprehensive list and alignment of amino acid sequences of capsids of known AAV serotypes is provided by Marsic et al. (2014) Molecular Therapy 22(11):1900-1909. Genomic sequences of various serotypes of AAV, as well as sequences of the native ITRs, rep proteins, and capsid subunits are known in the art. Such sequences may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), NC_001863 (AAV3B), NC_001829 (AAV4), U89790 (AAV4), NC_006152 (AAV5), NC_001862 (AAV6), AF513851 (AAV7), AF513852 (AAV8), and NC_006261 (AAV8); the disclosures of which are incorporated by reference herein. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99: 11854; Moris et al. (2004) Virology 33:375-383; international patent publications WO 00/28061, WO 99/61601, WO 98/11244; WO 2013/063379; WO 2014/194132; WO 2015/121501, and U.S. Pat. Nos. 6,156,303 and 7,906,111.

As discussed herein, a “recombinant adeno-associated virus” or “rAAV” is distinguished from a wild-type AAV by replacement of all or part of the endogenous viral genome with a non-native sequence. Incorporation of a non-native sequence within the virus defines the viral vector as a “recombinant” vector, and hence a “rAAV vector.” An rAAV vector can include a heterologous polynucleotide encoding a desired RNA or protein or polypeptide (e.g., an RNA molecule disclosed herein). A recombinant vector sequence may be encapsidated or packaged into an AAV capsid and referred to as an “rAAV vector,” an “rAAV vector particle,” “rAAV viral particle” or simply a “rAAV.”

The present disclosure provides for an rAAV vector comprising a polynucleotide sequence not of AAV origin (e.g., a polynucleotide heterologous to AAV). The heterologous polynucleotide may be flanked by at least one, and sometimes by two, AAV terminal repeat sequences (e.g., inverted terminal repeats). The heterologous polynucleotide flanked by ITRs, also referred to herein as a “vector genome,” typically encodes an RNA or a polypeptide of interest, or a gene of interest, such as a target for therapeutic treatment. Delivery or administration of an rAAV vector to a subject (e.g., a patient) provides encoded RNAs/proteins/peptides to the subject. Thus, an rAAV vector can be used to transfer/deliver a heterologous polynucleotide for expression for, e.g., treating a variety of diseases, disorders and conditions.

rAAV vector genomes generally retain 145 base ITRs in cis to the heterologous nucleic acid sequence that replaced the viral rep and cap genes. Such ITRs are useful to produce a recombinant AAV vector; however, modified AAV ITRs and non-AAV terminal repeats including partially or completely synthetic sequences can also serve this purpose. ITRs form hairpin structures and function to, for example, serve as primers for host-cell-mediated synthesis of the complementary DNA strand after infection. ITRs also play a role in viral packaging, integration, etc. ITRs are the only AAV viral elements which are required in cis for AAV genome replication and packaging into rAAV vectors. An rAAV vector genome optionally comprises two ITRs which are generally at the 5′ and 3′ ends of the vector genome comprising a heterologous sequence (e.g., a transgene encoding a gene of interest, or a nucleic acid sequence of interest including, but not limited to, an antisense, and siRNA, a CRISPR molecule, among many others). A 5′ and a 3′ ITR may both comprise the same sequence, or each may comprise a different sequence. An AAV ITR may be from any AAV including by not limited to serotypes 1, 2, 3, 4, 5, 6, 7, 8, 9, 10 or 11 or any other AAV.

An rAAV vector of the disclosure may comprise an ITR from an AAV serotype (e.g., wild-type AAV2, a fragment or variant thereof) that differs from the serotype of the capsid (e.g., AAV8, Olig001). Such an rAAV vector comprising at least one ITR from one serotype, but comprising a capsid from a different serotype, may be referred to as a hybrid viral vector (see U.S. Pat. No. 7,172,893). An AAV ITR may include the entire wild type ITR sequence, or be a variant, fragment, or modification thereof, but will retain functionality.

In some embodiments, an rAAV vector genome is linear, single-stranded and flanked by AAV ITRs. Prior to transcription and translation of the heterologous gene, a single stranded DNA genome of approximately 4700 nucleotides must be converted to a double-stranded form by DNA polymerases (e.g., DNA polymerases within the transduced cell) using the free 3′-OH of one of the self-priming ITRs to initiate second-strand synthesis. In some embodiments, full length-single stranded vector genomes (i.e., sense and anti-sense) anneal to generate a full length-double stranded vector genome. This may occur when multiple rAAV vectors carrying genomes of opposite polarity (i.e., sense or anti-sense) simultaneously transduce the same cell. Regardless of how they are produced, once double-stranded vector genomes are formed, the cell can transcribe and translate the double-stranded DNA and express the heterologous gene.

The efficiency of transgene expression from an rAAV vector can be hindered by the need to convert a single stranded rAAV genome (ssAAV) into double-stranded DNA prior to expression. This step can be circumvented by using a self-complementary AAV genome (scAAV) that can package an inverted repeat genome that can fold into double-stranded DNA without the need for DNA synthesis or base-pairing between multiple vector genomes. See, e.g., U.S. Pat. No. 8,784,799; McCarty, (2008) Molec. Therapy 16(10):1648-1656; and McCarty et al., (2001) Gene Therapy 8:1248-1254; McCarty et al., (2003) Gene Therapy 10:2112-2118.

A viral capsid of an rAAV vector may be from a wild type AAV or a variant AAV such as AAV1, AAV2, AAV3, AAV3A, AAV3B, AAV4, AAV5, AAV6, AAV7, AAV8, AAV9, AAV10, AAVrh10, AAVrh74 (see WO2016/210170), AAV12, AAV2i8, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAV avian AAV, bovine AAV, canine AAV, equine AAV, primate AAV, non-primate AAV, snake AAV, goat AAV, shrimp AAV, ovine AAV and variants thereof (see, e.g., Fields et al., VIROLOGY, volume 2, chapter 69 (4^{th} ed., Lippincott-Raven Publishers). Capsids may be derived from a number of AAV serotypes disclosed in U.S. Pat. No. 7,906,111; Gao et al. (2004) J. Virol. 78:6381; Morris et al. (2004) Virol. 33:375; WO 2013/063379; WO 2014/194132; and include true type AAV (AAV-TT) variants disclosed in WO 2015/121501, and RHM4-1, RHM15-1 through RHM15-6, and variants thereof, disclosed in WO 2015/013313. A full complement of AAV cap proteins includes VP1, VP2, and VP3. The ORF comprising nucleotide sequences encoding AAV VP capsid proteins may comprise less than a full complement AAV Cap proteins or the full complement of AAV cap proteins may be provided.

In some embodiments, an rAAV vector comprising a capsid protein encoded by a nucleotide sequence derived from more than one AAV serotype (e.g., wild type AAV serotypes, variant AAV serotypes) is referred to as a “chimeric vector” or “chimeric capsid” (See U.S. Pat. No. 6,491,907, the entire disclosure of which is incorporated herein by reference). In some embodiments, a chimeric capsid protein is encoded by a nucleic acid sequence derived from 2, 3, 4, 5, 6, 7, 8, 9, 10 or more AAV serotypes. In some embodiments, a recombinant AAV vector includes a capsid sequence derived from e.g., AAV1, AAV2, AAV3, AAV3A, AAV3B, AAV4, AAV5, AAV6, AAV7, AAV8, AAV9, AAV10, AAV11, AAVrh74, AAVrh10, AAV2i8, or variant thereof, resulting in a chimeric capsid protein comprising a combination of amino acids from any of the foregoing AAV serotypes (see, Rabinowitz et al. (2002) J. Virology 76(2):791-801). Alternatively, a chimeric capsid can comprise a mixture of a VP1 from one serotype, a VP2 from a different serotype, a VP3 from yet a different serotype, and a combination thereof. For example a chimeric virus capsid may include an AAV1 cap protein or subunit and at least one AAV2 cap protein or subunit. A chimeric capsid can, for example include an AAV capsid with one or more B19 cap subunits, e.g., an AAV cap protein or subunit can be replaced by a B19 cap protein or subunit. For example, in one embodiment, a VP3 subunit of an AAV capsid can be replaced by a VP2 subunit of B19. In some embodiments, a chimeric capsid is an Olig001 capsid as described in WO2021221995 and WO2014052789, which are incorporated herein by reference.

In some embodiments, chimeric vectors have been engineered to exhibit altered tropism or tropism for a particular tissue or cell type. The term “tropism” refers to preferential entry of the virus into certain cell (e.g., oligodendrocytes) or tissue types and/or preferential interaction with the cell surface that facilitates entry into certain cell or tissue types. AAV tropism is generally determined by the specific interaction between distinct viral capsid proteins and their cognate cellular receptors (Lykken et al. (2018) J. Neurodev. Disord. 10:16). Preferably, once a virus or viral vector has entered a cell, sequences (e.g., heterologous sequences such as a transgene) carried by the vector genome (e.g., an rAAV vector genome) are expressed.

A “tropism profile” refers to a pattern of transduction of one or more target cells in various tissues and/or organs. For example, a chimeric AAV capsid may have a tropism profile characterized by efficient transduction of oligodendrocytes or oligodendrocyte progenitor cells with only low transduction of neurons, astrocytes and other CNS cells. See WO2014/052789, incorporated herein by reference. Such a chimeric capsid may be considered “specific for oligodendrocytes or oligodendrocyte progenitor cells” exhibiting tropism for oligodendrocytes or oligodendrocyte progenitor cells, and referred to herein as “oligotropism,” if when administered directly into the CNS, preferentially transduces oligodendrocytes or oligodendrocyte progenitor cells over neurons, astrocytes and other CNS cell types. In some embodiments, at least about 80% of cells that are transduced by a capsid specific for oligodendrocytes or oligodendrocyte progenitor cells are oligodendrocytes or oligodendrocyte progenitor cells, e.g., at least about 85%, 90%, 95%, 96%, 97%, 98% 99% or more of the transduced cells are oligodendrocytes or oligodendrocyte progenitor cells.

In some embodiments, an rAAV vector is useful for treating or preventing a “disorder associated with oligodendrocyte dysfunction.” As used herein, the term “associated with oligodendrocyte dysfunction” refers to a disease, disorder or condition in which oligodendrocytes are damaged, lost or function improperly compared to otherwise identical normal oligodendrocytes. The term includes diseases, disorders and conditions in which oligodendrocytes are directly affected as well as diseases, disorders or conditions in which oligodendrocytes become dysfunctional secondary to damage to other cells. In some embodiments, a disorder associated with oligodendrocyte dysfunction is demyelination.

### 4. Uses and Treatment Methods

Gene Therapy

The nucleic acids, genetic constructs, expression cassettes, and expression vectors described herein may be used for gene therapy treatment and/or prevention of a disease, disorder or condition. In particular, it can be used for treating or preventing a disease, disorder or condition associated with deficiency or dysfunction of oligodendrocyte or myelin by increasing the expression of a transcription factor 7-like 2 protein, and of any other condition and or illness in which increasing the expression of the protein may produce a therapeutic benefit or improvement, e.g., a disease, disorder or condition mediated by, or associated with, a decrease in the level or function of the protein compared with the level or function of the protein in an otherwise healthy individual.

As used herein a disorder of myelin, a disease of myelin, a myelin-related disorder, a myelin-related disease, a myelin disorder, a disorder mediated by a deficiency in myelin, and a myelin disease are used interchangeably. They include any disease, condition (e.g., those occurring from traumatic spinal cord injury and cerebral infarction), or disorder related to demyelination, insufficient myelination and remyelination, or dysmyelination in a subject. Such a disorder can be inherited or acquired or both. It can arise from a myelination related disorder or demyelination resulting from a variety of neurotoxic insults. “Demyelination” as used herein, refers to the act of demyelinating, or the loss of the myelin sheath insulating the nerves, and is the hallmark of some neurodegenerative autoimmune diseases, including multiple sclerosis, transverse myelitis, chronic inflammatory demyelinating polyneuropathy, and Guillain-Barre Syndrome. Leukodystrophies are caused by inherited enzyme deficiencies, which cause abnormal formation, destruction, and/or abnormal turnover of myelin sheaths within the CNS white matter. Both acquired and inherited myelin disorders share a poor prognosis leading to major disability. Thus, some embodiments of the present disclosure can include methods for the treatment of neurodegenerative autoimmune diseases in a subject. Remyelination of neurons requires oligodendrocytes. The term “remyelination”, as used herein, refers to the re-generation of the nerve's myelin sheath by replacing myelin producing cells or restoring their function.

Myelin related diseases or disorders which may be treated or ameliorated by the methods of the present invention include diseases, disorders or injuries which relate to dysmyelination or demyelination in a subject's brain cells, e.g., CNS neurons. Such diseases include, but are not limited to, diseases and disorders in which the myelin which surrounds the neuron is either absent, incomplete, not formed properly, or is deteriorating. Such disease include, but are not limited to, multiple sclerosis (MS), neuromyelitis optica (NMO), progressive multifocal leukoencephalopathy (PML), encephalomyelitis (EPL), central pontine myelolysis (CPM), adrenoleukodystrophy, Alexander's disease, Pelizaeus Merzbacher disease (PMD), Wallerian Degeneration, optic neuritis, transverse myelitis, amyotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Marie-Charcot-Tooth disease and Bell's palsy.

Myelin related diseases or disorders which may be treated or ameliorated by the methods of the present invention include a disease or disorder characterized by a myelin deficiency. Insufficient myelination in the central nervous system has been implicated in a wide array of neurological disorders. Among these are forms of cerebral palsy in which a congenital deficit in forebrain myelination in children with periventricular leukomalacia, contributes to neurological morbidity (Goldman et al., 2008) Goldman, S. A., Schanz, S., and Windrem, M. S. (2008). Stem cell-based strategies for treating pediatric disorders of myelin. Hum Mol Genet. 17, R76-83. At the other end of the age spectrum, myelin loss and ineffective repair may contribute to the decline in cognitive function associated with senescence (Kohama et al., 2011) Kohama, S. G., Rosene, D. L., and Sherman, L. S. (2011) Age (Dordr). Age-related changes in human and non-human primate white matter: from myelination disturbances to cognitive decline. Therefore, it is contemplated that effective compositions and methods of enhancing myelination and/or remyelination may have substantial therapeutic benefits in halting disease progression and restoring function in a wide array of myelin-related disorders.

In some embodiments, the compositions of the present invention can be administered to a subject that does not have, and/or is not suspected of having, a myelin related disorder in order to enhance or promote a myelin dependent process. In some embodiments, compositions described herein can be administered to a subject to promote myelination of CNS neurons in order to enhance cognition, which is known to be a myelin dependent process, in cognitive healthy subjects. In certain embodiments, compositions described herein can be administered in combination with cognitive enhancing (nootropic) agents. Exemplary agents include any drugs, supplements, or other substances that improve cognitive function, particularly executive functions, memory, creativity, or motivation, in healthy individuals. Non limiting examples include racetams (e.g., piracetam, oxiracetam, and aniracetam), nutraceuticals (e.g., Bacopa monnieri, Panax ginseng, Ginkgo biloba, and GABA), stimulants (e.g., amphetamine pharmaceuticals, methylphenidate, eugeroics, xanthines, and nicotine), L-Theanine, Tolcapone, Levodopa, Atomoxetine, and Desipramine.

The overall dosage of a therapeutic agent (e.g., a protein, a polynucleotide encoding the protein, or a vector, such as an rAAV vector, or a cell) will be a therapeutically effective amount depending on several factors including the overall health of a subject, the subject's disease state, severity of the condition, the observation of improvements and the formulation and route of administration of the selected agent(s). Determination of a therapeutically effective amount is within the capability of those skilled in the art. The exact formulation, route of administration and dosage can be chosen by the individual physician in view of the subject's condition.

In certain embodiments, the cell or nucleotide compositions described herein may be administered in an amount effective to enhance myelin production in the CNS of a subject by an increase in the amount of one or more myelin proteins (e.g., MBP, MAG, MOG, MOBP, PLP1, GPR37, ASPA, CNP, MYRF, BCAS1, PLP1, UGT8, TF, LPAR1, and FA2H) of at least 5%, 10%, 20%, 25%, 30%, 40%, 50%, 60%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, or 1000% as compared to the level of myelin proteins of an untreated subject.

In other embodiments, the cell or nucleotide compositions may be administered in an amount effective to promote survival of CNS neurons in a subject by an increase in the number of surviving neurons of at least 5%, 10%, 20%, 25%, 30%, 40%, 50%, 60%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, or 1000% as compared to the number of surviving neurons in an untreated CNS neurons or subject.

Another strategy for treating a subject suffering from myelin-related disorder is to administer a therapeutically effective amount of a cell or nucleotide composition described herein along with a therapeutically effective amount of an oligodendrocyte differentiation and/or proliferation inducing agent(s) and/or anti-neurodegenerative disease agent. Examples of anti-neurodegenerative disease agents include L-dopa, cholinesterase inhibitors, anticholinergics, dopamine agonists, steroids, and immunomodulators including interferons, monoclonal antibodies, and glatiramer acetate. Therefore, in a further aspect of the disclosure, the compositions described herein can be administered as part of a combination therapy with adjunctive therapies for treating neurodegenerative and myelin related disorders.

The phrase “combination therapy” embraces the administration of oligodendrocyte precursor differentiation inducing compositions described herein and a therapeutic agent as part of a specific treatment regimen intended to provide a beneficial effect from the co-action of these therapeutic agents. When administered as a combination, the oligodendrocyte precursor differentiation inducing compound and a therapeutic agent can be formulated as separate compositions. Administration of these therapeutic agents in combination typically is carried out over a defined time period (usually minutes, hours, days or weeks depending upon the combination selected).

The genetic nucleic acids, genetic constructs, expression cassettes, and expression vectors of the present application may be administered by intracerebral delivery, intrathecal delivery, intranasal delivery, or via direct infusion into the brain ventricles.

The genetic constructs of the present application may also be administered directly to the airways in the form of an aerosol. For use as aerosols, the compounds of the present application in solution or suspension may be packaged in a pressurized aerosol container together with suitable propellants, for example, hydrocarbon propellants like propane, butane, or isobutane with conventional adjuvants. The materials of the present application also may be administered in a non-pressurized form such as in a nebulizer or atomizer.

In one embodiment, the one or more genetic constructs may activate transcription of one or more of the genes described herein via a CRISPR-Cas9 guided nuclease (Gimenez et al., “CRISPR-on System for the Activation of the Endogenous human INS gene,” Gene Therapy 23: 543-547 (2016); Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339(6121):819-23 (2013); and Gaj et al., “ZFN, TALEN, and CRISPR/Cas-based Methods for Genome Engineering,” Cell 31(7):397-405 (2013), which are hereby incorporated by reference in their entirety). CRISPR-Cas9 is a genetic technique which allows for sequence-specific control of gene expression in prokaryotic and eukaryotic cells by guided nuclease double-stranded DNA cleavage. It is based on the bacterial immune system-derived CRISPR (clustered regularly interspaced palindromic repeats) pathway.

In the embodiments described supra, the one or more genetic constructs may be packaged in a suitable delivery vehicle or carrier for delivery to the subject. Suitable delivery vehicles include, but are not limited to viruses, virus-like particles, bacteria, bacteriophages, biodegradable microspheres, microparticles, nanoparticles, exosomes, liposomes, collagen minipellets, and cochleates. These and other biological gene delivery vehicles are well known to those of skill in the art (see, e.g., Seow and Wood, “Biological Gene Delivery Vehicles: Beyond Viral Vectors,” Mol. Therapy 17(5):767-777 (2009), which is hereby incorporated by reference in its entirety).

In one embodiment, the genetic construct is packaged into a therapeutic expression vector to facilitate delivery. Suitable expression vectors are well known in the art and include, without limitation, viral vectors such as adenovirus vectors, adeno-associated virus vectors, retrovirus vectors, lentivirus vectors, or herpes virus vectors.

The viral vectors or other suitable expression vectors comprise sequences encoding the genetic constructs of the present application and any suitable promoter and/or enhancer for expressing the genetic construct. Suitable promoters include, for example, and without limitation, the U6 or HI RNA pol III promoter sequences and the cytomegalovirus promoter. Selection of other suitable promoters is within the skill in the art. The expression vectors may also comprise inducible or regulatable promoters for expression of the inhibitory nucleic acid molecules in a tissue or cell-specific manner.

Gene therapy vectors carrying the therapeutic genetic construct or nucleic acid molecule are administered to a subject by, for example, intravenous injection, local administration (U.S. Pat. No. 5,328,470 to Nabel et al., which is hereby incorporated by reference in its entirety) or by stereotactic injection (see, e.g., Chen et al., “Gene Therapy for Brain Tumors: Regression of Experimental Gliomas by Adenovirus Mediated Gene Transfer In vivo,” Proc. Nat'l. Acad. Sci. USA 91:3054-3057 (1994), which is hereby incorporated by reference in its entirety). The pharmaceutical preparation of the therapeutic vector can include the therapeutic vector in an acceptable diluent, or can comprise a slow release matrix in which the therapeutic delivery vehicle is imbedded. Alternatively, where the complete therapeutic delivery vector can be produced intact from recombinant cells, e.g., retroviral vectors, the pharmaceutical preparation can include one or more cells which produce the therapeutic delivery system. Gene therapy vectors typically utilize constitutive regulatory elements which are responsive to endogenous transcriptions factors.

Another suitable approach for the delivery of the genetic construct of the present disclosure, involves the use of liposome delivery vehicles or nanoparticle delivery vehicles.

In another embodiment of the present application, the delivery vehicle is a nanoparticle. A variety of nanoparticle delivery vehicles are known in the art and are suitable for delivery of the genetic constructs of the present application (see, e.g., van Vlerken et al., “Multi-functional Polymeric Nanoparticles for Tumour-Targeted Drug Delivery,” Expert Opin. Drug Deliv. 3(2):205-216 (2006), which is hereby incorporated by reference in its entirety). Suitable nanoparticles include, without limitation, poly(beta-amino esters) (Sawicki et al., “Nanoparticle Delivery of Suicide DNA for Epithelial Ovarian Cancer Cell Therapy,” Adv. Exp. Med. Biol. 622:209-219 (2008), which is hereby incorporated by reference in its entirety), polyethylenimine-alt-poly(ethylene glycol) copolymers (Park et al., “Degradable Polyethylenimine-alt-Poly(ethylene glycol) Copolymers As Novel Gene Carriers,” J. Control Release 105(3):367-80 (2005) and Park et al., “Intratumoral Administration of Anti-KITENIN shRNA-Loaded PEI-alt-PEG Nanoparticles Suppressed Colon Carcinoma Established Subcutaneously in Mice,” J Nanosci. Nanotechnology 10(5):3280-3 (2010), which are hereby incorporated by reference in their entirety), poly(d,l-lactide-coglycolide) (Chan et al., “Antisense Oligonucleotides: From Design to Therapeutic Application,” Clin. Exp. Pharm. Physiol. 33: 533-540 (2006), which is hereby incorporated by reference in its entirety), and liposome-entrapped siRNA nanoparticles (Kenny et al., “Novel Multifunctional Nanoparticle Mediates siRNA Tumor Delivery, Visualization and Therapeutic Tumor Reduction In vivo,” J. Control Release 149(2):111-116 (2011), which is hereby incorporated by reference in its entirety). Other nanoparticle vehicles suitable for use in the present application include microcapsule nanotube devices disclosed in U.S. Patent Publication No. 2010/0215724 to Prakash et al., which is hereby incorporated by reference in its entirety.

In another embodiment, the genetic construct is contained in a liposome delivery vehicle. The term “liposome” means a vesicle composed of amphiphilic lipids arranged in a spherical bilayer or bilayers. Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

Several advantages of liposomes include: their biocompatibility and biodegradability, incorporation of a wide range of water and lipid soluble drugs; and they afford protection to encapsulated molecules from metabolism and degradation. Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Methods for preparing liposomes include those disclosed in Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., which are hereby incorporated by reference in their entirety.

As disclosed herein, in another embodiment, the genetic construct, expression cassette, or expression vector can be administered in association with a glial progenitor cell-targeted fusogen or a glial progenitor cell-selective surface-binding moiety. For example, the genetic construct, expression cassette, or expression vector can be in or associated with a fusosome.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a liposome and a cytoplasm of a target cell, or a lumen of a viral vector and a cytoplasm of a target cell). In some embodiments, the fusogen comprises a protein or a complex of two or more proteins having a targeting domain or binding moiety. In some examples, the targeting domain or binding moiety specifically targets or binds to a molecule on glial progenitor cell or a glial progenitor cell. Examples of the molecule include, but not limited to, CD140a, NG2/CSPG4, A2B5 gangliosides, 04 sulfatides, or CD133. A targeting domain or binding moiety can be a receptor ligand, a peptide/polypeptide, an antibody, or an antigen-binding portion thereof that specifically binds to a molecule or marker on a glial progenitor cell or a glial progenitor cell. Non-limiting examples of human and non-human fusogens are described in, e.g., US 20210198698 and US 20210137839, which are incorporated by reference in their entireties.

As used herein, “fusosome” refers to a bilayer of amphipathic lipids enclosing a lumen or cavity and a fusogen that interacts with the amphipathic lipid bilayer. In some embodiments, the fusosome comprises a nucleic acid. In some embodiments, the fusosome is a membrane enclosed preparation. In some embodiments, the fusosome is derived from a source cell.

Fusosomes can take various forms. For example, in some embodiments, a fusosome described herein is derived from a source cell. A fusosome may be or comprise, e.g., an extracellular vesicle, a microvesicle, a nanovesicle, an exosome, a microparticle, or any combination thereof. In some embodiments, a fusosome is released naturally from a source cell, and in some embodiments, the source cell is treated to enhance formation of fusosomes. In some embodiments, the fusosome is between about 10-10,000 nm in diameter, e.g., about 30-100 nm in diameter. In some embodiments, the fusosome comprises one or more synthetic lipids.

In some embodiments, the fusosome is or comprises a virus, e.g., a retrovirus, e.g., a lentivirus. For instance, in some embodiments, the fusosome's bilayer of amphipathic lipids is or comprises the viral envelope. The viral envelope may comprise a fusogen, e.g., a fusogen that is endogenous to the virus or a pseudotyped fusogen. In some embodiments, the fusosome's lumen or cavity comprises a viral nucleic acid, e.g., a retroviral nucleic acid, e.g., a lentiviral nucleic acid. The viral nucleic acid may be a viral genome. In some embodiments, the fusosome further comprises one or more viral non-structural proteins, e.g., in its cavity or lumen.

Fusosomes may have various structures or properties that facilitate delivery of a payload to a target cell. For instance, in some embodiments, the fusosome and the source cell together comprise nucleic acid(s) sufficient to make a particle that can fuse with a target cell. In embodiments, these nucleic acid(s) encode proteins having one or more of (e.g., all of) the following activities: gag polyprotein activity, polymerase activity, integrase activity, protease activity, and fusogen activity.

Cell Therapy

Also within scope of this disclosure is a host cell comprising the genetic construct, cassette, or expression vector described above, or a progeny cell of the host cell. The host cell can be a stem cell or a progenitor cell. Example of the stem cell include embryonic stem cells, ES-like stem cells, fetal stem cells, adult stem cells, pluripotent stem cells, induced pluripotent stem cells, multipotent stem cells, oligopotent stem cells, unipotent stem cells and others. In some embodiments, the host cell is a glial progenitor cell, such as an oligodendrocyte progenitor cell. The host cell or a progeny thereof can be used as a therapeutic cell or agent for treating the disorders or conditions described herein.

Suitable methods of introducing cells (such as the above-described host cells or progenies thereof) into the striatum, forebrain, brain stem, and/or cerebellum of a subject are well known to those of skill in the art and include, but are not limited to, injection, deposition, and grafting as described herein.

In one embodiment, the glial progenitor cells are transplanted bilaterally into multiple sites of the subject as described U.S. Pat. No. 7,524,491 to Goldman, Windrem et al., “Neonatal Chimerization With Human Glial Progenitor Cells Can Both Remyelinate and Rescue the Otherwise Lethally Hypomyelinated Shiverer Mouse,” Cell Stem Cell 2:553-565 (2008), Han et al., “Forebrain Engraftment by Human Glial Progenitor Cells Enhances Synaptic Plasticity and Learning Adult Mice,” Cell Stem Cell 12:342-353 (2013), and Wang et al., “Human iPSCs-Derived Oligodendrocyte Progenitor Cells Can Myelinate and Rescue a Mouse Model of Congenital Hypomyelination,” Cell Stem Cell 12:252-264 (2013), which are hereby incorporated by reference in their entirety. Methods for transplanting nerve tissues and cells into host brains are described by, NEURAL GRAFTING IN THE MAMMALIAN CNS, Ch. 3-8 (Bjorklund and Stenevi eds., Elsevier, Amsterdam 1985); U.S. Pat. No. 5,082,670 to Gage et al.; and U.S. Pat. No. 6,497,872 to Weiss et al., which are hereby incorporated by reference in their entirety. Typical procedures include intraparenchymal, intracallosal, intrathecal, intracerebral, intranasal, and intravenous transplantation as well as direct infusion into brain ventricles.

Intraparenchymal transplantation is achieved by injection or deposition of tissue within the host brain so as to be apposed to the brain parenchyma at the time of transplantation. The two main procedures for intraparenchymal transplantation are: 1) injecting the donor cells within the host brain parenchyma or 2) preparing a cavity by surgical means to expose the host brain parenchyma and then depositing the graft into the cavity (NEURAL GRAFTING IN THE MAMMALIAN CNS, Ch. 3 (Bjorklund and Stenevi eds., Elsevier, Amsterdam 1985), which is hereby incorporated by reference in its entirety). Both methods provide parenchymal apposition between the donor cells and host brain tissue at the time of grafting, and both facilitate anatomical integration between the graft and host brain tissue. This is of importance if it is required that the donor cells become an integral part of the host brain and survive for the life of the host.

Glial progenitor cells can also be delivered intracallosally as described in U.S. Patent Application Publication No. 20030223972 to Goldman, which is hereby incorporated by reference in its entirety. The glial progenitor cells can also be delivered directly to the forebrain subcortex, specifically into the anterior and posterior anlagen of the corpus callosum. Glial progenitor cells can also be delivered to the cerebellar peduncle white matter to gain access to the major cerebellar and brainstem tracts. Glial progenitor cells can also be delivered to the spinal cord.

Alternatively, the cells may be placed in a ventricle, e.g., a cerebral ventricle. Grafting cells in the ventricle may be accomplished by injection of the donor cells or by growing the cells in a substrate such as 30% collagen to form a plug of solid tissue which may then be implanted into the ventricle to prevent dislocation of the graft cells. For subdural grafting, the cells may be injected around the surface of the brain after making a slit in the dura.

Suitable techniques for glial cell delivery are described supra. In one embodiment, said preparation of glial progenitor cells is administered to one or more sites of the brain, brain stem, spinal cord, or combinations thereof.

Delivery of the cells to the subject can include either a single step or a multiple step injection directly into the nervous system. Although adult and fetal oligodendrocyte precursor cells disperse widely within a transplant recipient's brain, for widespread disorders, multiple injections sites can be performed to optimize treatment. Injection is optionally directed into areas of the central nervous system such as white matter tracts like the corpus callosum (e.g., into the anterior and posterior anlagen), dorsal columns, cerebellar peduncles, cerebral peduncles. Such injections can be made unilaterally or bilaterally using precise localization methods such as stereotaxic surgery, optionally with accompanying imaging methods (e.g., high resolution MRI imaging). One of skill in the art recognizes that brain regions vary across species; however, one of skill in the art also recognizes comparable brain regions across mammalian species.

The cellular transplants are optionally injected as dissociated cells but can also be provided by local placement of non-dissociated cells. In either case, the cellular transplants optionally comprise an acceptable solution. Such acceptable solutions include solutions that avoid undesirable biological activities and contamination. Suitable solutions include an appropriate amount of a pharmaceutically-acceptable salt to render the formulation isotonic. Examples of the pharmaceutically-acceptable solutions include, but are not limited to, saline, Ringer's solution, dextrose solution, and culture media. The pH of the solution is preferably from about 5 to about 8, and more preferably from about 7 to about 7.5.

The injection of the dissociated cellular transplant can be a streaming injection made across the entry path, the exit path, or both the entry and exit paths of the injection device (e.g., a cannula, a needle, or a tube). Automation can be used to provide a uniform entry and exit speed and an injection speed and volume.

The number of glial progenitor cells administered to the subject can range from about 10²-10⁸ at each administration (e.g., injection site), depending on the size and species of the recipient, and the volume of tissue requiring cell replacement. Single administration (e.g., injection) doses can span ranges of 10³-10⁵, 10⁴-10⁷, and 10⁵-10⁸ cells, or any amount in total for a transplant recipient patient.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

Since the CNS is an immunologically privileged site, administered cells, including xenogeneic, can survive and, optionally, no immunosuppressant drugs or a typical regimen of immunosuppressant agents are used in the treatment methods. However, optionally, an immunosuppressant agent may also be administered to the subject. Immunosuppressant agents and their dosing regimens are known to one of skill in the art and include such agents as Azathioprine, Azathioprine Sodium, Cyclosporine, Daltroban, Gusperimus Trihydrochloride, Sirolimus, and Tacrolimus. Dosages ranges and duration of the regimen can be varied with the disorder being treated; the extent of rejection; the activity of the specific immunosuppressant employed; the age, body weight, general health, sex and diet of the subject; the time of administration; the route of administration; the rate of excretion of the specific immunosuppressant employed; the duration and frequency of the treatment; and drugs used in combination. One of skill in the art can determine acceptable dosages for and duration of immunosuppression. The dosage regimen can be adjusted by the individual physician in the event of any contraindications or change in the subject's status.

### 5. Pharmaceutical Compositions

The present disclosure provides a pharmaceutical composition, or medicament, for preventing or treating an inherited or acquired disorder of myelin. In some embodiments, a pharmaceutical composition comprises one or more of the above-described protein molecule, polynucleotide, expression cassette, expression vector (e.g., viral vector genome, expression vector, rAAV vector), and host cell.

The pharmaceutical composition further comprises a pharmaceutically-acceptable carrier, adjuvant, diluent, excipient and/or other medicinal agents. A pharmaceutically acceptable carrier, adjuvant, diluent, excipient or other medicinal agent is one that is not biologically or otherwise undesirable, e.g., the material may be administered to a subject without causing undesirable biological effects which outweigh the advantageous biological effects of the material. Any suitable pharmaceutically acceptable carrier or excipient can be used in the preparation of a pharmaceutical composition according to the invention (See e.g., Remington The Science and Practice of Pharmacy, Adeboye Adejare (Editor) Academic Press, November 2020).

A pharmaceutical composition is typically sterile, pyrogen-free and stable under the conditions of manufacture and storage. A pharmaceutical composition may be formulated as a solution (e.g., water, saline, dextrose solution, buffered solution, or other pharmaceutically sterile fluid), microemulsion, liposome, or other ordered structure suitable to accommodate a high product (e.g., viral vector particles, microparticles or nanoparticles) concentration.

In some embodiments, a pharmaceutical composition comprising the above-described protein, polynucleotide, expression cassette, expression vector, vector genome, host cell, or rAAV vector of the disclosure is formulated in water or a buffered saline solution. A carrier may be a solvent or dispersion medium containing, for example, water, ethanol, polyol (for example, glycerol, propylene glycol, and liquid polyethylene glycol, and the like), and suitable mixtures thereof. Proper fluidity can be maintained, for example, by use of a coating such as lecithin, by maintenance of a required particle size, in the case of dispersion, and by the use of surfactants. In some embodiments, it may be preferable to include isotonic agents, for example, a sugar, a polyalcohol such as mannitol, sorbitol, or sodium chloride in the composition. Prolonged adsorption of an injectable composition can be brought about by including, in the composition, an agent which delays absorption, e.g., a monostearate salt and gelatin. In some embodiments, a nucleic acid, vector and/or host cell of the disclosure may be administered in a controlled release formulation, for example, in a composition which includes a slow-release polymer or other carrier that protects the product against rapid release, including an implant and microencapsulated delivery system.

In some embodiments, a pharmaceutical composition of the disclosure is a parenteral pharmaceutical composition, including a composition suitable for intravenous, intraarterial, subcutaneous, intradermal, intraperitoneal, intramuscular, intraarticular, intraparenchymal (IP), intrathecal (IT), intracerebroventricular (ICV) and/or intracisternal magna (ICM) administration. In some embodiments, a pharmaceutical composition of this disclosure is formulated for administration by ICV injection. In some embodiments, a vector (e.g., a viral vector such as AAV) may be formulated in 350 mM NaCl and 5% D-sorbitol in PBS.

### 6. Methods of Administration

The above-described molecule, or polynucleotide, or vector (e.g., vector genome, rAAV vector) may be administered to a subject (e.g., a patient) or a target cell in order to treat the subject. Administration of a vector to a human subject, or an animal in need thereof, can be by any means known in the art for administering a vector. Examples of a target cell include cells of the CNS, preferably oligodendrocytes or the progenitor cells thereof.

A vector can be administered in addition to, and as an adjunct to, the standard of care treatment. That is, the vector can be co-administered with another agent, compound, drug, treatment or therapeutic regimen, either simultaneously, contemporaneously, or at a determined dosing interval as would be determined by one skilled in the art using routine methods. Uses disclosed herein include administration of an rAAV vector of the disclosure at the same time, in addition to and/or on a dosing schedule concurrent with, the standard of care for the disease as known in the art.

In some embodiments, a combination composition includes one or more immunosuppressive agents. In some embodiments, a combination composition includes an rAAV vector comprising a transgene (e.g., a polynucleotide encoding an RNA molecule disclosed herein) and one or more immunosuppressive agents. In some embodiments, a method includes administering or delivering an rAAV vector comprising the transgene to a subject and administering an immunosuppressive agent to the subject either prophylactically prior to administration of the vector, or after administration of the vector (i.e., either before or after symptoms of a response against the vector and/or the protein provided thereby are evident).

In one embodiment, a vector of the disclosure (e.g., an rAAV vector) is administered systemically. Exemplary methods of systemic administration include, but are not limited to, intravenous (e.g., portal vein), intraarterial (e.g., femoral artery, hepatic artery), intravascular, subcutaneous, intradermal, intraperitoneal, transmucosal, intrapulmonary, intralymphatic and intramuscular administration, and the like, as well as direct tissue or organ injection. One skilled in the art would appreciate that systemic administration can deliver a nucleic acid to all tissues. In some embodiments, direct tissue or organ administration includes administration to areas directly affected by oligodendrocyte deficiency (e.g., brain and/or central nervous system). In some embodiments, vectors of the disclosure, and pharmaceutical compositions thereof, are administered to the brain parenchyma (i.e., by intraparenchymal administration), to the spinal canal or the subarachnoid space so that it reaches the cerebrospinal fluid (CSF) (i.e., by intrathecal administration), to a ventricle of the brain (i.e., by intracerebroventricular administration) and/or to the cisterna magna of the brain (i.e., by intracisternal magna administration).

Accordingly, in some embodiments, a vector of the present disclosure is administered by direct injection into the brain (e.g., into the parenchyma, ventricle, cisterna magna, etc.) and/or into the CSF (e.g., into the spinal canal or subarachnoid space) to treat a disorder of myelin. A target cell of a vector of the present disclosure includes a cell located in the cortex, subcortical white matter of the corpus callosum, striatum and/or cerebellum. In some embodiments, a target cell of a vector of the present disclosure is an oligodendrocyte or a progenitor cell thereof. Additional routes of administration may also comprise local application of a vector under direct visualization, e.g., superficial cortical application, or other stereotaxic application.

In some embodiments, a vector of the disclosure is administered by at least two routes. For example, a vector is administered systemically and also directly into the brain. If administered via at least two routes, the administration of a vector can be, but need not be, simultaneous or contemporaneous. Instead, administration via different routes can be performed separately with an interval of time between each administration.

The above-described protein, or polynucleotide encoding the protein, or a vector genome, or a vector (e.g., an rAAV vector) comprising the polynucleotide may be used for transduction of a cell ex vivo or for administration directly to a subject (e.g., directly to the CNS of a patient with a disease). In some embodiments, a transduced cell (e.g., a host cell) is administered to a subject to treat or prevent a disease, disorder or condition (e.g., cell therapy for the disease). For example, an rAAV vector comprising a therapeutic nucleic acid (e.g., encoding a protein) can be preferably administered to an oligodendrocyte or a progenitor cell thereof in a biologically-effective amount.

The dosage amount of a vector depends upon, e.g., the mode of administration, disease or condition to be treated, the stage and/or aggressiveness of the disease, individual subject's condition (age, sex, weight, etc.), particular viral vector, stability of protein to be expressed, host immune response to the vector, and/or gene to be delivered. Generally, doses range from at least 1×10⁸, or more, e.g., 1×10⁹, 1×10¹⁰, 1×10¹¹, 1×10¹², 1×10¹³, 1×10¹⁴, 1×10¹⁵ or more vector genomes (vg) per kilogram (kg) of body weight of the subject to achieve a therapeutic effect.

In some embodiments, a polynucleotide encoding a protein described herein may be administered as a component of a DNA molecule (e.g., a recombinant nucleic acid) having a regulatory element (e.g., a promoter) appropriate for expression in a target cell (e.g., oligodendrocytes). The polynucleotide may be administered as a component of a plasmid or a viral vector, such as an rAAV vector. An rAAV vector may be administered in vivo by direct delivery of the vector (e.g., directly to the CNS) to a patient in need of treatment. An rAAV vector may be administered to a patient ex vivo by administration of the vector in vitro to a cell from a donor patient in need of treatment, followed by introduction of the transduced cell back into the donor (e.g., cell therapy).

### 7. Kit

The present disclosure provides a kit with packaging material and one or more components therein. A kit typically includes a label or packaging insert including a description of the components or instructions for use in vitro, in vivo or ex vivo, of the components therein. A kit can contain a collection of such components, e.g., the above-described polynucleotide, nucleic acid, expression cassette, expression vector (e.g., viral vector genome, expression vector, rAAV vector), and host cell or progenies thereof, and optionally a second active agent such as a compound, therapeutic agent, drug or composition.

A kit refers to a physical structure that contains one or more components of the kit. Packaging material can maintain the components in a sterile manner and can be made of material commonly used for such purposes (e.g., paper, glass, plastic, foil, ampules, vials, tubes, etc.).

A label or insert can include identifying information of one or more components therein, dose amounts, clinical pharmacology of the active ingredients(s) including mechanism of action, pharmacokinetics and pharmacodynamics. A label or insert can include information identifying manufacture, lot numbers, manufacture location and date, expiration dates. A label or insert can include information on a disease (e.g., an inherited or acquired disorder of myelin such as HD) for which a kit component may be used. A label or insert can include instructions for a clinician or subject for using one or more of the kit components in a method, use or treatment protocol or therapeutic regimen. Instructions can include dosage amounts, frequency of duration and instructions for practicing any of the methods, uses, treatment protocols or prophylactic or therapeutic regimens described herein.

A label or insert can include information on potential adverse side effects, complications or reaction, such as a warning to a subject or clinician regarding situations where it would not be appropriate to use a particular composition.

### 8. Definitions

Unless otherwise defined, all technical and scientific terms used herein have the meaning commonly understood by one of ordinary skill in the art to which this invention belongs. The terminology used herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. The following terms have the meanings given:

As used herein, the term “about,” or “approximately” refers to a measurable value such as an amount of the biological activity, homology or length of a polynucleotide or polypeptide sequence, dose, time, temperature, and the like, and is meant to encompass variations of 25%, 20%, 19%, 18%, 17%, 16%, 15%, 14%, 13%, 12%, 11%, 10%, 9%, 8%, 7%, 6%, 5%, 4%, 3%, 2% 1%, 0.5% or even 0.1%, in either direction (greater than or less than) of the specified amount unless otherwise stated, otherwise evident from the context, or except where such number would exceed 100% of a possible value.

As used herein, the term “homologous,” or “homology,” refers to two or more reference entities (e.g., a nucleic acid or polypeptide sequence) that share at least partial identity over a given region or portion. For example, when an amino acid position in two peptides is occupied by identical amino acids, the peptides are homologous at that position. Notably, a homologous peptide will retain activity or function associated with the unmodified or reference peptide and the modified peptide will generally have an amino acid sequence “substantially homologous” with the amino acid sequence of the unmodified sequence. When referring to a polypeptide, nucleic acid or fragment thereof, “substantial homology” or “substantial similarity,” means that when optimally aligned with appropriate insertions or deletions with another polypeptide, nucleic acid (or its complementary strand) or fragment thereof, there is sequence identity in at least about 70% to 99% of the sequence. The extent of homology (identity) between two sequences can be ascertained using computer program or mathematical algorithm known in the art. Such algorithms that calculate percent sequence homology (or identity) generally account for sequence gaps and mismatches over the comparison region or area.

A nucleic acid or polynucleotide refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA.

An isolated or recombinant nucleic acid refers to a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid described above can be used to express the protein of this disclosure. For this purpose, one can operatively linked the nucleic acid to suitable regulatory sequences to generate an expression vector.

A “recombinant nucleic acid” is a combination of nucleic acid sequences that are joined together using recombinant technology and procedures used to join together nucleic acid sequences.

The terms “heterologous” DNA molecule and “heterologous” nucleic acid, as used herein, each refer to a molecule that originates from a source foreign to the particular host cell or, if from the same source, is modified from its original form. Thus, a heterologous gene in a host cell includes a gene that is endogenous to the particular host cell but has been modified through, for example, the use of shuffling or recombination. When used to describe two nucleic acid segments, the terms mean that the two nucleic acid segments are not from the same gene or, if form the same gene, one or both of them are modified from the original forms. The terms also include non-naturally occurring multiple copies of a naturally occurring DNA molecule. Thus, the terms refer to a nucleic acid segment that is foreign or heterologous to the cell, or homologous to the cell but in a position within the host cell nucleic acid in which the element is not ordinarily found. Exogenous DNA segments are expressed to yield exogenous RNAs or polypeptides. A “homologous DNA molecule” is a DNA molecule that is naturally associated with a host cell into which it is introduced.

A “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein or RNA desired, and the like. The expression vector can be introduced into host cells to produce an RNA or a polypeptide of interest. A promoter is defined as a DNA sequence that directs RNA polymerase to bind to DNA and initiate RNA synthesis. A strong promoter is one which causes RNAs to be initiated at high frequency.

A “promoter” is a nucleotide sequence which initiates and regulates transcription of a polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

“Operably linked” refers to an arrangement of elements wherein the components so described are configured so as to perform their usual function. Thus, a given promoter operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence. Thus, the term “operably linked” is intended to encompass any spacing or orientation of the promoter element and the DNA sequence of interest which allows for initiation of transcription of the DNA sequence of interest upon recognition of the promoter element by a transcription complex.

As used here, the term “genetic construct” or “nucleic acid construct,” refers to a non-naturally occurring nucleic acid molecule resulting from the use of recombinant DNA technology (e.g., a recombinant nucleic acid). A genetic or nucleic acid construct is a nucleic acid molecule, either single or double stranded, which has been modified to contain segments of nucleic acid sequences, which are combined and arranged in a manner not found in nature. A nucleic acid construct may be a “cassette” or a “vector” (e.g., a plasmid, an rAAV vector genome, an expression vector, etc.), that is, a nucleic acid molecule designed to deliver exogenously created DNA into a host cell.

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. It also may include sequences required for proper translation of the nucleotide sequence. The coding region usually codes for an RNA or protein of interest. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development.

A vector refers to a nucleic acid molecule capable of transporting another nucleic acid to which it has been linked. The vector may or may not be capable of autonomous replication or integrate into a host DNA. Examples of the vector include a plasmid, cosmid, or viral vector. The vector includes a nucleic acid in a form suitable for expression of a nucleic acid of interest in a host cell. Preferably the vector includes one or more regulatory sequences operatively linked to the nucleic acid sequence to be expressed.

As used herein, the term “overexpressing,” “overexpress,” “overexpressed,” or “overexpression,” when referring to the production of a nucleic acid or a protein in a host cell means that the nucleic acid or protein is produced in greater amounts than it is produced in its naturally occurring environment. It is intended that the term encompass overexpression of endogenous, as well as exogenous or heterologous nucleic acids and proteins. As such, the terms and the like are intended to encompass increasing the expression of a nucleic acid or a protein in a cell to a level greater than that the cell naturally contains. In certain embodiments, the expression level or amount of the nucleic acid or protein in a cell is increased by at least 5%, 10%, 20%, 25%, 30%, 40%, 50%, 60%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, or 1000% as compared to the level or amount that the cell naturally contains.

In the context of a mutant or diseased cell, the terms “overexpressing,” “overexpress,” “overexpressed,” and “overexpression,” and the like are intended to encompass increasing the expression of a nucleic acid or a protein to a level greater than that a mutant cell, a diseased cell, a wildtype cell, or a non-diseased cell contains. In certain embodiments, the expression level or amount of the nucleic acid or protein in a mutant or diseased cell is increased by at least 5%, 10%, 20%, 25%, 30%, 40%, 50%, 60%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, or 1000% as compared to the level or amount that a mutant cell, a diseased cell, a wildtype cell, or a non-diseased cell contains.

As used herein, the term “prevent” or “prevention” refers to delay of onset, and/or reduction in frequency and/or severity of one or more sign or symptom of a particular disease, disorder or condition. In some embodiments, prevention is assessed on a population basis such that an agent is considered to “prevent” a particular disease, disorder or condition if a statistically significant decrease in the development, frequency and/or intensity of one or more sign or symptom of the disease, disorder or condition is observed in a population susceptible to the disease, disorder or condition. Prevention may be considered complete when onset of disease, disorder or condition has been delayed for a predefined period of time.

As used herein, the term “therapeutically effective amount” refers to an amount that produces the desired therapeutic effect for which it is administered. In some embodiments, the term refers to an amount that is sufficient, when administered to a population suffering from or susceptible to a disease, disorder or condition in accordance with a therapeutic dosing regimen, to treat the disease, disorder or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment.

As used herein, the term “stem cells” refers to cells with the ability to both replace themselves and to differentiate into more specialized cells. Their self-renewal capacity generally endures for the lifespan of the organism. A pluripotent stem cell can give rise to all the various cell types of the body. A multipotent stem cell can give rise to a limited subset of cell types. For example, a hematopoietic stem cell can give rise to the various types of cells found in blood, but not to other types of cells. Multipotent stem cells can also be referred to as somatic stem cells, tissue stem cells, lineage-specific stem cells, and adult stem cells. The non-stem cell progeny of multipotent stem cells are progenitor cells (also referred to as restricted-progenitor cells). Progenitor cells give rise to fully differentiated cells, but a more restricted set of cell types than stem cells. Progenitor cells also have comparatively limited self-renewal capacity; as they divide and differentiate they are eventually exhausted and replaced by new progenitor cells derived from their upstream multipotent stem cell.

As used herein, “therapeutic cells” refers to a cell population that ameliorates a condition, disease, and/or injury in a patient. Therapeutic cells may be autologous (i.e., derived from the patient), allogeneic (i.e., derived from an individual of the same species that is different from the patient) or xenogeneic (i.e., derived from a different species than the patient). Therapeutic cells may be homogenous (i.e., consisting of a single cell type) or heterogeneous (i.e., consisting of multiple cell types). The term “therapeutic cell” includes both therapeutically active cells as well as progenitor cells capable of differentiating into a therapeutically active cell.

### EXAMPLES

The examples below are intended to exemplify the practice of embodiments of the disclosure but are by no means intended to limit the scope thereof.

### Introduction to the Examples

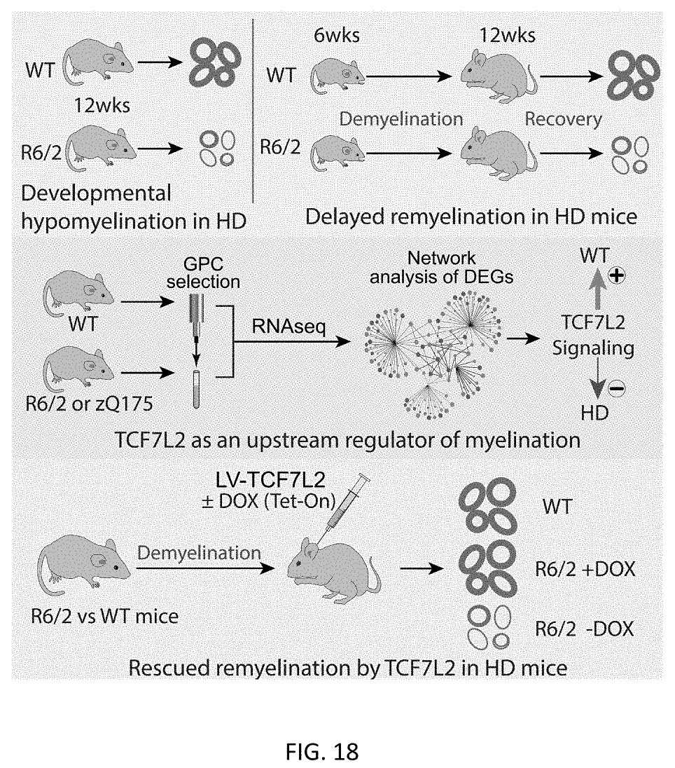

The Examples presented here explore whether HD was associated with a failure of homeostatic myelin maintenance and adult remyelination, that would serve to link the impaired myelinogenesis of HD hGPCs with the white matter involution of HD patients. Using the R6/2 mouse model of HD, the ultrastructure of adult R6/2 and wild-type callosal white matter were compared, followed by a comparison of their responses to cuprizone-induced demyelination. An age dependent and progressive hypomyelination was observed in untreated R6/2 mice, while cuprizone-treated R6/2 mice further demyelinated by cuprizone treatment (FIG. 18, top panel). RNA-Sequencing of both callosal white matter and isolated GPCs, as derived from R6/2 and zQ175 mice—the latter a longer-lived model of later-onset HD—then revealed in both models a systematic down-regulation of genes associated with oligodendrocyte differentiation and myelinogenesis, relative to controls. Gene co-expression and network analysis predicted repressed Tcf712 signaling as a major driver of this expression pattern. Proteomic analysis of callosal and striatal white matter derived from both R6/2 and zQ175 mice then confirmed that TCF7L2-regulated myelin proteins were down-regulated relative to wild-type controls, and that relative suppression of myelin protein expression increased with age in both HD models (FIG. 18, middle panel). TCF7L2 was overexpressed via lentivirus in R6/2 striatum, and found that this was sufficient to rescue oligodendroglial gene expression, and furthermore was accompanied by the upregulation of a number of metabotropic and myelinogenic genes required for both astrocytic and oligodendrocyte differentiation. Proteomic analysis of callosal and striatal white matter derived from both R6/2 and zQ175 mice then confirmed that TCF7L2-regulated myelin proteins were down-regulated relative to wild-type controls, and that the relative suppression of myelin protein expression increased with age in both HD models. On that basis, the ability of lentiviral Tcf712 expression to then modulate the remyelination of cuprizone-treated R6/2 mice was assessed. These studies proved that TCF7L2 overexpression was sufficient to restore the normal time course and efficacy of remyelination (FIG. 18, bottom panel). Together, these data suggest that the impaired expression of Tcf712-dependent genes may be a fundamental cause of the impaired glial differentiation and white matter failure of HD, and that the induced overexpression of Tcf712 may be a viable strategy for relief of white matter failure in HD.

### Materials and Methods for Examples

Animals. All experiments were approved by the Institutional Animal Care and Use Committee of the University of Rochester. Wild-type females with ovary transplants from R6/2+(120 CAG) donor mice were purchased from Jackson Laboratories (Bar Harbor, Me.). zQ175(190Q) breeders were obtained from Charles River by way of CHDI foundation. These mice were bred to PDGFRa-EGFP mice and genotyped following weaning and double heterozygous mice were further analyzed to determine their CAG repeat number through PCR with primers encoding a product spanning the repeat region as previously described (Benraiss et al., “Sustained Mobilization Of Endogenous Neural Progenitors Delays Disease Progression In A Transgenic Model Of Huntington's Disease,” Cell Reports 36: 109308, (2013), which is hereby incorporated by reference in its entirety). All experiments included 4-8 mice/group, as indicated. For the remyelination studies, mice were fed with 0.2% (w/w) Cuprizone in chow (Bio-serv) ad libitum for 6 weeks. All samples included equal numbers of males and females.

Transmission Electron Microscopy and Image Processing. The mice were perfused with a 2.0% paraformaldehyde/2.5% glutaraldehyde in 0.1M sodium cacodylate buffer (pH 7.4) which contained 0.2M sucrose. After 24 hours of primary fixation the tissue were sectioned coronally, rinsed in the same buffer, and post-fixed for 90 minutes in cacodylate buffered 1.5% osmium tetroxide mixed with an equal amount of 1.5% potassium ferrocyanide. The sections were rinsed twice in distilled water and dehydrated in a graded series of ethanol to 100%, transitioned into propylene oxide, infiltrated with EPON/Araldite resin, embedded, and polymerized for 48 hours at 60° C. Epoxy embedded tissue blocks were cut at one micron with a glass knife onto glass slides and stained with Toluidine Blue on a hot plate to specifically identify the pre-determined region of cross-sectional myelinated corpus collosum. Using a diamond knife in an ultramicrotome, thin sections (70 nm) of targeted areas were cut and collected onto carbon coated nickel grids. The grids were stained with aqueous uranyl acetate and lead citrate and examined using a Hitachi 7650 TEM with an attached Gatan 11-megapixel Erlangshen digital camera and Digital micrograph software. For g-ratio (axon diameter/fiber diameter ratio), the 20 images taken randomly for each sample were processed using the Image J software. Using the G Ratio calculator (Cellular Imaging Facility, Univ. of Lausanne), an Image J plugin, the image was converted to a binary image and all axon of any given micrograph were measured for the G ratio. Due to the irregular shapes of the elements, the approximate diameters were calculated as the average of two diameters measured for each axon (Madsen et al., “Mitochondrial DNA Double-Strand Breaks in Oligodendrocytes Cause Demyelination, Axonal Injury, and CNS Inflammation,” J Neurosci 37: 10185-10199 (2017), which is hereby incorporated by reference in its entirety).

Striatal Tissue Dissociation. The mice were euthanized with carbon dioxide, transcardially perfused with sterile Hank's Balanced Salt Solution (HBSS), and the brain removed. The brains were immersed in ice-cold sterile HBSS for about 5 minutes to facilitate the microdissection. Under a dissecting microscope, the sub-ventricular zone was removed and discarded and the striata from each mouse was dissected and placed in sterile HBSS on ice. The striata from all the mice of the litter were pooled together as per genotype. The striatal tissues were transferred to a petri dish containing sterile HBSS then chopped into small pieces using sterile disposable scalpels, transferred into a sterile tube and then incubated in a papain/DNase dissociation solution at 37° C. for 50 minutes. Minimum essential media plus 0.5% BSA (MEM-BSA) containing 5% serum was then added to inactivate the papain. The tissue was triturated by repeated pipetting in order to achieve a single cell suspension. The cells were then pelleted, resuspended into MEM-BSA, and overlaid first onto a 90% Percoll gradient followed by a second 30% Percoll gradient and the solution centrifuged. The myelin and debris were removed from the tube and the cell pellet was resuspended in MEM containing 20 U/ml DNase.

Flow Cytometry and Cell Sorting. Flow cytometry for PDGFRa-EGFP glial progenitor cells was performed on freshly dissociated striatal cells from wild-type, R6/2 or Q175 mice. For FACS sorting, mouse striata were pooled together (n=3-4/group for R6/2 and 12 week zQ175 and WT controls; 4-8/group for 1 year-old zQ175 and WT). All samples were resuspended in MEM containing 20 U/ml DNase to a concentration of 1-1.5×10⁶ cells/ml and then passed over a 35 μm tube top cell strainer prior to flow cytometry. DAPI was added at 1 μg/ml. Flow cytometry analysis and FACS was performed on a BD FACSAria IIIU (Becton Dickinson, San Jose, Calif.). The cells were analyzed by forward and side scatter, for EGFP fluorescence through a 530±30 nm band-pass filter and for DAPI fluorescence through a 450±50 nm band-pass. Non-fluorescent cells were used to set the background fluorescence; a false positive rate of 0.5% was accepted. The EGFP⁺ and EGFP⁻ striatal cells isolated by FACS were pelleted, frozen on dry ice and stored at −80° C. until the time of RNA extraction.

RNA Preparation, Amplification, and Labeling. RNA was extracted from the pelleted/frozen cells using the Qiagen RNeasy Plus Mini kit. The RNA concentration was determined using a Nanodrop. A portion of the RNA was then used for bioanalysis to confirm the integrity of the RNA. RNA isolated from EGFP⁺ cells was used to generate sequencing libraries using the TruSeq RNA v2 kit, and sequenced on an Illumina HiSeq 2500 platform for approximately 45 million 2×125 bp reads per sample.

RNA-Seq Analysis of FACS Isolated GPCs. Reads were demultiplexed and cleaned using Trimmomatic (Bolger et al., “Trimmomatic: A Flexible Trimmer For Illumina Sequence Data,” Bioinformatics 30: 2114-2120 (2014), which is hereby incorporated by reference in its entirety). Reads were aligned to mouse genome GRCm38.p6 and mapped to Ensembl reference 92 via STAR 2.5.2b (Dobin et al., “STAR: Ultrafast Universal RNA-Seq Aligner,” Bioinformatics 29: 15-21 (2013), which is hereby incorporated by reference in its entirety), with quantMode set to TranscriptomeSAM. Gene abundances and expected counts were then calculated using RSEM 1.3.0 (Li and Dewey, “RSEM: Accurate Transcript Quantification From RNA-Seq Data With Or Without A Reference Genome,” BMC Bioinformatics 12: 323 (2011), which is hereby incorporated by reference in its entirety). Expected counts were imported into R via tximport for differential expression analysis (Soneson et al., “Differential Analyses For RNA-Seq: Transcript-Level Estimates Improve Gene-Level Inferences,” F1000Research 4: 1521 (2015); R Core Team, “R: A Language And Environment For Statistical Computing,” R Foundation for Statistical Computing Vienna, Austria (2017), which are hereby incorporated by reference in their entirety). Differential expression between Control and HD model Glia was conducted at each timepoint using DESeq2 following regression of a strain effect from the generalized linear model (Risso et al., “GC-Content Normalization For RNA-Seq Data,” BMC Bioinformatics 12: 480 (2011); Love et al., “Moderated Estimation Of Fold Change And Dispersion For RNA-Seq Data With Deseq2,” Genome biology 15: 550 (2014); which are hereby incorporated by reference in their entirety). Genes with an adjusted p-value <0.01 were considered significant. Only moderately expressed genes were kept for functional analysis, as they were more likely to be biologically significant (Mean transcripts per million (TPM)>1 in any group). Differentially expressed genes between both groups were analyzed in Ingenuity Pathway Analysis (IPA) (QIAGEN) for functional enrichment.

For functional analysis and inference of gene interactivity across all timepoints and models of HD, gene ontology networks were constructed following Weighted Gene Expression Correlated Network Analysis (WGCNA) (Langfelder and Horvath, “WGCNA: An R Package For Weighted Correlation Network Analysis.” BMC Bioinformatics 9: 559 (2008); which is hereby incorporated by reference in its entirety). Five modules were discovered from WGCNA. These modules were then filtered on significantly differentially expressed genes and fed into IPA. Three modules were enriched for terms relevant to glial maturity and myelination with the black module being most prominent. Significant terms were filtered by biological relevance and used to construct a functional IPA term network where term and gene nodes are connected via undirected edges. Network visualization was carried out in Cytoscape (Shannon, “Cytoscape: A Software Environment for Integrated Models of Biomolecular Interaction Networks,” Genome Res 13: 2498-2504 (2003); which is hereby incorporated by reference in its entirety) with the determination of neighborhoods occurring in Gephi (Bastian et al., “Gephi: An Open Source Software for Exploring and Manipulating Networks,” Proc. Third Int'l ICWSM Conference 3(1): 361-362 (2009); which is hereby incorporated by reference in its entirety). Nodes were clustered within their respective neighborhood and aesthetically repositioned slightly. Gene expression data are available via GEO, accession number GSE181370.

Western immunoblotting 12-week old R6/2 mice and their wildtype littermate controls (n=3 each) were perfused with HBSS and their corpora callosa were homogenized and proteins were extracted in radioimmunoprecipitation assay buffer (RIPA buffer, Sigma) in the presence of protease and phosphatase inhibitors (Halt™ Protease and Phosphatase Inhibitor Cocktail Thermo Scientific). 10 μg of protein was loaded on an electrophoresis gel (4-12% SDS-PAGE gels, NuPAGE™ 4-12% Bis-Tris Gel, Invitrogen) and transferred onto Immobilon membranes (Immobilon-FL Transfer Membrane PVDF, Millipore). Following the transfer, blots were stained with Ponceau (Sigma the incubated in Blocking buffer (SuperBlock™ Blocking Buffer in TBS, Thermo Scientific) then in TCF7L2 antibody (Cell Signaling Technologies, Clone C48H11) or β-Actin Antibody (Cell Signaling Technologies). The membrane was then treated with HRP-conjugated Goat anti-Rabbit Secondary Antibody (Cell Signaling Technologies) followed by SuperSignal™ West Pico PLUS Chemiluminescent Substrate and image with ChemiDoc Imaging System (BioRad). Image J was used to quantify the band intensities, and data were normalized to the expression of the housekeeping protein ß-Actin.

Quantitative Mass Spectrometry (MS) Callosal tissue was collected as above (n=4) and samples were processed as previously described (Hutti et al., “Global Analysis of Protein Degradation in Prion Infected Cells,” Sci Rep 10: 10800, 2020, which is hereby incorporated by reference in its entirety). Briefly, the whole corpus callosum or A2B5 FACS immuno-sorted striatal glial progenitor cells were pelleted and lysed in 50 uL of 5% SDS, 100 mM, sonicated, then briefly centrifuged to remove debris. Samples (15 μg from each sample were then reduced, alkylated, and digested overnight with trypsin using S-Traps (Protifi). Samples were then frozen and desiccated in a Speed Vac (Labconco), then resuspended in 0.1% trifluoroacetic acid prior to analysis. Peptide samples were then placed in a homemade 30 cm C18 column with 1.8 μm beads (Sepax), then loaded onto High-Performance Liquid Chromatography Easy nLC-1200 HPLC, Thermo Fisher), connected to a mass spectrometer (Fusion Lumos Tribrid, Thermo Fisher). Raw Data analysis was performed by a SEQUEST search engine within the Proteome Discoverer software platform, version 2.4 (Thermo Fisher), using the SwissProt Mus musculus database. Search parameter included up to 2 missed cleavages by Trypsin, a tolerance of 10 ppm for MS1 mass, and a tolerance of 0.6 Da for MS2 mass whereas oxidation of methionine was set as a variable modification and Carbamidomethyl was set as a fixed modification. Finally, relative protein abundance between samples was performed using Minora node and Percolator for false discovery rate, filtering out peptides which had a q-value greater than 0.01. Raw data is available at the Proteomics Identifications Database (https://www.ebi.ac.uk/pride/).

FACS isolation of striatal GPCs Striata were processed as above until debris was removed via Percoll gradient. Cells were then blocked for 5 minutes and stained with APC-conjugated A2B5 for 20 minutes. A2B5 was used to extract striatal GPCs, since PDGFRa expression was found to diminish with age in these models, while A2B5 expression was maintained and remained selectively expressed by GPCs in the white matter, as previously reported (Gard and Pfeiffer, “Two Proliferative Stages of the Oligodendrocyte Lineage (A2B5+O4− and O4+GalC−) Under Different Mitogenic Control,” Neuron 5: 615-625 (1990); Roy et al., “Identification, Isolation, and Promoter-defined Separation of Mitotic Oligodendrocyte Progenitor Cells from the Adult Human Subcortical White Matter,” J Neurosci 19: 9986-9995 (1999), which are hereby incorporated by reference in their entirety). The cells were washed and then FACS isolated for A2B5 positivity before pelleting, desiccating, and flash freezing. These pellets were then submitted for quantitative mass spectrometry.

Assessment of Human TCF7L2 Isoform Distribution. Raw Fastq files from published HD hGPC dataset (Osipovitch et al., “Human ESC-Derived Chimeric Mouse Models of Huntington's Disease Reveal Cell-Intrinsic Defects in Glial Progenitor Cell Differentiation,” Cell Stem Cell 24: 107-122 e107 (2019), which is hereby incorporated by reference in its entirety) were obtained from GSE105041 and aligned to GRCh38 using Ensembl 95 gene annotations via STAR 2.5.4b in 2-pass mode across all samples and quantified with RSEM 1.3.1. Isoform abundances were imported into R via Tximport (1.8.0).

Viral Construct and Injection. Human TCF7L2 (NM_030756.5) was cloned into pTANK-TRE-CAG-rtTA3G-WPRE under the control of tetracycline inducible promoter (Osipovitch et al., “Human ESC-Derived Chimeric Mouse Models of Huntington's Disease Reveal Cell-Intrinsic Defects in Glial Progenitor Cell Differentiation,” Cell Stem Cell 24: 107-122 e107 (2019), which is hereby incorporated by reference in its entirety). Viral particles pseudotyped with Vesicular Stomatitis Virus G glycoprotein were produced and titrated limit dilution on 293HEK cells (3.8 10⁹ cfu/ml). One microliter of viral suspension was injected into the striatum of ten weeks old R6/2 mice bilaterally in the following coordinates from the bregma: coordinates: +1.1 mm anterior/posterior, ±1.5 mm medial/lateral, −2.3 mm dorsal/ventral from the dura. Immediately after the injection, a cohort of mice were given doxycycline (introduced into their water ad lib) to allow TCF7L2 expression while others were not given doxycycline, thus serving as matched controls. At 12 weeks of age, all the mice were sacrificed and their striatum were dissected and processed for RNA extraction.

Histology. Animals were killed using sodium pentobarbital and perfused transcardially with saline followed by 4% paraformaldehyde, and their brains were processed for immunocytochemistry as previously described (Benraiss et al., “Cell-Intrinsic Glial Pathology Is Conserved Across Human And Murine Models Of Huntington's Disease,” Cell Reports 36: 109308 (2016); which is hereby incorporated by reference in its entirety). Sagittal sections (20 μm) spanning corpus callosum were processed for immunostaining with anti-EGFP (Chicken anti-EGFP; Rockland), in combination with anti-Olig2 (Goat anti-olig2; R&D Systems), anti-Ng2 (Rabbit anti-NG2; Millipore), or anti-GFAP (Mouse monoclonal anti-human GFAP; Covance Research).

QPCR. RNA was isolated using TRIZOL, cleaned via RNeasy Mini kit (Qiagen, Germany) following manufacturer's instructions and quantified on a nanodrop spectrophotometer. First-strand cDNA was synthetized using TaqMan Reverse Transcription Reagents (Applied Biosystems, USA). Real-time PCR samples were prepared in triplicate with 5 ng of RNA in FastStart Universal SybrGreen Mastermix (Roche Diagnostics, Germany) and amplified on a CFX Connect Real-Time System Thermocycler (Bio-Rad, USA). Primer sequences are listed in Table 1. Melting-curve analysis was performed following each PCR to confirm reaction specificity. Results were normalized within samples to 18S gene expression. Fold changes were calculated using the AACt method (Pfaffl, “A New Mathematical Model for Relative Quantification in Real-time RT-PCR,” Nucleic Acids Research 29: e45 (2001); which is hereby incorporated by reference in its entirety).

TABLE 1
Primers Used for qPCR Validation

| Name | Gene ID | Forward (5′-3′) | Reverse (5′-3′) |
|---|---|---|---|
| Cyp51a1 | 13121 | GTTCCTGACCTGCTTGAGTT | GAGGGTGAAGGTGGGTTATTT |
| | | (SEQ ID NO. 1) | (SEQ ID NO. 2) |
| Mbp | 17196 | CGACACCTCCTTATCCCTCTAA | ATCAACCATCACCTGCCTTC |
| | | (SEQ ID NO. 3) | (SEQ ID NO. 4) |
| Sox10 | 20665 | CCGTGTGCTACTGTGTCATT | TCCTCAGTGCGTCCTTAGT |
| | | (SEQ ID NO. 5) | (SEQ ID NO. 6) |
| Mag | 17136 | GCAGGACAGGAAACAGTGA | GAGCTCTAATGTAGGCAAGGAA |
| | | (SEQ ID NO. 7) | (SEQ ID NO. 8) |
| Tcf712 | 21416 | GGGACACCACTCAACAATGA | GGTACAGCAAGCAACCTCTAA |
| | | (SEQ ID NO. 9) | (SEQ ID NO. 10) |
| Mog | 17441 | CCTCTTTGCTGTGGACAGATT | GAGCTGAGGAAAGACAGTTGAG |
| | | (SEQ ID NO. 11) | (SEQ ID NO. 12) |
| Trf | 22041 | GCCTGCACTTTCCACAAAC | GGGCATGAAGACCAAGAGAA |
| | | (SEQ ID NO. 13) | (SEQ ID NO. 14) |
| Myrf | 225908 | CTTCCTTCTGGTCATCTCCTTG | GAGAGTGAAGGTGGGCAAATA |
| | | (SEQ ID NO. 15) | (SEQ ID NO. 16) |
| Plp1 | 18823 | CTCACTCCCTTCTCCTTGATAAC | GCTCCTTAGGTCACAGTCTTATT |
| | | (SEQ ID NO. 17) | (SEQ ID NO. 18) |
| Hmgcr | 15357 | GCCACACTCTGCACTAAAGATA | CTGTTCCCACACTCTAAGTTCTG |
| | | (SEQ ID NO. 19) | (SEQ ID NO. 20) |
| Srebf1 | 20787 | CTTTCCCTTGAGGACCTTTGT | CAATTTCCAGATCTGCCACTAGA |
| | | (SEQ ID NO. 21) | (SEQ ID NO. 22) |
| Srebpf2 | 20788 | AGGTCTAGGGATGGGTGAAA | GTGGGAAGGAACAGGACAATTA |
| | | (SEQ ID NO. 23) | (SEQ ID NO. 24) |
| Ctnnb1 | 12387 | GGACCAGGTGGTGGTTAATAAG | CATCTGAGGAGAACGCATGATAG |
| | | (SEQ ID NO. 25) | (SEQ ID NO. 26) |
| Dkk1 | 13380 | AGCACCTTGGATGGGTATTC | CTGATGACCGGAGACAAACA |
| | | (SEQ ID NO. 27) | (SEQ ID NO. 28) |
| Fzd8 | 14370 | CCTCGACTCTTCCTACGTAAAC | GAATACCCAAACCGTGCAATC |
| | | (SEQ ID NO. 29) | (SEQ ID NO. 30) |
| Lrp6 | 16974 | CTGACTGCCTCCAACGTAAA | CTGTACATGGTCTGCCTCATC |
| | | (SEQ ID NO. 31) | (SEQ ID NO. 32) |
| Kaiso/ | 56805 | GGTGGCTAGACCACTTTATACC | CATCTCCTCCTATCTGCCATTAC |
| Zbtb33 | | (SEQ ID NO. 33) | (SEQ ID NO. 34) |
| Stk11 | 20869 | GACATCATCTACACTCAGGACTTC | CTCTGTGCCGTTCATACACA |
| | | (SEQ ID NO. 35) | (SEQ ID NO. 36) |
| Tle1 | 21885 | GGCAGTCTCACTTGGCAATA | TGGGACCAGGAGGGAATTA |
| | | (SEQ ID NO. 37) | (SEQ ID NO. 38) |
| Rn18S | 19791 | GCAATTATTCCCCATGAACG | GGCCTCACTAAACCATCCAA |
| | | (SEQ ID NO. 39) | (SEQ ID NO. 40) |

Statistical Analysis. Data were analyzed using GraphPad Prism V8 (GraphPad Software Inc., La Jolla, Calif., USA). Data were analyzed using GraphPad Prism 8.0 (GraphPad, San Diego, Calif.). Unpaired t tests were used to compare two groups, while 2-Way ANOVA (followed by Tukey's post-hoc comparison tests) was used to compare four or more groups. Frequency distribution plots of the number of myelinated axons as a function of their diameters were analyzed using non-linear regression, with a Lorentzian distribution curve fitting model. Quantitative results are shown as mean±S.E.M., and statistical significance was accepted at p<0.05.

### Example 1—Ultrastructural Image Analysis Revealed a Progressive Hypomyelination in HD Mouse Model

Human ESC-derived mHTT-expressing GPCs manifest delayed oligodendrocytic differentiation after neonatal transplantation into hypomyelinated shiverer mice (Osipovitch et al., “Human ESC-Derived Chimeric Mouse Models of Huntington's Disease Reveal Cell-Intrinsic Defects in Glial Progenitor Cell Differentiation,” Cell Stem Cell 24: 107-122 e107 (2019), which is hereby incorporated by reference in its entirety). This differentiation delay is due to a cell autonomous, mHTT-dependent block in terminal glial differentiation from the bipotential hGPCs. This finding suggested that the homeostatic maintenance of mature myelin, as well as the remyelination of lesions after adult demyelination, might be impaired in vivo, as each of these processes requires unimpeded oligodendrocytic differentiation from the parenchymal GPC pool. Next, the myelin maintenance and repair was evaluated to determine if it was disrupted in vivo, in the R6/2 mouse model of juvenile HD. These mice harbor the first exon of mutant human Huntingtin (mHTT), with a 120 CAG repeat expansion. They display symptoms as early as 8 weeks, and deteriorate rapidly thereafter, with premature death at 16-17 weeks.

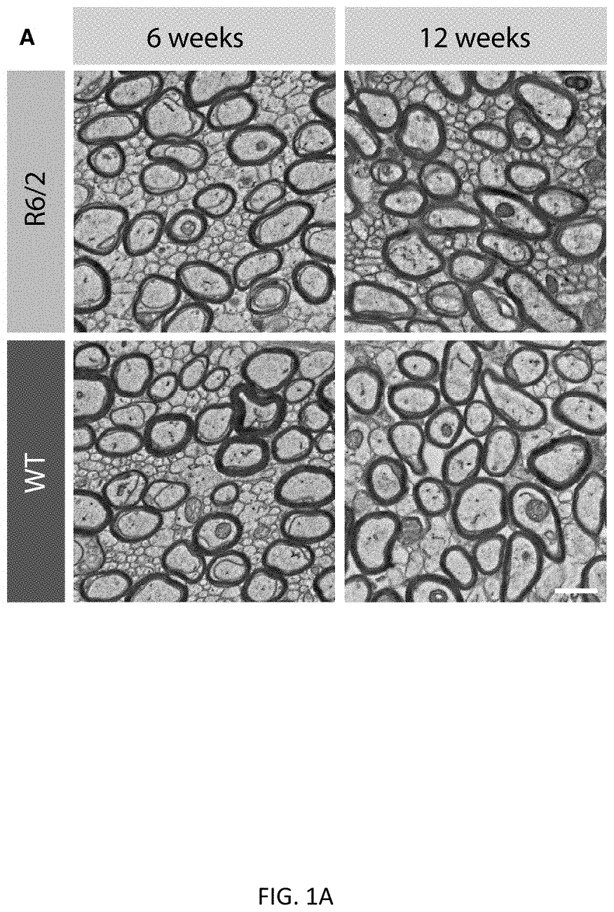

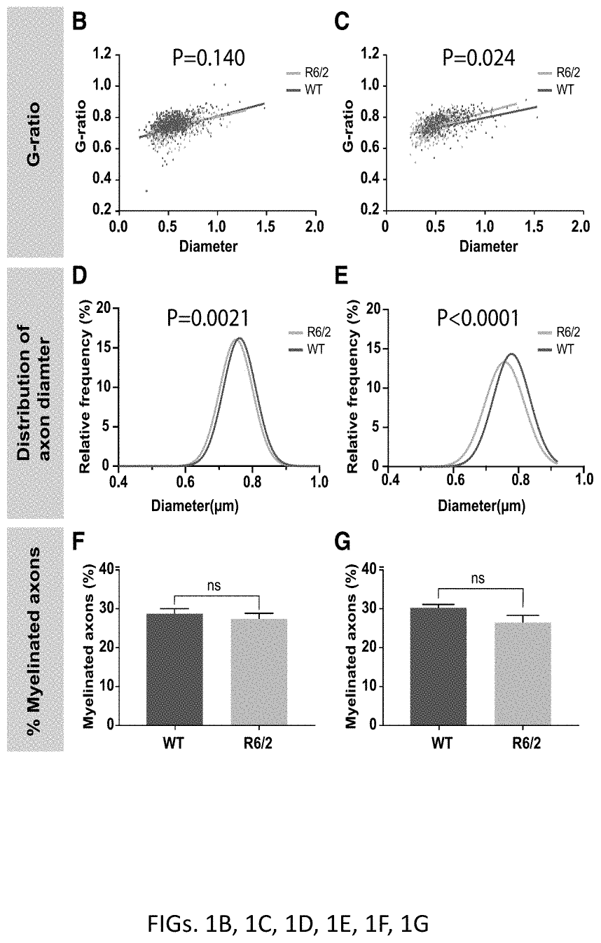

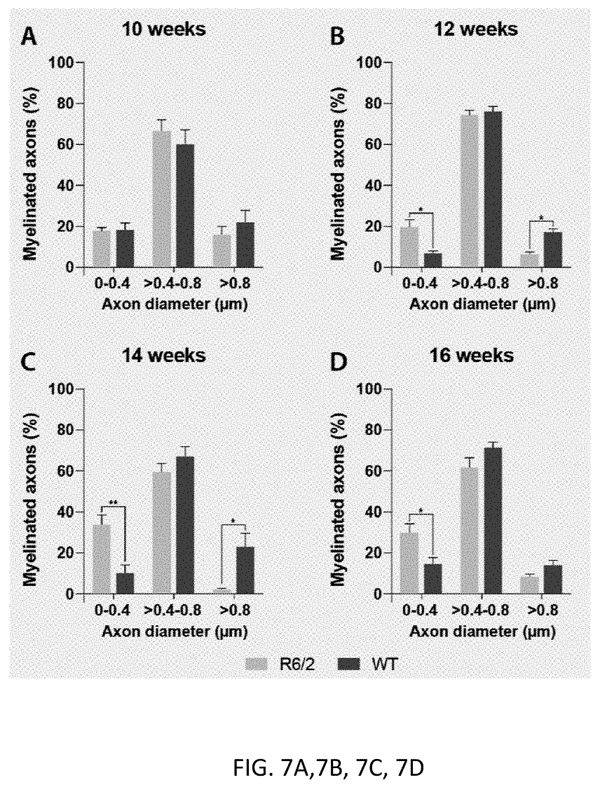

To assess myelination in the R6/2 brain, we first examined the ultrastructure of callosal myelin in both R6/2 mice and their wild-type controls, at two discrete timepoints: pre-symptomatic (6 weeks) and diseased (12 weeks; n=4 mice for all groups). Analysis of electron microscopic (EM) images of the corpus callosum was performed using the ImageJ plugin G-ratio calculator to measure the G-ratio of inner and the outer diameter of myelin sheaths (Goebbels et al., “Elevated Phosphatidylinositol 3,4,5-Trisphosphate In Glia Triggers Cell—Autonomous Membrane Wrapping And Myelination,” J Neurosci 30: 8953-8964 (2010), which is hereby incorporated by reference in its entirety). The percentage of myelinated fibers within each studied field was also scored (FIG. 1), as was the size distribution of the ensheathed axons, as a function of genotype (FIGS. 7A-7D). At 6-weeks, the g-ratio of myelinated axons in R6/2 mice did not significantly differ from that of wild-type mice (F[1, 1219]=2.18 p=0.14 by linear regression; FIGS. 1A and 1B). In contrast, by 12 weeks, the average g-ratio of myelinated R6/2 callosal axons was significantly higher than that of WT controls, suggesting that R6/2 axons have fewer and/or thinner myelin wraps per axon than WT mice (F[1, 799]=5.09 p=0.024; FIGS. 1A and 1C). Lorentzian non-linear regression analysis (Motulsky & Brown, “Detecting Outliers When Fitting Data With Nonlinear Regression—A New Method Based on Robust Nonlinear Regression and The False Discovery Rate,” BMC Bioinformatics 7: 123 (2006), which is hereby incorporated by reference in its entirety) of the frequency of myelinated fibers as a function of diameter, revealed a shift toward smaller axons in R6/2 compared to WT mice; this difference was already significant at 6 weeks of age, and widened further by 12 weeks (6 weeks: F[3, 66]=35.8, p<0.0021; 12 weeks: F[3, 38]=8.83, p=0.0001; FIGS. 1D and 1E, see also FIGS. 7A-7D). However, the proportion of myelinated fibers did not differ between R6/2 and WT mice at either timepoint (6 weeks, p=0.58; 12 weeks: p=0.09 by t test) (FIGS. 1F and 1G). These data indicate that relative to WT mice, R6/2 HD mice acquire a progressive, age-related loss of callosal myelin, reflected in the tonic decline in both the caliber and myelin thickness of their callosal axons.

### Example 2—Remyelination is Significantly Delayed in HD Model R6/2 Mice

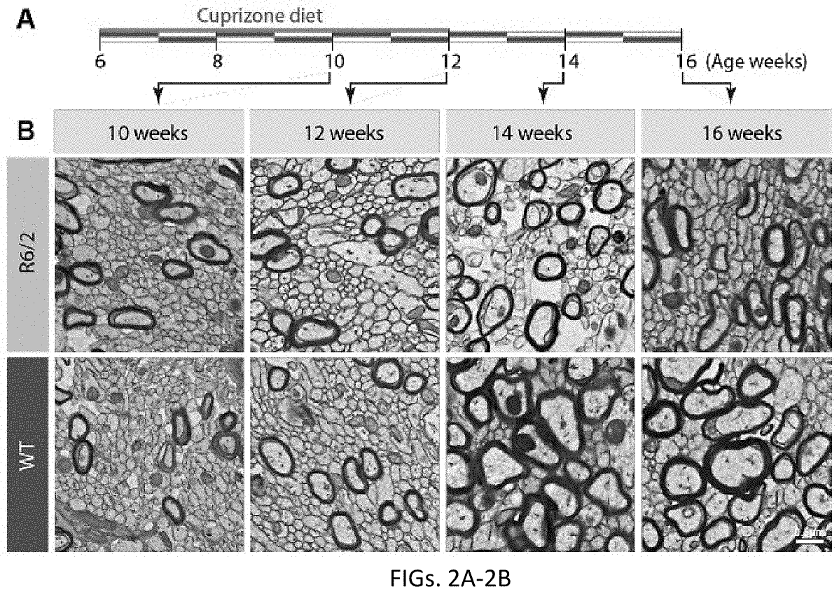

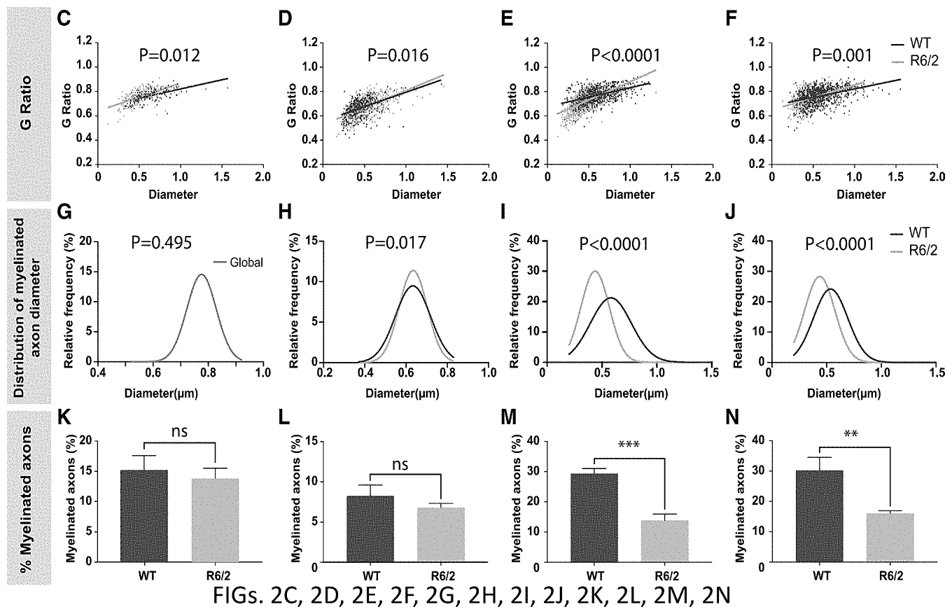

Cellular pathology leading to the developmental hypomyelination of HD mice may manifest in impaired remyelination. The copper chelator cuprizone, a well-established oral demyelinating toxin, was administered to both R6/2 and WT mice for up to 6 weeks, beginning at 6 weeks of age (Stidworthy et al., “Quantifying The Early Stages Of Remyelination Following Cuprizone-Induced Demyelination,” Brain Pathol 13: 329-339 (2003); which is hereby incorporated by reference in its entirety). The mice were sacrificed at one of 4 time points (n=4 mice/genotype/time point): at either 4 weeks into the diet at 10 weeks of age; at 12 weeks, immediately at the end of cuprizone diet; or either 2 or 4 weeks thereafter, at 14 or 16 weeks (FIG. 2A). All the brains were processed for transmission electron microscopy and imaged as noted earlier, with quantitative assessment of axonal diameter, g-ratio and myelinated fiber frequency. Four weeks into the cuprizone diet, the callosa of both R6/2 and WT mice displayed fewer myelinated axons that differed in their g-ratios (FIG. 2C; F(1, 315)=6.35, p=0.012 by linear regression), but not in their distributions as a function of diameter (FIG. 2G; F(3,34)=0.26, p=0.50 by non-linear regression), nor in their percentages of total myelinated axons (FIG. 2K; p=0.63 by t-test). Among the cuprizone-treated mice, remyelination was relatively deficient and temporally delayed in R6/2 mice relative to WT controls, at all studied time points. Indeed, by 12 weeks of age, the efficacy of remyelination differed significantly in R6/2 compared to WT mice (FIGS. 2D-2N). The remyelinated callosal axons of cuprizone-treated R6/2 HD mice had higher g-ratios—indicating thinner myelin—than their WT littermates, both immediately after the cessation of the cuprizone diet at 12 weeks, and at 2 and 4 weeks thereafter, post-recovery (12 weeks: F(1,812)=2.412, p=0.016; 14 weeks: F(1,1603)=77.80, p<0.0001; 16 weeks: F(1,1398)=10.69, p<0.001 by linear regression; FIGS. 2D-2F). Non-linear regression analysis of the frequency of myelinated fibers as a function of diameter revealed that after cuprizone treatment, larger diameter fibers were less likely to be myelinated in HD mice than in WT (12 weeks: F(3,24)=3.79, by non-linear regression, p=0.017; 14 weeks: F(3,20)=45.86, p<0.0001; 16 weeks: F(3,24)=14.66, p<0.0001) (FIGS. 2H-2J and FIG. 7).

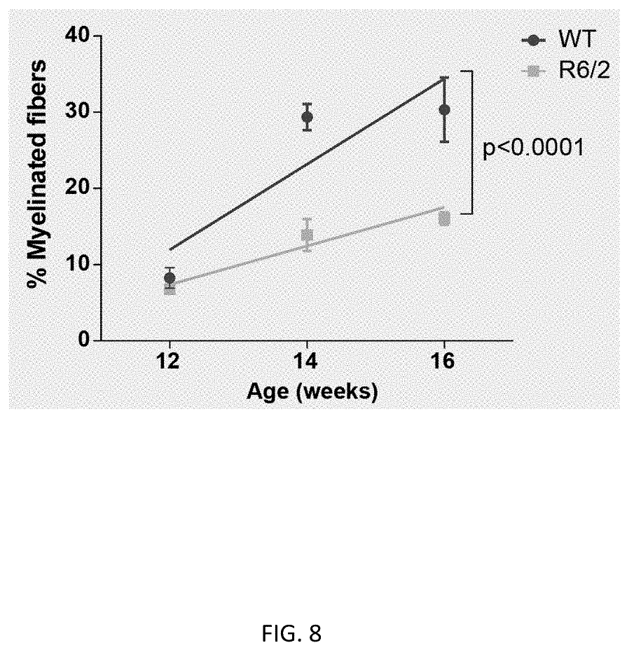

The percentage of remyelinated axons did not differ between WT and R6/2 mice at the earlier 10 and 12 week timepoints, when the mice were still on cuprizone (10 weeks p=0.62; 12 weeks p=0.29 by t test); in contrast, the proportion of remyelinated axons became significantly higher in WT than in R6/2 mice at 2 and 4 weeks after cuprizone cessation, further indicating the significantly more rapid recovery and remyelination of WT mice (14 weeks p<0.001; 16 weeks p<0.01 by t test; FIGS. 2L-2N). Regression analysis of the number of remyelinated axons as a function of recovery time showed that with recovery, the percentage of myelinated fibers was consistently and significantly lower in R6/2 compared to WT littermates (F(2,116)=16.15, p<0.0001 by Linear regression; FIG. 8). Thus, remyelination was significantly delayed and ultimately deficient in cuprizone-demyelinated R6/2 mice, relative to WT controls.

### Example 3—Proteomic Analysis of HD Mouse White Matter Reveals Diminished Myelin Protein Expression

To complement the ultrastructural analyses with a biochemical assessment of the relative myelination of HD and WT brains, mass spectrometry of the callosal white matter was performed to more definitively establish and characterize the hypomyelination of HD mouse brain. Two different HD mouse models were used to ensure that any disease-associated correlates to hypomyelination were not model-specific. In particular, besides R6/2, the zQ175 mouse was used, which expresses full length mutant HTT, harboring roughly 190 CAG repeats. zQ175 mice develop milder symptoms than R6/2 mice; they have normal life spans, with most motor and behavioral symptoms beginning only after a year of age. As such, these mice reflect a later-onset form of HD (Menalled et al., “Comprehensive Behavioral and Molecular Characterization of a New Knock-in Mouse Model of Huntington's Disease: zQ175,” PLoS one, 7: e49838 (2012); Carty et al., “Characterization of HTT Inclusion Size, Location, and Timing in the zQ175 Mouse Model of Huntington's Disease: an in vivo High-Content Imaging Study,” PloS one 10: e0123527 (2015), which are both hereby incorporated by reference in their entirety).

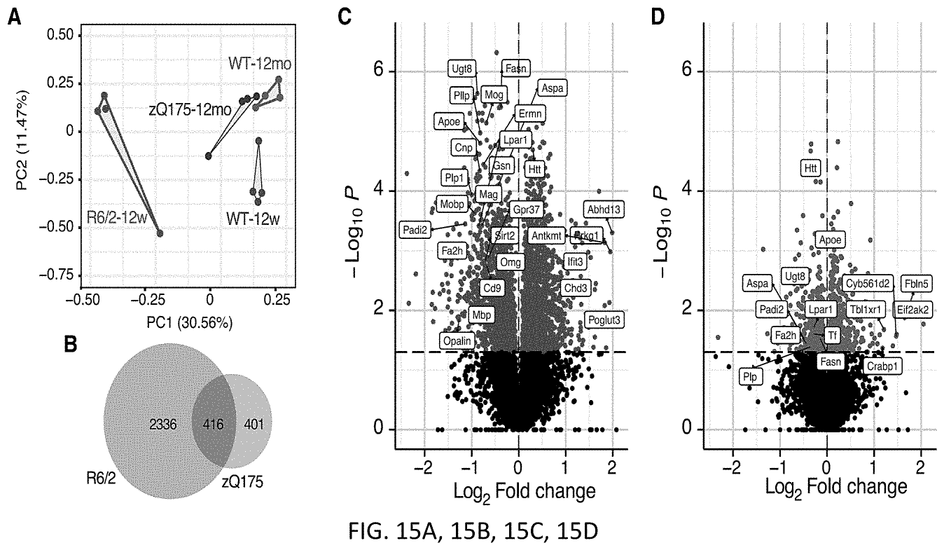

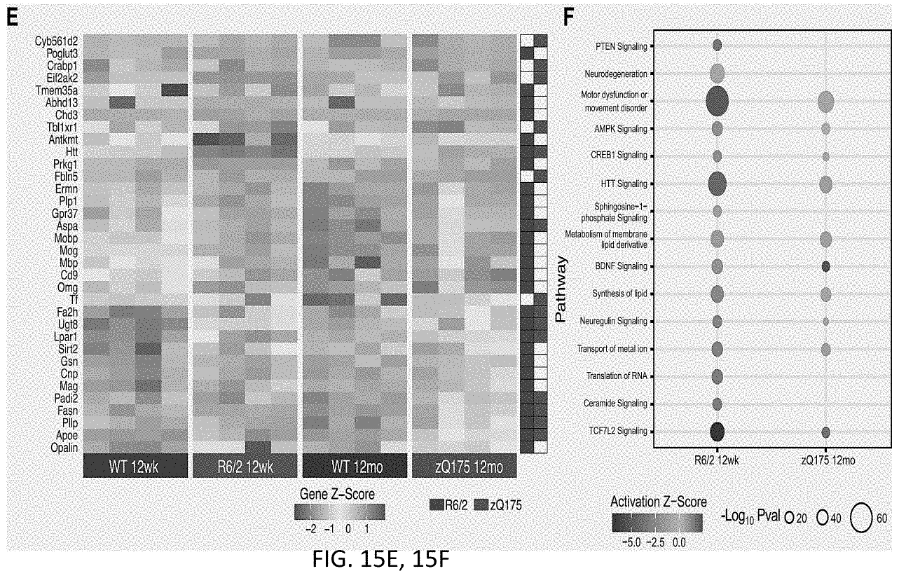

Using these two distinct transgenics, the corpus callosa of 12-week old R6/2 and 12-month old zQ175 mice, in addition to their WT littermate controls, were sampled so as to identify conserved differences between HD and WT mice in their white matter proteomes (n=4 mice/group). Principal component analysis (PCA) of these samples revealed sharp segregation of diseased R6/2 callosal white matter from WT, and lesser but nonetheless overt segregation of zQ175 white matter from that of its WT controls (FIG. 15A). Differential expression analysis between these proteomes revealed 2,443 unique dysregulated peptides in R6/2 callosal white matter (1,076 downregulated and 1,367 upregulated; FDR <0.05), and 722 in zQ175 white matter (304 downregulated, 418 upregulated) (FIGS. 15B, 15C, and 15D). Of these differentially expressed cohorts, 416 peptides were found to be dysregulated in both HD models (FIG. 15B). A large number of myelin proteins were differentially downregulated in R6/2 callosal white matter, including Mbp, Mag, Mog, Mobp, Plp1, Gpr37, Aspa, and Cnp (FIGS. 15C and 15E). Myelin protein downregulation was also evident in 12-month old zQ175 callosal white matter, but not to the sharp extent noted in R6/2 mice, with Ugt8, Tf, Aspa, Lpar1, and Fa2h among those downregulated (FIGS. 15D and 15E).

Ingenuity pathway analysis of the differentially expressed proteins in R6/2 callosal white matter revealed predicted activation of HTT, PTEN, CREB1, and AMPK signaling, in concert with signatures consistent with both neurodegeneration and neurodegenerative movement disorders (FIG. 15F). At the same time, TCF7L2, BDNF, S1P, neuregulin, and ceramide signaling were all predicted to be repressed in R6/2 white matter. Functional repression of lipid synthesis and metabolism, metal ion transport, and RNA translation were also predicted in R6/2 mice. Similarly, while 1 year-old zQ175 callosal white matter showed milder functional dysregulation than R6/2, AMPK signaling and neurodegenerative movement disorders were also predicted to be activated in zQ175, while TCF7L2 and neuregulin signaling, lipid synthesis and metabolism, and metal iron transport were all predicted to be repressed (FIG. 15F). Together, these proteomic data provide a biochemical correlate to the finding that HD is associated with hypomyelination, with a largely shared molecular signature across HD genotypes.

### Example 4—GPCs Derived from Different HD Models Shared Common Transcriptional Defects

The transcriptional basis for the apparent defect of R6/2 and zQ175 HD mice in both myelin maintenance and restorative myelinogenesis was defined. Previously, it was identified that human GPCs derived from HD-derived pluripotent stem cells in vitro exhibited impaired oligodendroglial differentiation, reflected in their down-regulation of a critical set of glial transcription factors, including, OLIG2, NKX2.2, SOX10 and MYRF (Osipovitch et al., “Human ESC-Derived Chimeric Mouse Models of Huntington's Disease Reveal Cell-Intrinsic Defects in Glial Progenitor Cell Differentiation,” Cell Stem Cell 24: 107-122 e107 (2019), which is hereby incorporated by reference in its entirety). To understand if the defective developmental myelinogenesis and remyelination failure of R6/2 mice is transcriptionally altered, high-throughput RNA sequencing was used to examine the transcriptional profiles of CD140a/PDGFRA-defined GPCs, which comprise the principal source of oligodendrocytes in both the murine and human CNS (Sim et al., “Complementary Patterns Of Gene Expression By Human Oligodendrocyte Progenitors And Their Environment Predict Determinants Of Progenitor Maintenance And Differentiation,” Ann Neurol 59: 763-779 (2011); which is hereby incorporated by reference in its entirety). In addition, to ensure that any differential gene expression noted was not model-specific, a second mouse model was used, the zQ175 mouse which expresses full length mutant HTT with 190 CAG repeats. Unlike the R6/2 mice, zQ175 mice develop milder symptoms beginning at 1 year of age, and have normal life spans, and as such represent a later-onset form of HD (Menalled et al., “Comprehensive Behavioral And Molecular Characterization Of A New Knock-In Mouse Model Of Huntington's Disease: zQ175,” PloS one 7: e49838 (2012); Carty et al., “Characterization of HTT Inclusion Size, Location, and Timing in the zQ175 Mouse Model Of Huntington's Disease: an in vivo High-Content Imaging Study,” PloS one 10: e0123527 (2015); which are hereby incorporated by reference in their entirety).

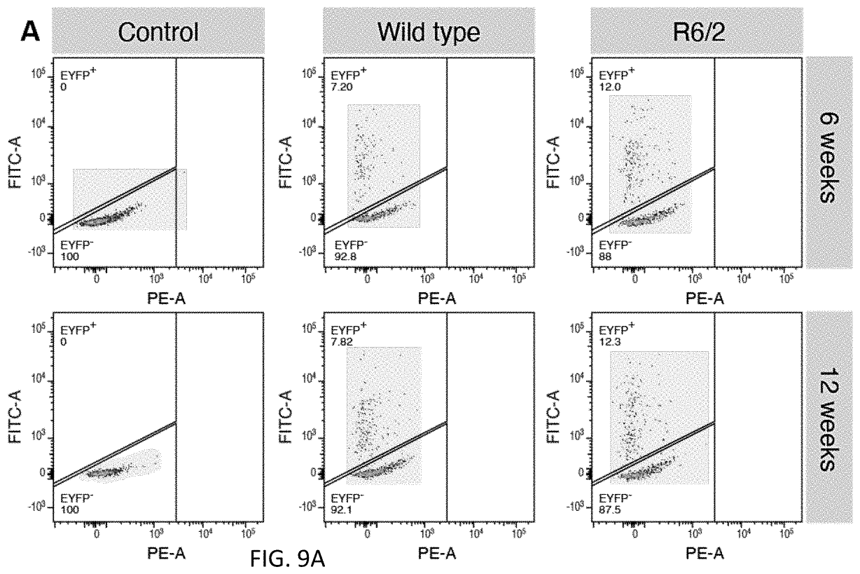

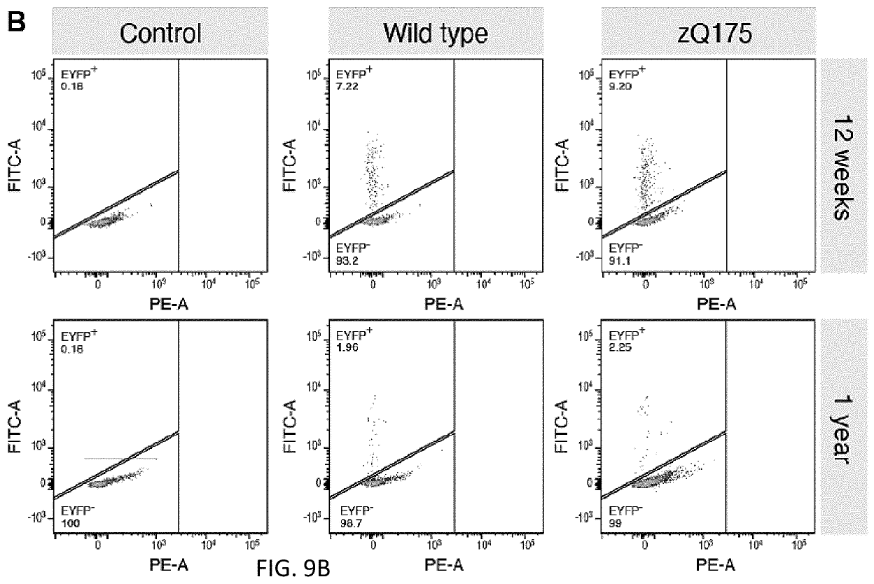

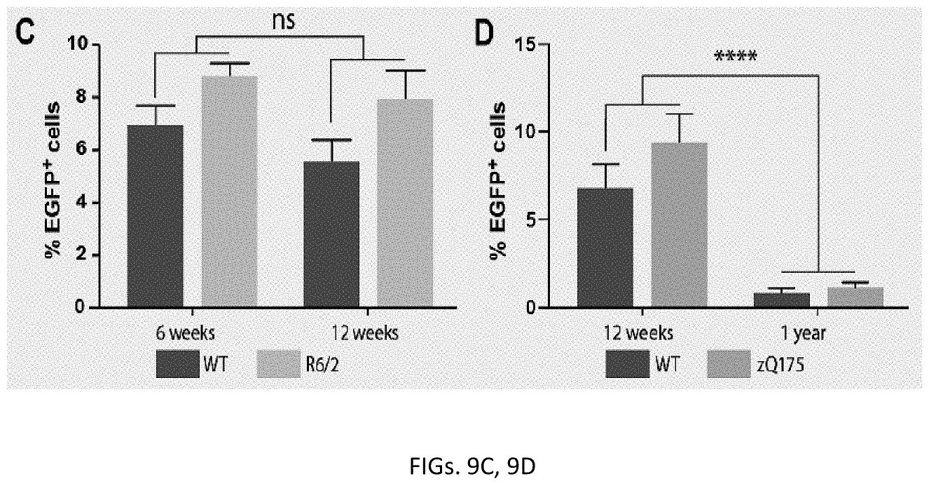

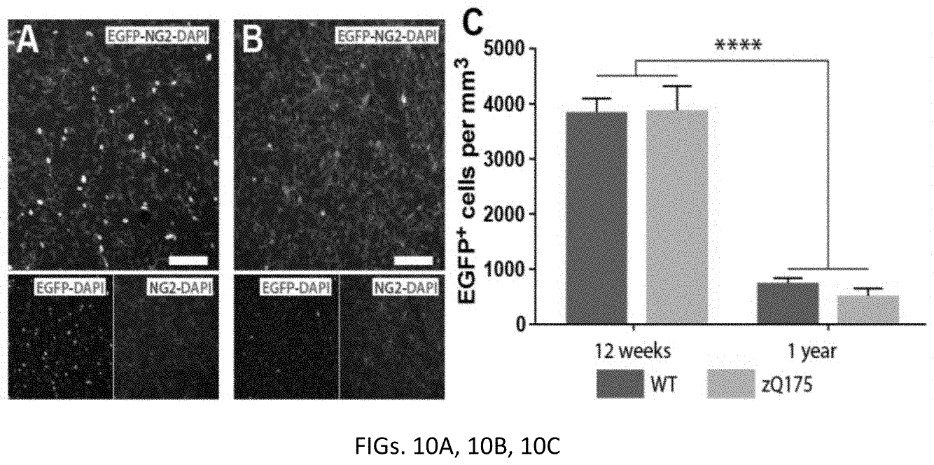

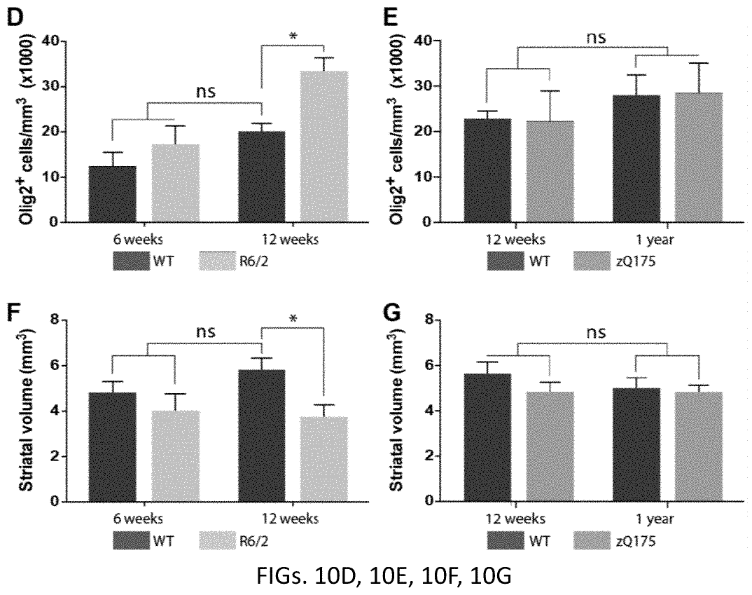

To reliably identify and isolate GPCs from zQ175 mice, each mouse line was bred to PDGFRA-EGFP reporter mice (Hamilton et al., “Evolutionary Divergence of Platelet-Derived Growth Factor Alpha Receptor Signaling Mechanisms,” Mol Cell Biol 23: 4013-4025 (2003); which is hereby incorporated by reference in its entirety), to yield bigenic GPC reporters for each HD line. Both presymptomatic (6 weeks old for R6/2 and 12 weeks for zQ175) and symptomatic (12 weeks for R6/2 and 1 year for zQ175) mice were analyzed. GPCs were acutely isolated via FACS from the striata of HD transgenic mice and their littermate controls at each timepoint; 3-8 mice were pooled for each sample, depending upon the age of the group (FIG. 9). In that regard, 1 year old mice—both zQ175 as well as their WT littermate controls—harbored significantly fewer EGFP⁺ cells than younger mice (age effect: p<0.0001, F(1, 20)=53.9 by 2-way ANOVA; FIG. 9). This age-related fall in the EGFP counts was confirmed by stereological estimation of the total EGFP⁺ cells in the striata of zQ175 at both timepoints (age effect: p<0.0001, F(1, 23)=155.0; FIG. 10). Yet stereological counts of Olig2 did not differ between 12-week and 1-year old mice, in either WT or zQ175 mice (genotype effect: p=0.91, F(1, 22)=0.01; age effect: p=0.29, F(1, 22)=1.16; FIG. 10). Thus, the drop in EGFP⁺ cells appeared due not to a loss of GPCs, but rather to an age-related downregulation of PDGFRa expression in aged mice. Indeed, 12-week old R6/2 mice showed an increase of Olig⁺ cell density—likely due to preserved numbers in the setting of reduced striatal volume in diseased mice—again suggesting that the fall in PDGFRA-driven EGFP expression in the 1 year-old mice was a reflection of age, and not disease (FIG. 10).

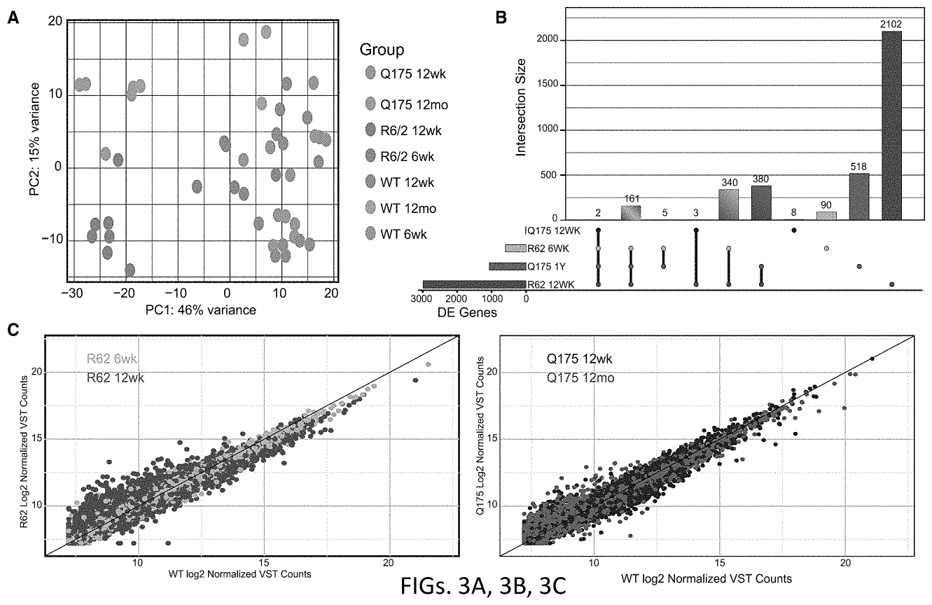

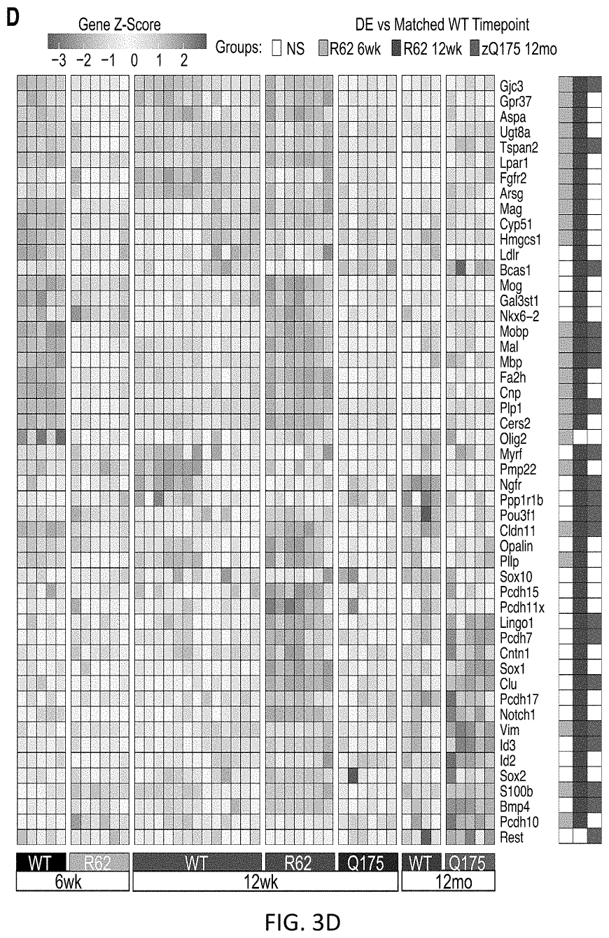

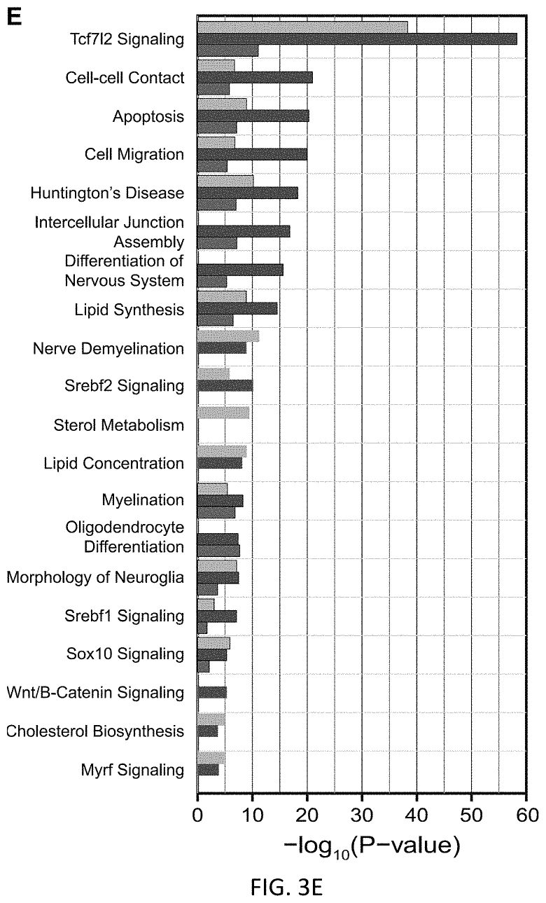

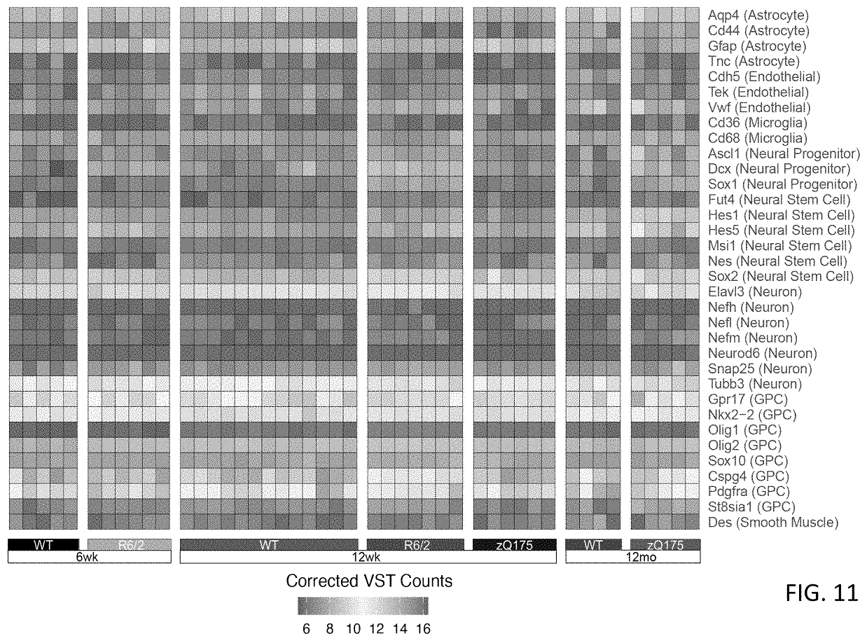

Following batch correction, principal component analysis of these RNA-Seq samples revealed a tight clustering of control and disease mice, that was more prominent at later timepoints, (FIG. 3A). All groups displayed transcriptional signatures consistent with GPC phenotype (FIG. 11). In R6/2, 598 genes were differentially expressed at 6 weeks (FDR adjusted p<0.01), whereas 2988 genes were dysregulated at 12 weeks. In contrast, zQ175 exhibited milder transcriptional dysregulation, as only 13 genes were dysregulated at 12 weeks and 1066 at one year. Many dysregulated genes were shared between both R6/2 and zQ175 at both presymptomatic and diseased stages (FIG. 3B). Scatter plots of normalized, variance-stabilizing transformed counts of transcripts showed larger expression fold-changes in R6/2 than in zQ175 mice (FIG. 3C).

Among the differentially expressed genes, several myelinogenic genes were downregulated in both R6/2 and zQ175. These included Myrf, Bcas1, Plp1, Mbp, and Mobp (FIG. 3D). Expression of genes prominent in early progenitors and astrocytes was found to be enriched in both disease models; these included Vim, Bmp4, S100b, Id3, Clu, and Lingo1. Functional analysis by IPA of the R6/2 and zQ175 differentially expressed gene sets showed a significant enrichment of terms involved in myelinogenesis, including myelination, synthesis of lipids, and differentiation of oligodendrocytes (FIG. 3E). Upstream signaling associated with myelination was also disrupted in both models, including SOX10-dependent and TCF7L2-dependent transcription, each of which was significantly suppressed in HD GPCs.

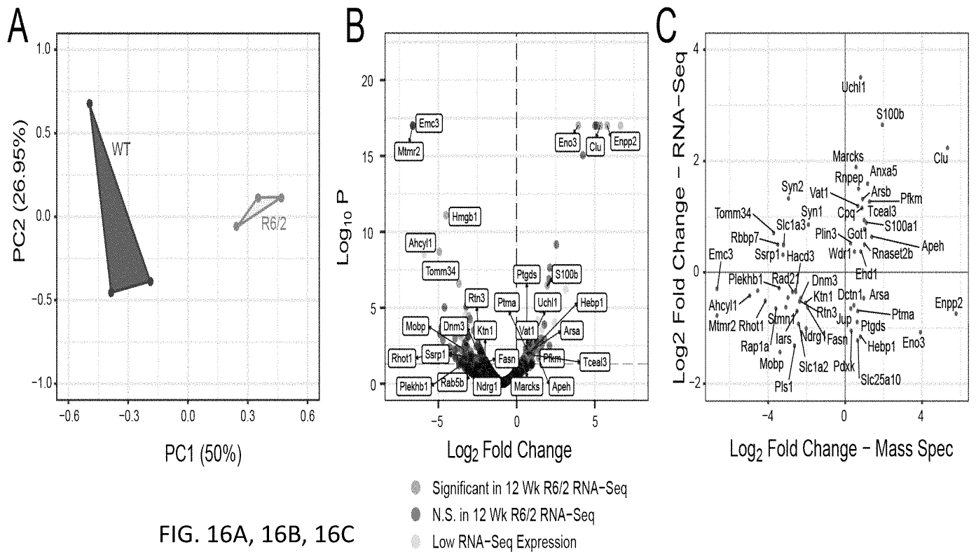

Next, correlation of the transcriptional and proteomic data was performed by assaying 12-week-old striatal R6/2 and WT littermate control GPCs via mass spectrometry. This model and time point were chosen because its differential gene expression predicted the greatest degree of relative dysmyelination. Striatal GPCs were dissected, dissociated, and isolated by sorting on A2B5, which targets ganglioside epitopes enriched on GPCs (n=3) (Gard and Pfeiffer, “Two Proliferative Stages of the Oligodendrocyte Lineage (A2B5+O4− and O4+GalC−) under Different Mitogenic Control,” Neuron 5: 615-625 (1990); Roy et al., “Identification, Isolation, and Promoter-defined Separation of Mitotic Oligodendrocyte Progenitor Cells from the Adult Human Subcortical White Matter,” J Neurosci 19: 9986-9995 (1999), which are hereby incorporated by reference in their entirety). Unlike PDGFRa, A2B5 is not cleaved by Papain, which was used for enzymatic dissociation of tissue in this study, and thus was ideal for acute dissociation and isolation of GPCs. PCA of the detected peptides revealed tight clustering of R6/2 striatal GPCs, and their segregation from WT GPCs (FIG. 16A). Differential expression analysis revealed 212 proteins dysregulated in R6/2 striatal GPCs compared to WT GPCs (FIG. 16B). Inspection of the intersection of differentially expressed proteins and genes revealed that 69.6% of the genes and transcripts displayed concordant directionality (FIG. 16C). Among these, R6/2 striatal GPCs exhibited sharply lower levels of the myelin- and oligodendrocyte-enriched proteins Mobp, Fasn, and Ndrg1, among others, consistent with the disease-associated suppression of myelinogenic programs in HD GPCs.

### Example 5—Network Analysis Revealed mHTT-Length Dependent Deficit in Myelination

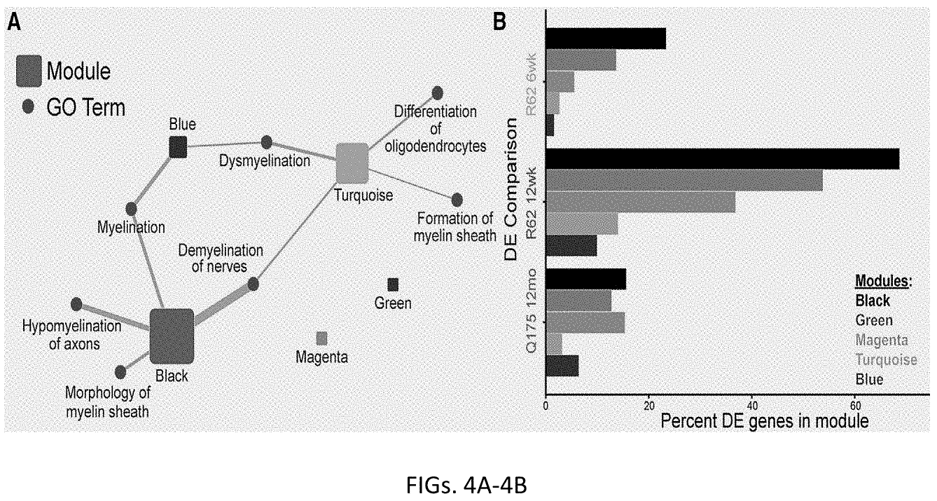

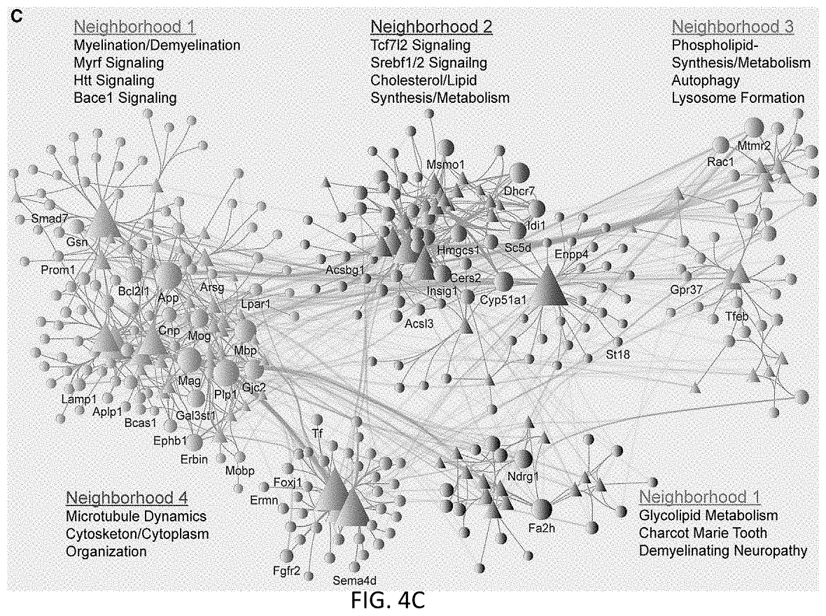

To understand whether the shared patterns of differential gene expression by R6/2 and zQ175 GPCs might identify potential upstream regulators of their shared cellular pathology. Data was analyzed across all conditions using weighted gene correlation network analysis (WGCNA; (Langfelder and Horvath, “WGCNA: an R Package for Weighted Correlation Network Analysis,” BMC Bioinformatics 9: 559 (2008); which is hereby incorporated by reference in its entirety)). This analysis yielded five discrete modules, termed Black, Turquoise, Blue, Magenta, and Green (FIG. 4A). Ingenuity Pathway Analysis (IPA) of the differentially-expressed genes within each module showed a selective predominance of myelin-related terms in the black module (FIG. 4B). As such, applicant proceeded to further investigate the black module through construction of a functional IPA network.

Modularity analysis (Bastian et al., “Gephi: An Open Source Software for Exploring and Manipulating Networks,” Proc. Third Int'l ICWSM Conference 3(1): 361-362 (2009); which is hereby incorporated by reference in its entirety) within the black module produced five neighborhoods of closely-related genes and IPA terms. “Neighborhood 1” harbored several myelination-associated genes and terms, including Cnp, Mog, Mbp, Bcas1, Mobp and Plp1 (FIG. 4C). “Neighborhood 2” included TCF7L2 signaling and associated downstream targets, including cholesterol biosynthetic pathways enriched by the differential expression of Cyp51a1, Hmgcs1, Idi1, and Dhcr7. “Neighborhood 3” segregated terms concerning autophagy and lysosomal pathways, while “neighborhood 4” contained terms addressing morphology and cytoskeletal remodeling. Lastly, “neighborhood 5's” terms consisted of glycolipid metabolism and terms referable to peripheral myelination and demyelination.

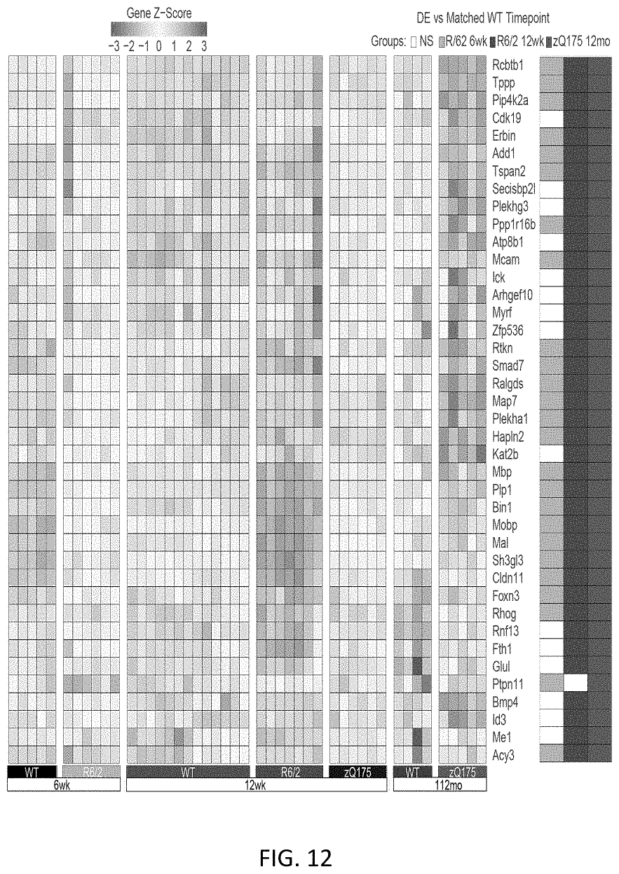

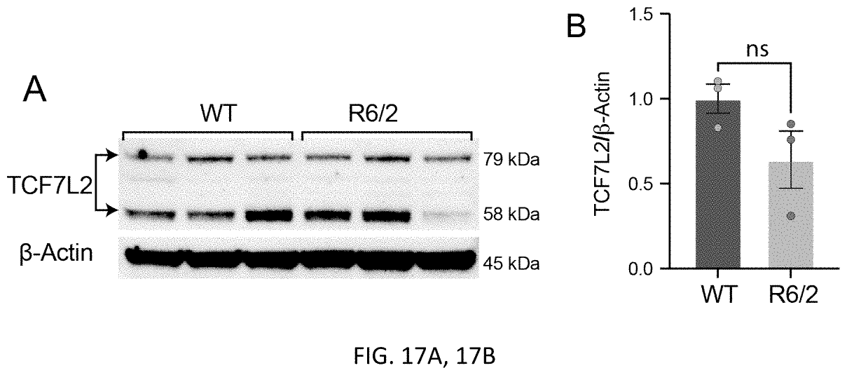

The black module was thus heavily weighted in genes and accompanying terms associated with oligodendrocyte differentiation and subsequent myelination. Among these was the most significantly dysregulated signaling pathway from IPA analysis, TCF7L2 (FIG. 3E), whose activation was predicted to be strongly suppressed in HD models (Activation Z-Scores: 6 week R6/2=−7.99; 12 week R6/2=−11.15; 1 year zQ175=−6.57). This predicted repression was due to dysregulation of 38 TCF7L2 signaling pathway genes (FIG. 12). These included transcripts involved in myelination and oligodendrocyte differentiation, cell cycle progression, and lipid and phospholipid synthesis (FIG. 12). Together, these data suggest a causal link between TCF7L2 signaling and the deficient myelinogensis of HD GPCs, and further suggest that TCF7L2-dependent transcription may be deficient in mHTT-expressing GPCs, and hence comprise a rate limiting determinant of myelin formation and maintenance in HD. That said, neither Tcf712 gene expression, nor protein levels or isoforms per se, appeared dysregulated in models (FIGS. 17A and 17B), suggesting the involvement of additional, yet-to-be-identified partners or interactants with Tcf712 in determining its effective transcriptional tone.

### Example 6—TCF7L2 Overexpression Rescues Key Myelinating Genes In Vivo in R6/2 Mice

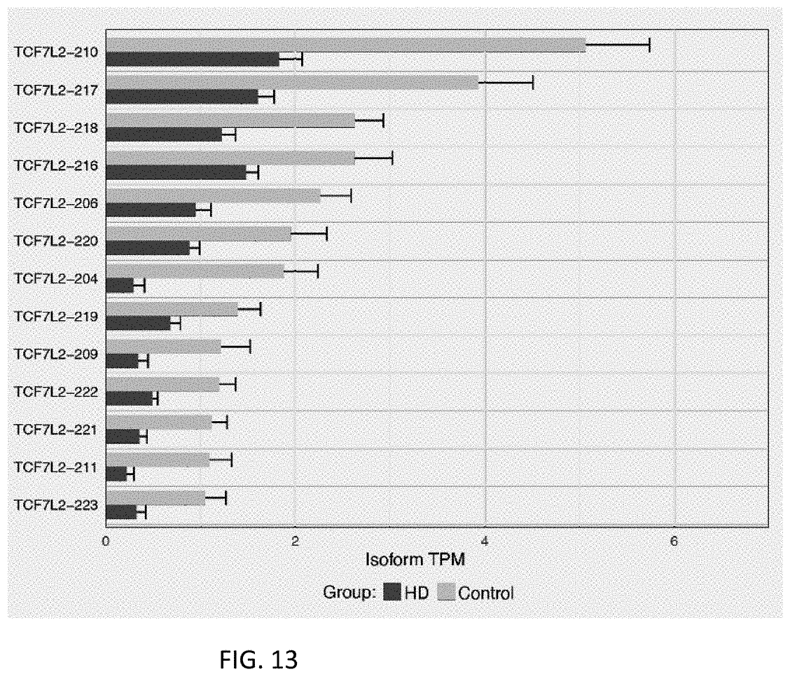

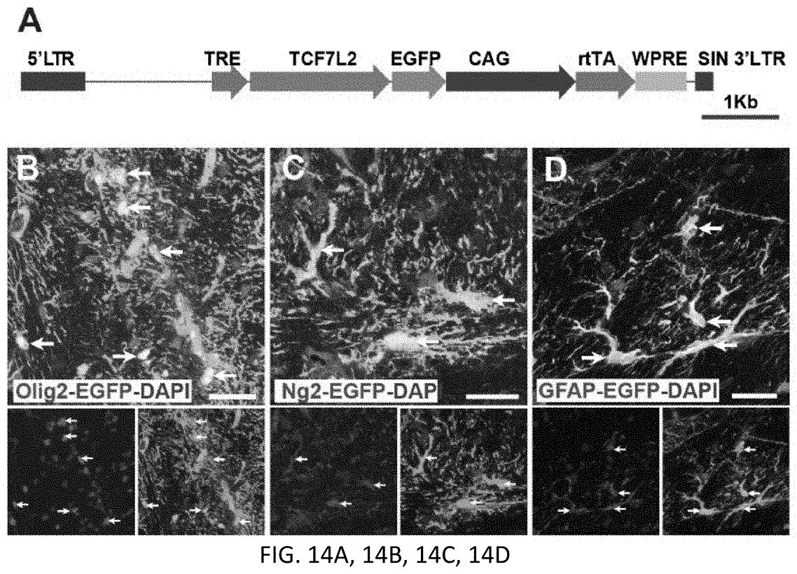

The possibility that the mHTT-dependent decline in TCF7L2 signaling might be causal in the myelination deficiency of HD, was assessed by determining if TFC7L2 overexpression is sufficient to rescue myelinogenic gene expression in R6/2 mouse-derived GPCs. Since TCF7L2 has multiple splice variants that play different roles during development (Helgason et al., “Refining the Impact of TCF7L2 Gene Variants On Type 2 Diabetes And Adaptive Evolution,” Nature Genetics 39: 218-225 (2007); Young et al., “Developmentally Regulated Tcf712 Splice Variants Mediate Transcriptional Repressor Functions During Eye Formation,” Elife 8 (2019), which are hereby incorporated by reference in their entirety). TCF7L2 isoform expression was surveyed in published HD and control hESC-derived GPC RNA-Seq data sets (Osipovitch et al., “Human ESC-Derived Chimeric Mouse Models of Huntington's Disease Reveal Cell-Intrinsic Defects in Glial Progenitor Cell Differentiation,” Cell Stem Cell 24: 107-122 e107 (2019), which is hereby incorporated by reference in its entirety). The TCF7L2-210 isoform was identified as the most prominently enriched GPC isoform (FIG. 13). The human transcript was used, as it is the more therapeutically relevant, and analysis in Blastn revealed its 90.5% homology with the murine transcript. The coding sequence was then inserted into a Tet-On lentiviral system to drive forced expression in vivo (FIG. 14A). The resulting lentiviral TCF7L2 was then injected intrastriatally into 10 week-old R6/2 mice (as 3.8×10⁶ cfu/1 μl). The mice were then split equally into two groups, one treated with doxycycline and the other untreated controls (n=5 mice per group). Two weeks after lentiviral TCF7L2 injection, RT-QPCR was done on dissected striatal tissue to assess treatment-associated changes in the expression of predicted TCF7L2-dependent targets. In particular, expression of myelinogenic genes such as Myrf, Mag, Plp1, Mbp and Trf was assessed. The lipid biosynthetic genes Srebf1, Srebf2, and Hmgcr were monitored, in recognition of TCF7L2's roles in regulating myelinogenesis and lipid metabolism.

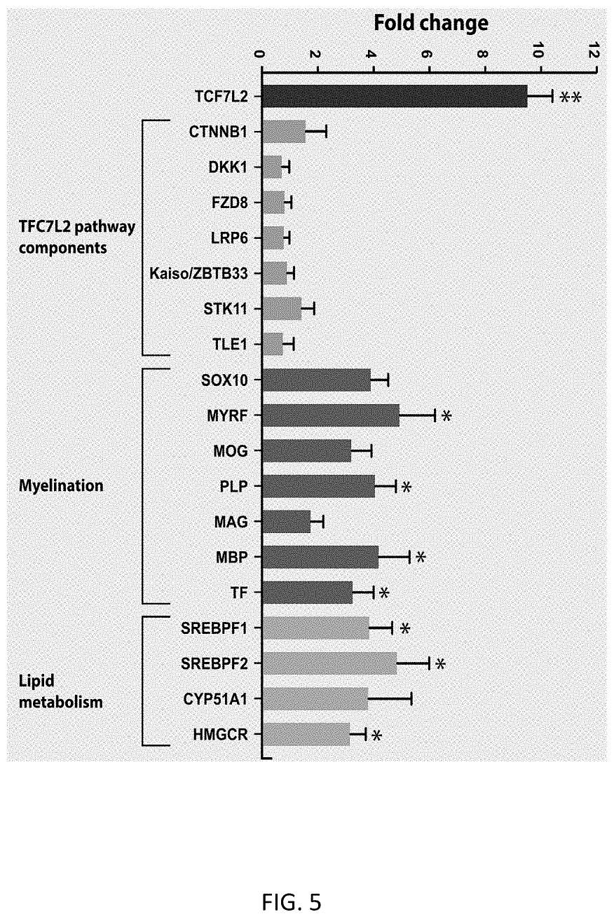

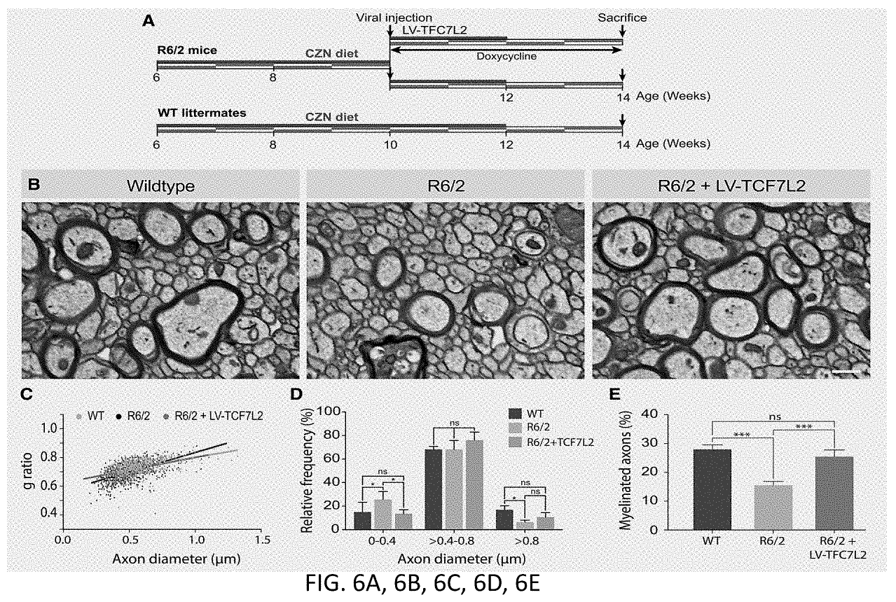

The expression levels of these TCF7L2 targets were significantly increased, whereas other WNT signaling-associated genes that are not targets of TCF7L2 (e.g., Ctnnb1 (Hammond et al., “The Wnt Effector Transcription Factor 7-like 2 Positively Regulates Oligodendrocyte Differentiation In A Manner Independent Of Wnt/Beta-Catenin Signaling,” J Neurosci 35: 5007-5022 (2015); which is hereby incorporated by reference in its entirety), Dkk1, Lrp6 (Su et al., “Effects Of The Extracellular Matrix On Myelin Development And Regeneration In The Central Nervous System,” Tissue Cell 69: 101444 (2021); which is hereby incorporated by reference in its entirety), Fzd8, Kaiso/Zbtb33 (Zhao et al., “Dual Regulatory Switch Through Interactions Of Tcf712/Tcf4 With Stage-Specific Partners Propels Oligodendroglial Maturation,” Nature communications 7: 10883 (2016), which is hereby incorporated by reference in its entirety), Stk11 (Nguyen-Tu et al., “Transcription Factor-7-Like 2 (TCF7L2) Gene Acts Downstream Of The Lkb1/Stk11 Kinase to Control mTOR Signaling, Beta Cell Growth, and Insulin Secretion,” J Biol Chem 293: 14178-14189 (2018); which is hereby incorporated by reference in its entirety), and Tle1 (Dugas et al., “Functional Genomic Analysis of Oligodendrocyte Differentiation,” J Neurosci 26: 10967-10983 (2006); which is hereby incorporated by reference in its entirety) remained unchanged (FIG. 5). These data indicate that the overexpression of TCF7L2 was sufficient to rescue the expression of key myelin biosynthetic and metabolic genes that are otherwise down-regulated in R6/2 HD mice.

### Example 7—TCF7L2 Rescues the Remyelination Defect of R6/2 Mice In Vivo

To determine if lentiviral overexpression of TCF7L2 (LV-TCF7L2) was sufficient to rescue the remyelination defect of R6/2 mice, a cohort of wild-type mice (n=3) were subjected to cuprizone demyelination beginning at 6 weeks of age. At 10 weeks of age, the mice were then given a stereotaxic injection of LV-TCF7L2 (FIG. 14) in the corpus callosum, just above the fornix, the area most rapidly and completely demyelinated by cuprizone (Schmidt et al., “Regional Heterogeneity of Cuprizone-Induced Demyelination: Topographical Aspects of The Midline of the Corpus Callosum,” J Mol Neurosci 49: 80-88 (2013); which is hereby incorporated by reference in its entirety). Immediately after the viral injection, the mice received doxycycline orally, so as to activate the expression of TCF7L2. They were then killed two weeks later, at 12 weeks of age, and their brains cryosectioned and immunolabeled. In these WT control mice, the LV-TCF7L2 transduced callosal cells, which were readily identified by their EGFP reporter, were found to be comprised largely of oligodendroglial lineage cells, expressing OLIG2⁺ and NG2⁺, with some expressing astrocytic GFAP (FIG. 14). This established the ability of the LV-TCF7L2 vector to efficiently transduce and express in normal callosal GPCs and their progeny.

Next, to assess whether forced expression of TCF7L2 would then potentiate oligodendrocytic differentiation and improve remyelination in HD mice, a group of R6/2 mice and their wild-type littermate controls were fed cuprizone beginning at 6 weeks of age (R6/2, n=8; wildtype, n=4) (FIG. 6A). An age-matched group of R6/2 controls was left on a normal diet (n=4). Of note, it has been shown that doxycycline itself does not affect myelination (Osipovitch et al., “Human ESC-Derived Chimeric Mouse Models of Huntington's Disease Reveal Cell-Intrinsic Defects in Glial Progenitor Cell Differentiation,” Cell Stem Cell 24: 107-122 e107 (2019), which is hereby incorporated by reference in its entirety). R6/2 mice then received an intra-callosal injection of LV-TCF7L2-EGFP at 10 weeks. At 12 weeks of age, all mice were returned to a normal diet, and all were killed two weeks later (FIG. 6A).

Electron microscopy image analysis of remyelinated callosal axons, performed as above (FIG. 1 and FIG. 2), showed that myelin sheaths of R6/2 mice exposed to cuprizone and treated with LV-TCF7L2 have significantly lower g-ratios than those of untreated R6/2 mice. Importantly, remyelinated axons of LV-TCF7L2-treated R6/2 were substantially normalized, with g-ratios similar to those of wild-type mice (WT vs R6/2, p<0.0001; WT vs R6/2+LV-TCF7L2, p=0.34; R6/2 vs R6/2+LV-TCF7L2, p=0.0001; all by linear regression) (FIGS. 6B-6C). Analysis of the distribution of remyelinated fibers as a function of diameter showed that the myelinated axons of R6/2 mice treated with LV-TCF7L2 had larger axonal diameters, with a distribution similar to those of wild-type mice (treatment effect: F [4, 27]=6.05; p=0.001 by 2-way ANOVA) (FIG. 6D). In addition, LV-TCF7L2 also restored the relative distribution of myelinated axonal size to that of wild-type mice (F [2.57]=22.07; p<0.0001, one-way ANOVA; FIG. 6E). Taken together, this data indicates that the forced expression of TCF7L2 is sufficient to rescue the myelination defect of R6/2 mice.

### Discussion of Examples

Huntington's disease (HD) is characterized by defective oligodendroglial differentiation and white matter disease. Here, applicant investigated the role of GPC dysfunction in adult myelin maintenance in HD. It was first noted a progressive, age-related loss of myelin in both R6/2 and zQ175 HD mice, compared to wild-type controls. As adults, R6/2 mice then manifested a significant delay in remyelination following cuprizone demyelination. RNA-Sequencing and proteomic analysis of callosal white matter and GPCs isolated from both R6/2 and zQ175 mice revealed a systematic down-regulation of genes associated with oligodendrocyte differentiation and myelinogenesis relative to controls. Gene co-expression and network analysis predicted repressed TCF7L2 signaling as a major driver of this expression pattern. In vivo TCF7L2 overexpression proved sufficient to restore both myelin gene expression and normal remyelination in demyelinated R6/2 mice. These data causally link impaired TCF7L2-dependent transcription to the poor development and homeostatic retention of myelin in HD, and provide a mechanism for its therapeutic restoration.

Huntington's disease (HD) has long been mainly considered a neuronal disease because of its associated prominent loss of striatal and cortical neurons. However, an increasing body of research suggests that glial and white matter pathology are not only present at early stages, but also play a contributory role in the pathogenesis of HD (Poudel et al., “Longitudinal Change in White Matter Microstructure In Huntington's Disease: The IMAGE-HD Study,” Neurobiol Dis 74: 406-412 (2015); McColgan et al., “Brain Regions Showing White Matter Loss in Huntington's Disease Are Enriched for Synaptic and Metabolic Genes,” Biol Psychiatry 83: 456-465 (2018); which are hereby incorporated by reference in their entirety). Applicant has previously shown that HD-derived human glial progenitors and their derived astrocytes exhibit aberrant patterns of gene expression in an mHtt-dependent manner (Osipovitch et al., “Human ESC-Derived Chimeric Mouse Models of Huntington's Disease Reveal Cell-Intrinsic Defects in Glial Progenitor Cell Differentiation,” Cell Stem Cell 24: 107-122 e107 (2019), which is hereby incorporated by reference in its entirety). When transplanted into hypomyelinated shiverer mouse hosts, these cells showed delayed and ultimately deficient myelination, that could be rescued by MYRF-SOX10 expression in vivo. The retention of an oligodendrocytic differentiation defect by these HD hGPCs after transplantation into shiverer suggested the cell-intrinsic nature of their maturational defect. This concept was confirmed in a mouse model of HD, by targeted depletion of mutant Htt in resident GPCs, which rescued their myelinogenic competence, and partially reversed the motor deficit of HD mice (Ferrari Bardile et al., “Intrinsic Mutant HTT-Mediated Defects in Oligodendroglia Cause Myelination Deficits and Behavioral Abnormalities in Huntington Disease,” PNAS 116: 9622-9627 (2019); which is hereby incorporated by reference in its entirety).

However, whether such a myelination defect also occurs in adults and in vivo, and whether it stems from a downregulation of myelinogenic transcription factors is not clear. To this end, applicant investigated myelination in R6/2 HD mice, and showed that the g-ratio, a reliable predictor of functional and structural axonal myelination, was significantly higher (indicating thin and/or deficient myelin sheaths (Hildebrand and Hahn, “Relation Between Myelin Sheath Thickness and Axon Size in Spinal Cord White Matter of Some Vertebrate Species,” J Neurol Sci 38: 421-434 (1978); Chomiak and Hu, “What is the Optimal Value of the g-ratio for Myelinated Fibers in the Rat CNS? A Theoretical Approach,” PloS one 4: e7754 (2009), which are hereby incorporated by reference in their entirety) in 12 weeks old R6/2 mice, relative to healthy WT controls. This suggests that the maintenance of myelin sheath thickness and integrity is a continuous process that is affected by mutant Htt in the adult as the disease progress.

Remyelination was also investigated by subjecting adult mice to a cuprizone-containing diet (Blakemore, “Demyelination of the Superior Cerebellar Peduncle in the Mouse Induced by Cuprizone,” Journal of the Neurological Sciences 20: 63-72 (1973); Stidworthy et al., “Quantifying the Early Stages of Remyelination Following Cuprizone-Induced Demyelination,” Brain Pathol 13: 329-339 (2003); which are hereby incorporated by reference in their entirety). As the mice recovered from a 6-week treatment with dietary cuprizone, the R6/2 mice displayed a significant and progressive delay in their remyelination relative to WT controls, as quantified by both their higher average g-ratios and lower myelinated fiber densities in the corpus callosum. Interestingly, an analogous process of delayed remyelination in response to demyelination was noted in the YAC128 mouse, another full length mutant HTT model of HD (Teo et al., “Impaired Remyelination in a Mouse Model of Huntington Disease,” Mol Neurobiol 56: 6873-6882 (2019); which is hereby incorporated by reference in its entirety).

To better define the mechanistic basis for this observation, the gene expression of GPCs was profiled in two models of HD, the R6/2 and zQ175 mice, which are models of juvenile and adult onset HD, respectively. GPCs from both models were isolated from both presymptomatic and disease-manifest mice, thereby improving the specification and isolation of those pathways responsible for the progressive deficit in remyelination of these animals. Indeed, gene expression analysis showed that HD GPCs displayed profound changes in gene expression in diseased mice—with significantly more dysregulation in R6/2 than in zQ175, likely due to the more severe phenotype of the former. Together, these data in three very distinct models—R6/2, zQ175 and YAC128—all suggested a cell-intrinsic defect in oligodendroglial maturation and myelination that is maintained in vivo.

Pathway analysis then revealed that TCF7L2 signaling was by far the most dysregulated pathway in HD and GPCs across models, and that this pathway was strongly predicted to be repressed. Tcf712 is a member of the TCF/LEF family, a key downstream effector of the Wnt/β-catenin signaling in Wnt-activated (Arce et al., “Diversity of LEF/TCF Action in Development and Disease,” Oncogene 25:7492-7504 (2006); which is hereby incorporated by reference in its entirety). Tcf712 regulates myelinogenic as well as cholesterol biosynthetic genes (Saher et al., “High Cholesterol Level Is Essential For Myelin Membrane Growth,” Nat Neurosci 8: 468-475 (2005); Fancy et al., “Dysregulation of the Wnt Pathway Inhibits Timely Myelination and Remyelination in The Mammalian CNS,” Genes Dev 23: 1571-1585 (2009); Zhao et al., “Dual Regulatory Switch Through Interactions of Tcf712/Tcf4 with Stage-Specific Partners Propels Oligodendroglial Maturation,” Nature Communications 7: 10883 (2016); which are hereby incorporated by reference in their entirety), both of which are disrupted in HD (Valenza et al., “Cholesterol Defect is Marked Across Multiple Rodent Models of Huntington's Disease and is Manifest in Astrocytes,” J Neurosci 30: 10844-10850 (2010); Benraiss et al., “Cell-intrinsic Glial Pathology is Conserved Across Human and Murine Models Of Huntington's Disease,” Cell Reports 36: 109308 (2021); which are hereby incorporated by reference in their entirety), suggesting the importance of TCF7L12-dependent transcription in mHTT-expressing glial and oligodendroglial progenitor cells (Huang et al., “Mutant Huntingtin Downregulates Myelin Regulatory Factor-Mediated Myelin Gene Expression and Affects Mature Oligodendrocytes,” Neuron 85: 1212-1226 (2015); Zhao et al., “Dual Regulatory Switch Through Interactions of Tcf712/Tcf4 with Stage-Specific Partners Propels Oligodendroglial Maturation,” Nature Communications 7: 10883 (2016); Osipovitch et al., “Human ESC-Derived Chimeric Mouse Models of Huntington's Disease Reveal Cell-Intrinsic Defects in Glial Progenitor Cell Differentiation,” Cell Stem Cell 24: 107-122 e107 (2019), which are hereby incorporated by reference in their entirety). TCF7L2 controls oligodendrocytic differentiation and remyelination through multiple mechanisms, and its own expression is tightly regulated during the development of oligodendrocytes (Fu et al., “Tcf712 is Tightly Controlled During Myelin Formation,” Cell Mol Neurobiol 32: 345-352 (2012); Zhao et al., “Dual Regulatory Switch Through Interactions of Tcf712/Tcf4 with Stage-Specific Partners Propels Oligodendroglial Maturation,” Nature communications 7: 10883 (2016); Weng et al., “Transcription Factor 7 like 2 Promotes Oligodendrocyte Differentiation and Remyelination,” Mol Med Rep 16: 1864-1870 (2017); which are hereby incorporated by reference in their entirety). Previous work has demonstrated that TCF7L2 represses the bone morphogenetic protein signaling pathway, which has been shown to inhibit oligodendrocyte differentiation while promoting astrocyte differentiation (Mabie et al., “Bone Morphogenetic Proteins Induce Astroglial Differentiation of Oligodendroglial-Astroglial Progenitor Cells,” J Neurosci 17: 4112-4120 (1997); Sim et al., “Bone Morphogenetic Proteins Induce Astroglial Differentiation of Oligodendroglial-Astroglial Progenitor Cells,” J Neurosci 17: 4112-4120 (2006); Morell et al., “Inducible Expression of Noggin Selectively Expands Neural Progenitors in the Adult SVZ,” Stem Cell Res 14: 79-94 (2015); Zhang et al., “The Wnt Effector TCF712 Promotes Oligodendroglial Differentiation by Repressing Autocrine BMP4-Mediated Signaling,” J Neurosci 41: 1650-1664 (2021); which are hereby incorporated by reference in their entirety). TCF7L2 also acts as an effector of Wnt signaling, a critical pathway for oligodendrogenesis (Fancy et al., “Dysregulation of the Wnt Pathway Inhibits Timely Myelination and Remyelination in The Mammalian CNS,” Genes & Development 23: 1571-1585 (2009b); which is incorporated by reference in its entirety). Importantly, at the onset of oligodendroglial differentiation, TCF7L2 interacts with transcriptional co-repressor Kaiso/Zbtb33 to block β-catenin signaling, consolidating oligodendrocytic fate, and then drives further oligodendrocyte maturation via interaction with Sox10 (Zhao et al., “Dual Regulatory Switch Through Interactions of Tcf712/Tcf4 with Stage-Specific Partners Propels Oligodendroglial Maturation,” Nature Communications 7: 10883 (2016); which is hereby incorporated by reference in its entirety). Yet beyond its cell-autonomous effects on oligodendroglial differentiation, Tcf712 signaling may participate in driving oligodendrocytic fate through paracrine mechanisms as well. As noted, Tcf2's effects may include the relief not only of the differentiation block of mHtt-expressing GPCs, but also a rescue of astrocytic cholesterol synthesis and lipidogenesis, which may then in turn support oligodendrocytic myelinogenesis.

Interestingly, TCF7L2 gene expression per se was not significantly downregulated in either R6/2 or zQ175 mice, so that it seems unlikely that mHtt is acting to suppress its transcription. Rather, the data here suggest that other checkpoints within Wnt-regulated pathways downstream of TCF7L2, critical for the induction of oligoneogenesis and myelin formation, might be pathologically rate-limited in HD, and yet compensated for by TCF7L2 over-expression. As such, additional modulators of Tcf712-dependent transcription may be causally involved in the repression of downstream Tcf712 signaling in HD white matter, such that Tcf712 overexpression remains sufficient to overcome that repression to rescue myelination. Indeed, a number of partners have been identified for Tcf712-dependent transcriptional activation (Zhao et al., “Dual Regulatory Switch Through Interactions of Tcf712/Tcf4 with Stage-Specific Partners Propels Oligodendroglial Maturation,” Nature Communications 7: 10883 (2016); which is hereby incorporated by reference in its entirety), whose relative levels of expression may modulate Tcf712-dependent gene expression, the levels and activities of which have yet to be examined in HD. Regardless though of the identities of these operative transcriptional modulators, the data argue strongly that overexpression of TCF7L2 is both necessary and sufficient for the correct expression of both myelinogenic and lipid biosynthetic genes in HD GPCs, and can rescue the remyelination deficit of cuprizone-treated R6/2 mice.

Together, these data indicate that HD, as manifested in two distinct transgenic mouse models of the disease, is associated with a progressive age-associated loss in forebrain myelin, relative to WT mice, as well as in impaired remyelination after adult demyelination. Together, these findings suggest a loss in homeostatic white matter maintenance. This was underlined by profound dysregulation of oligodendroglial lineage-associated gene expression predicted to be driven by upstream TCF7L2 signaling. Importantly, forced glial over-expression of TCF7L2 proved sufficient to restore the functional transcription of key myelinogenic and lipid biosynthetic genes, and to the in vivo restoration of myelin architecture and abundance. As such, this work may provide a novel and effective strategy for the therapeutic rescue of glial dysfunction, and hence of both the synaptic and white matter pathology of HD.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. A method of treating a subject having a condition mediated by a deficiency in myelin, said method comprising:
introducing to the subject in need thereof a transcription factor 7-like 2 (TCF7L2) and
expressing a transcription factor 7-like 2 protein in one or more cells of the subject or administering to the subject a host cell comprising a genetic construct or expression vector encoding the transcription factor 7-like 2 protein.

2. A method of increasing oligodendrocyte production from glial progenitor cells, said method comprising:
expressing a transcription factor 7-like 2 protein in a population of glial progenitor cells, and
maintaining the population of glial progenitor cells under conditions permitting development and differentiation thereof.

3. The method of claim 1, wherein said expressing is carried out by administering to the subject or the population of glial progenitor cells a genetic construct comprising:
a nucleic acid molecule encoding the transcription factor 7-like 2 protein and
a promoter and/or enhancer for a gene which is selectively or specifically expressed by glial progenitor cells, said nucleic acid molecule being operatively linked to and under the regulatory control of the promoter and/or enhancer.

4. The method of claim 3, wherein the gene selectively or specifically expressed by glial progenitor cells is selected from the group consisting of PDGFRA, ZNF488, GPR17, OLIG2, CSPG4, and SOX10.

5. The method of claim 3, wherein the genetic construct is administered in an expression vector.

6. The method of claim 5, wherein the expression vector is a viral vector, plasmid vector, or bacterial vector.

7. The method of claim 6, wherein the viral vector is selected from the group consisting of a lentiviral vector, an adenoviral vector, an adeno-associated viral vector, and a vaccinia vector.

8. The method of claim 3, wherein the genetic construct is administered in association with a glial progenitor cell-targeted fusogen or a glial progenitor cell-selective surface-binding moiety.

9. The method of claim 8, wherein the genetic construct is in a particle comprising the progenitor cell-targeted fusogen or the glial progenitor cell-selective surface-binding moiety.

10. The method of claim 9, wherein the particle is one selected from the group consisting of a virus, a virus-like particle, and a lipid particle.

11. The method of claim 8, wherein the glial progenitor cell-targeted fusogen or a glial progenitor cell-selective surface-binding moiety is directed against CD140a, NG2/CSPG4, A2B5 gangliosides, 04 sulfatides, or CD133.

12. The method of claim 1, wherein said condition is selected from the group consisting of pediatric leukodystrophies, lysosomal storage diseases, congenital dysmyelination, cerebral palsy, inflammatory demyelination, post-infectious and post-vaccinial leukoencephalitis, radiation- or chemotherapy-induced demyelination, and vascular demyelination.

13. (canceled)

14. The method of claim 1, wherein said subject has a condition with defect in myelination or remyelination.

15. The method of claim 14, wherein said condition is selected from the group consisting of multiple sclerosis, neuromyelitis optica, transverse myelitis, optic neuritis, subcortical stroke, diabetic leukoencephalopathy, hypertensive leukoencephalopathy, age-related white matter disease, white matter dementia, Binswanger's disease, spinal cord injury, radiation- or chemotherapy induced demyelination, post-infectious and post-vaccinial leukoencephalitis, periventricular leukomalacia, and cerebral palsy.

16. The method of claim 1, wherein the condition is a neurodegenerative disease.

17. The method of claim 16, wherein the condition is Huntington's disease.

18. The method of claim 1, wherein the condition is a neuropsychiatric disease.

19. The method of claim 18, wherein the condition is schizophrenia.

20. The method of claim 3, wherein said administering is carried out using intracerebral delivery, intrathecal delivery, intranasal delivery, or via direct infusion into brain ventricles.

21. The method of claim 1, wherein the subject is mammalian.

22. The method of claim 21, wherein the subject is a human.

23. The method of claim 1, wherein the condition is characterized by downregulation of one or more genes selected from the group consisting of Myrf, Bcas1, Plp1, Mbp, and Mobp.

24. A genetic construct or an expression vector comprising the genetic construct, the genetic construct comprising:
a nucleic acid molecule encoding a transcription factor 7-like 2 protein and
a promoter and/or enhancer for a gene selectively or specifically expressed by glial progenitor cells, said nucleic acid molecule being operatively linked to and under the regulatory control of the promoter and/or enhancer.

25. The genetic construct of claim 24, wherein the gene selectively or specifically expressed by glial progenitor cells is selected from the group consisting of PDGFRA, ZNF488, GPR17, OLIG2, CSPG4, and SOX10.

26. (canceled)

27. The expression vector of claim 24, wherein the expression vector is a viral vector, plasmid vector, or bacterial vector.

28. The expression vector for claim 27, wherein the viral vector is selected from the group consisting of a lentiviral vector, an adenoviral vector, an adeno-associated viral vector, and a vaccinia vector.

29. The genetic construct according to claim 24, wherein the genetic construct is in association with a glial progenitor cell-targeted fusogen or a glial progenitor cell-selective surface-binding moiety.

30. The genetic construct of claim 29, wherein the glial progenitor cell-targeted fusogen or a glial progenitor cell-selective surface-binding moiety is directed against CD140a, NG2/CSPG4, A2B5 gangliosides, 04 sulfatides, or CD133.

31. A host cell comprising the genetic construct or expression vector of claim 24, or a progeny of the host cell.


SEQUENCE LISTING (81 sequences)

SEQ ID NO: 1 - DNA, 20 bp, synthetic construct
  source 1..20: mol_type = other DNA
gttcctgacctgcttgagtt

SEQ ID NO: 2 - DNA, 21 bp, synthetic construct
  source 1..21: mol_type = other DNA
gagggtgaaggtgggttattt

SEQ ID NO: 3 - DNA, 22 bp, synthetic construct
  source 1..22: mol_type = other DNA
cgacacctccttatccctctaa

SEQ ID NO: 4 - DNA, 20 bp, synthetic construct
  source 1..20: mol_type = other DNA
atcaaccatcacctgccttc

SEQ ID NO: 5 - DNA, 20 bp, synthetic construct
  source 1..20: mol_type = other DNA
ccgtgtgctactgtgtcatt

SEQ ID NO: 6 - DNA, 19 bp, synthetic construct
  source 1..19: mol_type = other DNA
tcctcagtgcgtccttagt

SEQ ID NO: 7 - DNA, 19 bp, synthetic construct
  source 1..19: mol_type = other DNA
gcaggacaggaaacagtga

SEQ ID NO: 8 - DNA, 22 bp, synthetic construct
  source 1..22: mol_type = other DNA
gagctctaatgtaggcaaggaa

SEQ ID NO: 9 - DNA, 20 bp, synthetic construct
  source 1..20: mol_type = other DNA
gggacaccactcaacaatga

SEQ ID NO: 10 - DNA, 21 bp, synthetic construct
  source 1..21: mol_type = other DNA
ggtacagcaagcaacctctaa

SEQ ID NO: 11 - DNA, 21 bp, synthetic construct
  source 1..21: mol_type = other DNA
cctctttgctgtggacagatt

SEQ ID NO: 12 - DNA, 22 bp, synthetic construct
  source 1..22: mol_type = other DNA
gagctgaggaaagacagttgag

SEQ ID NO: 13 - DNA, 19 bp, synthetic construct
  source 1..19: mol_type = other DNA
gcctgcactttccacaaac

SEQ ID NO: 14 - DNA, 20 bp, synthetic construct
  source 1..20: mol_type = other DNA
gggcatgaagaccaagagaa

SEQ ID NO: 15 - DNA, 22 bp, synthetic construct
  source 1..22: mol_type = other DNA
cttccttctggtcatctccttg

SEQ ID NO: 16 - DNA, 21 bp, synthetic construct
  source 1..21: mol_type = other DNA
gagagtgaaggtgggcaaata

SEQ ID NO: 17 - DNA, 23 bp, synthetic construct
  source 1..23: mol_type = other DNA
ctcactcccttctccttgataac

SEQ ID NO: 18 - DNA, 23 bp, synthetic construct
  source 1..23: mol_type = other DNA
gctccttaggtcacagtcttatt

SEQ ID NO: 19 - DNA, 22 bp, synthetic construct
  source 1..22: mol_type = other DNA
gccacactctgcactaaagata

SEQ ID NO: 20 - DNA, 23 bp, synthetic construct
  source 1..23: mol_type = other DNA
ctgttcccacactctaagttctg

SEQ ID NO: 21 - DNA, 21 bp, synthetic construct
  source 1..21: mol_type = other DNA
ctttcccttgaggacctttgt

SEQ ID NO: 22 - DNA, 23 bp, synthetic construct
  source 1..23: mol_type = other DNA
caatttccagatctgccactaga

SEQ ID NO: 23 - DNA, 20 bp, synthetic construct
  source 1..20: mol_type = other DNA
aggtctagggatgggtgaaa

SEQ ID NO: 24 - DNA, 22 bp, synthetic construct
  source 1..22: mol_type = other DNA
gtgggaaggaacaggacaatta

SEQ ID NO: 25 - DNA, 22 bp, synthetic construct
  source 1..22: mol_type = other DNA
ggaccaggtggtggttaataag

SEQ ID NO: 26 - DNA, 23 bp, synthetic construct
  source 1..23: mol_type = other DNA
catctgaggagaacgcatgatag

SEQ ID NO: 27 - DNA, 20 bp, synthetic construct
  source 1..20: mol_type = other DNA
agcaccttggatgggtattc

SEQ ID NO: 28 - DNA, 20 bp, synthetic construct
  source 1..20: mol_type = other DNA
ctgatgaccggagacaaaca

SEQ ID NO: 29 - DNA, 22 bp, synthetic construct
  source 1..22: mol_type = other DNA
cctcgactcttcctacgtaaac

SEQ ID NO: 30 - DNA, 21 bp, synthetic construct
  source 1..21: mol_type = other DNA
gaatacccaaaccgtgcaatc

SEQ ID NO: 31 - DNA, 20 bp, synthetic construct
  source 1..20: mol_type = other DNA
ctgactgcctccaacgtaaa

SEQ ID NO: 32 - DNA, 21 bp, synthetic construct
  source 1..21: mol_type = other DNA
ctgtacatggtctgcctcatc

SEQ ID NO: 33 - DNA, 22 bp, synthetic construct
  source 1..22: mol_type = other DNA
ggtggctagaccactttatacc

SEQ ID NO: 34 - DNA, 23 bp, synthetic construct
  source 1..23: mol_type = other DNA
catctcctcctatctgccattac

SEQ ID NO: 35 - DNA, 24 bp, synthetic construct
  source 1..24: mol_type = other DNA
gacatcatctacactcaggacttc

SEQ ID NO: 36 - DNA, 20 bp, synthetic construct
  source 1..20: mol_type = other DNA
ctctgtgccgttcatacaca

SEQ ID NO: 37 - DNA, 20 bp, synthetic construct
  source 1..20: mol_type = other DNA
ggcagtctcacttggcaata

SEQ ID NO: 38 - DNA, 19 bp, synthetic construct
  source 1..19: mol_type = other DNA
tgggaccaggagggaatta

SEQ ID NO: 39 - DNA, 20 bp, synthetic construct
  source 1..20: mol_type = other DNA
gcaattattccccatgaacg

SEQ ID NO: 40 - DNA, 20 bp, synthetic construct
  source 1..20: mol_type = other DNA
ggcctcactaaaccatccaa

SEQ ID NO: 41 - DNA, 1389 bp, Homo sapiens
  source 1..1389: mol_type = genomic DNA
cccgcccgcccccagcccgcccgggtgcccgctccgccccccgggctatgtaaagcggccgggctcgggttgtgccaccgctggactcccgtgtccctccgcgcaggcgggcggccccggagcgctggtgccggcagaggcggcgacggtggcgcccctcctcatcatggtgagaggccgggcggggccgggcacggggtagcaccagggcgggaaacgagtgtcggggcccctcgggaggaagcgtcttgcaaacgaggacccggggctccgggttcgacttccagttttcttggtactcagggcggccctgaggtctgggagaagcggaggggttttggggtgccggagaggccgctgtaaagggaaacggtggtgtttctcaggagggaactcgggaccgcagaagctgctgcgcctctgggagcctgggtgggcctgggccgggagcccgggagctgctcccgaccccacgcaggcccgcgctggggcagccgcttcggcgccccctccccgggcccagtctcatcacgcggaaccgctgccccgcgcccgggtgcgggcgggaggtggagccagttcagaggaggggaccctttacggtaaaagctgccgtcccttgtggcttctagaagataggaaaacaagtggctccgagaggcgcttctgacacacagcacagcggcgggtagagctggatcctaattgcaggtgccctgtgctgccctgttctggacaggcacaaagaccacgactttgtgggggccgggagagggtagaggaggaggtggcaggcttggtgaaagagggcctttgtccagtgctctcctgccctgccccccaccagcccttctgtgggaccattgtacccgcatgccagacaagagagtattatctgttgcggccattgttgggggaagggaggcagtcttggggtaacccttctccactcccccccttctctgtgcactcagccgtggcagctgtggcttggcccagacacagagacccccacctccaaagaggacgtccttagtaggtgccaagcatataagagtgaggccagtcccagaaccgcaggctcctggcgcgccccgcatgcctccagcacgtttacctttccgaagtggcaggaatggggaaagcgcacgcttaggagagcttcagacaagcttccctcttcctcctccacgaccagaagcggaaaggtgctcccggaccgaaagggaaagaaggtccagcactgccccgcttgggaaggcacccacaaccagtctagggactaggggtaaggccggcggggagcccgcgaatgacctgggctgacatctcttcccctccttacacagaacagaggcttctcccgaaaaagccacacattcctgccca

SEQ ID NO: 42 - DNA, 2198 bp, Homo sapiens
  source 1..2198: mol_type = genomic DNA
ggatacggaagagatccaatcgcagacccaagatccccacccaggttatggtgggcagaccccagatgccagggccacccattcagcatccctccctggaccccaggacctgctactgctgggtgtctggactccatcctgcacagcactgtgctccatctgccctggggtgtctcatcatcagctgtgtgcagggcaaggggcccaaacaaaggcccagcagtcactggctaagctgccgactggctctctgtgcctccccaagaccctatgtgcccagcagggggcaacagctcagggtcagctgaccgaatgcctcggtgaatgaatgactctacaagagaggaagggagcctcggtgggcatcatctcccctcgactactggccagagccctggctcttacaccccagcgacgggaagcagttgtggcctgtggcttcagtcttcatcaccacaatccctgaagcccacccttgcccagacacctgtgccccagccccaaccccaggccacctcctcagcaggtctggggctgagctgccccacctggcgcctatggcggccagcccatgccccctgcggtgcctctgtcccagactcagcatgtaggccccatgaccccactccacattctggtgactcctcctgagcgtcaggacaacactcaacccacgaggaattatttctgtctcaaagatgcaggaatcagctcaacgcctcaaaactccatcaccacggtcaatgcccttgaagccatcgacagtgatcaccccaataacagaaggtctgtgagcccagaaatgccctgctcagggtggttagcttcaagccaccacctttccaaccagcctgggccagttcttccagacagccgcctgcgggcacaacaggaaagagacctgcgccccggctcagacacctcacacccagctggctctcaggccagacaaactgggaagcccatctctcttgaaggaagtccagatgggaaacagcttctcaacagaccagatcacagcatcagatctaaaggtggccttcagaattctttttcaggttgaattaggatcaaatctaagaattctaaattcaaaatgcagcagaaaaacaaaacacacacacacacggagcctaagttctggagtgacatgtgcttgggttcaaatcctggctctgttgcttcctactgtttgttgatgggtgagtttcttcatttgcctgagcctcagtttccttgtctgtaaaatggggcaataatcccagctgcacagggtgatgtgaagagacaaatttaagacactgccccttaaatgctagccacatacatacagttttcaatgtttaaacaacaaaatgtaaagtcttttgaaaccaggaagggtgatttggtttcccatgttgctggatgtatcattttcagaaagacagagagaaatgaactttgttcactcagtctcagaggcggccgccggcagcattcaaaggcaccccagcccggagccaccccagggaggagccccaggccagcggtcagattcatgggcttccgtgcagaaggggagctgcaccggcgagcacccggcctctgagctgagccgcatcctcacggacaggacagcgccccattatgaggctcctgcagctgttcctcgctccagataaaggccatgatttattctgtgtgcccaaatggggcctcattatacagggcaggacacaaggaccctacagcaagtgtcctcaaagagtcgcctctcactccgtgagcaagactcctcggcctcccaccctccgttcacaggccccctccgccgtctgcgggcgcaggcctgggagcgccgcctgttgccatgacagccggcccctccctgccccccatcagtaggaaatcatccccttctgaaacgtcctgttgtgtccctcagctccagcccaagccccccacccagcccccgcctgctctgagtctctgagacagtcacacactcagactatgtggccaagctgggggcggggggcatgggctagggacacactagaatattcacgctccggtggcagcagcagcagcagccagaggagcagcccgacacaacaagggacccctcaggaatgaagcagcctttcagggccagaggggctgtggtctcccttcctctccttaaatagccagcgttccacccacagcggcaaggg

SEQ ID NO: 43 - DNA, 2119 bp, Homo sapiens
  source 1..2119: mol_type = genomic DNA
gttgacctctcttctttgcacagtctctccgcaaaattttcccccaaccatttgctttcttgcgccaccccccaccggggcactatccctggagtcagctcctcaggcctttaaaccttccagaatgtcacacatggaaacctttagcaaatgtttgttaatgatcataacaaaggcatcattcaaattaggcaggtaattactaccagaaggacaactgggtgctcgcttgctcatccattgctttctgctttaaactcccgaggagtcgctgcctggagacactcccgcccggactgcttctcgggccccattaaacggctctgaaatgaactgtgaagttaccatttgtgggcggagagcgctggggccctgcggacggaagaccctggcttgaccggactaacagtgtcgccgaagcaaagcagcccctctgggtaggctgtctgtctcgcctcctaccctctggggcaactagcctaaaaacccggttctcaacttaacagcatgtggcccagcacccgcttttgccttttattccgcactctgatttttggggggatcgtttgttcccgctcattttctacaaacattacggtatcctgttagcattccgaacaaggggctgttcatatatttgccttcaatgatttcctgaagggacatgtggaagtaatagcgagcggtgcagtcacccaagctgaagatgcacgagagcgggtcgcgtagaagaactgcggcaatgggacccccagcgcagccccaggcgccctgttttcgaggctgacatcccaagatcctgtcctggttaggcacagcaccgcgcccgttggagtacttctctgtgactctcccctgtccactgcactgtgtccaccctcctggtgaaggaggagacggctgttggacatgccagtcccaacctggccactttggccattttcagagtgcgtgtgttggggtggggttgagggagaaggatgaaggatgacccccaaccccaacaggagaattcaaaaatcagagagcgatgaaggtgaacgcgctcccctcccccgctgtcgctgggaagccccactctgcccaggtctggttcctggagcatcagcgccccctttccctcgccggcaggatcgtttcctcccgacactggcctctcgccttgggcgcagggtttggggctcagctgtcaattccaaggaattcgtagggttcgacccacgcggcgggagggaacggagggagaatcaaaaggcatcctcccccacgacgcggaggcggtggtacgattcctcaaacgcccaagcttgagctttttttgtatgcgagatagaagccagggcaacctcgcccagataaccccgaaaacaaaggcacgaccagataagtgacttcaagggagtaaaggtcgttctatcggagaacgttccaaacctacacagaccctcttttctttgtaccgtatttcagaccacccagtcttgtacacacacacacaccacacaaaacaaaaacccatcattgtcatattggactcaacagtttgcccaatcctattaaataactagcaactatatagcccttcccctaaaagacccctagttcaaaacggcgcaaccgcttggagatttctcccgagggccctatttctcgttgggccgagtttgtagaagggacatttcttgattctagatttatattctctctgggtattaaatgcaattttgtatgttccttttcttcttttcaaagagaatacaaaactatagcagtctaagaaaataaccccaaaatggggaggcaaactcattctggaaatgatgggctgtttgtagtttcacaaaacctctttcccggcagagcaccaacacctcccccttccaccaccccccatcccatctggtctgcttctccccgccccccagttgttgtcgaagtctgggggttgggactggaccccctgattgcgtaagagcaaaaagcgaaggcgcaatctggacactgggagattcggagcgcagggagtttgagagaaacttttattttgaagagaccaaggttgagggggggcttatttcctgacagctatttacttagagcaaatgattagttttagaag

SEQ ID NO: 44 - DNA, 1516 bp, Mus musculus
  source 1..1516: mol_type = genomic DNA
cggaggggaaagtcaaaggttcacagaagaccctccatcaaggggaggatgtgcagtagttgtatacaaaccgtgaagtactgggatgtgcagaatgagagaggaagaagaaaaggcgatatgagttgaacacctactgtatgccaggccctaaggtaggcatttgagacatattaaaacacttaatgcttataaattcaaacttgtaagtgaaaaactgaaccacaggaaaaaaaggagtagttaaaggccagtcaagtaggagaattgggatcaagaacacatcagaccaggcatggtagtaagagcacagagcatgggctcaatgattcggcttgccctcgcctggatcaactcacccaggccctttaatccctctcagcctcagtttctctatcgaaatggggctcggttctatcagaagtcatgagacgctggacttgtgctatgattgttgctgctcaggggtaaggattggaaagagggtgcacagtggaacgtgggcacagcagggagcccagtggagggatatgcgtgaaagaagagaggaacgggagtgttcctctatgcgtctcctagaaaaaacccaggccaggtcccaatgcagctggggctggccaaacacaggcacggggaaaagccacagtaactctgctttttccagctcctggttgggactaggcaggctcttgcctgggaaggagccaggaggagaaggccccagcctgaagctgttaggtaggagctaaatccagggccagatttccaggaggtggtggggcaagtgaaaacaccaagatttgggggtcaggaggcctgggatcctggcaggaaatggccaggcactacctttgaccttggattctgagctgggctcctcctccacaaaatgaagggaagggtgctcagcccctaccttgcagggttgttttgaagcatgtgttaccacagggctgagcttctgcaccacaagatccagacctcttgtctttacaggcagagaatgtacagtctgtggaaagagaagggcaaacccagagccctgcctagcggtctgctttagtggaatcctcggggacctgtgaccttgaaggcacctaaacccctacatcaccgcaagggcagaagagggctggcagctctcacttttatttcccgttctcaagagatcctgtccacagagttctgggggtctaaagacttaagagacattgtcccaaacccttaaaatggggcatgggtctgccttggcaggaaaggtccttcaatcctaaccaggagcaagagcccagcacggcccctttaagaaagttggaggtgggagcagacagagggagcccccgcccaaaatctaagcgcgggtctggcgcctgccccgcccccggagttaagtactggacagtccttgcgccctagcccgtgcgaggagccctgctccctcgactttggacttgaattgcgacttgcgacttgcgactcggtgctgtccagattcagcacgctctgttccttcgcc

SEQ ID NO: 45 - DNA, 4025 bp, Homo sapiens
  source 1..4025: mol_type = other DNA
gtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttttgggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaaccctccattttcagtccggcagcacacattactctgcgtacaaaacgattgaacaccagattgcagttcagtatctccagatgaaatggccactgcttgatgtccaggcagggagcctccagagtagacaagccctcaaggatgcccggtccccatcaccggcacacattgtctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatgatgcaaatactccaaagaagtgtcgggcactgttcgggcttgaccgacagactttatggtgcaaaccgtgcaggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 46 - protein, 602 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTLHFQSGSTHYSAYKTIEHQIAVQYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNDANTPKKCRALFGLDRQTLWCKPCRRKKKCVRYIQGEGSCLSPPSSDGSLLDSPPPSPNLLGSPPRDAKSQTEQTQPLSLSLKPDPLAHLSMMPPPPALLLAEATHKASALCPNGALDLPPAALQPAAPSSSIAQPSTSSLHSHSSLAGTQPQPLSLVTKSLE

SEQ ID NO: 47 - DNA, 4007 bp, Homo sapiens
  source 1..4007: mol_type = other DNA
gtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttttgggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaacctatctccagatgaaatggccactgcttgatgtccaggcagggagcctccagagtagacaagccctcaaggatgcccggtccccatcaccggcacacattgtctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatgaacacagcgaatgtttcctaaatccttgcctttcacttcctccgattacagacctgagcgctcctaagaaatgccgagcgcgctttggccttgatcaacagaataactggtgcggcccttgcaggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 48 - protein, 596 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNEHSECFLNPCLSLPPITDLSAPKKCRARFGLDQQNNWCGPCRRKKKCVRYIQGEGSCLSPPSSDGSLLDSPPPSPNLLGSPPRDAKSQTEQTQPLSLSLKPDPLAHLSMMPPPPALLLAEATHKASALCPNGALDLPPAALQPAAPSSSIAQPSTSSLHSHSSLAGTQPQPLSLVTKSLE

SEQ ID NO: 49 - DNA, 4024 bp, Homo sapiens
  source 1..4024: mol_type = other DNA
gtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttttgggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaacctatctccagatgaaatggccactgcttgatgtccaggcagggagcctccagagtagacaagccctcaaggatgcccggtccccatcaccggcacacattgtcagccccctcccttgctgcactcagggacatgactgtcagcacttctaccccccctcagacttcactgtcagcactcaagtcttcagggacatgaaaaggagccactccttacaaaaagttggggagccctggtgtattgagtctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 50 - protein, 489 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTYLQMKWPLLDVQAGSLQSRQALKDARSPSPAHIVSPLPCCTQGHDCQHFYPPSDFTVSTQVFRDMKRSHSLQKVGEPWCIESNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNGEKKSAFATYKVKAAASAHPLQMEAY

SEQ ID NO: 51 - DNA, 3922 bp, Homo sapiens
  source 1..3922: mol_type = other DNA
gtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttttgggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaacctatctccagatgaaatggccactgcttgatgtccaggcagggagcctccagagtagacaagccctcaaggatgcccggtccccatcaccggcacacattgtctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatgaacacagcgaatgtttcctaaatccttgcctttcacttcctccgattacaggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 52 - protein, 455 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNEHSECFLNPCLSLPPITGEKKSAFATYKVKAAASAHPLQMEAY

SEQ ID NO: 53 - DNA, 3956 bp, Homo sapiens
  source 1..3956: mol_type = other DNA
gtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttttgggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaacctatctccagatgaaatggccactgcttgatgtccaggcagggagcctccagagtagacaagccctcaaggatgcccggtccccatcaccggcacacattgtctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatgacctgagcgctcctaagaaatgccgagcgcgctttggccttgatcaacagaataactggtgcggcccttgcaggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 54 - protein, 579 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNDLSAPKKCRARFGLDQQNNWCGPCRRKKKCVRYIQGEGSCLSPPSSDGSLLDSPPPSPNLLGSPPRDAKSQTEQTQPLSLSLKPDPLAHLSMMPPPPALLLAEATHKASALCPNGALDLPPAALQPAAPSSSIAQPSTSSLHSHSSLAGTQPQPLSLVTKSLE

SEQ ID NO: 55 - DNA, 3883 bp, Homo sapiens
  source 1..3883: mol_type = other DNA
gtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttttgggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaacctatctccagatgaaatggccactgcttgatgtccaggcagggagcctccagagtagacaagccctcaaggatgcccggtccccatcaccggcacacattgtctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 56 - protein, 442 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNGEKKSAFATYKVKAAASAHPLQMEAY

SEQ ID NO: 57 - DNA, 4095 bp, Homo sapiens
  source 1..4095: mol_type = other DNA
gtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttttgggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaacctatctccagatgaaatggccactgcttgatgtccaggcagggagcctccagagtagacaagccctcaaggatgcccggtccccatcaccggcacacattgtctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccagctttctgtcttctaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatgaacacagcgaatgtttcctaaatccttgcctttcacttcctccgattacagacctgagcgctcctaagaaatgccgagcgcgctttggccttgatcaacagaataactggtgcggcccttgcagatgcaaatactccaaagaagtgtcgggcactgttcgggcttgaccgacagactttatggtgcaaaccgtgcaggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 58 - protein, 476 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSSFLSSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNEHSECFLNPCLSLPPITDLSAPKKCRARFGLDQQNNWCGPCRCKYSKEVSGTVRA

SEQ ID NO: 59 - DNA, 3956 bp, Homo sapiens
  source 1..3956: mol_type = other DNA
gtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttttgggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaacctatctccagatgaaatggccactgcttgatgtccaggcagggagcctccagagtagacaagccctcaaggatgcccggtccccatcaccggcacacattgtctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatgatgcaaatactccaaagaagtgtcgggcactgttcgggcttgaccgacagactttatggtgcaaaccgtgcaggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 60 - protein, 579 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNDANTPKKCRALFGLDRQTLWCKPCRRKKKCVRYIQGEGSCLSPPSSDGSLLDSPPPSPNLLGSPPRDAKSQTEQTQPLSLSLKPDPLAHLSMMPPPPALLLAEATHKASALCPNGALDLPPAALQPAAPSSSIAQPSTSSLHSHSSLAGTQPQPLSLVTKSLE

SEQ ID NO: 61 - DNA, 4020 bp, Homo sapiens
  source 1..4020: mol_type = other DNA
gtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttttgggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaacctatctccagatgaaatggccactgcttgatgtccaggcagggagcctccagagtagacaagccctcaaggatgcccggtccccatcaccggcacacattgtctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatgaacacagcgaatgtttcctaaatccttgcctttcacttcctccgattacagacctgagcgctcctaagaaatgccgagcgcgctttggccttgatcaacagaataactggtgcggcccttgcagtctttgaatttggaatattacaatggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 62 - protein, 455 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNEHSECFLNPCLSLPPITDLSAPKKCRARFGLDQQNNWCGPCSL

SEQ ID NO: 63 - DNA, 4032 bp, Homo sapiens
  source 1..4032: mol_type = other DNA
gtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttttgggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaacctatctccagatgaaatggccactgcttgatgtccaggcagggagcctccagagtagacaagccctcaaggatgcccggtccccatcaccggcacacattgtctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatgaacacagcgaatgtttcctaaatccttgcctttcacttcctccgattacagacctgagcgctcctaagaaatgccgagcgcgctttggccttgatcaacagaataactggtgcggcccttgcagtctttgaatttggaatattacaatggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 64 - protein, 450 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNEHSECFLNPCLSLPPITDLSAPKKCRARFGLDQQ

SEQ ID NO: 65 - DNA, 3934 bp, Homo sapiens
  source 1..3934: mol_type = other DNA
gtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttttgggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaacctatctccagatgaaatggccactgcttgatgtccaggcagggagcctccagagtagacaagccctcaaggatgcccggtccccatcaccggcacacattgtctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatgaacacagcgaatgtttcctaaatccttgcctttcacttcctccgattacaggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 66 - protein, 459 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNEHSECFLNPCLSLPPITGEKKSAFATYKVKAAASAHPLQMEAY

SEQ ID NO: 67 - DNA, 3781 bp, Homo sapiens
  source 1..3781: mol_type = other DNA
gtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttttgggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaacctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 68 - protein, 407 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNGEKKSAFATYKVKAAASAHPLQMEA

SEQ ID NO: 69 - DNA, 3952 bp, Homo sapiens
  source 1..3952: mol_type = other DNA
gtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttttgggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaaccctccattttcagtccggcagcacacattactctgcgtacaaaacgattgaacaccagattgcagttcagtatctccagatgaaatggccactgcttgatgtccaggcagggagcctccagagtagacaagccctcaaggatgcccggtccccatcaccggcacacattgtctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 70 - protein, 465 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTLHFQSGSTHYSAYKTIEHQIAVQYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNGEKKSAFATYKVKAAASAHPLQMEAY

SEQ ID NO: 71 - DNA, 3756 bp, Homo sapiens
  source 1..3756: mol_type = other DNA
agataaagtgcctgcctcctgcactcggttgatcattttccctcatggctgaccgtaatttctttgctactcttagcggcagctctgtccctgctgcccaaggtaacgggtgagggaggggactttgctgaggtgccctgagctggctggggaacatggaccttcccccttccttcacggattctgtagtggcaaagattagaaagtagaaggagtttttaaaattttttttaaaaaattaaatctgcaaccagctgaactctagattattagtggacttttttgttttttatttttaagatttttttagggatgtgtgtacagggggaaggattaacaagataattctaggcaatatgagcgacaactttgagcattttgagcctactcggccaggaatctggagccatttcgatctttttagatgctttttttcagttctttaagtgaaaggtttaaagggaggggaagctgggaggaaaaagaaaagtgagaaaagcagaaagggaggaggaagaaaaactggcccagtcccatctcgaaggtacagagaaagtttttttagaggtggagagagggaaaaccaaggctgttatgtaccctagggacacaattttagtgggaatggaagatttgagtggtaagggaggcgcagtggcctccggggtctctctgttccagcagttgggcggttggcacctcaatttgaattgctggtctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatgaacacagcgaatgtttcctaaatccttgcctttcacttcctccgattacagacctgagcgctcctaagaaatgccgagcgcgctttggccttgatcaacagaataactggtgcggcccttgcagtctttgaatttggaatattacaatggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 72 - protein, 338 aa, Homo sapiens
MYPRDTILVGMEDLSGKGGAVASGVSLFQQLGGWHLNLNCWSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNEHSECFLNPCLSLPPITDLSAPKKCRARFGLDQQNNWCGPCS

SEQ ID NO: 73 - DNA, 3288 bp, Homo sapiens
  source 1..3288: mol_type = other DNA
attgtgctcttccctatcaagcgagataattctgtgaatggaactgtgcgtgagcatcttgtctggcggcgctgctggtgtgtgctgctcccctgtgccgcgggtgcgcggcggcggcgcgggctgcagggcgggtgctgccctccagtggagcccgcggccggcgcgggccctgggcagcatctgggcgcaggcagcatgcccgcgagccggcagcgggaaggacagcgccgcaaccctctctagatgtctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatgaacacagcgaatgtttcctaaatccttgcctttcacttcctccgattacagacctgagcgctcctaagaaatgccgagcgcgctttggccttgatcaacagaataactggtgcggcccttgcagtctttgaatttggaatattacaatggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 74 - protein, 299 aa, Homo sapiens
MSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNEHSECFLNPCLSLPPITDLSAPKKCRARFGLDQQNNWCGPCSL

SEQ ID NO: 75 - DNA, 4003 bp, Homo sapiens
  source 1..4003: mol_type = other DNA
gtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttttgggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaaccctccattttcagtccggcagcacacattactctgcgtacaaaacgattgaacaccagattgcagttcagtatctccagatgaaatggccactgcttgatgtccaggcagggagcctccagagtagacaagccctcaaggatgcccggtccccatcaccggcacacattgtctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatgaacacagcgaatgtttcctaaatccttgcctttcacttcctccgattacaggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 76 - protein, 482 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTLHFQSGSTHYSAYKTIEHQIAVQYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNEHSECFLNPCLSLPPITGEKKSAFATYKVKAAASAHPLQMEAY

SEQ ID NO: 77 - DNA, 4076 bp, Homo sapiens
  source 1..4076: mol_type = other DNA
gtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttttgggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaaccctccattttcagtccggcagcacacattactctgcgtacaaaacgattgaacaccagattgcagttcagtatctccagatgaaatggccactgcttgatgtccaggcagggagcctccagagtagacaagccctcaaggatgcccggtccccatcaccggcacacattgtctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatgaacacagcgaatgtttcctaaatccttgcctttcacttcctccgattacagacctgagcgctcctaagaaatgccgagcgcgctttggccttgatcaacagaataactggtgcggcccttgcaggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttccttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattctaccaaaatttttatcaacagctgtttaaagtctttgtagcgtttaaaaaatatatatatatacataactgttatgtagttcggatagcttagttttaaaagactgattaaaaaacaaaaagaaaaaaaaagcaattttgaagcagccctccagaaggagttggttctgtattatttgtattaaatacgagcttgcgaaccaatcattttacatctggtttttaaaccgtaagggcaccatgaatgcagtgccgttactttttttttttttttctgtgtgaaacaactcttattgtgatgttacttgttattgtttaaatgtacagaaacaaagggtaaaaatgtgttaatataccttgttccatggtgttgttcttttggggggaggggacgctactcaacacttaatagaatcacaacgctgttgggccagtagtatttattgctttagagattgcttgtcgtacctgtatgtcgtccctttttaaatatgttttcctttttcttgaaactgtataaagtttttttcccccttagcataagcatcttatatataacaactcatttgtacaaggtttttaagtttatatataaaatgtgtatatatatttttgtttcccctttttgacttttttttttctgtatgaaacccagatgtcaccaaatggacattaatagttgcattaaggatcagtagcattaacaaaagttgctttaaaagccattatgtaaaacaagacttgaaaatgagtgagggaattttagcgacactgtctgagcagcagtgggaaccatcttcgtttcccctttgaactcccagtgggatgccctaccctgcgcccttaggacccggactgaccgtgtacaaaactttacgtgccaaaattctcagtgaatttagctttctccctctttttgatgctgtaatttttgttcatcatgttttgctgtgatgttacataggtagatttgtatgtagttttaatgtcacctataacaaaatgtgtttggtagcagattgtccagaaagcattttaaatgaagaggtataaacccttaagggccaaaattctgtatattagattactcttaaacgaaaaaccagctgccgcttttatgtacacatattacatacgagtaggcagcagactttaaaaataaaaaaaacctaggcatgttgatgttgcaaaatgctgtataaagctgaaacctgttcattcagtgccattgtagttgacatgaagcgattgtaaaactgtctccgatttttctctggtttattaaaatgctaactataacattttttgtgaatactttgaatgtttcctaacagttgtgatgttactgttccgttttatgctcttattccaagttcatttttaatggtttggaagccatttttgtaatgaataaatgttcatgctgtacagtatctgtagcatgccgttctggattaataaaagcaacttagtatgtgcagataaa

SEQ ID NO: 78 - protein, 619 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTLHFQSGSTHYSAYKTIEHQIAVQYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNEHSECFLNPCLSLPPITDLSAPKKCRARFGLDQQNNWCGPCRRKKKCVRYIQGEGSCLSPPSSDGSLLDSPPPSPNLLGSPPRDAKSQTEQTQPLSLSLKPDPLAHLSMMPPPPALLLAEATHKASALCPNGALDLPPAALQPAAPSSSIAQPSTSSLHSHSSLAGTQPQPLSLVTKSLE

SEQ ID NO: 79 - protein, 596 aa, Homo sapiens
MPQLNGGGGDDLGANDELISFKDEGEREEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNEHSECFLNPCLSLPPITDLSAPKKCRARFGLDQQNNWCGPCRRKKKCVRYIQGEGSCLSPPSSDGSLLDSPPPSPNLLGSPPRDAKSQTEQTQPLSLSLKPDPLAHLSMMPPPPALLLAEATHKASALCPNGALDLPPAALQPAAPSSSIAQPSTSSLHSHSSLAGTQPQPLSLVTKSLE

SEQ ID NO: 80 - protein, 442 aa, Mus musculus; identical to SEQ ID NO: 56

SEQ ID NO: 81 - DNA, 4127 bp, Mus musculus
  source 1..4127: mol_type = other DNA
ctcgccctgtcaataatctccgctcccagactactccgttcctccggatttcgatccccctttttctatctgtcaatcagcgccgcctttgaactgaaaagctcagtctaacttcaactcactcaaatccgagcggcacgagatccttcggtgtcttcggcttcccccccctttgctcttatatctgacttcttcttgttgttgttgttgttttttttacccccttttttatttattattattttttttttttgcacattgatcggatccttgggaacgagagaaaaaaagaaacccaaactcacgcgtgcagaagatctccccccttcccctcccctcctccctcttttcccctccccaggagagaaagaccccaaagcaggaaaaaaaaaagttaaccttggactcgtctttttcttgcagtatttttttgggggggactcgcaaaactttttttttttttttttttttttttttgctttttttcctccttcatttttcttccaaaattgctgctgctggtgggtgaaaaatgccgcagctgaacggcggtggaggagatgacctaggcgctaacgacgagctgatctccttcaaagacgaaggcgagcaggaggagaagaactcggaaaactcctcggcggaaagggatttagccgatgtcaagtcctcgctggtcaatgaatcagagacgaatcaaaacagctcctccgattccgaggcggaaagacggcctccgcctcgctccgaaagtttccgagataaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaacctatcttcagatgaaatggccactgcttgatgtccaagcaggaagcctccagagcagacaaaccctcaaggatgctcgttcgccgtcgccagcacacatcgtttcgaacaaagtaccggtggtgcaacacccccaccatgtccacccactcacgcctctcatcacgtacagcaatgaacacttcaccccgggaaatccacctccgcacttaccagctgacgtagaccccaaaacaggaatcccaaggcctccgcaccctccagatatctctccatattacccgctgtcgcccggcaccgtaggacaaatcccccatccgctaggatggttagtaccacagcaaggtcagcctgtgtacccaatcacgacaggaggattcagacacccctaccccacagcgctgacagtcaacgcatctatgtctaggttccctccccatatggtccctccccatcacactctgcacacgaccggcatccctcacccggccatcgtcacaccgacagtcaagcaggaatcctcccagagtgacgtcggctcactccacagctcaaagcatcaggactccaaaaaggaagaagagaagaagaagccccacataaagaagccccttaatgcattcatgttgtatatgaaagagatgagagcgaaggtggtggccgaatgcacattgaaagagagtgcagccatcaaccagattctcgggcgcaggtggcacgccctgtccagggaagaacaggcaaaatactacgagctggcccggaaggaacgacagcttcacatgcagctgtaccctggctggtctgcacgggataactatgggaagaagaagaagagaaaaagagacaagcagccgggggaaaccaacgaacacagcgaatgtttcctaaatccttgcctttcgcttcctccgatcacagacctgagcgctcctaagaaatgccgagcgcgctttggccttgatcaacagaataactggtgcggcccctgcaggagaaaaaaaaagtgcgttcgctacatacaaggtgaaggcagctgcctcagcccaccctcttcagatggaagcttactagactcgcctcccccctcaccgcatctgctaggctcccctccccaagacgccaagtcacagactgagcagacccagccgctctcgctgtccctgaagcctgatcctctggcccacctgtccatgatgcctccgccacccgcgctcctgttggccgaagctgcccacggcaaggcctctgccctctgtcccaatggggctctggacctgccacctgccgctctgcagccgtccatggtcccttcctcatcgctcgcacaaccatcaacttcttccttacattcccacaactcgctggctggaacgcaaccccagcctctgtctctggtgaccaagtctttagaatagctccgcctcctcgtcctccatgcccctgcttgattgaagtgtttccttctggttcttggttcgcccttccccgcttggaaagcttttgttttgtactctttaattttgtgccgccgtggctacatgagttaatgtttatggagttcattggtcaatatttgacccattcttatttcaatttctccttttaaatatgtagatgagaaaaacctcatgattctaccaaaatttttatcaacagctgtttaaagtctttgtagcgtttaaaaaatatattatatatacataactgttatgtagttcggatagcttagttttaaaagactgatttaaaaaacaaaaagaaaaaaaagagagagaagcaattttgaagcaccctccagaaggagttggttctgtgttatttgtattaaatacgagcttgcgaaccaatcactttacaccttggtatttaaaccacgagggcacaaggaatgcagtgccgtttctttctttctttttttctttcttttttttttcctcccagtgtgaaacaacttttaccttgatgttacttgttattgtttaaatgtacagaaacaaagggttaaaatgtgttaatataccttgttccatggtgttgttcttttgggggggaggggacaccactcaacaatgaatggaatcaacactgttggaccagtagtatttattgctttagaggttgcttgctgtacctgtatgtccgtccaattttaaatatgttttcccttttttttgaaactgtgtaaagtcccccccaaccttagcgtaagcatcttatatataacaactcatttgtacaaggtttttaagtttatatataaaatgtgtatatatttttttgtttccttttttttttttttttggcgtacgttcgttcgttcgtttgtttgtttctgtataaaacccagatgccaccaaatggacattgacattcgcattaaggatcagtagcattaacaaaacttgctttaaaagccattctgtaaaacaagacttgaacattagcgagagggtctgagcagccgtctgagcagccgtgggaactgccccgtttcccccttgacctcctagtcaactgccctgtgctcttagaacatggactgaccgtgtgcaaaacttttatgtgccaaaattctcagtgactttagctttctccctcctttttgatgctgtactttctgttcgccatgttttgctgtgatgttacatagatagatttgtatgtagttttaatgtcacctataacaaaatgtgtttggtagcagactgtccagaaagcattttaaatgaagaggtctaaacccttaagggccaaaaaaaatcctgtatattagattactcttaaatgaaaaagaaaaaaagaaaaaaaaaaaccccagctgccgcttttatgtatgcatattacatacaagtaggtagtgacctttaaaaacaagaaaacctaggcatgccgatgttgtaaaatgctgtataaagctgagacctgttccttcagtgccatcgtagttgacatgaagcgattgtaaaactgtctccggttttctctggtttattaaaatgctaactataacattttcttttctttttttttttttgtgaatactttgaatgtctccctaacagttgtggtgttgctgttctgttcgatgcttattccaagttcatttttttgaatggttttgaagccattttgtaatgaattaaatgtccatgctgtacagtacctgtagcatgcctgccgttctggattaataaaagcaacttagtatgtgca